(19) United States
(12) Patent Application Publication
(10) Pub. No.: US 2010/0062995 A1
(43) Pub. Date: Mar. 11, 2010

(54) Transition state structure of human 5'methylthioadenosine phosphorylase

(76) Inventor: Vern L. Schramm, New Rochelle, NY (US)

Correspondence Address: AMSTER, ROTHSTEIN & EBENSTEIN LLP, 90 PARK AVENUE, NEW YORK, NY, 10016, US

(21) Appl. No.: 12/311,091

(22) PCT Filed: Sep. 18, 2007
(86) PCT No.: PCT/US07/20163

§ 371 Date: Sep. 29, 2009

Related U.S. Application Data
(60) Provisional application No. 60/847,312, filed on Sep. 26, 2006.

(51) Int. Cl. A61K 31/7076; C07H 19/20; C07H 11/04; C12N 5/00

(52) U.S. Cl. 514/48; 536/26.71; 536/117; 435/375; 514/45

(57) ABSTRACT

Provided are methods of designing a putative inhibitor of a human 5′-methylthioadenosine phosphorylase (MTAP). Also provided are methods of inhibiting a human MTAP.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. GM41916 awarded by The National Institutes of Health.

### BACKGROUND OF THE INVENTION

(1) Field of the Invention

The present invention generally relates to enzyme inhibitors. More specifically, the invention relates to methods of designing transition state inhibitors of 5′-methylthioadenosine phosphorylase.

(2) Description of the Related Art

### References Cited

Anisimov, V.; Paneth, P. ISOEFF98, J. Math. Chem. 1999, 26, 75-86.
Appleby, T. C.; Erion, M. D.; Ealick, S. E. Structure 1999, 7, 629-641.
Bagdassarian, C. K., Schramm, V. L., and Schwartz, S. D. J. Am. Chem. Soc 1996 118, 8825-8836.
Berti, P. J.; Tanaka, K. S. E. Adv. Phys. Org. Chem. 2002, 37, 239-314.
Birck, M. R.; Schramm, V. L. J. Am. Chem. Soc. 2004, 126, 2447-2453.
Flükiger, P.; Lüthi, H. P.; Portmann, S.; Weber, J. MOLEKEL 4.0, 2000 Swiss Center for Scientific Computing, Manno, Switzerland. Frisch, M. J.; et al. Gaussian 03, Revision B.04; Gaussian, Inc.: Pittsburgh, Pa., 2003.
Gawlita, E.; Lantz, M.; Paneth, P.; Bell, A. F.; Tonge, P. J.; Anderson, V. E., J. Am. Chem. Soc, 2000; 122, 11660-11669.
Harasawa, H.; Yamada, Y.; Kudoh, M.; Sugahara, K.; Soda, H.; Hirakata, Y.; Sasaki, H.; Ikeda, S.; Matsuo, T.; Tomonaga, M.; Nobori, T; Kamihira, S. Leukemia 2002, 16, 1799.
Kung, P. P.; Zehnder, L. R.; Meng, J. J.; Kupchinsky, S. W.; Skalitzky, D.; Johnson, M. C.; Maegley, K. A.; Ekker, A.; Kuhn, L. A.; Rose, P. W.; Bloom, L. A. Bioorg. Med. Chem. Lett. 2005, 48, 2829-2833.
Lee, J. E.; Singh, V.; Evans, G. B.; Tyler, P. C.; Furneaux, R. H.; Cornell, K. A.; Riscoe, M. K.; Schramm, V. L.; Howell, P. L. J. Biol. Chem. 2005, 280, 18274-18273.
Lewandowicz, A.; Schramm, V. L. Biochemistry 2004, 43, 1458-1468.
Parkin, D. W.; Mentch, F.; Banks, G. A., Horenstein, B. A.; Schramm, V. L. Biochemistry 1991, 30, 4586-4594.
Pu, J.; Ma, S.; Garcia-Viloca, M.; Gao, J.; Truhlar, D. G.; Kohen, A., J. Am. Chem. Soc. 2005, 127, 14879-14886.
Rose, I. A. Methods Enzymol. 1980, 64, 47-59.
Schramm, V. L.; Shi, W. Curr. Opin. Struct. Biol. 2001, 11, 657-665.
Shi, W; Basso, L. A.; Santos, D. S.; Tyler, P. C.; Furneaux, R. H.; Blanchard, J. S.; Almo, A. C.; Schramm, V. L. Biochemistry 2001, 40, 8204-8215.
Singh, V.; Shi, W.; Evans, G. B.; Tyler, P. C.; Furneaux, R. H.; Schramm, V. L. Biochemistry 2004, 43, 9-18.
Singh, V.; Lee, J. E.; Nunez, S.; Howell, L. P.; Schramm, V. L. Biochemistry 2005a, 44, 11647-11659.
Singh, V. Evans, G. B.; Len; D. H.; Painter, G. F.; Tyler, P. C.; Furneaux, R. H.; Lee, J. E.; Howell, P. L.; Schramm, V. L. J. Biol. Chem. 2005b, 280, 18265-18273.
Tabor, C. W.; Tabor, H. Methods Enzymol. 1983, 94, 294-297.
PCT Patent Publication WO ______, filed Jul. 26, 2006, entitled Transition State Structure of 5′-Methylthioadenosine/S-Adenosylhomocysteine Nucleosidases.
U.S. Pat. No. 7,098,334.

Kinetic isotope effects (KIE) are the method of choice for studying the transition states of enzymatic reactions and have been used to establish the properties of transition states of N-ribosyltransferases for purine and pyrimidine nucleosides (Singh et al., 2005a; Birck and Schramm, 2004; Lewandowicz and Schramm, 2004). In one KIE approach, heavy isotopes (³H, ¹⁴C and ¹⁵N) are substituted at positions expected to experience bond vibrational difference on conversion of reactants to the transition state. To determine the transition state structure of an enzymatic reaction (k_{cat}/Kₘ), KIEs are corrected for the commitment factors to obtain intrinsic KIEs, that is, KIEs on the bond-breaking step. The intrinsic KIEs originate from the vibrational difference between the free substrate in solution and at the transition state. KIEs provide a boundary condition for computational modeling of the enzymatic transition state. The transition state for an enzyme catalyzed reaction is approximated by correlating the calculated KIEs with the intrinsic KIEs.

Most N-ribosyl transferases have dissociative S_{N}1 transition states which are characterized by the formation of a ribosyl oxacarbenium ion with increased positive charge on the anomeric carbon and decreased negative charge on the ribosyl ring oxygen. Among the few exceptions is the transition state of thymidine phosphorylase, which has an S_{N}2 mechanism (Birck and Schramm, 2004). Another common feature of the N-ribosyl transferases is that dissociation of the N-glycosidic bond is accompanied by an increase in the pK_{α} of the leaving group. An active site general acid, for example Asp197 for E. coli 5′-methylthioadenosine nucleosidase (MTAN) (Lee et al., 2005) and Asp198 in bacterial purine nucleoside phosphorylase (PNP) (Shi et al., 2001), is often present to protonate N7 of the leaving group and stabilize the transition state. Transition state analyses of MTANs have shown that N7 is protonated at the transition state of E. coli MTAN but not at the transition state of S. pneumoniae MTAN. The higher activation barrier for the S. pneumoniae MTAN is reflected in a k_{cat} for S. pneumoniae MTAN of 0.25 s⁻¹, 16 fold less than that of E. coli MTAN (Lee et al., 2005; Singh et al. 2005b).

It would be desirable to have a transition state analysis of similar enzymes, in particular 5′-methylthioadenosine phosphorylase (MTAP), due to the importance of these enzymes in disease (see, e.g., Harasawa et al., 2002). Such transition state analysis would aid in the design of inhibitors for the enzymes. The present invention addresses that need.

### SUMMARY OF THE INVENTION

Accordingly, the inventor has determined the transition state structure of 5′-methylthioadenosine phosphorylase.

Thus, the present invention is directed to methods of designing a putative inhibitor of a human 5′-methylthioadenosine phosphorylase (MTAP). The methods comprise designing a chemically stable compound that resembles (a) the molecular electrostatic potential at the van der Walls surface computed from the wave function of the transition state of the MTAP and (b) the geometric atomic volume of the MTAP transition state. In these methods, the compound is the putative inhibitor.

The invention is also directed to methods of inhibiting a human MTAP. The methods comprise designing a MTAP inhibitor by the above method then contacting the MTAP with the inhibitor.

### BRIEF DESCRIPTION OF THE DRAWINGS

FIG. 1 shows a scheme for phosphorolysis of MTA by human MTAP and the proposed transition state of the reaction. Details of this transition state are presented in Table 2.

FIG. 2 is a graph of experimental results showing the forward commitment to catalysis for the MTAP-MTA complex. The complex of human MTAP and ¹⁴C-MTA was diluted with a large excess of unlabeled MTA and varying concentrations of sodium arsenate. The subsequent reaction partitions bound ¹⁴C-MTA to product (forward commitment) or permits release into free, unbound MTA. Zero commitment extrapolates through the origin as indicated by the arrow on the ordinate. The forward commitment was calculated by plotting the amount of labeled adenine formed following addition of chase solution, containing saturating amounts of sodium arsenate, divided by amount of labeled MTA on the active site before dilution with chase solution. The line is drawn from an ordinary least square fit of the data to the Michaelis-Menten equation. The intercept value is 0.21 and the forward commitment factor was calculated from the intercept using the expression: (intercept/1-intercept). The forward commitment to catalysis for human MTAP is 0.265±0.027.

FIG. 3 is cartoons showing analysis of the reaction coordinate for the arsenolysis of MTA by MTAP. Panel A shows the molecular electrostatic potential surfaces (MEPs) MTA reactant, transition state and products. The transition state is shown as a fully dissociated anionic adenine and a ribosyl-phosphate interaction is shown at 2.0 Å, assuming that arsenate and phosphate are equivalent in transition state interactions. MEPs were calculated at the HF/STO3G level (Gaussian 98/cube) for the geometry optimized at the B1LYP/6-31G(d,p) level of theory and visualized with Molekel 4.0 at a density of 0.05 electron/Å³. The stick models have the same geometry as the MEP surfaces. For purpose of visualization, the ribosyl 3-hydroxyl is shown without ionization to show the geometry of the hydroxyl group. The ribosyl-arsenate hydrolyzes following bond formation and the products are shown as the hydrolysis products of MTR and neutral adenine. Panel B shows the intrinsic kinetic isotope effects (KIEs) experimentally determined and a cartoon of the resulting transition state.

FIG. 4 is a stick figure of the human MTAP transition state with no constraints.

FIG. 5 is a stick figure of the transition state of human MTAP as a phosphate nucleophile (B1LYP/6-31G**).

### DETAILED DESCRIPTION OF THE INVENTION

The present invention is based on the determination of the transition state of the human 5′-methylthioadenosine phosphorylase (MTAP) (see Example). Based on this work and similar work with 5′-methylthioadenosine/S-adenosylhomocysteine nucleosidase (see PCT Patent Publication WO ______, filed Jul. 26, 2006), the inventor concludes that a compound designed to resemble the charge and geometry of the 5′-methylthioadenosine phosphorylase transition state is likely to be an inhibitor of that enzyme.

Thus, the invention is directed to methods of designing a putative inhibitor of a human 5′-methylthioadenosine phosphorylase (MTAP). The methods comprise designing a chemically stable compound that resembles (a) the molecular electrostatic potential at the van der Walls surface computed from the wave function of the transition state of the MTAP and (b) the geometric atomic volume of the MTAP transition state. In these methods, the compound is the putative inhibitor.

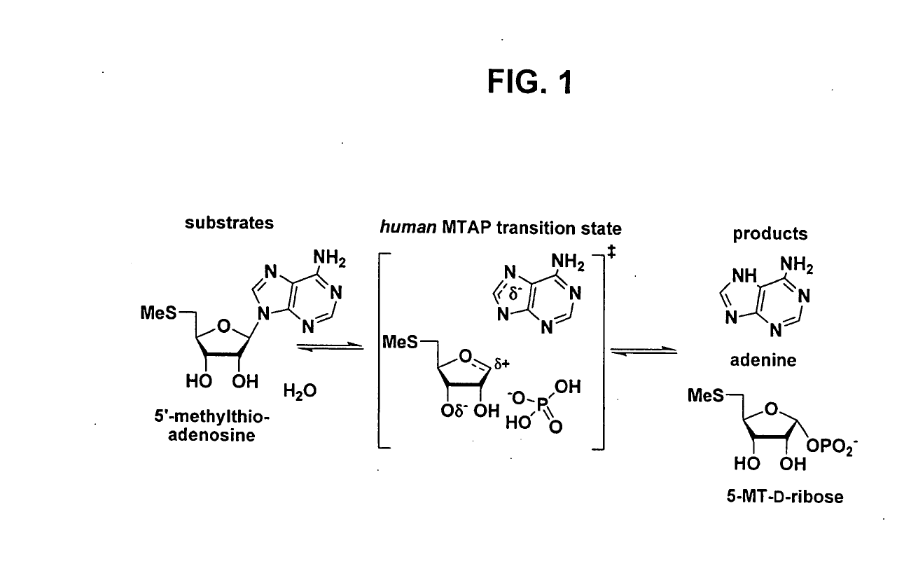

As used herein, MTAP is an enzyme that catalyzes the reversible phosphorolysis of the N-glycosidic bond of MTA to form 5′-methylthioribose-1-phosphate (MTR-1-P) and adenine (FIG. 1). Several inhibitors of MTAP have already been identified. Those inhibitors also inhibit 5′-methylthioadenosine/S-adenosylhomocysteine nucleosidase (MTAN). See, e.g., U.S. Pat. No. 7,098,334.

The determination of the molecular electrostatic potential at the van der Walls surface computed from the wave function of the transition state and the geometric atomic volume for any chemically stable compound is within the scope of the art. See, e.g., Example.

As used herein, a compound resembles the MTAP transition state molecular electrostatic potential at the van der Walls surface computed from the wave function of the transition state and the geometric atomic volume if that compound has an Sₑ and S_{g}>0.5, where Sₑ and S_{g} are determined as in Formulas (1) and (2) on page 8831 of Bagdassarian et al., 1996.

In some preferred embodiments, the compound comprises a purine moiety. In other preferred embodiments, the compound comprises a deazapurine moiety.

In additional preferred embodiments, the compound comprises a moiety resembling the molecular electrostatic potential surface of the ribosyl group at the transition state. In some of these embodiments, the compound comprises a moiety resembling methylthioribose at the transition state. In other of these embodiments, the compound comprises a moiety resembling S-homocysteinyl ribose at the transition state. Preferred examples of moieties resembling the molecular electrostatic potential surface of the ribosyl group at the transition state are substituted iminoribitols, substituted hydroxypyrrolidines, substituted pyridines or substituted imidazoles. In more preferred embodiments, the substituent is an aryl- or alkyl-substituted thiol group, most preferably a methylthiol group.

In other preferred embodiments of these methods, the compound comprises an atomic moiety inserted into the inhibitor providing a compound that mimics the C1′-N9 ribosyl bond distance of a 5′-methylthioadenosine or S-adenosylhomocysteine at the transition state. Preferably, the atomic moiety is a methylene, a substituted methylene, an ethyl, or a substituted ethyl bridge.

Preferably, the compounds designed using these methods exhibit a similarity value (Sₑ) to the transition state greater than to either substrate (see Bagdassarian et al., 1996). Sₑ can be determined by any known method, for example as described in Bagdassarian et al., 1996.

When compounds are designed by these methods, they can then be synthesized and tested for inhibitory activity to 5′-methylthioadenosine phosphorylase by known methods, e.g., as described in the Example below, and in U.S. Pat. No. 7,098,334.

The invention is also directed to methods of inhibiting an MTAP. The methods comprise identifying a compound that has inhibitory activity to the MTAP by the above-described methods, then contacting the MTAP with the compound.

In these methods, the compound and the MTAP (and MTAP substrates) can be in vitro, e.g., in a test tube, or in vivo, e.g., in a live prokaryotic or mammalian cell. Preferably, the MTAP is in a human cell, most preferably a cancer cell in a human.

Where a human with cancer is treated with the MTAP inhibitor, they are preferably also treated with an inhibitor of de novo adenosine monophosphate synthesis, for example L-alanosine, to assure killing of the cancer cell, as in Harasawa et al., 2002. Other non-limiting examples of a useful inhibitor of de novo adenosine monophosphate synthesis here are anti-folate compounds such as methotrexate.

Preferred embodiments of the invention are described in the following example. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the example.

### Example

### Transition State Structure of Human 5′-Methylthioadenosine Phosphorylase

### Example Summary

Kinetic isotope effects (KIEs) and computer modeling using density functional theory were used to approximate the transition state of human 5′-methylthioadenosine phosphorylase (MTAP). KIEs were measured on the arsenolysis of 5′-methylthioadenosine (MTA) catalyzed by MTAP and were corrected for the forward commitment to catalysis. Intrinsic KIEs were obtained for [1′-³H], [1′-¹⁴C], [2′-³H], [4′-³H], [5′-³H], [9-¹⁵N] and [Me-³H₃] MTAs. The primary intrinsic KIEs (1′-¹⁴C and 9-¹⁵N) suggest that MTAP has a dissociative S_{N}1 transition state with cationic center at the anomeric carbon and insignificant bond order to the leaving group. The 9-¹⁵N intrinsic KIE of 1.037 also establishes an anionic character to the adenine leaving group, whereas the α-primary KIE of 1.029 indicates significant nucleophilic participation at the transition state. Computational matching of the calculated EIEs to the intrinsic isotope effects places the oxygen nucleophile 2.0 Å from the anomeric carbon. The 4′-³H KIE is sensitive to the polarization of the 3′-OH group. Calculations suggest that a 4′-³H KIE of 1.045 is consistent with ionization of the 3′-OH group, indicating formation of a zwitterion at the transition state. The transition state has cationic character at the anomeric carbon and is anionic at the 3′-OH oxygen, with an anionic leaving group. The isotope effects predicted a 3′-endo conformation for the ribosyl zwitterion corresponding to a H1′-C1′-C2′-H2′ torsional angle of 33°. The [Me ³H₃] and [5′-³H] KIEs arise predominantly from the negative hyperconjugation of the lone pairs of sulfur with the σ* (C—H) antibonding orbitals. Human MTAP is characterized by a late, S_{N}1 transition state with significant participation of the phosphate nucleophile.

### INTRODUCTION

In this study the transition state of human MTAP is explored by KIE measurements and computational modeling using density functional methods implemented in Gaussian 03 (Frisch et al., 2003). MTAP is a purine salvage enzyme found in mammals. It catalyzes the reversible phosphorolysis of the N-glycosidic bond of MTA to form 5′-methylthioribose-1-phosphate (MTR-1-P) and adenine (FIG. 1). Disruption of MTAP has been shown to affect purine salvage, methionine and polyamine pathways and it has been proposed to be an anti-cancer drug target (Tabor, 1983; Singh et al., 2004; Harasawa et al., 2002). This study suggests that human MTAP has a dissociative S_{N}1 transition state, and in this respect is similar to other members of the N-ribosyl transferase family. However, the ribosyl group at the transition state of MTAP exists as a stabilized zwitterion, cationic at the anomeric carbon and anionic at the oxygen of the 3′-hydroxyl. The positive charge on the anomeric carbon at the transition state is stabilized by low bond order to the attacking anionic phosphate or arsenate nucleophile. Further, the N7 of adenine is not protonated at the transition state. The catalytic acceleration therefore comes from the stabilization of the methylthioribosyl zwitterion by a phosphate anion and distortion or ionization of the 3′-OH group.

Material and Methods Expression and Purification of human MTAP. Details of the DNA manipulation, protein expression and purification procedure for human MTAP have been described previously (Singh et al., 2004). Briefly, the enzyme was overexpressed in E. coli using pQE32 expression vector. The overexpressed MTAP, His₆ tagged at the N-terminus, was purified using Ni-NTA resin column using a 30-300 mM imidazole gradient. The purified protein was concentrated, dialyzed against 100 mM Tris, pH 7.9, 50 mM NaCl and 2 mM DTT, and stored at −80° C.

Enzymes and reagents for MTA synthesis. The reagents and the enzymes used in the synthesis of MTAs from glucose have been described previously elsewhere (Singh et al., 2005a).

Synthesis of radiolabeled MTAs. Isotopically labeled [1′-³H]MTA, [1′-¹⁴C]MTA, [2′-³H]MTA, [3′-³H]MTA, [4′-³H]MTA, [5′-³H]MTA, [methyl-³H₃]MTA and [8-¹⁴C]MTA were synthesized from the corresponding ATP molecules in two steps using the procedure described elsewhere (Singh et al., 2005a).

Measurement of kinetic isotope effects. The KIEs were measured by mixing ³H and ¹⁴C labeled substrates with ³H:¹⁴C in 4:1 ratio. The MTAP assays for measuring KIEs were performed in triplicates of 1 mL reactions (100 mM Tris-HCl pH 7.5, 50 mM KCl, 250 μM MTA (including label), 15 mM sodium arsenate and 1.0-5.0 nM human MTAP) containing >10⁵ cpm of ¹⁴C. After 20-30% completion of the reaction, 750 μL of the reaction was resolved on charcoal-Sepharose (acid-washed powdered charcoal and Sepharose in 1:4 ratio made into a slurry in 1 mM 5-methylthioribose (MTR) and settled in Pasteur pipettes). The remainder of the reaction mixture was allowed to react to completion and then applied to the column. Columns were washed with 2 volumes of 1 mM MTR and radioactive methylthioribose was eluted with 6 volumes of 15 mM MTR containing 50% ethanol. Each 1.0 mL of eluate was mixed with 9.0 mL scintillation fluid and counted for at least 3 cycles at 10 minutes per cycle. The ³H to ¹⁴C ratio was determined for partial and complete reactions and the KIEs were corrected to 0% hydrolysis by the equation:

$$KIE = \frac{ln ( 1 - f )}{ln [ ( 1 - f \frac{{R}_{f}}{{R}_{o}} ) ]}$$

where f is the fraction of reaction progress and R_{f} and Rₒ are ratios of heavy to light isotope at partial and total completion of reaction, respectively.

Forward commitment factor. The isotope partition method (Rose, 1980) was used to measure the forward commitment to catalysis. The 20 μL “Pulse solution” containing 20 μM human MTAP in 100 mM Tris pH 7.5, 50 mM KCl, 1 mM DTT and 200 μM of [8-¹⁴C]MTA containing 10⁵ cpm was incubated for 10 seconds and then diluted with 180 μL of “Chase solution” containing a large excess of unlabeled MTA (2.6 mM) in 1 Tris pH 7.5, 50 mM KCl, 1 mM DTT and various concentrations of sodium arsenate (0.1, 0.3, 0.5, 1.0, 2.0, 4.0, 7.0, 20 and 40 mM). The samples were incubated for 20 sec to allow a few turnovers and then quenched with 1N HCl. Product formation was measured by reverse phase HPLC using C-18 Deltapak column by 25% methanol and 50% of 100 mM ammonium acetate pH 5.0 and scintillation counting. The forward commitment to catalysis is calculated from the fraction of bound MTA converted to product following dilution in excess MTA. This procedure uses [8-¹⁴C]MTA as a stoichiometric label for the catalytic site and the commitment factors are independent of any KIE, although none would be expected from [8-¹⁴C]MTA.

Computational modeling of the transition state. The in vacuo determination of the MTAP transition state used hybrid density functional methods implemented in Gaussian 03 (Frisch et al., 2003). The substrate and the transition state were modeled using the one-parameter Becke (B1) exchange functional, the LYP correlation functional and the 6-31G(d,p) basis set (Shi et al, 2001). The same level of theory and basis set were also used for the computation of bond frequencies. During the calculations the 5′-methylthio group was constrained by freezing the O4′-C4′-C5′-S and C4′-C5-S—C^{Me} torsion angles. The properties of the leaving group at the transition state were modeled separately.

Equilibrium isotope effects (EIEs) were calculated from the computed frequencies of the substrate and the transition state intermediate using ISOEFF 98 software (Anisimov and Paneth, 1999). All 3N-6 vibrational modes were used to calculate the isotope effects, but only those that exhibit shifts due to the isotopic substitution contribute to isotope effects. The isotope effects were calculated at the temperature of 298 K.

The applied geometric constraints were optimized iteratively to generate a transition state model for which the primary and the β-secondary EIEs closely match the intrinsic KIEs. The secondary intrinsic KIEs at other positions were then explored systematically to obtain group properties that matched the experimental intrinsic KIEs. Constrained molecules impose energetically unfavorable positions relative to vacuum conditions for transition state searches. These reflect the forces imposed by the enzymatic environment. Clearly, this approach yields an approximation of the transition state. Frequencies for unconstrained and constrained transition states are provided in the Supplementary Materials.

The contribution of solvent to the state of the free reactant has been tested in a closely related reaction, hydrolysis of the N-ribosidic bond of 5′-methylthioadenosine by S. pneumoniae methylthioadenosine nucleosidase. The effects of changing the implicit solvent (by changing the empirical parameter of the dielectric constant) on isotope effects were examined by the Self Consistent Reaction Field (SCRF) method using the polarization continuum model for 5′-methylthioadenosine at the transition state. Changing the dielectric constant from 4.9 (for chloroform) to 78.8 (for water) has no effect on the calculated EIEs (data not shown). Explicit solvent water interactions are also unlikely to influence KIEs by hydroxyl group interactions. Gawlita et al. have shown that desolvation of primary and secondary hydroxyls does not cause isotope effects on the neighboring CH bonds (Gawlita et al., 2000). Based on these analyses, no corrections for solvent interactions have been applied.

The natural bond orbital (NBO) calculations were performed on optimized structures by including the pop=(nbo, full) keyword in the route section of input files and the molecular electrostatic potential (MEP) surfaces were visualized using Molekel 4.0 (Flükiger et al., 2000).

### Results and Discussion

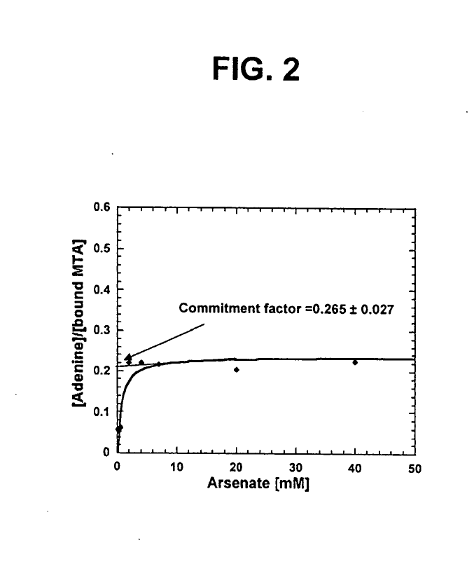

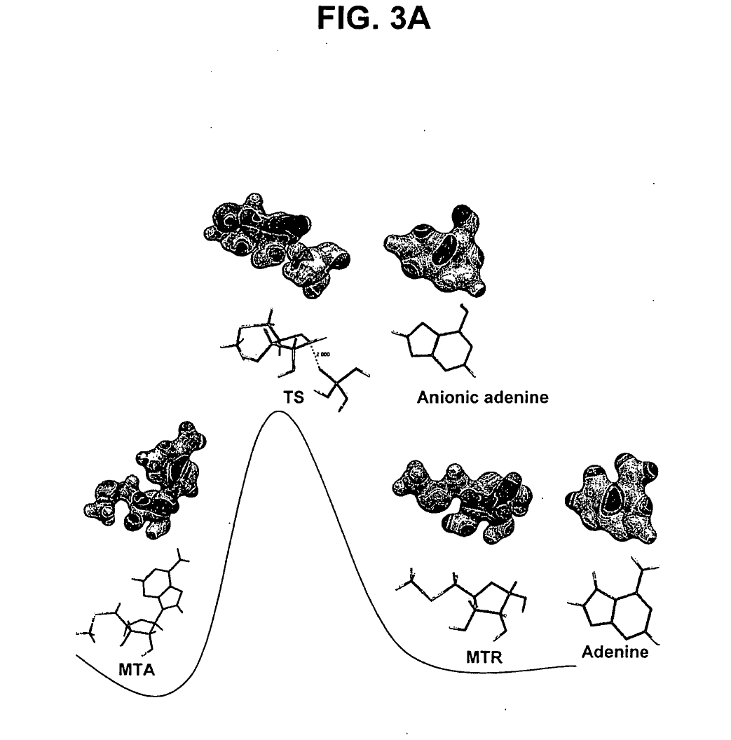

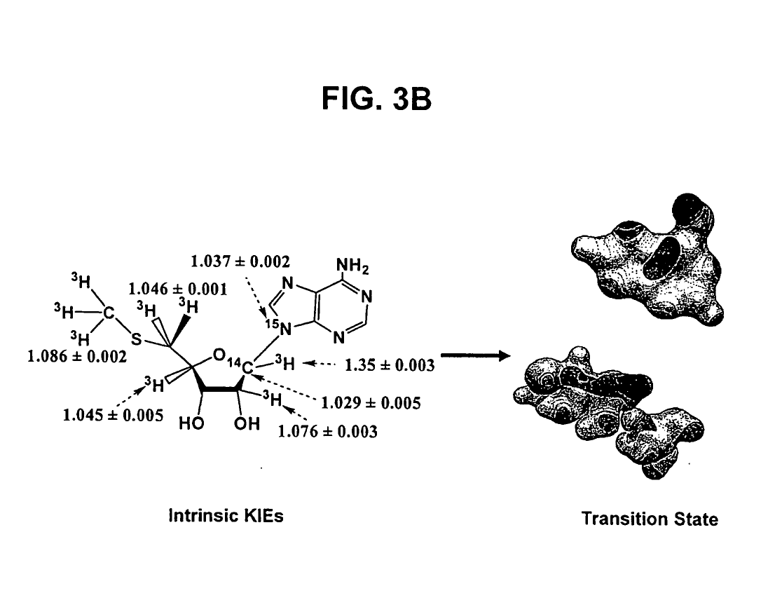

Experimental kinetic isotope effects (KIEs) and commitment factors. Human MTAP catalyzes the reversible phosphorolysis of the N-glycosidic bond of MTA to 5-methylthioribose 1-phosphate and adenine. To avoid kinetic complexity associated with the transition state analyses for reversible reactions, the KIEs for the human MTAP were measured on the physiologically irreversible reaction of arsenolysis. The products of arsenolysis are adenine and 5-methylthioribose 1-arsenate. Methylthioribose 1-arsenate is unstable and rapidly decomposes to form methylthioribose and arsenate. Although the possibility of an on-enzyme equilibrium cannot be ruled out, an intrinsic KIE of 1.036 for ¹⁵N9 (equal to the theoretical maximum of 1.036 for complete dissociation of N-glycosidic bond (data not shown) and a large 1′-³H (the largest reported for any N-ribosyl transferases) preclude the existence of such equilibrium. Therefore the KIEs reported in Table 1 are intrinsic KIEs. The KIEs were measured for MTAs labeled at [1′-³H], [1′-¹⁴C], [2′-³H], [4′-³H], [5′-³H₂], [9-¹⁵N] and [Me-³H₃] using competitive conditions. 5′-¹⁴C MTA was used as a remote control for measuring tritium isotope effects and 5′-³H MTA was used for the same purpose for measuring 1′-¹⁴C and 9-¹⁵N/5′-¹⁴C KIE. The 1′-¹⁴C and 9-¹⁵N/5′-¹⁴C KIEs were corrected for the 5′-³H KIE. The measured KIEs were also corrected for the external forward commitment of 0.21±0.027 (FIG. 2) using Northorp's equation modified for irreversible reactions:

$${}^{T} ( V / K ) = \frac{{}^{T} k + {C}_{f}}{1 + {C}_{f}} ;$$

Where ^{T}(V/K) is an observed tritium isotope effect, C_{f} is the forward commitment to catalysis. The intrinsic KIEs were obtained by correcting the observed KIEs (column 3, Table 1) for the forward commitment.

TABLE 1
KIEs measured at pH 7.5 for arsenolysis
of MTA by human MTAP.^{a}

| | | | | Calcu- |
|---|---|---|---|---|
| | | | Intrinsic | lated |
| Substrate | Type of KIE | KIE^{b} | KIEs | KIEs |
| [1′-³H] | α-secondary | 1.290 ± 0.003 | 1.350 ± 0.003 | 1.200 |
| [1′-¹⁴C] | primary | 1.025 ± 0.005^{b} | 1.030 ± 0.005 | 1.030 |
| [2′-³H] | β-secondary | 1.063 ± 0.003 | 1.076 ± 0.003 | 1.076 |
| [9-¹⁵N/5′-¹⁴C] | primary | 1.031 ± 0.002^{c} | 1.037 ± 0.002 | 1.036 |
| [4′-³H] | γ-secondary | 1.037 ± 0.005 | 1.045 ± 0.005 | 0.997 |
| [5′-³H] | δ-secondary | 1.038 ± 0.001 | 1.046 ± 0.001 | 1.050 |
| [Me-³H] | remote | 1.073 ± 0.002 | 1.086 ± 0.002 | 1.028 |

^{a}Experimental KIEs are corrected to 0% substrate depletion.
^{b}The 1′-¹⁴C KIE was corrected for 5′-³H KIE according to expression KIE = KIE_{observed} × 5′-³H KIE
^{c}The 9-¹⁵N KIE was corrected for 5′-³H KIE according to expression KIE = KIE_{observed} × 5′-³H KIE.

Computation of the transition state. The transition state of human MTAP was modeled using the B1LYP functional and 6-31G (d,p) basis sets. The modeling was performed using a 5-methylthioribosyl oxacarbenium ion, anionic adenine as a leaving group and a neutral phosphate nucleophile. The calculated KIE values were tried both with arsenate and phosphate and the differences were within the standard error limits of the experimental KIEs, therefore we elected to use phosphate, the physiological nucleophile. The initial transition state model generated by an in vacuo calculation without imposing any external constraints predicted a S_{N}2-like transition state, which is characterized by a large 1′-¹⁴C KIE with significant bond order to the leaving group and the phosphate nucleophile. It had a single imaginary frequency of 295 i cm⁻¹ (see Supplementary Information). The experimental intrinsic KIE of 1.029 for 1′-¹⁴C MTA suggests that the anomeric carbon has a small but significant bond order to either to the leaving group or to an attacking phosphate nucleophile or to both at the transition state. The 1′-¹⁴C KIE together with the 9-¹⁵N intrinsic KIE of 1.036, which is consistent with the complete dissociation of the N-glycosidic bond at the transition state, suggests that the 1′-¹⁴C KIE of 1.029 arises entirely from increased bonding to the oxygen of phosphate nucleophile. Therefore, in the subsequent modeling of the MTAP transition state, additional distance constraints were applied to the leaving group and to the phosphate nucleophile. The 5-methylthio group, being away from the reaction center, was constrained by fixing the O4′-C4′-C5′-S and C4′-C5′-C^{me} torsional angles. The calculations were performed by increasing the C1′-N9 distance in increments to 4.0 Å, where the bond order is negligible. The leaving group was not included in subsequent calculations. Application of bond constraints to the transition state resulted in appearance of two imaginary frequencies (see Supplementary Information below). The in vacuo transition state of an unconstrained reaction is the highest point on the potential energy surface (PES) and is characterized by a single imaginary frequency. The enzymatic PES is expected to differ from that in vacuo. The enzymatic transition state model is generated by correlating the theoretical KIEs to intrinsic KIEs. This coincidence locates the transition state for the enzymatic PES. This structure is no longer a transition state or a maxima on the in vacuo PES and hence has more than one imaginary frequency. Frequencies are obtained from the second derivative matrix of potential energy with respect with to Cartesian coordinates. For the dissociative S_{N}1 transition state with no significant bond order to the N-glycosidic bond, the relatively small imaginary frequencies have little effect on the primary (1′-¹⁴C and 9-¹⁵N) KIEs. Therefore, the calculated KIEs were similar to the EIEs. Since the intrinsic KIEs were closely related to a fully dissociated, stabilized ribooxacarbenium ion, the subsequent TS models were optimized as a TS intermediate. All 3N-6 normal modes of the transition state model and the substrate were used to calculate equilibrium isotope effect (EIEs) using ISOEFF98 (Anisimov and Paneth, 1999). The applied external constraints were iteratively optimized until the calculated EIEs correlated with the intrinsic KIEs. At the transition state the oxygen of the phosphate nucleophile is 2.0 Å from the anomeric carbon. The leaving group was modeled separately and is discussed below with 9-¹⁵N KIE along with the properties of the transition state.

Intrinsic 9-¹⁵N, 1′-¹⁴C and 1′-³H KIEs. The 9-¹⁵N intrinsic KIE of 1.037 measured for human MTAP is close to the theoretical maximum ¹⁵N isotope effect of 1.040 and also within experimental error for the 9-¹⁵N isotope effect of 1.036 calculated for the complete dissociation of N-glycosidic bond (Data not shown). Calculations on the adenine leaving group to study the effect of protonation at nitrogens N1, N3, N7 or N9 on the 9-¹⁵N isotope effect shows that the protonation of N7 decreases the 9-¹⁵N isotope effect from 1.036 to 1.025 (Data not shown). Therefore an intrinsic KIE of 1.037 for 9-¹⁵N MTA suggests that the dissociation of the N-glycosidic bond is complete and the N7 is not protonated at the transition state of human MTAP. The activation of the leaving group in the form of N7 protonation is a recurrent feature in the transition states of N-ribosyltransferases. Among the few exceptions are the transition states of S. pneumoniae MTAN and a mutant AMP nucleosidase (Parkin et al., 1991). Therefore, protonation of N7 is not required for cleavage of the N-glycosidic bond and the catalytic acceleration originates from formation of a methylthioribose cation at the transition state.

Crystallographic evidence also suggests that leaving group activation in the form of protonation of N7 is modest in MTAP. Crystal structures of human MTAP with MTA (its substrate, not protonated at N7) and MT-ImmA (a transition state analog with protonated N7) shows equivalent O^{Asp220}—N7 distances within the crystallographic errors in these two structures (It is 3.0 Å in the MTA structure and 2.9 Å in the structure of MT-ImmA with human MTAP). However, the ionization of Asp220 is not revealed by crystallography and could make an important difference in binding energy of MTA and MT-ImmA.

The α-primary 1′-¹⁴C intrinsic KIE is the most useful probe for determining the mechanism of nucleophilic substitution reaction (S_{N}1 vs S_{N}2) of N-ribosyltransferases (Berti and Tanaka, 2002). A 1′-¹⁴C KIE of 1.00 to 1.030 indicates dissociative S_{N}1 transition states, 1.030 to 1.080 indicates significant associative interactions in S_{N}1 transition states and a KIE of greater than 1.080 indicates the properties of an S_{N}2 transition with a neutral reaction center (anomeric carbon in the case of ribosyltransferases). An intrinsic KIE of 1.029 for human MTAP indicates an S_{N}1 transition state with significant bond order to the phosphate nucleophile. The transition state consistent with the kinetic isotope effects predicted a C1′-O^{phosphate} bond distance of 2.0 Å. The small primary 1′-¹⁴C KIE indicates a change in hybridization at the anomeric carbon as it changes from sp^{2.83} hybridized in the substrate to sp^{2.40} at the transition state. These changes cause increased cationic character at the transition state (positive charge on O4′ and C1′ increase by +0.20 and +0.25 respectively) relative to the reactant state. This sharing of charge is characteristic of ribooxacarbenium ions (Berti and Tanaka, 2002). The change in hybridization also creates a partially empty 2p_{z} orbital on C1′ that hyperconjugates with the σ (C2′-H2′) electrons and lone pair of O4′ and stabilizes the transition state by partially neutralizing the positive charge on C1′.

The large 1′-³H intrinsic KIE of 1.35 is consistent with the dissociative S_{N}1 transition state as indicated by the 1′-¹⁴C and 9-¹⁵N KIEs. The large 1′-³H KIE arises mainly from a substantial decrease in bending frequencies for the out-of-plane bending modes due to increase steric freedom of C1′-H1′ following dissociation of the C1′-N9 bond. The 1′-³H KIE is also influenced by van der Waal interactions with active site residues and by the orientation of base in the reactant MTA (Data not shown). Polarization of the 2′-hydroxyl and rotation of the H1′-C1′-C2′-H2′ and H2′-C2′-O—H torsion angles also have a small influence on the 1′-³H KIE (Flükiger et al., 2000). Although all these factors are difficult to model together, the large 1′-³H KIE is consistent with the dissociative transition state. Quantum mechanical tunneling is known to influence ³H-secondary kinetic isotope effects in hydride transfer reactions (Pu et al., 2005) but are unlikely to be coupled to the reaction coordinate motion of C—N bond cleavage. Possible contributions from H-tunneling were therefore ignored.

The 4′-³H KIE and evidence for a 5′-methylthioribosyl zwitterion. For dissociative S_{N}1 transition states, theoretical calculations predict a large inverse isotope for 4′-³H MTA (Data not shown). This arises from the interaction of the σ (C4′-H4′) bond with the relatively electron deficient O4′ and cationic center at the anomeric carbon (Singh et al., 2005a). Previously, normal intrinsic 4′-³H KIEs of 1.015 and 1.010 were measured for S. pneumoniae and E. coli MTANs (Singh et al., 2005a). In those cases the polarization of the 3′-OH increases the electron density on the ring oxygen (O4′) due to unequal charge sharing, causing the hyperconjugation of the lone pair of O4′ to σ*(C4′-H4′) antibonding orbital to increase to give a normal 4′-³H KIE isotope effect. Glu174 was recognized as the residue responsible for the polarization of 3′-OH in MTANs (Singh et al., 2005a). Human MTAP also has a larger normal intrinsic 4′-³H KIE of 1.045 suggesting a similar mechanism. The crystal structure of human MTAP with MT-ImmA (a transition state analogue) shows that one of the phosphate oxygens is strongly hydrogen bonded to the 3′-OH (O^{hydroxyl}—O^{phosphate} distance is 2.6 Å). Ionization of the 3′-hydroxyl creates an anionic center at this oxygen. The transition state therefore is zwitterionic with a partial positive charge on the anomeric carbon and a negative charge on the oxygen of the 3′-hydroxyl.

The transition state for human MTAP was solved without ionizing the ribosyl 3-hydroxyl group. Ionization of the 3-hydroxyl group forms a reactive 3-oxyanion, which extracts a proton from the ribosyl 2-hydroxyl group in the in vacuo calculations and causes isotope effects unrelated to the enzymatic reaction coordinate. Experimental intrinsic KIEs, studies with substrate analogues, mutational and crystallographic studies do not support ionization or strong polarization of the 2-hydroxyl at the transition state. The effect of 3-hydroxyl polarization on the isotope effect pattern is discussed in the text and is expected to influence 2-³H, 3-³H and 4-³H IEs with largest IE expected at the 3-³H position. The effect of polarization on 2-³H and 4-³H IEs is discussed in the paper.

The 4′-³H intrinsic KIE of 1.045 for human MTAP is also influenced by the phosphate nucleophile at the transition state. Participation of phosphate partially neutralizes the positive charge on the anomeric carbon and increases the occupancy of partially empty p-orbital on the anomeric carbon due to increased bonding character between the anomeric carbon and the oxygen of a phosphate nucleophile. The occupancy of the 2pz-orbital increases from 0.65 for S. pneumoniae MTAN to 0.85 in human MTAP whereas the positive charge on the anomeric carbon decreased from 0.58 in S. pneumoniae MTAN to 0.55 in human MTAP. These changes increase the intrinsic 4′-³H KIE to 1.045 in human MTAP from 1.015 and 1.010 as measured for S. pneumoniae and E. coli MTAN, respectively (Singh et al., 2005a). The increased occupancy of the p-orbital as well as the partial neutralization of the positive charge on the anomeric carbon causes the nₚ (O4′) top-orbital (C1′) hyperconjugation to decrease and there is a corresponding increase in the nₚ (O4′) to σ*(C4′-H4′) bond.

The phosphate nucleophile is also hydrogen-bonded to both the 2′-hydroxyl and the 3″-hydroxyl of MTA/MT-ImmA in the active site of human MTAP (Singh et al., 2004). In the crystal structure of MTA with sulfate (an analogue of phosphate) the 0-0 bond distance between the oxygens of sulfate and the 2′-hydroxyl and the 3′-hydroxyl are 3.0 Å and 2.4 Å, respectively. These distances change to 2.8 Å and 2.6 Å, respectively in the crystal structure of human MTAP with MT-ImmA (a transition state analogue). As the reaction approaches the transition state the O—O bond distance between oxygen of 3′-hydroxyl and the nucleophile appears to increase and with a decrease in the O—O bond distance between 2′-hydroxyl and the phosphate nucleophile. The KIE analysis also supports the movement of a nucleophile towards the anomeric carbon and away from the 3′-hydroxyl. Transition state analysis suggests that ionization of the 3′-hydroxyl results from motion relative to the basic phosphate molecule. Formation of an anion at the 3′-hydroxyl stabilizes a water molecule observed in the crystal structure of transition state analogue with human MTAP. This interaction is absent in the crystal structure of human MTAP with the MTA substrate (Appleby et al., 1999).

Kinetic analysis with substrate analogues provide additional evidence concerning the importance of the 3′-hydroxyl in catalysis (Kung et al., 2005). The k_{cat}/Kₘ of human MTAP for MTA is 3.2×10⁶ M⁻¹ s⁻¹. It decreases to 9×10⁴ M⁻¹ for 2′-deoxy MTA and to 10³ M⁻¹ s⁻¹ for 3′-deoxy MTA. This change is predominantly a k_{cat} effect which decreases from 4.6 for MTA to 0.28 s⁻¹ and 0.004 s⁻¹ for 2′- and 3′-deoxy MTA, respectively, whereas the Kₘ only increased ˜2 fold. The ˜1000 fold decrease in k_{cat} for 3′-deoxy MTA relative to MTA suggests that the oxygen of the 3′-hydroxyl is important in stabilizing the transition state. The formation of an oxyanion from ionization of the 3′-hydroxyl further stabilizes the positive charge at the anomeric carbon.

The 2′-³H KIE and ribosyl puckering. The positive hyperconjugation of σ (C2′-H2′) bonding electrons to a partially empty 2p_{z} orbital on the anomeric carbon at the transition state is the predominant factor that influences the magnitude of the 2′-³H KIE (data not shown). The contribution of this hyperconjugation to the total 2′-³H intrinsic KIE is dependent on the ribose puckering at the transition state. Its magnitude depends on the extent of overlap between the C2′-H2′ sigma bond and the 2p_{z} orbital. The magnitude of this effect varies as cos² θ function of this overlap (data not shown). If the entire 2′-³H KIE originates from the hyperconjugative interaction between σ (C2′-H2′) and the p-orbital, an intrinsic 2′-³H KIE of 1.076 for human MTAP corresponds to the H2′-C2′-C1′-H1′ torsion angle of 33° and a small 3-endo pucker corresponding to the O4′-C1′-C2′-C3′ torsion angle of −13°. However, the 2′-³H KIE is also influenced by polarization of the 2′-OH and 3′-OH and rotation of H2′-C2′-O—H torsional bond, whereas only positive hyperconjugation is influenced by puckering of the ribose (data not shown). To determine the fraction of the 2′-³H intrinsic KIE that comes exclusively from a (C2′-H2′) to 2p_{z} transfer, a calculation was performed by constraining the ribose sugar O4′-C1′-C2′-C3′ torsion angle to a value obtained from the crystal structure of MT-ImmA (a transition state analogue inhibitor of human MTAP (Singh et al., 2004) with human MTAP, and leaving the H2′-C2′-C1′-H 1′ torsional angle unconstrained. The H2′-C2′-C1′-H1 torsional angle of 29.6° was obtained from the calculation and this torsional angle corresponds to a 2′-³H IE of 1.063, implying that 1.063 of 1.078 comes from positive hyperconjugation of σ (C2′-H2′) electrons to partially empty 2p_{z} orbital and the rest comes from the effects described above.

Remote KIEs. Large normal intrinsic KIEs of 1.086 and 1.046 were measured for [Me-³H₃] MTA and [5′-³H₂] MTA, respectively. These isotope effects ([Me-³H₃] and [5′-³H₂]) arise from the net increase in the negative hyperconjugation, in the bound state, between the lone pairs (nₚ) of sulfur and the σ* (C^{Me}H) antibonding orbitals (data not shown). In the unbound substrate, the methylthio group is free to rotate, canceling or reducing this hyperconjugation.

Transition state space. How well has this computational approach covered the possible geometric conformations of the transition state? The magnitude of primary (1′-¹⁴C and 9-¹⁵N) KIEs is only sensitive to the degree of dissociation of the C—N bond, the bond order to the nucleophile and reaction coordinate motion at the transition state. Therefore the geometry of the reaction coordinate (namely C1′-N9 and C1′-O^{phosphate} distance) is a unique fit to the intrinsic KIE values. Ribose pucker is interpreted from the magnitude of the 2′-³H KIE, which is proportional to the extent of hyperconjugation from σ (C2′-H2′) to the anomeric carbon. This isotope provides a unique conformation for the ribose pucker. The charge on the leaving group adenine is uniquely predicted by the magnitude of the 9-¹⁵N KIE. Therefore, the geometry of the reaction coordinate, ribose pucker and the ionization of the leaving group adenine are uniquely described by the intrinsic KIE. The 5′-methio group can adopt multiple conformations to give the same KIE values. Therefore computation alone is inadequate. Structural data from the crystal structure of human MTAP with the transition state analogue, MT-ImmA, was used to position the 5′-methio group and therefore provide the origin of isotope effects for this specific geometry.

### CONCLUSIONS

Human MTAP has a late dissociative S_{N}1 transition state, in which dissociation to the leaving group is complete and there is a significant bonding to the phosphate nucleophile. The formation of a ribosyl oxacarbenium ion is accompanied by the polarization or ionization of the 3′-OH by a phosphate resulting in the formation of a 5-methylthioribosyl zwitterion at the transition state. The leaving group adenine is anionic with no bond order to the ribosyl zwitterion. The transition state of human MTAP therefore exists as a 5-methylthioribosyl zwitterion where the positive charge on the anomeric carbon is stabilized by an anionic 3-oxygen as well as by a phosphate nucleophile providing stabilization of the transition state. This is the first report to suggest the existence of a zwitterion-anion pair at an enzymatic transition state with the participation of a phosphate nucleophile, although the hydrolytic reaction of MTA catalyzed by S. pneumoniae MTAN has a similar ribosyl group at the transition state.

Many purine N-ribosyl transferases form transition states with neutral leaving groups and a cationic ribosyl group, thus generating a unit charge difference between leaving group and the ribosyl group. Human MTAP also has a unit charge difference but generates it with a net neutral (zwitterionic) ribosyl group and an anionic purine leaving group. The cationic anomeric carbon is sandwiched between two structures with anionic character to facilitate the migration of the electrophilic center commonly seen in N-ribosyl transferases (Schramm and Shi, 2001).

TABLE 2
Geometric and electronic changes in representative models of the substrate
and the transition state calculated using B1LYP/6-31G** (human_MTAP).

| | | | Bond Order | Hyperconjugation (kcals/mol)^{b} | | | | | Orbital Changes^{c} | | | |
|---|---|---|---|---|---|---|---|---|---|---|---|---|
| Bond | Bond Length | | Change^{a} | Substrate | | TS | | | GS | Carbon | TS | Carbon |
| Type | GS | TS | Δ(σ − σ*) | σ→ | →σ* | σ→ | →σ* | ΔΣ(TS − GS) | hybrid | cont. (%) | hybrid | cont. (%) |
| C1′-H1′ | 1.0936 | 1.0848 | −0.01584 | 10.95 | 8.10 | 7.12 | 6.97 | −1.28 | sp^{2.81} | 63.04 | sp^{1.81} | 64.22 |
| C2′-H2′ | 1.0973 | 1.0980 | +0.01244 | 5.37 | 11.23 | 12.65^{d} | 7.93 | −0.11 | sp^{2.72} | 62.09 | sp^{2.86} | 63.72 |
| C4′-H4′ | 1.0964 | 1.0916 | −0.00471 | 7.02 | 11.50^{e} | 8.44 | 5.59 | 3.60 | sp^{2.91} | 62.45 | sp^{2.83} | 63.98 |
| C1′-N9 | 1.4582 | NA | NA | 8.84 | 28.64 | NA | NA | NA | sp^{3.27} | 35.70 | NA | NA |
| C5′-H5′ (R) | 1.0910 | 1.0933 | −0.00232 | 5.39 | 5.66 | 6.44 | 4.88^{g} | 4.39 | sp^{3.04} | 63.86 | sp^{3.10} | 64.04 |
| C5′-H5′ (S) | 1.0914 | 1.0912 | −0.00337 | 5.61 | 4.21^{h} | 4.01 | 4.52 | −0.30 | sp^{3.02} | 63.42 | sp^{2.80} | 64.35 |
| C^{S}-H(A) | 1.0920 | 1.0906 | −0.00208 | 3.39 | 0.89 | 0.67 | 4.63ⁱ | 1.18 | sp^{3.02} | 62.71 | sp^{2.77} | 62.59 |
| C^{S}-H(B) | 1.0893 | 1.0907 | −0.00205 | <0.50 | 4.31 | 2.87 | 1.04ⁱ | 0.01 | sp^{2.78} | 62.71 | sp^{2.92} | 63.78 |
| C^{S}-H(C) | 1.0872 | 1.0906 | −0.00360 | 0.50 | 5.27^{j} | <0.50 | 3.85ⁱ | −1.81 | sp^{2.74} | 62.22 | sp^{2.76} | 63.25 |
| C3′-H3′ | 1.0908 | 1.0941 | −0.00103 | 6.62 | 9.42 | 5.60 | 8.02^{k} | 1.29 | sp^{2.67} | 63.49 | sp^{2.75} | 64.17 |

^{a}Calculated by subtracting the number of electrons occupying the σ* orbital from the number occupying the σ orbital and listed as change between Substrate and Transition state (TS) (Substrate − TS).
^{b}Sum of second order perturbation contributions calculated by NBO analysis.
Cutoff = 0.5 Kcal/mol.
^{c}Hybridization of the carbon atom and contribution of the carbon atom to the bond in percent.
GS = ground state of substrate,
TS = transition state.
Lp1 is the sp-type lone pair; and Lp2 is p-type lone pair: ^{d}Lp2(C1′); ^{f}Lp(O3′); ^{g}Lp2(S); ⁱLp2(S); ^{k}Lp2(O3′) are better acceptors in the transition state; While ^{e}Lp2(O4′); ^{h}Lp2(S); ^{j}Lp2(S) are better acceptors in the substrate.

### Supplementary Materials

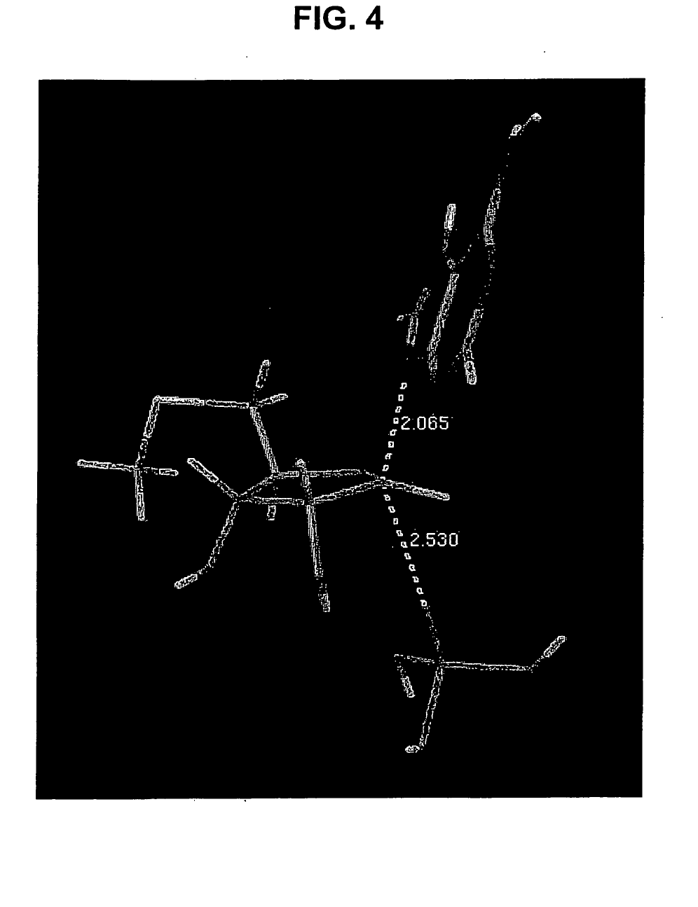

Frequencies for the transition state with no constraints, as shown in FIG. 4

****** 1 imaginary frequencies (negative Signs) ******
Harmonic frequencies (cm**−1), IR intensities (KM/Mole),
Raman scattering activities (A**4/AMU), Raman depolarization ratios,
reduced masses (AMU), force constants (mDyne/A) and normal coordinates:

| | 1 | 2 | 3 |
|---|---|---|---|
| | ?A | ?A | ?A |
| Frequencies | −295.1749 | 5.3883 | 8.3840 |
| Red. masses | 8.2116 | 5.0363 | 4.3317 |
| Frc consts | 0.4215 | 0.0001 | 0.0002 |
| IR Inten | 406.5632 | 0.8907 | 2.4410 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.16 | 0.07 | 0.01 | 0.01 | −0.03 | 0.04 | 0.00 | −0.03 | 0.00 |
| 2 | 6 | 0.55 | 0.15 | −0.04 | 0.00 | −0.01 | 0.03 | 0.00 | −0.05 | 0.00 |
| 3 | 8 | 0.16 | 0.02 | −0.02 | −0.01 | −0.01 | 0.05 | 0.00 | −0.06 | −0.01 |
| 4 | 6 | 0.06 | −0.05 | −0.03 | −0.01 | −0.02 | 0.06 | −0.01 | −0.05 | −0.03 |
| 5 | 6 | 0.04 | 0.00 | −0.03 | −0.01 | −0.04 | 0.06 | 0.01 | −0.02 | −0.02 |
| 6 | 7 | −0.19 | −0.09 | 0.09 | −0.01 | −0.01 | 0.01 | 0.00 | −0.05 | −0.01 |
| 7 | 6 | −0.11 | −0.04 | 0.06 | 0.00 | 0.00 | 0.00 | 0.02 | 0.01 | 0.02 |
| 8 | 6 | −0.12 | 0.00 | 0.03 | −0.01 | 0.00 | −0.02 | 0.00 | 0.00 | −0.01 |
| 9 | 7 | −0.19 | 0.00 | 0.05 | −0.03 | 0.00 | −0.02 | −0.03 | −0.07 | −0.06 |
| 10 | 6 | −0.19 | −0.05 | 0.07 | −0.03 | −0.01 | 0.00 | −0.03 | −0.10 | −0.05 |
| 11 | 6 | −0.08 | 0.03 | −0.01 | 0.00 | 0.01 | −0.04 | 0.02 | 0.06 | 0.01 |
| 12 | 7 | −0.04 | 0.02 | −0.01 | 0.02 | 0.01 | −0.04 | 0.05 | 0.12 | 0.05 |
| 13 | 6 | −0.01 | −0.01 | 0.02 | 0.03 | 0.01 | −0.02 | 0.06 | 0.13 | 0.08 |
| 14 | 7 | −0.04 | −0.04 | 0.05 | 0.03 | 0.00 | 0.00 | 0.05 | 0.08 | 0.06 |
| 15 | 7 | −0.09 | 0.02 | −0.02 | −0.01 | 0.02 | −0.06 | 0.01 | 0.05 | −0.02 |
| 16 | 6 | 0.00 | 0.01 | −0.01 | 0.01 | −0.01 | 0.08 | −0.03 | −0.05 | −0.06 |
| 17 | 16 | 0.00 | 0.00 | 0.00 | −0.03 | −0.02 | 0.07 | 0.02 | −0.05 | −0.03 |
| 18 | 6 | 0.00 | −0.01 | 0.00 | −0.03 | 0.07 | −0.22 | 0.03 | −0.14 | 0.28 |
| 19 | 8 | 0.04 | −0.03 | −0.03 | −0.01 | −0.08 | 0.05 | 0.02 | 0.01 | −0.01 |
| 20 | 8 | 0.07 | 0.00 | −0.14 | 0.04 | −0.05 | 0.04 | −0.01 | −0.01 | 0.01 |
| 21 | 1 | 0.01 | −0.01 | 0.02 | 0.05 | 0.01 | −0.02 | 0.09 | 0.18 | 0.12 |
| 22 | 1 | −0.23 | −0.06 | 0.08 | −0.04 | −0.01 | 0.01 | −0.05 | −0.15 | −0.08 |
| 23 | 1 | 0.14 | 0.06 | 0.08 | 0.00 | −0.01 | 0.02 | 0.00 | −0.05 | 0.02 |
| 24 | 1 | 0.04 | 0.15 | 0.24 | 0.02 | −0.01 | 0.02 | 0.00 | −0.03 | 0.00 |
| 25 | 1 | −0.02 | 0.06 | −0.03 | −0.03 | −0.03 | 0.08 | 0.02 | −0.02 | −0.05 |
| 26 | 1 | 0.06 | −0.10 | −0.05 | 0.00 | 0.00 | 0.06 | −0.01 | −0.07 | −0.02 |
| 27 | 1 | 0.12 | 0.01 | −0.25 | 0.04 | −0.05 | 0.06 | −0.01 | −0.01 | 0.01 |
| 28 | 1 | 0.01 | 0.04 | −0.02 | 0.00 | −0.01 | 0.12 | −0.02 | −0.06 | −0.11 |
| 29 | 1 | −0.01 | 0.02 | 0.00 | 0.04 | −0.02 | 0.07 | −0.08 | −0.04 | −0.04 |
| 30 | 1 | 0.00 | −0.02 | −0.01 | −0.19 | 0.11 | −0.27 | 0.20 | −0.18 | 0.34 |
| 31 | 1 | −0.01 | −0.01 | 0.02 | −0.03 | 0.09 | −0.28 | 0.04 | −0.16 | 0.36 |
| 32 | 1 | 0.00 | 0.00 | 0.00 | 0.13 | 0.07 | −0.29 | −0.15 | −0.13 | 0.36 |
| 33 | 1 | 0.05 | −0.12 | −0.04 | −0.02 | −0.05 | 0.05 | 0.03 | −0.01 | −0.01 |
| 34 | 1 | −0.08 | 0.11 | 0.02 | −0.03 | 0.01 | −0.07 | −0.01 | −0.01 | −0.05 |
| 35 | 1 | −0.08 | 0.05 | −0.01 | 0.00 | 0.02 | −0.08 | 0.02 | 0.09 | −0.01 |
| 36 | 15 | 0.01 | 0.00 | 0.00 | 0.01 | 0.02 | −0.02 | −0.03 | 0.04 | −0.02 |
| 37 | 8 | 0.02 | −0.01 | −0.02 | 0.00 | −0.01 | 0.05 | −0.02 | −0.01 | 0.01 |
| 38 | 8 | 0.00 | 0.00 | 0.00 | −0.13 | 0.23 | −0.03 | −0.10 | 0.12 | −0.04 |
| 39 | 8 | 0.00 | −0.01 | 0.00 | 0.14 | 0.00 | −0.24 | 0.05 | 0.07 | −0.09 |
| 40 | 8 | −0.01 | 0.00 | 0.00 | 0.03 | −0.11 | 0.12 | −0.04 | 0.01 | 0.01 |
| 41 | 1 | −0.23 | 0.03 | 0.02 | −0.04 | 0.00 | −0.03 | −0.04 | −0.10 | −0.09 |
| 42 | 1 | −0.01 | 0.03 | 0.00 | −0.20 | 0.31 | 0.04 | −0.13 | 0.13 | −0.02 |
| 43 | 1 | 0.00 | 0.02 | −0.02 | 0.01 | −0.20 | 0.25 | −0.06 | −0.04 | 0.06 |
| 44 | 1 | 0.00 | −0.01 | 0.01 | 0.14 | 0.02 | −0.28 | 0.04 | 0.10 | −0.11 |

| | 4 | 5 | 6 |
|---|---|---|---|
| | ?A | ?A | ?A |
| Frequencies | 10.5015 | 12.6058 | 21.9139 |
| Red. masses | 5.6772 | 6.5366 | 7.2871 |
| Frc consts | 0.0004 | 0.0006 | 0.0021 |
| IR Inten | 0.9357 | 1.1601 | 2.3609 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.01 | 0.07 | −0.01 | −0.05 | −0.03 | −0.02 | 0.00 | −0.07 | −0.09 |
| 2 | 6 | −0.02 | 0.06 | −0.01 | −0.05 | 0.01 | −0.03 | 0.01 | −0.03 | −0.10 |
| 3 | 8 | −0.01 | 0.07 | −0.01 | −0.02 | 0.05 | −0.01 | 0.02 | −0.01 | −0.09 |
| 4 | 6 | −0.01 | 0.07 | −0.02 | 0.03 | 0.05 | 0.02 | 0.03 | −0.02 | −0.06 |
| 5 | 6 | 0.00 | 0.08 | −0.02 | −0.01 | −0.01 | 0.01 | 0.01 | −0.06 | −0.06 |
| 6 | 7 | −0.01 | 0.05 | −0.01 | −0.04 | −0.04 | −0.02 | 0.02 | −0.02 | −0.06 |
| 7 | 6 | −0.03 | 0.00 | −0.02 | −0.01 | 0.03 | 0.01 | 0.01 | −0.02 | −0.05 |
| 8 | 6 | −0.02 | −0.01 | 0.00 | −0.03 | 0.00 | −0.03 | 0.04 | 0.08 | 0.05 |
| 9 | 7 | 0.01 | 0.03 | 0.02 | −0.06 | −0.09 | −0.09 | 0.06 | 0.14 | 0.08 |
| 10 | 6 | 0.01 | 0.06 | 0.01 | −0.06 | −0.11 | −0.08 | 0.05 | 0.08 | 0.02 |
| 11 | 6 | −0.04 | −0.06 | −0.01 | −0.01 | 0.05 | −0.01 | 0.03 | 0.10 | 0.08 |
| 12 | 7 | −0.07 | −0.09 | −0.03 | 0.02 | 0.14 | 0.05 | 0.00 | 0.02 | 0.02 |
| 13 | 6 | −0.08 | −0.07 | −0.05 | 0.03 | 0.16 | 0.09 | −0.02 | −0.08 | −0.07 |
| 14 | 7 | −0.07 | −0.03 | −0.04 | 0.02 | 0.12 | 0.08 | −0.02 | −0.11 | −0.11 |
| 15 | 7 | −0.03 | −0.08 | 0.01 | −0.03 | 0.03 | −0.05 | 0.06 | 0.21 | 0.18 |
| 16 | 6 | −0.02 | 0.07 | −0.03 | 0.10 | 0.03 | 0.06 | 0.04 | −0.01 | −0.02 |
| 17 | 16 | 0.03 | 0.07 | 0.00 | 0.18 | 0.02 | 0.12 | 0.06 | −0.03 | 0.02 |
| 18 | 6 | 0.04 | 0.00 | 0.26 | 0.18 | 0.19 | −0.05 | 0.06 | −0.01 | 0.02 |
| 19 | 8 | 0.01 | 0.11 | −0.01 | −0.02 | 0.00 | −0.01 | 0.00 | −0.07 | −0.08 |
| 20 | 8 | −0.03 | 0.08 | −0.01 | −0.08 | −0.03 | −0.05 | −0.01 | −0.08 | −0.11 |
| 21 | 1 | −0.11 | −0.10 | −0.06 | 0.06 | 0.23 | 0.14 | −0.05 | −0.15 | −0.12 |
| 22 | 1 | 0.04 | 0.10 | 0.02 | −0.08 | −0.17 | −0.11 | 0.07 | 0.10 | 0.02 |
| 23 | 1 | −0.02 | 0.06 | −0.01 | −0.08 | 0.02 | −0.06 | 0.01 | −0.03 | −0.12 |
| 24 | 1 | −0.01 | 0.06 | −0.01 | −0.05 | −0.07 | −0.01 | 0.00 | −0.08 | −0.08 |
| 25 | 1 | 0.02 | 0.07 | −0.03 | 0.00 | −0.04 | 0.05 | 0.01 | −0.07 | −0.04 |
| 26 | 1 | −0.01 | 0.07 | −0.01 | 0.04 | 0.11 | 0.00 | 0.03 | −0.01 | −0.07 |
| 27 | 1 | −0.02 | 0.09 | −0.01 | −0.08 | 0.01 | −0.06 | −0.01 | −0.06 | −0.11 |
| 28 | 1 | −0.01 | 0.05 | −0.07 | 0.09 | −0.03 | 0.07 | 0.04 | −0.03 | −0.03 |
| 29 | 1 | −0.05 | 0.08 | −0.02 | 0.11 | 0.09 | 0.06 | 0.04 | 0.02 | −0.02 |
| 30 | 1 | 0.17 | −0.04 | 0.30 | 0.03 | 0.20 | −0.13 | 0.05 | −0.03 | −0.01 |
| 31 | 1 | 0.05 | −0.02 | 0.33 | 0.25 | 0.20 | −0.03 | 0.08 | −0.02 | 0.07 |
| 32 | 1 | −0.11 | 0.02 | 0.32 | 0.27 | 0.27 | −0.10 | 0.06 | 0.04 | 0.02 |
| 33 | 1 | 0.02 | 0.08 | −0.01 | −0.04 | −0.01 | −0.01 | −0.01 | −0.08 | −0.08 |
| 34 | 1 | −0.01 | −0.04 | 0.02 | −0.05 | −0.05 | −0.08 | 0.10 | 0.26 | 0.20 |
| 35 | 1 | −0.04 | −0.11 | 0.00 | −0.02 | 0.07 | −0.03 | 0.06 | 0.22 | 0.20 |
| 36 | 15 | 0.04 | −0.07 | −0.01 | −0.05 | −0.08 | −0.02 | −0.07 | 0.01 | 0.04 |
| 37 | 8 | 0.00 | 0.03 | 0.01 | −0.05 | −0.01 | −0.06 | −0.02 | 0.00 | −0.11 |
| 38 | 8 | 0.00 | 0.02 | 0.01 | 0.01 | −0.12 | 0.01 | 0.00 | 0.16 | 0.18 |
| 39 | 8 | 0.03 | −0.18 | −0.18 | −0.12 | −0.13 | −0.01 | −0.09 | −0.02 | 0.01 |
| 40 | 8 | 0.13 | −0.21 | 0.12 | −0.02 | −0.10 | −0.01 | −0.21 | −0.11 | 0.16 |
| 41 | 1 | 0.03 | 0.03 | 0.03 | −0.08 | −0.13 | −0.12 | 0.08 | 0.20 | 0.14 |
| 42 | 1 | −0.03 | 0.12 | 0.05 | 0.04 | −0.09 | 0.00 | 0.08 | 0.12 | 0.12 |
| 43 | 1 | 0.15 | −0.18 | 0.20 | −0.01 | −0.05 | −0.03 | −0.21 | −0.08 | 0.04 |
| 44 | 1 | 0.05 | −0.24 | −0.19 | −0.11 | −0.18 | 0.02 | −0.12 | −0.01 | 0.13 |

| | 7 | 8 | 9 |
|---|---|---|---|
| | ?A | ?A | ?A |
| Frequencies | 31.1416 | 40.5795 | 51.3711 |
| Red. masses | 6.9727 | 6.0662 | 6.1879 |
| Frc consts | 0.0040 | 0.0059 | 0.0096 |
| IR Inten | 1.4935 | 1.7800 | 1.2108 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | −0.01 | 0.06 | 0.01 | −0.06 | 0.03 | −0.11 | 0.01 | 0.06 |
| 2 | 6 | −0.05 | −0.06 | 0.07 | 0.01 | −0.01 | 0.02 | −0.01 | −0.01 | 0.07 |
| 3 | 8 | −0.07 | −0.07 | 0.07 | 0.06 | 0.05 | 0.04 | 0.03 | −0.03 | 0.02 |
| 4 | 6 | 0.01 | −0.03 | 0.04 | 0.10 | 0.04 | 0.07 | 0.01 | −0.01 | −0.04 |
| 5 | 6 | 0.07 | 0.02 | 0.04 | 0.13 | 0.01 | 0.06 | −0.12 | 0.00 | −0.01 |
| 6 | 7 | −0.07 | −0.07 | 0.00 | 0.04 | −0.04 | 0.06 | 0.01 | 0.08 | 0.18 |
| 7 | 6 | −0.04 | −0.06 | −0.03 | 0.06 | −0.03 | 0.04 | 0.04 | 0.04 | 0.10 |
| 8 | 6 | −0.05 | 0.06 | 0.01 | 0.04 | −0.02 | 0.01 | 0.00 | 0.05 | 0.03 |
| 9 | 7 | −0.08 | 0.11 | 0.06 | 0.01 | −0.02 | 0.02 | −0.05 | 0.10 | 0.09 |
| 10 | 6 | −0.09 | 0.02 | 0.05 | 0.01 | −0.03 | 0.05 | −0.04 | 0.12 | 0.18 |
| 11 | 6 | −0.02 | 0.11 | 0.00 | 0.06 | 0.00 | −0.02 | 0.02 | −0.01 | −0.09 |
| 12 | 7 | 0.01 | 0.03 | −0.06 | 0.09 | 0.00 | −0.02 | 0.08 | −0.05 | −0.12 |
| 13 | 6 | 0.01 | −0.09 | −0.10 | 0.11 | −0.01 | 0.01 | 0.12 | −0.03 | −0.04 |
| 14 | 7 | −0.01 | −0.14 | −0.08 | 0.09 | −0.03 | 0.04 | 0.10 | 0.01 | 0.07 |
| 15 | 7 | −0.02 | 0.24 | 0.05 | 0.04 | 0.01 | −0.05 | −0.02 | −0.04 | −0.19 |
| 16 | 6 | 0.03 | −0.09 | −0.04 | 0.09 | 0.04 | 0.08 | 0.10 | −0.04 | 0.01 |
| 17 | 16 | 0.13 | −0.03 | −0.11 | −0.10 | 0.07 | −0.07 | 0.08 | −0.01 | −0.06 |
| 18 | 6 | 0.14 | 0.04 | −0.04 | −0.11 | −0.14 | −0.05 | 0.08 | −0.06 | 0.02 |
| 19 | 8 | 0.09 | 0.11 | 0.10 | 0.14 | 0.11 | 0.11 | −0.18 | −0.01 | −0.13 |
| 20 | 8 | −0.02 | 0.04 | 0.10 | −0.10 | −0.04 | −0.03 | −0.16 | 0.02 | 0.03 |
| 21 | 1 | 0.03 | −0.16 | −0.15 | 0.13 | −0.01 | 0.01 | 0.17 | −0.06 | −0.06 |
| 22 | 1 | −0.11 | 0.03 | 0.08 | −0.02 | −0.03 | 0.07 | −0.08 | 0.15 | 0.23 |
| 23 | 1 | −0.07 | −0.07 | 0.10 | −0.03 | 0.00 | −0.02 | 0.04 | −0.01 | 0.09 |
| 24 | 1 | 0.02 | −0.05 | 0.03 | 0.00 | −0.17 | 0.07 | −0.14 | 0.02 | 0.13 |
| 25 | 1 | 0.13 | 0.01 | −0.03 | 0.22 | −0.05 | 0.05 | −0.18 | 0.01 | 0.04 |
| 26 | 1 | 0.00 | 0.01 | 0.08 | 0.09 | 0.05 | 0.10 | 0.03 | 0.02 | −0.13 |
| 27 | 1 | −0.03 | 0.07 | 0.12 | −0.11 | 0.08 | −0.06 | −0.13 | −0.01 | −0.05 |
| 28 | 1 | 0.03 | −0.12 | −0.07 | 0.09 | 0.12 | 0.20 | 0.08 | −0.07 | 0.12 |
| 29 | 1 | 0.00 | −0.13 | −0.04 | 0.20 | −0.05 | 0.05 | 0.20 | −0.06 | −0.01 |
| 30 | 1 | 0.14 | 0.09 | 0.02 | 0.00 | −0.07 | 0.09 | 0.13 | −0.06 | 0.05 |
| 31 | 1 | 0.19 | 0.07 | −0.08 | −0.23 | −0.11 | −0.21 | 0.07 | −0.06 | 0.01 |
| 32 | 1 | 0.08 | 0.00 | −0.01 | −0.11 | −0.36 | −0.01 | 0.03 | −0.10 | 0.04 |
| 33 | 1 | 0.13 | 0.10 | 0.09 | 0.17 | 0.07 | 0.10 | −0.27 | 0.00 | −0.11 |
| 34 | 1 | −0.03 | 0.29 | 0.07 | 0.01 | 0.01 | −0.05 | −0.08 | 0.02 | −0.16 |
| 35 | 1 | 0.00 | 0.27 | 0.04 | 0.05 | 0.02 | −0.07 | −0.01 | −0.08 | −0.27 |
| 36 | 15 | −0.03 | −0.02 | 0.00 | −0.09 | 0.01 | −0.05 | 0.00 | −0.02 | −0.01 |
| 37 | 8 | −0.08 | −0.01 | 0.15 | −0.11 | 0.13 | −0.10 | 0.03 | −0.06 | −0.05 |
| 38 | 8 | −0.12 | −0.12 | −0.13 | −0.01 | −0.01 | 0.01 | 0.01 | 0.04 | 0.03 |
| 39 | 8 | 0.01 | −0.01 | −0.02 | −0.21 | −0.10 | −0.08 | 0.03 | 0.02 | 0.01 |
| 40 | 8 | 0.10 | 0.05 | −0.07 | −0.03 | −0.05 | 0.01 | −0.08 | −0.03 | 0.00 |
| 41 | 1 | −0.09 | 0.18 | 0.09 | −0.01 | −0.01 | 0.01 | −0.08 | 0.13 | 0.07 |
| 42 | 1 | −0.21 | −0.07 | −0.06 | 0.02 | 0.04 | −0.01 | 0.05 | −0.07 | −0.02 |
| 43 | 1 | 0.10 | 0.01 | 0.06 | 0.00 | 0.05 | −0.03 | −0.07 | 0.00 | −0.08 |
| 44 | 1 | 0.04 | −0.02 | −0.14 | −0.18 | −0.17 | −0.04 | −0.01 | 0.06 | 0.07 |

| | 10 | 11 | 12 |
|---|---|---|---|
| | ?A | ?A | ?A |
| Frequencies | 63.9749 | 71.0958 | 79.3691 |
| Red. masses | 5.9967 | 3.8869 | 6.6773 |
| Frc consts | 0.0145 | 0.0116 | 0.0248 |
| IR Inten | 1.6941 | 2.1238 | 1.9012 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.01 | −0.04 | 0.00 | 0.00 | 0.03 | −0.01 | −0.02 | 0.08 | −0.03 |
| 2 | 6 | −0.05 | 0.02 | −0.02 | −0.02 | 0.04 | −0.01 | 0.00 | 0.05 | −0.02 |
| 3 | 8 | −0.03 | 0.07 | 0.02 | 0.02 | 0.06 | −0.01 | 0.08 | 0.07 | −0.06 |
| 4 | 6 | 0.06 | 0.08 | 0.07 | 0.05 | 0.07 | 0.00 | 0.00 | 0.06 | −0.08 |
| 5 | 6 | 0.07 | 0.01 | 0.06 | −0.08 | −0.01 | 0.01 | −0.01 | 0.08 | −0.07 |
| 6 | 7 | −0.04 | 0.00 | −0.04 | 0.00 | −0.05 | −0.05 | 0.05 | −0.12 | 0.04 |
| 7 | 6 | −0.05 | 0.01 | −0.02 | −0.01 | −0.03 | −0.03 | 0.08 | −0.10 | 0.02 |
| 8 | 6 | −0.04 | 0.00 | −0.01 | 0.00 | −0.02 | −0.02 | 0.06 | −0.06 | 0.00 |
| 9 | 7 | −0.03 | −0.01 | −0.02 | 0.00 | −0.04 | −0.03 | 0.03 | −0.07 | 0.01 |
| 10 | 6 | −0.03 | −0.01 | −0.04 | 0.00 | −0.05 | −0.04 | 0.02 | −0.10 | 0.03 |
| 11 | 6 | −0.04 | 0.01 | 0.02 | 0.00 | 0.01 | 0.00 | 0.09 | −0.01 | −0.01 |
| 12 | 7 | −0.06 | 0.02 | 0.02 | 0.00 | 0.01 | 0.00 | 0.13 | −0.03 | −0.03 |
| 13 | 6 | −0.06 | 0.02 | 0.01 | −0.01 | −0.01 | −0.01 | 0.14 | −0.08 | −0.02 |
| 14 | 7 | −0.06 | 0.02 | −0.01 | −0.01 | −0.03 | −0.03 | 0.12 | −0.11 | 0.01 |
| 15 | 7 | −0.03 | 0.01 | 0.03 | 0.01 | 0.06 | 0.04 | 0.08 | 0.10 | 0.01 |
| 16 | 6 | 0.12 | 0.04 | 0.10 | 0.18 | 0.04 | 0.12 | −0.09 | 0.12 | −0.06 |
| 17 | 16 | 0.00 | 0.10 | −0.07 | 0.01 | 0.08 | −0.02 | −0.08 | 0.05 | 0.11 |
| 18 | 6 | −0.01 | −0.06 | 0.00 | −0.01 | −0.13 | 0.02 | −0.08 | 0.11 | 0.02 |
| 19 | 8 | 0.08 | 0.07 | 0.08 | −0.14 | −0.13 | −0.12 | −0.02 | 0.11 | −0.09 |
| 20 | 8 | −0.07 | −0.03 | −0.04 | 0.10 | 0.00 | 0.02 | −0.04 | 0.11 | −0.01 |
| 21 | 1 | −0.08 | 0.03 | 0.01 | −0.01 | −0.01 | −0.01 | 0.17 | −0.09 | −0.03 |
| 22 | 1 | −0.02 | −0.01 | −0.05 | 0.00 | −0.05 | −0.05 | 0.00 | −0.11 | 0.04 |
| 23 | 1 | −0.07 | 0.03 | −0.07 | −0.06 | 0.03 | −0.01 | −0.06 | 0.04 | 0.00 |
| 24 | 1 | −0.01 | −0.12 | 0.00 | 0.02 | 0.10 | −0.06 | −0.03 | 0.08 | −0.03 |
| 25 | 1 | 0.13 | −0.05 | 0.07 | −0.18 | 0.00 | 0.12 | −0.01 | 0.09 | −0.08 |
| 26 | 1 | 0.06 | 0.15 | 0.09 | 0.07 | 0.14 | −0.09 | 0.00 | −0.02 | −0.09 |
| 27 | 1 | −0.05 | −0.02 | −0.09 | 0.09 | −0.07 | 0.06 | −0.03 | 0.09 | −0.03 |
| 28 | 1 | 0.11 | 0.07 | 0.25 | 0.15 | 0.06 | 0.36 | −0.07 | 0.15 | −0.17 |
| 29 | 1 | 0.27 | −0.04 | 0.06 | 0.42 | 0.00 | 0.06 | −0.19 | 0.21 | −0.04 |
| 30 | 1 | 0.10 | 0.04 | 0.17 | 0.11 | −0.07 | 0.16 | −0.16 | −0.02 | −0.16 |
| 31 | 1 | −0.09 | −0.01 | −0.20 | −0.12 | −0.11 | −0.13 | −0.06 | 0.05 | 0.20 |
| 32 | 1 | −0.05 | −0.31 | 0.05 | −0.03 | −0.35 | 0.06 | −0.02 | 0.32 | −0.04 |
| 33 | 1 | 0.10 | −0.04 | 0.07 | −0.23 | −0.11 | −0.10 | −0.04 | 0.13 | −0.09 |
| 34 | 1 | −0.02 | −0.01 | 0.02 | 0.01 | −0.01 | 0.01 | 0.04 | −0.02 | −0.04 |
| 35 | 1 | −0.04 | 0.02 | 0.05 | 0.01 | 0.07 | 0.06 | 0.09 | 0.11 | 0.00 |
| 36 | 15 | 0.05 | −0.09 | −0.01 | −0.02 | 0.01 | 0.04 | −0.05 | −0.08 | 0.03 |
| 37 | 8 | 0.11 | −0.27 | −0.04 | −0.05 | 0.03 | 0.10 | −0.04 | −0.16 | 0.09 |
| 38 | 8 | 0.00 | 0.03 | 0.01 | −0.07 | −0.04 | −0.03 | −0.17 | −0.06 | −0.06 |
| 39 | 8 | 0.22 | 0.08 | 0.06 | −0.02 | −0.01 | 0.01 | 0.08 | 0.02 | 0.04 |
| 40 | 8 | −0.18 | −0.06 | −0.04 | 0.08 | 0.03 | 0.02 | −0.09 | −0.02 | −0.02 |
| 41 | 1 | −0.02 | −0.02 | −0.02 | 0.00 | −0.04 | −0.03 | 0.01 | −0.04 | 0.01 |
| 42 | 1 | 0.02 | −0.14 | −0.04 | −0.12 | −0.01 | 0.01 | −0.24 | −0.15 | −0.03 |
| 43 | 1 | −0.23 | −0.17 | −0.12 | 0.09 | 0.03 | 0.09 | −0.11 | −0.09 | 0.00 |
| 44 | 1 | 0.14 | 0.22 | 0.08 | 0.00 | −0.03 | −0.04 | 0.05 | 0.10 | −0.01 |

| | 13 | 14 | 15 |
|---|---|---|---|
| | ?A | ?A | ?A |
| Frequencies | 84.5642 | 96.5252 | 106.8549 |
| Red. masses | 8.1656 | 1.4645 | 4.1954 |
| Frc consts | 0.0344 | 0.0080 | 0.0282 |
| IR Inten | 9.9356 | 0.1682 | 0.4938 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.12 | 0.02 | −0.01 | 0.01 | 0.02 | 0.01 | 0.06 | 0.09 | 0.08 |
| 2 | 6 | 0.11 | 0.02 | −0.02 | 0.00 | 0.01 | 0.02 | 0.02 | 0.04 | 0.09 |
| 3 | 8 | 0.03 | −0.01 | 0.00 | 0.00 | 0.00 | 0.01 | −0.03 | 0.00 | 0.09 |
| 4 | 6 | 0.03 | −0.02 | 0.01 | −0.01 | 0.00 | 0.01 | −0.05 | 0.01 | 0.06 |
| 5 | 6 | 0.06 | −0.01 | 0.00 | −0.01 | 0.01 | 0.01 | −0.06 | 0.03 | 0.06 |
| 6 | 7 | 0.06 | 0.18 | −0.01 | 0.00 | 0.00 | 0.00 | 0.01 | −0.03 | 0.00 |
| 7 | 6 | 0.03 | 0.14 | 0.01 | 0.00 | 0.00 | 0.00 | 0.02 | −0.03 | −0.01 |
| 8 | 6 | 0.06 | 0.10 | 0.03 | 0.00 | 0.00 | 0.00 | 0.01 | −0.04 | −0.02 |
| 9 | 7 | 0.10 | 0.14 | 0.04 | 0.00 | 0.00 | 0.00 | 0.00 | −0.06 | −0.03 |
| 10 | 6 | 0.11 | 0.18 | 0.02 | 0.00 | 0.00 | 0.00 | 0.01 | −0.04 | −0.01 |
| 11 | 6 | 0.02 | −0.02 | 0.01 | 0.00 | 0.00 | 0.00 | 0.02 | −0.01 | 0.00 |
| 12 | 7 | −0.03 | 0.00 | 0.02 | 0.01 | 0.00 | 0.00 | 0.02 | −0.02 | −0.01 |
| 13 | 6 | −0.04 | 0.09 | 0.03 | 0.01 | −0.01 | 0.00 | 0.02 | −0.03 | −0.01 |
| 14 | 7 | −0.02 | 0.15 | 0.01 | 0.01 | 0.00 | 0.00 | 0.02 | −0.03 | 0.00 |
| 15 | 7 | 0.02 | −0.21 | −0.06 | 0.00 | 0.01 | 0.00 | 0.03 | 0.07 | 0.04 |
| 16 | 6 | 0.01 | −0.02 | −0.02 | 0.00 | 0.00 | 0.00 | −0.07 | −0.01 | 0.00 |
| 17 | 16 | 0.03 | −0.01 | −0.03 | 0.00 | 0.01 | −0.01 | 0.01 | 0.04 | −0.09 |
| 18 | 6 | 0.03 | 0.00 | 0.00 | 0.00 | 0.01 | 0.01 | 0.01 | 0.10 | 0.04 |
| 19 | 8 | 0.07 | −0.07 | 0.02 | −0.01 | −0.02 | 0.00 | −0.06 | −0.09 | 0.04 |
| 20 | 8 | 0.19 | −0.02 | 0.00 | 0.04 | 0.02 | 0.03 | 0.16 | 0.07 | 0.14 |
| 21 | 1 | −0.09 | 0.10 | 0.04 | 0.01 | −0.01 | 0.00 | 0.03 | −0.04 | −0.02 |
| 22 | 1 | 0.15 | 0.21 | 0.02 | 0.00 | 0.00 | 0.00 | 0.00 | −0.05 | −0.02 |
| 23 | 1 | 0.10 | 0.02 | −0.01 | 0.00 | 0.00 | 0.02 | 0.02 | 0.03 | 0.13 |
| 24 | 1 | 0.12 | 0.08 | −0.03 | 0.02 | 0.04 | 0.00 | 0.07 | 0.19 | 0.03 |
| 25 | 1 | 0.03 | 0.01 | 0.00 | −0.03 | 0.02 | 0.01 | −0.14 | 0.09 | 0.08 |
| 26 | 1 | 0.02 | −0.03 | 0.02 | 0.00 | 0.00 | 0.00 | −0.05 | −0.02 | 0.06 |
| 27 | 1 | 0.19 | −0.08 | 0.01 | 0.04 | −0.01 | 0.03 | 0.21 | −0.13 | 0.08 |
| 28 | 1 | 0.01 | −0.01 | −0.04 | 0.00 | 0.00 | 0.00 | −0.06 | −0.02 | −0.02 |
| 29 | 1 | −0.01 | −0.03 | −0.01 | −0.01 | −0.01 | 0.00 | −0.09 | −0.06 | 0.01 |
| 30 | 1 | 0.04 | 0.00 | 0.01 | 0.01 | 0.01 | 0.01 | 0.04 | 0.10 | 0.05 |
| 31 | 1 | 0.04 | 0.00 | 0.00 | 0.01 | 0.01 | 0.01 | 0.08 | 0.10 | 0.09 |
| 32 | 1 | 0.01 | −0.01 | 0.01 | −0.01 | 0.01 | 0.01 | −0.07 | 0.15 | 0.07 |
| 33 | 1 | 0.09 | −0.10 | 0.02 | −0.01 | 0.01 | 0.00 | −0.07 | −0.14 | 0.04 |
| 34 | 1 | 0.06 | 0.01 | 0.04 | 0.00 | −0.01 | 0.00 | 0.02 | −0.06 | −0.01 |
| 35 | 1 | 0.00 | −0.26 | −0.07 | 0.00 | 0.00 | 0.00 | 0.03 | 0.07 | 0.05 |
| 36 | 15 | −0.13 | −0.08 | 0.02 | 0.00 | −0.01 | −0.02 | −0.01 | −0.02 | −0.07 |
| 37 | 8 | −0.14 | −0.12 | 0.08 | −0.02 | 0.00 | 0.01 | 0.02 | −0.02 | −0.15 |
| 38 | 8 | −0.28 | −0.09 | −0.11 | 0.07 | −0.05 | 0.03 | 0.02 | 0.05 | −0.01 |
| 39 | 8 | −0.06 | −0.03 | 0.00 | −0.01 | −0.01 | −0.02 | −0.06 | −0.03 | −0.04 |
| 40 | 8 | −0.13 | −0.04 | −0.02 | −0.09 | 0.04 | −0.06 | −0.10 | −0.07 | −0.01 |
| 41 | 1 | 0.13 | 0.12 | 0.05 | 0.00 | 0.00 | 0.00 | 0.00 | −0.06 | −0.03 |
| 42 | 1 | −0.37 | −0.17 | −0.06 | −0.01 | 0.58 | 0.18 | 0.14 | −0.32 | −0.14 |
| 43 | 1 | −0.12 | −0.03 | −0.04 | −0.23 | −0.46 | 0.35 | −0.03 | 0.25 | −0.40 |
| 44 | 1 | −0.08 | 0.00 | −0.02 | 0.14 | −0.13 | −0.41 | −0.17 | 0.03 | 0.28 |

| | 16 | 17 | 18 |
|---|---|---|---|
| | ?A | ?A | ?A |
| Frequencies | 127.3797 | 139.0005 | 154.4462 |
| Red. masses | 5.4803 | 1.8140 | 1.2016 |
| Frc consts | 0.0524 | 0.0206 | 0.0169 |
| IR Inten | 3.3268 | 143.7682 | 4.3637 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.03 | 0.11 | 0.06 | 0.00 | 0.01 | 0.00 | 0.00 | −0.01 | 0.03 |
| 2 | 6 | 0.03 | 0.01 | 0.09 | −0.03 | 0.00 | 0.00 | 0.02 | −0.01 | 0.02 |
| 3 | 8 | 0.02 | −0.06 | 0.03 | −0.01 | 0.00 | 0.00 | 0.02 | −0.01 | 0.02 |
| 4 | 6 | 0.05 | −0.01 | −0.05 | 0.00 | 0.01 | 0.00 | 0.03 | 0.00 | 0.01 |
| 5 | 6 | 0.02 | 0.09 | −0.03 | −0.01 | 0.01 | 0.00 | 0.01 | 0.00 | 0.01 |
| 6 | 7 | 0.00 | 0.05 | −0.03 | −0.01 | −0.07 | −0.03 | 0.00 | 0.03 | 0.00 |
| 7 | 6 | −0.02 | 0.03 | −0.03 | 0.01 | −0.02 | −0.01 | 0.00 | 0.02 | −0.01 |
| 8 | 6 | −0.01 | 0.00 | −0.04 | 0.01 | 0.05 | 0.03 | 0.00 | 0.00 | −0.01 |
| 9 | 7 | 0.00 | −0.01 | −0.05 | −0.01 | 0.05 | 0.03 | 0.00 | −0.02 | −0.03 |
| 10 | 6 | 0.00 | 0.01 | −0.05 | −0.02 | −0.04 | −0.02 | 0.00 | 0.00 | −0.03 |
| 11 | 6 | −0.02 | 0.00 | −0.01 | 0.02 | 0.06 | 0.03 | 0.00 | 0.01 | 0.00 |
| 12 | 7 | −0.05 | −0.01 | −0.02 | 0.03 | 0.06 | 0.05 | −0.02 | −0.01 | −0.01 |
| 13 | 6 | −0.06 | 0.00 | −0.03 | 0.02 | −0.01 | −0.01 | −0.02 | −0.01 | −0.02 |
| 14 | 7 | −0.04 | 0.03 | −0.03 | 0.00 | −0.05 | −0.03 | −0.01 | 0.02 | −0.01 |
| 15 | 7 | 0.00 | 0.04 | 0.04 | −0.02 | −0.16 | −0.06 | 0.00 | 0.01 | 0.02 |
| 16 | 6 | 0.09 | −0.02 | −0.02 | 0.00 | 0.01 | 0.00 | 0.05 | −0.01 | 0.03 |
| 17 | 16 | 0.02 | −0.13 | 0.17 | 0.00 | 0.01 | 0.01 | −0.01 | 0.00 | −0.02 |
| 18 | 6 | 0.00 | −0.24 | −0.08 | −0.01 | 0.00 | 0.00 | −0.01 | −0.03 | −0.01 |
| 19 | 8 | 0.01 | 0.15 | −0.05 | −0.01 | 0.01 | −0.01 | 0.00 | 0.01 | −0.01 |
| 20 | 8 | 0.02 | 0.18 | 0.13 | 0.01 | 0.03 | 0.03 | −0.03 | 0.00 | 0.01 |
| 21 | 1 | −0.08 | −0.01 | −0.04 | 0.02 | −0.02 | −0.02 | −0.03 | −0.02 | −0.03 |
| 22 | 1 | 0.01 | 0.01 | −0.06 | −0.03 | −0.08 | −0.03 | 0.00 | −0.01 | −0.03 |
| 23 | 1 | 0.04 | −0.02 | 0.17 | −0.04 | −0.01 | 0.01 | 0.02 | −0.01 | 0.03 |
| 24 | 1 | 0.03 | 0.15 | 0.05 | 0.01 | 0.02 | −0.03 | −0.01 | −0.02 | 0.04 |
| 25 | 1 | 0.01 | 0.13 | −0.07 | −0.01 | 0.01 | 0.00 | 0.01 | −0.01 | 0.02 |
| 26 | 1 | 0.05 | 0.01 | −0.09 | 0.00 | 0.02 | 0.00 | 0.03 | 0.00 | −0.01 |
| 27 | 1 | 0.06 | 0.07 | 0.06 | 0.02 | −0.01 | 0.02 | −0.02 | 0.01 | 0.00 |
| 28 | 1 | 0.09 | −0.04 | −0.02 | 0.00 | 0.01 | 0.01 | 0.04 | 0.01 | 0.10 |
| 29 | 1 | 0.09 | 0.07 | −0.01 | 0.01 | 0.01 | 0.00 | 0.12 | −0.03 | 0.01 |
| 30 | 1 | −0.03 | −0.14 | 0.01 | 0.00 | 0.01 | 0.01 | −0.04 | −0.39 | −0.41 |
| 31 | 1 | −0.15 | −0.20 | −0.33 | −0.01 | 0.01 | −0.02 | 0.00 | −0.21 | 0.53 |
| 32 | 1 | 0.15 | −0.50 | −0.12 | 0.00 | −0.02 | 0.00 | 0.02 | 0.51 | −0.10 |
| 33 | 1 | −0.01 | 0.17 | −0.05 | −0.01 | −0.02 | −0.01 | −0.02 | 0.02 | 0.00 |
| 34 | 1 | 0.02 | −0.01 | 0.02 | 0.10 | 0.86 | 0.39 | 0.03 | 0.14 | 0.08 |
| 35 | 1 | −0.01 | 0.03 | 0.06 | 0.04 | −0.02 | −0.07 | 0.01 | 0.02 | 0.03 |
| 36 | 15 | −0.01 | −0.01 | −0.04 | 0.00 | 0.00 | −0.01 | 0.00 | 0.00 | 0.00 |
| 37 | 8 | 0.00 | −0.01 | −0.06 | 0.00 | 0.00 | −0.01 | 0.00 | 0.00 | 0.00 |
| 38 | 8 | 0.02 | 0.02 | 0.00 | 0.02 | 0.01 | 0.01 | 0.00 | 0.00 | 0.00 |
| 39 | 8 | −0.03 | −0.02 | −0.02 | 0.00 | 0.00 | 0.00 | 0.00 | 0.00 | 0.00 |
| 40 | 8 | −0.08 | −0.03 | −0.02 | −0.01 | 0.00 | 0.00 | 0.00 | 0.00 | 0.00 |
| 41 | 1 | 0.01 | −0.04 | −0.07 | −0.04 | −0.05 | −0.06 | −0.01 | −0.06 | −0.06 |
| 42 | 1 | 0.06 | −0.08 | −0.05 | 0.04 | −0.02 | −0.01 | 0.00 | 0.00 | 0.00 |
| 43 | 1 | −0.06 | 0.06 | −0.17 | 0.00 | 0.02 | −0.03 | 0.00 | 0.00 | 0.00 |
| 44 | 1 | −0.06 | 0.00 | 0.09 | −0.01 | 0.00 | 0.02 | 0.00 | −0.01 | −0.01 |

| | 19 | 20 | 21 |
|---|---|---|---|
| | ?A | ?A | ?A |
| Frequencies | 160.5304 | 187.1775 | 194.7224 |
| Red. masses | 2.3618 | 1.4531 | 5.9466 |
| Frc consts | 0.0359 | 0.0300 | 0.1328 |
| IR Inten | 27.5137 | 43.9722 | 16.1753 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.02 | −0.01 | 0.03 | 0.00 | 0.00 | 0.00 | 0.03 | −0.01 | −0.13 |
| 2 | 6 | 0.06 | 0.02 | 0.02 | 0.01 | 0.00 | 0.00 | −0.17 | −0.03 | −0.13 |
| 3 | 8 | 0.03 | 0.00 | 0.02 | 0.01 | 0.00 | 0.00 | −0.11 | 0.03 | −0.10 |
| 4 | 6 | −0.01 | −0.02 | 0.02 | 0.00 | 0.00 | 0.00 | −0.01 | 0.05 | −0.05 |
| 5 | 6 | −0.02 | −0.01 | 0.02 | 0.00 | 0.00 | 0.00 | 0.04 | −0.01 | −0.07 |
| 6 | 7 | 0.03 | 0.09 | 0.04 | 0.00 | 0.00 | 0.00 | −0.04 | −0.03 | 0.09 |
| 7 | 6 | 0.03 | 0.09 | 0.04 | 0.00 | 0.00 | 0.00 | −0.01 | 0.09 | 0.16 |
| 8 | 6 | 0.02 | 0.00 | −0.02 | 0.00 | 0.00 | 0.00 | −0.04 | 0.08 | 0.12 |
| 9 | 7 | 0.00 | −0.09 | −0.09 | 0.00 | 0.00 | 0.00 | −0.07 | −0.01 | 0.07 |
| 10 | 6 | 0.01 | −0.04 | −0.06 | 0.00 | 0.00 | 0.00 | −0.08 | −0.09 | 0.04 |
| 11 | 6 | 0.02 | 0.00 | 0.00 | 0.00 | 0.00 | 0.00 | −0.03 | −0.02 | 0.00 |
| 12 | 7 | −0.02 | −0.10 | −0.07 | 0.00 | 0.00 | 0.00 | −0.01 | −0.19 | −0.13 |
| 13 | 6 | −0.01 | −0.06 | −0.06 | 0.00 | 0.00 | 0.00 | 0.03 | −0.07 | 0.00 |
| 14 | 7 | 0.02 | 0.09 | 0.03 | 0.00 | 0.00 | 0.00 | 0.05 | 0.12 | 0.19 |
| 15 | 7 | 0.03 | 0.04 | 0.09 | 0.00 | 0.00 | 0.00 | −0.06 | 0.08 | 0.00 |
| 16 | 6 | −0.04 | −0.01 | −0.01 | 0.00 | 0.00 | 0.00 | 0.04 | 0.03 | −0.02 |
| 17 | 16 | 0.01 | 0.00 | −0.01 | 0.00 | 0.00 | 0.00 | 0.01 | 0.01 | 0.02 |
| 18 | 6 | 0.01 | 0.03 | 0.01 | 0.00 | 0.00 | 0.00 | 0.00 | −0.07 | −0.02 |
| 19 | 8 | −0.02 | −0.01 | 0.01 | 0.00 | 0.00 | 0.00 | 0.09 | 0.01 | 0.00 |
| 20 | 8 | −0.07 | −0.03 | −0.04 | −0.01 | −0.01 | −0.01 | 0.23 | −0.01 | 0.03 |
| 21 | 1 | −0.04 | −0.14 | −0.12 | 0.00 | 0.00 | 0.00 | 0.05 | −0.14 | −0.05 |
| 22 | 1 | 0.00 | −0.10 | −0.10 | 0.00 | 0.01 | 0.00 | −0.12 | −0.19 | −0.02 |
| 23 | 1 | 0.08 | 0.02 | 0.02 | 0.01 | 0.00 | 0.01 | −0.18 | −0.01 | −0.19 |
| 24 | 1 | −0.04 | −0.02 | 0.09 | 0.00 | 0.00 | 0.01 | 0.10 | 0.04 | −0.29 |
| 25 | 1 | −0.03 | 0.00 | 0.02 | 0.00 | 0.00 | 0.00 | 0.09 | −0.03 | −0.09 |
| 26 | 1 | −0.01 | −0.04 | 0.01 | 0.00 | −0.01 | 0.00 | −0.02 | 0.12 | −0.01 |
| 27 | 1 | −0.07 | 0.03 | −0.06 | −0.01 | 0.01 | 0.00. | 0.19 | −0.12 | 0.16 |
| 28 | 1 | −0.03 | −0.01 | −0.05 | 0.00 | 0.00 | 0.00 | 0.03 | 0.01 | 0.01 |
| 29 | 1 | −0.08 | −0.01 | 0.00 | 0.00 | 0.00 | 0.00 | 0.07 | 0.06 | −0.03 |
| 30 | 1 | 0.02 | 0.21 | 0.20 | 0.00 | 0.01 | 0.01 | 0.00 | −0.17 | −0.13 |
| 31 | 1 | 0.03 | 0.12 | −0.24 | 0.00 | 0.01 | −0.01 | −0.06 | −0.12 | 0.10 |
| 32 | 1 | −0.02 | −0.21 | 0.06 | 0.00 | −0.01 | 0.00 | 0.04 | 0.05 | −0.06 |
| 33 | 1 | −0.03 | −0.01 | 0.01 | −0.01 | −0.01 | 0.00 | 0.15 | 0.01 | −0.01 |
| 34 | 1 | 0.14 | 0.58 | 0.32 | 0.00 | −0.01 | 0.00 | −0.09 | 0.24 | 0.07 |
| 35 | 1 | 0.05 | 0.09 | 0.09 | 0.00 | 0.00 | 0.00 | −0.05 | 0.01 | −0.12 |
| 36 | 15 | 0.00 | 0.00 | 0.01 | −0.02 | 0.01 | 0.05 | 0.00 | 0.01 | −0.01 |
| 37 | 8 | 0.00 | 0.00 | 0.01 | −0.01 | 0.01 | 0.01 | −0.01 | −0.02 | 0.01 |
| 38 | 8 | −0.03 | −0.01 | −0.01 | 0.02 | −0.05 | 0.06 | 0.05 | 0.02 | 0.03 |
| 39 | 8 | 0.00 | 0.00 | 0.01 | 0.05 | −0.02 | −0.09 | 0.00 | −0.01 | −0.04 |
| 40 | 8 | 0.01 | 0.01 | 0.01 | −0.06 | 0.05 | 0.01 | 0.00 | 0.01 | −0.02 |
| 41 | 1 | −0.03 | −0.25 | −0.21 | 0.00 | 0.00 | 0.00 | −0.09 | −0.04 | 0.03 |
| 42 | 1 | −0.06 | 0.04 | 0.02 | 0.14 | −0.38 | −0.07 | 0.13 | −0.16 | −0.05 |
| 43 | 1 | 0.00 | −0.03 | 0.06 | 0.00 | 0.34 | −0.36 | 0.01 | 0.05 | −0.07 |
| 44 | 1 | 0.02 | −0.01 | −0.04 | 0.26 | −0.17 | −0.67 | −0.03 | 0.01 | 0.04 |

| | 22 | 23 | 24 |
|---|---|---|---|
| | ?A | ?A | ?A |
| Frequencies | 217.5666 | 221.5844 | 234.5792 |
| Red. masses | 2.0385 | 2.2687 | 4.1986 |
| Frc consts | 0.0569 | 0.0656 | 0.1361 |
| IR Inten | 35.0906 | 13.2596 | 6.8355 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | −0.01 | 0.00 | 0.01 | 0.00 | −0.01 | 0.06 | −0.05 |
| 2 | 6 | −0.01 | 0.01 | −0.01 | 0.00 | 0.01 | 0.00 | −0.03 | 0.08 | −0.06 |
| 3 | 8 | −0.01 | 0.01 | −0.01 | −0.01 | 0.00 | 0.00 | −0.11 | 0.06 | −0.02 |
| 4 | 6 | −0.01 | 0.00 | 0.00 | −0.01 | 0.00 | 0.00 | −0.10 | 0.04 | 0.01 |
| 5 | 6 | −0.01 | 0.00 | 0.00 | −0.01 | 0.00 | 0.00 | −0.05 | 0.04 | 0.00 |
| 6 | 7 | 0.00 | −0.01 | 0.00 | 0.00 | 0.00 | 0.01 | 0.02 | 0.12 | 0.12 |
| 7 | 6 | 0.00 | 0.00 | 0.01 | 0.00 | 0.00 | 0.00 | −0.01 | −0.06 | −0.01 |
| 8 | 6 | 0.00 | 0.00 | 0.01 | 0.00 | −0.01 | 0.00 | −0.02 | −0.14 | −0.07 |
| 9 | 7 | 0.00 | 0.00 | 0.01 | 0.00 | 0.00 | 0.00 | −0.02 | −0.10 | −0.04 |
| 10 | 6 | 0.00 | −0.01 | 0.00 | 0.00 | 0.00 | 0.01 | 0.02 | 0.10 | 0.10 |
| 11 | 6 | 0.00 | 0.00 | 0.00 | 0.00 | 0.00 | 0.00 | 0.01 | −0.01 | 0.00 |
| 12 | 7 | 0.00 | −0.01 | 0.00 | 0.00 | 0.00 | 0.00 | 0.05 | 0.08 | 0.07 |
| 13 | 6 | 0.00 | 0.00 | 0.00 | 0.00 | 0.00 | 0.00 | 0.04 | −0.01 | 0.01 |
| 14 | 7 | 0.00 | 0.00 | 0.01 | 0.00 | −0.01 | 0.00 | 0.02 | −0.09 | −0.02 |
| 15 | 7 | 0.00 | 0.00 | 0.00 | 0.00 | 0.00 | 0.00 | −0.01 | 0.01 | 0.00 |
| 16 | 6 | 0.00 | 0.00 | 0.00 | 0.00 | 0.00 | 0.00 | −0.04 | −0.01 | −0.02 |
| 17 | 16 | 0.01 | 0.00 | 0.00 | 0.00 | 0.00 | 0.00 | 0.05 | −0.01 | 0.00 |
| 18 | 6 | 0.00 | −0.02 | −0.01 | 0.00 | −0.01 | −0.01 | 0.04 | −0.14 | −0.06 |
| 19 | 8 | 0.00 | −0.01 | 0.01 | 0.00 | 0.00 | 0.01 | 0.00 | 0.01 | 0.11 |
| 20 | 8 | 0.01 | 0.00 | −0.01 | 0.01 | −0.01 | −0.01 | 0.07 | −0.02 | −0.08 |
| 21 | 1 | 0.01 | −0.01 | 0.00 | 0.00 | 0.00 | 0.00 | 0.05 | 0.02 | 0.03 |
| 22 | 1 | −0.01 | −0.01 | 0.00 | 0.00 | 0.00 | 0.01 | 0.04 | 0.22 | 0.19 |
| 23 | 1 | −0.01 | 0.01 | −0.01 | 0.00 | 0.01 | −0.01 | 0.02 | 0.10 | −0.09 |
| 24 | 1 | 0.00 | 0.01 | −0.01 | 0.00 | 0.01 | −0.01 | 0.00 | 0.09 | −0.06 |
| 25 | 1 | −0.01 | 0.00 | 0.00 | −0.01 | 0.00 | 0.00 | −0.04 | 0.05 | −0.03 |
| 26 | 1 | −0.01 | 0.01 | 0.00 | −0.01 | 0.00 | 0.00 | −0.10 | 0.06 | 0.03 |
| 27 | 1 | 0.00 | 0.01 | 0.01 | 0.00 | 0.02 | 0.02 | 0.07 | −0.07 | −0.08 |
| 28 | 1 | 0.00 | −0.01 | 0.00 | 0.00 | −0.01 | −0.01 | −0.05 | −0.09 | −0.06 |
| 29 | 1 | 0.00 | 0.00 | 0.00 | −0.01 | 0.00 | 0.00 | −0.09 | 0.01 | −0.01 |
| 30 | 1 | 0.01 | −0.03 | −0.02 | 0.01 | −0.02 | −0.02 | 0.07 | −0.27 | −0.17 |
| 31 | 1 | −0.01 | −0.03 | 0.01 | 0.00 | −0.02 | 0.00 | −0.08 | −0.21 | 0.06 |
| 32 | 1 | 0.01 | 0.00 | −0.01 | 0.01 | −0.01 | −0.01 | 0.11 | −0.06 | −0.10 |
| 33 | 1 | 0.01 | 0.05 | 0.02 | 0.00 | 0.00 | 0.01 | 0.09 | −0.11 | 0.08 |
| 34 | 1 | −0.01 | 0.01 | 0.00 | 0.00 | 0.01 | 0.00 | 0.02 | 0.27 | 0.11 |
| 35 | 1 | 0.00 | 0.00 | −0.01 | 0.00 | 0.01 | 0.00 | 0.04 | 0.16 | 0.05 |
| 36 | 15 | 0.01 | −0.04 | 0.01 | 0.00 | 0.06 | −0.01 | 0.00 | 0.00 | 0.02 |
| 37 | 8 | −0.03 | 0.15 | −0.05 | 0.00 | 0.10 | −0.03 | 0.05 | −0.04 | −0.10 |
| 38 | 8 | −0.02 | −0.09 | −0.04 | 0.03 | −0.13 | −0.09 | −0.07 | 0.03 | −0.01 |
| 39 | 8 | 0.11 | 0.05 | 0.04 | 0.05 | 0.09 | 0.00 | −0.04 | −0.01 | 0.06 |
| 40 | 8 | −0.05 | −0.08 | 0.05 | −0.07 | −0.08 | 0.13 | 0.06 | 0.02 | 0.01 |
| 41 | 1 | 0.00 | 0.00 | 0.01 | 0.00 | −0.01 | 0.00 | −0.03 | −0.19 | −0.11 |
| 42 | 1 | −0.21 | 0.65 | 0.20 | 0.11 | −0.63 | −0.23 | −0.12 | 0.04 | 0.02 |
| 43 | 1 | 0.07 | 0.40 | −0.47 | −0.17 | −0.45 | 0.46 | 0.06 | −0.01 | 0.09 |
| 44 | 1 | 0.06 | 0.17 | 0.05 | 0.04 | 0.13 | −0.02 | 0.13 | −0.14 | −0.35 |

| | 25 | 26 | 27 |
|---|---|---|---|
| | ?A | ?A | ?A |
| Frequencies | 238.4987 | 246.5373 | 259.2225 |
| Red. masses | 3.4372 | 3.9212 | 5.0448 |
| Frc consts | 0.1152 | 0.1404 | 0.1997 |
| IR Inten | 14.4402 | 4.6700 | 10.6215 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | −0.03 | −0.02 | −0.01 | 0.08 | 0.01 | −0.02 | −0.01 | −0.01 |
| 2 | 6 | −0.01 | −0.02 | −0.02 | 0.05 | 0.11 | 0.00 | −0.02 | −0.01 | −0.01 |
| 3 | 8 | 0.02 | −0.01 | −0.03 | −0.06 | 0.07 | 0.03 | −0.02 | −0.01 | −0.01 |
| 4 | 6 | 0.03 | −0.01 | −0.02 | −0.10 | 0.04 | 0.05 | 0.00 | −0.01 | −0.01 |
| 5 | 6 | 0.02 | −0.02 | −0.02 | −0.08 | 0.05 | 0.05 | 0.00 | −0.01 | −0.01 |
| 6 | 7 | 0.01 | 0.11 | 0.09 | 0.00 | −0.08 | −0.07 | −0.01 | −0.01 | 0.00 |
| 7 | 6 | −0.01 | −0.04 | −0.01 | 0.02 | 0.01 | 0.00 | 0.00 | 0.00 | 0.01 |
| 8 | 6 | −0.03 | −0.11 | −0.07 | 0.04 | 0.07 | 0.05 | −0.01 | 0.01 | 0.01 |
| 9 | 7 | −0.03 | −0.08 | −0.05 | 0.04 | 0.04 | 0.03 | −0.01 | 0.00 | 0.01 |
| 10 | 6 | 0.00 | 0.10 | 0.08 | 0.02 | −0.07 | −0.06 | −0.01 | −0.01 | 0.00 |
| 11 | 6 | −0.01 | −0.01 | −0.01 | 0.03 | 0.00 | 0.01 | −0.01 | 0.00 | 0.00 |
| 12 | 7 | 0.01 | 0.07 | 0.05 | 0.02 | −0.04 | −0.02 | 0.00 | 0.00 | 0.00 |
| 13 | 6 | 0.00 | 0.01 | 0.01 | 0.02 | −0.01 | −0.01 | 0.00 | 0.00 | 0.00 |
| 14 | 7 | −0.01 | −0.05 | −0.02 | 0.02 | 0.02 | 0.00 | 0.00 | 0.00 | 0.01 |
| 15 | 7 | −0.02 | 0.01 | 0.00 | 0.04 | −0.02 | 0.00 | −0.01 | 0.00 | −0.01 |
| 16 | 6 | 0.02 | 0.01 | 0.00 | −0.08 | −0.01 | −0.02 | 0.02 | −0.01 | 0.02 |
| 17 | 16 | −0.01 | 0.00 | 0.00 | 0.06 | 0.00 | 0.00 | −0.01 | −0.01 | 0.00 |
| 18 | 6 | −0.01 | 0.03 | 0.01 | 0.05 | −0.15 | −0.06 | −0.01 | 0.03 | 0.02 |
| 19 | 8 | 0.02 | 0.01 | −0.03 | −0.05 | −0.04 | 0.12 | 0.00 | 0.01 | −0.01 |
| 20 | 8 | 0.05 | −0.05 | 0.00 | −0.06 | −0.02 | −0.14 | 0.02 | −0.04 | −0.01 |
| 21 | 1 | 0.01 | 0.03 | 0.02 | 0.01 | −0.02 | −0.02 | 0.00 | −0.01 | 0.00 |
| 22 | 1 | 0.02 | 0.21 | 0.16 | 0.02 | −0.14 | −0.12 | −0.01 | −0.02 | −0.01 |
| 23 | 1 | 0.01 | −0.01 | −0.03 | 0.00 | 0.09 | 0.02 | 0.02 | 0.00 | −0.02 |
| 24 | 1 | 0.01 | −0.03 | −0.04 | −0.05 | 0.07 | 0.07 | −0.01 | −0.01 | −0.02 |
| 25 | 1 | 0.05 | −0.04 | −0.03 | −0.11 | 0.08 | 0.04 | 0.02 | −0.02 | −0.02 |
| 26 | 1 | 0.03 | 0.00 | −0.02 | −0.10 | 0.01 | 0.06 | 0.00 | 0.01 | −0.02 |
| 27 | 1 | −0.01 | 0.05 | 0.14 | −0.11 | 0.18 | −0.07 | −0.06 | 0.13 | 0.14 |
| 28 | 1 | 0.02 | 0.03 | 0.02 | −0.08 | −0.10 | −0.14 | 0.02 | 0.00 | 0.08 |
| 29 | 1 | 0.04 | 0.01 | −0.01 | −0.20 | 0.00 | 0.01 | 0.09 | −0.02 | 0.01 |
| 30 | 1 | −0.01 | 0.06 | 0.04 | 0.09 | −0.28 | −0.17 | −0.02 | 0.05 | 0.03 |
| 31 | 1 | 0.02 | 0.05 | −0.01 | −0.10 | −0.22 | 0.04 | 0.03 | 0.04 | 0.00 |
| 32 | 1 | −0.03 | 0.01 | 0.03 | 0.12 | −0.10 | −0.10 | −0.03 | 0.03 | 0.03 |
| 33 | 1 | 0.00 | 0.18 | −0.02 | 0.01 | 0.11 | 0.12 | −0.02 | 0.46 | 0.02 |
| 34 | 1 | 0.01 | 0.23 | 0.10 | 0.03 | −0.17 | −0.06 | −0.02 | 0.00 | −0.01 |
| 35 | 1 | 0.02 | 0.14 | 0.06 | 0.01 | −0.10 | −0.02 | −0.01 | 0.00 | −0.02 |
| 36 | 15 | 0.01 | −0.01 | −0.03 | 0.02 | −0.01 | −0.01 | 0.18 | 0.03 | 0.06 |
| 37 | 8 | −0.08 | 0.05 | 0.19 | −0.05 | 0.03 | 0.14 | 0.17 | 0.10 | 0.04 |
| 38 | 8 | 0.09 | −0.01 | 0.04 | 0.04 | 0.01 | 0.03 | −0.11 | 0.06 | −0.19 |
| 39 | 8 | 0.05 | −0.03 | −0.12 | 0.03 | −0.04 | −0.08 | −0.10 | −0.21 | 0.04 |
| 40 | 8 | −0.09 | 0.01 | −0.05 | −0.06 | 0.03 | −0.05 | −0.19 | 0.08 | 0.02 |
| 41 | 1 | −0.04 | −0.15 | −0.10 | 0.05 | 0.09 | 0.07 | 0.00 | 0.00 | 0.01 |
| 42 | 1 | 0.17 | −0.06 | −0.03 | 0.10 | −0.12 | −0.03 | −0.36 | −0.07 | −0.05 |
| 43 | 1 | −0.10 | 0.03 | −0.18 | −0.09 | −0.05 | −0.06 | −0.28 | −0.10 | −0.20 |
| 44 | 1 | −0.23 | 0.18 | 0.63 | −0.16 | 0.09 | 0.44 | 0.01 | −0.46 | 0.05 |

| | 28 | 29 | 30 |
|---|---|---|---|
| | ?A | ?A | ?A |
| Frequencies | 270.9643 | 285.6143 | 290.1637 |
| Red. masses | 4.7351 | 2.7792 | 1.4379 |
| Frc consts | 0.2048 | 0.1336 | 0.0713 |
| IR Inten | 6.6494 | 15.4608 | 111.2144 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.04 | 0.05 | −0.01 | 0.04 | 0.05 | −0.06 | 0.03 | 0.04 | 0.00 |
| 2 | 6 | 0.07 | 0.04 | −0.01 | 0.02 | 0.01 | −0.05 | 0.00 | 0.01 | 0.00 |
| 3 | 8 | −0.06 | −0.05 | −0.01 | −0.09 | −0.04 | −0.03 | −0.05 | −0.02 | 0.01 |
| 4 | 6 | −0.10 | −0.06 | −0.03 | −0.05 | −0.01 | −0.04 | −0.01 | 0.00 | −0.01 |
| 5 | 6 | −0.12 | 0.01 | −0.01 | 0.00 | 0.02 | −0.05 | 0.01 | 0.03 | −0.01 |
| 6 | 7 | 0.00 | −0.02 | −0.03 | 0.00 | 0.00 | 0.00 | 0.00 | 0.00 | 0.00 |
| 7 | 6 | 0.02 | 0.00 | −0.01 | 0.00 | −0.02 | −0.01 | 0.00 | 0.00 | 0.00 |
| 8 | 6 | 0.03 | 0.02 | 0.01 | 0.01 | −0.03 | −0.01 | 0.00 | −0.01 | 0.00 |
| 9 | 7 | 0.03 | 0.01 | 0.00 | 0.01 | 0.00 | 0.01 | 0.00 | 0.00 | 0.00 |
| 10 | 6 | 0.02 | −0.02 | −0.03 | 0.01 | 0.02 | 0.02 | 0.00 | 0.00 | 0.01 |
| 11 | 6 | 0.03 | 0.00 | 0.00 | 0.01 | −0.02 | −0.01 | 0.00 | 0.00 | 0.00 |
| 12 | 7 | 0.02 | −0.01 | −0.01 | 0.01 | −0.02 | −0.01 | 0.01 | 0.00 | 0.00 |
| 13 | 6 | 0.02 | 0.00 | −0.01 | 0.02 | 0.01 | 0.01 | 0.01 | 0.00 | 0.00 |
| 14 | 7 | 0.02 | 0.00 | −0.01 | 0.02 | 0.02 | 0.02 | 0.01 | 0.00 | 0.00 |
| 15 | 7 | 0.04 | −0.02 | 0.01 | 0.02 | 0.00 | 0.01 | 0.00 | 0.00 | 0.00 |
| 16 | 6 | 0.09 | −0.10 | 0.13 | 0.08 | −0.05 | 0.07 | 0.04 | −0.02 | 0.02 |
| 17 | 16 | −0.03 | −0.06 | −0.01 | −0.03 | −0.05 | 0.00 | −0.02 | −0.02 | 0.00 |
| 18 | 6 | −0.02 | 0.10 | 0.04 | −0.02 | 0.06 | 0.03 | −0.01 | 0.03 | 0.02 |
| 19 | 8 | −0.10 | 0.31 | 0.06 | 0.06 | −0.09 | 0.07 | 0.03 | −0.09 | 0.00 |
| 20 | 8 | 0.13 | −0.15 | −0.09 | −0.10 | 0.18 | −0.02 | −0.01 | 0.03 | −0.05 |
| 21 | 1 | 0.01 | 0.00 | −0.01 | 0.03 | 0.04 | 0.03 | 0.01 | 0.00 | 0.00 |
| 22 | 1 | 0.02 | −0.04 | −0.04 | 0.02 | 0.05 | 0.03 | 0.01 | 0.01 | 0.01 |
| 23 | 1 | 0.08 | 0.02 | 0.05 | 0.02 | 0.01 | −0.03 | −0.01 | 0.01 | 0.03 |
| 24 | 1 | −0.06 | 0.19 | 0.01 | 0.02 | 0.01 | −0.03 | 0.02 | 0.02 | 0.01 |
| 25 | 1 | −0.02 | 0.01 | −0.11 | −0.05 | 0.07 | −0.07 | −0.01 | 0.05 | −0.03 |
| 26 | 1 | −0.07 | 0.00 | −0.12 | −0.04 | 0.05 | −0.06 | −0.01 | 0.04 | −0.01 |
| 27 | 1 | 0.10 | −0.16 | −0.02 | −0.01 | 0.04 | −0.20 | −0.09 | 0.29 | 0.09 |
| 28 | 1 | 0.05 | −0.12 | 0.49 | 0.06 | −0.06 | 0.33 | 0.03 | −0.01 | 0.12 |
| 29 | 1 | 0.44 | −0.13 | 0.06 | 0.35 | −0.07 | 0.02 | 0.15 | −0.03 | 0.00 |
| 30 | 1 | −0.07 | 0.18 | 0.09 | −0.07 | 0.11 | 0.06 | −0.03 | 0.06 | 0.04 |
| 31 | 1 | 0.13 | 0.14 | 0.01 | 0.09 | 0.09 | 0.02 | 0.05 | 0.05 | 0.01 |
| 32 | 1 | −0.09 | 0.11 | 0.07 | −0.07 | 0.09 | 0.05 | −0.04 | 0.04 | 0.03 |
| 33 | 1 | −0.04 | 0.02 | 0.03 | 0.19 | −0.65 | 0.01 | 0.01 | 0.88 | 0.08 |
| 34 | 1 | 0.05 | −0.09 | −0.02 | 0.03 | 0.06 | 0.04 | 0.00 | 0.03 | 0.01 |
| 35 | 1 | 0.02 | −0.05 | 0.02 | 0.02 | 0.02 | 0.02 | 0.00 | 0.00 | −0.01 |
| 36 | 15 | −0.02 | −0.01 | −0.01 | 0.01 | 0.02 | 0.02 | −0.02 | −0.02 | −0.01 |
| 37 | 8 | −0.02 | −0.01 | 0.01 | 0.01 | 0.00 | 0.01 | −0.02 | −0.01 | −0.02 |
| 38 | 8 | 0.01 | 0.00 | 0.02 | 0.00 | −0.03 | −0.01 | 0.00 | 0.02 | 0.02 |
| 39 | 8 | 0.01 | 0.02 | 0.00 | 0.01 | −0.03 | −0.05 | 0.01 | 0.03 | 0.03 |
| 40 | 8 | 0.02 | −0.01 | −0.01 | −0.02 | 0.03 | 0.01 | 0.03 | −0.02 | −0.02 |
| 41 | 1 | 0.03 | 0.03 | 0.02 | 0.01 | −0.01 | 0.00 | 0.00 | −0.02 | 0.00 |
| 42 | 1 | 0.04 | 0.03 | 0.01 | −0.03 | 0.03 | 0.03 | 0.05 | −0.07 | −0.03 |
| 43 | 1 | 0.02 | 0.00 | 0.02 | −0.03 | 0.00 | 0.01 | 0.03 | −0.01 | 0.03 |
| 44 | 1 | 0.00 | 0.04 | −0.02 | −0.07 | 0.02 | 0.19 | 0.04 | 0.02 | −0.11 |

| | 31 | 32 | 33 |
|---|---|---|---|
| | ?A | ?A | ?A |
| Frequencies | 299.5965 | 303.2280 | 309.6838 |
| Red. masses | 4.8257 | 4.0539 | 3.7313 |
| Frc consts | 0.2552 | 0.2196 | 0.2108 |
| IR Inten | 184.2886 | 23.2311 | 225.1829 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.01 | 0.00 | 0.00 | 0.01 | 0.00 | −0.01 | −0.01 | −0.01 | 0.02 |
| 2 | 6 | 0.00 | 0.01 | 0.00 | 0.00 | 0.00 | −0.01 | −0.01 | −0.01 | 0.02 |
| 3 | 8 | −0.02 | 0.00 | 0.00 | −0.01 | −0.01 | −0.01 | 0.00 | 0.00 | 0.02 |
| 4 | 6 | −0.01 | 0.01 | 0.00 | 0.00 | 0.00 | −0.01 | 0.01 | 0.00 | 0.01 |
| 5 | 6 | 0.00 | 0.01 | 0.00 | 0.01 | 0.00 | −0.01 | 0.00 | 0.00 | 0.01 |
| 6 | 7 | 0.00 | 0.00 | 0.00 | 0.02 | 0.13 | 0.08 | −0.01 | −0.03 | −0.02 |
| 7 | 6 | 0.00 | −0.01 | −0.01 | 0.01 | 0.14 | 0.12 | −0.01 | −0.04 | −0.03 |
| 8 | 6 | 0.00 | −0.01 | −0.01 | 0.02 | 0.14 | 0.13 | −0.01 | −0.03 | −0.03 |
| 9 | 7 | 0.00 | 0.00 | 0.00 | 0.00 | −0.12 | −0.06 | 0.00 | 0.03 | 0.02 |
| 10 | 6 | 0.00 | 0.00 | 0.00 | 0.00 | −0.09 | −0.06 | 0.00 | 0.02 | 0.01 |
| 11 | 6 | 0.01 | 0.00 | 0.00 | 0.01 | 0.11 | 0.09 | −0.01 | −0.02 | −0.02 |
| 12 | 7 | 0.01 | 0.00 | 0.00 | 0.02 | 0.12 | 0.09 | −0.01 | −0.02 | −0.02 |
| 13 | 6 | 0.01 | 0.00 | 0.00 | −0.03 | −0.14 | −0.09 | 0.00 | 0.03 | 0.02 |
| 14 | 7 | 0.01 | 0.00 | 0.00 | −0.03 | −0.14 | −0.09 | 0.00 | 0.03 | 0.02 |
| 15 | 7 | 0.01 | 0.00 | 0.01 | −0.03 | −0.03 | −0.09 | 0.00 | 0.01 | 0.02 |
| 16 | 6 | −0.01 | 0.01 | 0.00 | 0.02 | −0.01 | 0.01 | −0.01 | 0.01 | −0.01 |
| 17 | 16 | 0.00 | 0.01 | 0.00 | −0.01 | −0.01 | 0.00 | 0.00 | 0.00 | 0.00 |
| 18 | 6 | 0.00 | −0.01 | 0.00 | 0.00 | 0.01 | 0.01 | 0.00 | 0.00 | 0.00 |
| 19 | 8 | 0.00 | 0.00 | 0.00 | 0.02 | −0.02 | 0.01 | −0.02 | 0.02 | −0.02 |
| 20 | 8 | −0.01 | −0.01 | −0.02 | 0.00 | 0.02 | 0.00 | 0.03 | −0.03 | 0.01 |
| 21 | 1 | 0.01 | 0.01 | 0.01 | −0.07 | −0.40 | −0.28 | 0.02 | 0.09 | 0.07 |
| 22 | 1 | 0.01 | 0.01 | 0.01 | −0.02 | −0.24 | −0.18 | 0.00 | 0.04 | 0.04 |
| 23 | 1 | 0.00 | 0.00 | 0.00 | 0.04 | 0.01 | −0.02 | −0.01 | −0.01 | 0.01 |
| 24 | 1 | −0.01 | 0.00 | 0.01 | 0.01 | 0.00 | −0.01 | 0.00 | 0.01 | 0.01 |
| 25 | 1 | 0.00 | 0.00 | 0.00 | 0.01 | 0.01 | −0.02 | 0.01 | −0.01 | 0.01 |
| 26 | 1 | −0.01 | 0.02 | 0.01 | 0.00 | 0.01 | −0.01 | 0.01 | 0.00 | 0.01 |
| 27 | 1 | −0.02 | 0.02 | 0.00 | 0.02 | −0.02 | −0.02 | 0.03 | −0.05 | 0.00 |
| 28 | 1 | −0.01 | 0.00 | −0.01 | 0.02 | 0.00 | 0.05 | 0.00 | 0.01 | −0.05 |
| 29 | 1 | −0.02 | 0.01 | 0.00 | 0.06 | −0.01 | 0.00 | −0.05 | 0.00 | 0.00 |
| 30 | 1 | 0.01 | −0.02 | −0.01 | −0.01 | 0.02 | 0.01 | 0.01 | −0.01 | 0.00 |
| 31 | 1 | −0.01 | −0.01 | 0.00 | 0.02 | 0.02 | 0.01 | −0.01 | −0.01 | 0.00 |
| 32 | 1 | 0.01 | −0.01 | −0.01 | −0.02 | 0.02 | 0.01 | 0.00 | −0.01 | 0.00 |
| 33 | 1 | −0.01 | 0.24 | 0.02 | 0.04 | 0.00 | 0.01 | −0.05 | 0.41 | 0.02 |
| 34 | 1 | 0.02 | 0.01 | 0.01 | −0.17 | −0.45 | −0.27 | 0.03 | 0.11 | 0.06 |
| 35 | 1 | 0.01 | −0.01 | 0.02 | −0.04 | −0.09 | −0.14 | 0.00 | 0.02 | 0.02 |
| 36 | 15 | −0.01 | 0.15 | −0.05 | −0.02 | 0.01 | 0.03 | −0.05 | 0.03 | 0.11 |
| 37 | 8 | 0.07 | −0.26 | 0.11 | 0.02 | −0.02 | −0.04 | 0.08 | −0.05 | −0.19 |
| 38 | 8 | 0.11 | −0.12 | −0.11 | −0.01 | −0.04 | 0.01 | −0.05 | −0.14 | 0.04 |
| 39 | 8 | −0.15 | 0.07 | −0.06 | 0.02 | −0.01 | −0.06 | 0.07 | −0.05 | −0.19 |
| 40 | 8 | −0.02 | −0.06 | 0.19 | 0.01 | 0.04 | 0.01 | 0.03 | 0.15 | 0.01 |
| 41 | 1 | 0.00 | 0.00 | 0.00 | 0.01 | −0.08 | −0.02 | −0.01 | 0.01 | 0.00 |
| 42 | 1 | 0.00 | 0.57 | 0.07 | −0.04 | 0.07 | 0.05 | −0.15 | 0.16 | 0.16 |
| 43 | 1 | 0.13 | 0.49 | −0.32 | 0.00 | 0.01 | 0.06 | −0.02 | −0.05 | 0.27 |
| 44 | 1 | −0.10 | −0.07 | −0.04 | −0.07 | 0.05 | 0.18 | −0.23 | 0.18 | 0.61 |

| | 34 | 35 | 36 |
|---|---|---|---|
| | ?A | ?A | ?A |
| Frequencies | 343.5123 | 349.6857 | 392.7984 |
| Red. masses | 3.7647 | 4.2560 | 7.9902 |
| Frc consts | 0.2617 | 0.3066 | 0.7263 |
| IR Inten | 4.7994 | 2.7297 | 9.3139 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.06 | −0.04 | −0.04 | −0.07 | −0.02 | −0.03 | −0.10 | 0.01 | −0.02 |
| 2 | 6 | −0.01 | −0.04 | −0.04 | 0.05 | −0.03 | −0.02 | −0.10 | 0.12 | −0.05 |
| 3 | 8 | 0.10 | 0.01 | −0.09 | 0.21 | 0.01 | −0.10 | 0.25 | 0.33 | −0.06 |
| 4 | 6 | −0.07 | −0.06 | −0.06 | −0.08 | −0.08 | −0.06 | 0.09 | 0.15 | 0.08 |
| 5 | 6 | −0.07 | −0.05 | −0.06 | −0.11 | −0.05 | −0.06 | −0.05 | 0.04 | 0.10 |
| 6 | 7 | −0.03 | −0.01 | 0.07 | 0.04 | 0.01 | −0.06 | 0.01 | −0.02 | 0.01 |
| 7 | 6 | 0.04 | 0.03 | −0.02 | −0.03 | −0.02 | 0.00 | 0.00 | −0.01 | 0.00 |
| 8 | 6 | 0.02 | 0.07 | −0.07 | −0.02 | −0.05 | 0.05 | −0.01 | 0.01 | −0.02 |
| 9 | 7 | −0.11 | 0.04 | −0.06 | 0.10 | −0.02 | 0.04 | −0.03 | 0.02 | −0.01 |
| 10 | 6 | −0.12 | 0.00 | 0.03 | 0.10 | 0.00 | −0.04 | −0.03 | −0.01 | 0.00 |
| 11 | 6 | 0.02 | 0.04 | −0.04 | −0.02 | −0.03 | 0.03 | −0.02 | 0.01 | −0.01 |
| 12 | 7 | −0.02 | 0.05 | −0.03 | −0.01 | −0.04 | 0.03 | −0.02 | 0.01 | −0.01 |
| 13 | 6 | 0.01 | 0.00 | −0.03 | −0.03 | 0.00 | 0.02 | −0.01 | 0.00 | 0.00 |
| 14 | 7 | 0.02 | 0.00 | −0.04 | −0.04 | 0.01 | 0.03 | −0.01 | 0.00 | 0.00 |
| 15 | 7 | 0.16 | −0.13 | 0.15 | −0.15 | 0.12 | −0.13 | 0.00 | −0.01 | 0.01 |
| 16 | 6 | −0.04 | 0.00 | 0.06 | −0.05 | −0.01 | 0.09 | 0.13 | −0.03 | −0.16 |
| 17 | 16 | 0.02 | 0.05 | 0.00 | 0.03 | 0.05 | 0.00 | −0.04 | −0.27 | −0.06 |
| 18 | 6 | 0.02 | −0.02 | −0.01 | 0.03 | −0.02 | −0.01 | −0.03 | 0.04 | 0.03 |
| 19 | 8 | 0.02 | −0.02 | 0.11 | 0.00 | −0.01 | 0.16 | −0.04 | −0.05 | 0.14 |
| 20 | 8 | 0.00 | 0.05 | 0.10 | −0.01 | 0.05 | 0.09 | 0.02 | −0.02 | 0.05 |
| 21 | 1 | 0.02 | −0.02 | −0.04 | −0.04 | 0.03 | 0.04 | −0.01 | 0.01 | 0.01 |
| 22 | 1 | −0.19 | −0.02 | 0.07 | 0.15 | 0.02 | −0.07 | −0.06 | −0.02 | 0.02 |
| 23 | 1 | −0.01 | −0.03 | −0.06 | 0.09 | −0.03 | −0.02 | −0.15 | 0.15 | −0.16 |
| 24 | 1 | −0.04 | 0.02 | −0.08 | −0.06 | 0.05 | −0.05 | −0.05 | 0.01 | −0.11 |
| 25 | 1 | −0.01 | −0.04 | −0.14 | −0.05 | −0.03 | −0.17 | −0.06 | 0.00 | 0.19 |
| 26 | 1 | −0.05 | −0.13 | −0.14 | −0.05 | −0.22 | −0.19 | 0.09 | 0.16 | 0.06 |
| 27 | 1 | −0.01 | −0.03 | 0.14 | −0.01 | −0.03 | 0.11 | 0.01 | −0.10 | 0.11 |
| 28 | 1 | −0.05 | −0.05 | 0.14 | −0.06 | −0.08 | 0.20 | 0.14 | 0.01 | −0.18 |
| 29 | 1 | 0.05 | 0.04 | 0.04 | 0.08 | 0.02 | 0.06 | 0.11 | −0.18 | −0.16 |
| 30 | 1 | 0.06 | −0.05 | −0.02 | 0.07 | −0.05 | −0.02 | −0.16 | 0.18 | 0.09 |
| 31 | 1 | −0.06 | −0.04 | −0.02 | −0.05 | −0.04 | −0.03 | 0.32 | 0.13 | 0.04 |
| 32 | 1 | 0.05 | −0.06 | −0.02 | 0.05 | −0.06 | −0.02 | −0.17 | 0.15 | 0.08 |
| 33 | 1 | 0.16 | 0.28 | 0.11 | 0.19 | 0.27 | 0.15 | 0.01 | −0.02 | 0.13 |
| 34 | 1 | 0.36 | −0.34 | 0.05 | −0.33 | 0.29 | −0.05 | 0.02 | −0.02 | 0.01 |
| 35 | 1 | 0.03 | −0.25 | 0.34 | −0.04 | 0.22 | −0.30 | −0.02 | −0.02 | 0.03 |
| 36 | 15 | 0.00 | 0.00 | 0.00 | −0.01 | 0.00 | 0.00 | 0.00 | 0.00 | 0.00 |
| 37 | 8 | 0.00 | 0.00 | −0.01 | 0.00 | −0.01 | 0.00 | 0.01 | −0.02 | 0.01 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | −0.01 | 0.01 | 0.00 | −0.01 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.18 | 0.09 | −0.08 | 0.16 | −0.06 | 0.07 | −0.04 | 0.00 | −0.03 |
| 42 | 1 | 0.00 | −0.02 | −0.01 | 0.01 | −0.01 | −0.01 | 0.00 | 0.02 | 0.00 |
| 43 | 1 | 0.00 | 0.00 | 0.01 | 0.01 | 0.01 | 0.00 | 0.01 | 0.02 | −0.02 |
| 44 | 1 | 0.00 | 0.00 | −0.01 | 0.00 | 0.00 | −0.02 | 0.00 | −0.01 | −0.01 |

| | 37 | 38 | 39 |
|---|---|---|---|
| | ?A | ?A | ?A |
| Frequencies | 443.7145 | 476.9207 | 480.1723 |
| Red. masses | 1.4745 | 2.1230 | 1.6019 |
| Frc consts | 0.1710 | 0.2845 | 0.2176 |
| IR Inten | 101.7497 | 118.5950 | 184.3847 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.03 | −0.07 | −0.04 | −0.02 | 0.05 | 0.03 |
| 2 | 6 | 0.00 | 0.00 | 0.00 | 0.06 | 0.03 | −0.10 | −0.04 | −0.02 | 0.07 |
| 3 | 8 | 0.00 | 0.00 | 0.00 | −0.03 | 0.07 | −0.01 | 0.02 | −0.04 | 0.01 |
| 4 | 6 | 0.00 | 0.00 | −0.01 | −0.05 | −0.02 | 0.09 | 0.03 | 0.01 | −0.06 |
| 5 | 6 | 0.00 | 0.00 | −0.01 | 0.04 | −0.06 | 0.05 | −0.03 | 0.04 | −0.03 |
| 6 | 7 | 0.01 | 0.03 | 0.03 | −0.01 | −0.01 | −0.03 | 0.01 | −0.03 | −0.01 |
| 7 | 6 | 0.00 | 0.00 | 0.00 | −0.01 | 0.01 | 0.01 | 0.01 | 0.01 | 0.00 |
| 8 | 6 | −0.01 | −0.03 | −0.02 | 0.00 | 0.00 | 0.01 | 0.00 | 0.01 | −0.01 |
| 9 | 7 | 0.02 | 0.13 | 0.10 | 0.01 | −0.02 | −0.01 | −0.01 | −0.01 | −0.03 |
| 10 | 6 | −0.01 | −0.06 | −0.04 | 0.02 | 0.02 | 0.00 | −0.01 | 0.01 | 0.01 |
| 11 | 6 | 0.00 | −0.01 | −0.01 | 0.01 | 0.00 | 0.01 | −0.01 | 0.01 | 0.01 |
| 12 | 7 | 0.00 | 0.01 | 0.00 | 0.02 | −0.01 | 0.01 | 0.00 | 0.00 | 0.01 |
| 13 | 6 | 0.00 | −0.01 | −0.01 | 0.02 | −0.03 | −0.02 | −0.01 | −0.03 | −0.01 |
| 14 | 7 | 0.00 | −0.01 | 0.00 | 0.02 | 0.03 | 0.02 | 0.01 | 0.03 | 0.02 |
| 15 | 7 | −0.01 | −0.01 | −0.01 | −0.02 | −0.05 | −0.03 | −0.05 | −0.06 | −0.03 |
| 16 | 6 | 0.00 | 0.00 | 0.00 | −0.06 | −0.04 | 0.08 | 0.04 | 0.03 | −0.05 |
| 17 | 16 | 0.00 | 0.00 | 0.00 | 0.01 | −0.02 | −0.01 | 0.00 | 0.02 | 0.01 |
| 18 | 6 | 0.00 | 0.00 | 0.00 | 0.01 | −0.01 | 0.00 | −0.01 | 0.01 | 0.00 |
| 19 | 8 | 0.00 | 0.00 | 0.00 | −0.03 | 0.05 | −0.08 | 0.01 | −0.03 | 0.06 |
| 20 | 8 | 0.00 | 0.00 | 0.00 | −0.02 | 0.08 | 0.07 | 0.01 | −0.05 | −0.04 |
| 21 | 1 | 0.00 | −0.02 | −0.01 | −0.01 | −0.09 | −0.07 | −0.02 | −0.11 | −0.07 |
| 22 | 1 | −0.02 | −0.10 | −0.07 | 0.04 | 0.03 | −0.01 | −0.02 | 0.01 | 0.02 |
| 23 | 1 | 0.01 | 0.00 | 0.00 | 0.04 | 0.06 | −0.21 | −0.04 | −0.05 | 0.14 |
| 24 | 1 | 0.00 | 0.00 | 0.00 | 0.05 | −0.12 | −0.07 | −0.03 | 0.08 | 0.04 |
| 25 | 1 | 0.00 | 0.00 | −0.01 | 0.08 | −0.12 | 0.10 | −0.06 | 0.08 | −0.06 |
| 26 | 1 | 0.00 | 0.00 | −0.01 | −0.07 | −0.03 | 0.18 | 0.05 | 0.02 | −0.12 |
| 27 | 1 | 0.00 | 0.00 | 0.01 | 0.03 | −0.04 | −0.01 | −0.02 | 0.03 | 0.01 |
| 28 | 1 | 0.00 | 0.00 | 0.00 | −0.06 | −0.08 | 0.08 | 0.04 | 0.05 | −0.06 |
| 29 | 1 | 0.01 | 0.01 | 0.00 | −0.06 | −0.09 | 0.08 | 0.04 | 0.06 | −0.05 |
| 30 | 1 | 0.00 | 0.00 | 0.00 | 0.01 | −0.01 | −0.01 | −0.01 | 0.01 | 0.01 |
| 31 | 1 | 0.00 | 0.00 | 0.00 | 0.02 | −0.01 | 0.00 | −0.01 | 0.01 | 0.00 |
| 32 | 1 | 0.00 | 0.00 | 0.00 | 0.01 | 0.00 | 0.00 | −0.01 | 0.00 | 0.00 |
| 33 | 1 | 0.01 | −0.02 | 0.00 | −0.18 | 0.19 | −0.05 | 0.11 | −0.11 | 0.04 |
| 34 | 1 | 0.01 | −0.19 | −0.09 | 0.07 | −0.09 | −0.04 | 0.05 | −0.08 | −0.03 |
| 35 | 1 | 0.05 | 0.27 | 0.20 | 0.13 | 0.62 | 0.45 | 0.13 | 0.69 | 0.49 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.11 | −0.70 | −0.53 | 0.05 | 0.15 | 0.13 | 0.03 | 0.25 | 0.17 |
| 42 | 1 | 0.00 | 0.00 | 0.00 | 0.00 | −0.01 | 0.00 | 0.00 | 0.01 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 40 | 41 | 42 |
|---|---|---|---|
| | ?A | ?A | ?A |
| Frequencies | 534.9542 | 539.8588 | 562.8280 |
| Red. masses | 2.6240 | 3.9225 | 4.6899 |
| Frc consts | 0.4424 | 0.6736 | 0.8753 |
| IR Inten | 62.7802 | 114.9418 | 26.9137 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.04 | −0.04 | 0.01 | −0.08 | −0.08 | 0.01 | −0.04 | −0.06 | −0.01 |
| 2 | 6 | 0.06 | 0.03 | −0.02 | 0.13 | 0.05 | −0.04 | 0.11 | 0.05 | −0.04 |
| 3 | 8 | −0.06 | −0.01 | 0.01 | −0.12 | −0.02 | 0.02 | −0.10 | −0.02 | 0.02 |
| 4 | 6 | 0.02 | 0.03 | −0.03 | 0.06 | 0.06 | −0.06 | 0.03 | 0.04 | −0.03 |
| 5 | 6 | −0.02 | −0.02 | −0.02 | −0.07 | −0.06 | −0.04 | −0.04 | −0.05 | −0.02 |
| 6 | 7 | 0.00 | 0.13 | 0.07 | −0.09 | −0.03 | −0.08 | 0.04 | −0.01 | −0.02 |
| 7 | 6 | −0.03 | 0.00 | 0.01 | −0.11 | 0.00 | 0.02 | 0.13 | 0.03 | −0.08 |
| 8 | 6 | 0.00 | 0.03 | 0.05 | −0.02 | −0.09 | 0.06 | 0.03 | 0.04 | −0.14 |
| 9 | 7 | 0.02 | 0.01 | 0.03 | 0.04 | −0.08 | 0.08 | −0.04 | 0.11 | −0.11 |
| 10 | 6 | 0.00 | −0.09 | −0.07 | 0.06 | 0.04 | 0.02 | −0.06 | 0.03 | −0.04 |
| 11 | 6 | 0.00 | −0.07 | −0.06 | 0.07 | 0.06 | −0.02 | −0.09 | −0.07 | 0.13 |
| 12 | 7 | 0.01 | −0.07 | −0.06 | 0.04 | 0.07 | −0.02 | 0.08 | −0.08 | 0.16 |
| 13 | 6 | 0.04 | 0.13 | 0.07 | 0.00 | −0.02 | −0.13 | 0.06 | −0.13 | 0.09 |
| 14 | 7 | −0.02 | −0.13 | −0.10 | −0.03 | 0.08 | 0.07 | 0.18 | 0.06 | −0.05 |
| 15 | 7 | 0.01 | 0.00 | 0.01 | 0.11 | −0.01 | −0.03 | −0.17 | 0.01 | 0.06 |
| 16 | 6 | 0.04 | 0.02 | −0.05 | 0.09 | 0.06 | −0.12 | 0.05 | 0.03 | −0.07 |
| 17 | 16 | 0.00 | 0.00 | 0.00 | −0.01 | 0.01 | 0.01 | −0.01 | 0.01 | 0.01 |
| 18 | 6 | −0.01 | 0.00 | 0.00 | −0.02 | 0.01 | 0.00 | −0.01 | 0.00 | 0.00 |
| 19 | 8 | 0.00 | −0.01 | 0.02 | −0.01 | −0.02 | 0.07 | −0.01 | 0.00 | 0.04 |
| 20 | 8 | −0.02 | −0.02 | 0.06 | −0.04 | −0.03 | 0.13 | −0.03 | 0.00 | 0.08 |
| 21 | 1 | 0.10 | 0.46 | 0.31 | 0.00 | −0.21 | −0.27 | −0.13 | −0.20 | 0.04 |
| 22 | 1 | −0.01 | −0.33 | −0.27 | 0.20 | 0.21 | 0.05 | −0.14 | 0.01 | 0.00 |
| 23 | 1 | 0.10 | 0.05 | −0.06 | 0.08 | 0.06 | −0.09 | 0.14 | 0.07 | −0.12 |
| 24 | 1 | −0.05 | −0.01 | 0.04 | −0.11 | 0.00 | 0.08 | −0.06 | −0.02 | 0.04 |
| 25 | 1 | 0.01 | −0.05 | −0.03 | 0.01 | −0.11 | −0.05 | 0.02 | −0.09 | −0.02 |
| 26 | 1 | 0.02 | 0.10 | −0.04 | 0.07 | 0.22 | −0.09 | 0.03 | 0.16 | −0.04 |
| 27 | 1 | 0.04 | −0.20 | −0.03 | 0.09 | −0.43 | −0.07 | 0.11 | −0.40 | −0.16 |
| 28 | 1 | 0.04 | 0.04 | −0.08 | 0.10 | 0.12 | −0.20 | 0.06 | 0.07 | −0.12 |
| 29 | 1 | 0.01 | 0.04 | −0.04 | 0.01 | 0.09 | −0.10 | 0.00 | 0.05 | −0.06 |
| 30 | 1 | −0.01 | 0.01 | 0.00 | −0.03 | 0.02 | 0.01 | −0.02 | 0.01 | 0.00 |
| 31 | 1 | 0.00 | 0.01 | 0.00 | 0.00 | 0.01 | 0.01 | 0.00 | 0.01 | 0.00 |
| 32 | 1 | −0.01 | 0.01 | 0.00 | −0.02 | 0.02 | 0.00 | −0.01 | 0.01 | 0.00 |
| 33 | 1 | 0.05 | 0.09 | 0.02 | 0.11 | 0.20 | 0.07 | 0.05 | 0.20 | 0.04 |
| 34 | 1 | 0.09 | 0.11 | 0.06 | 0.15 | −0.04 | −0.04 | −0.36 | −0.08 | 0.02 |
| 35 | 1 | 0.06 | 0.23 | 0.18 | 0.06 | −0.12 | −0.03 | −0.15 | −0.14 | −0.19 |
| 36 | 15 | 0.00 | 0.00 | 0.00 | 0.00 | 0.00 | 0.00 | 0.00 | 0.00 | 0.00 |
| 37 | 8 | 0.00 | 0.01 | 0.01 | −0.01 | 0.01 | 0.01 | −0.01 | 0.01 | 0.01 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.08 | 0.29 | 0.26 | 0.08 | −0.22 | 0.03 | −0.11 | 0.14 | −0.15 |
| 42 | 1 | 0.00 | −0.01 | 0.00 | 0.01 | −0.02 | −0.01 | 0.01 | −0.02 | −0.01 |
| 43 | 1 | 0.00 | 0.00 | 0.00 | 0.00 | −0.01 | 0.00 | 0.00 | −0.01 | 0.00 |
| 44 | 1 | 0.00 | 0.00 | 0.00 | 0.01 | 0.00 | −0.01 | 0.00 | 0.00 | −0.01 |

| | 43 | 44 | 45 |
|---|---|---|---|
| | ?A | ?A | ?A |
| Frequencies | 572.9814 | 602.4191 | 607.0140 |
| Red. masses | 2.5309 | 2.8878 | 4.5495 |
| Frc consts | 0.4896 | 0.6175 | 0.9877 |
| IR Inten | 3.5241 | 4.9829 | 6.0644 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.03 | 0.01 | −0.09 | −0.03 | 0.00 | 0.10 |
| 2 | 6 | 0.01 | −0.01 | −0.01 | 0.02 | −0.11 | −0.07 | −0.04 | 0.08 | 0.08 |
| 3 | 8 | −0.01 | −0.01 | 0.00 | 0.01 | −0.08 | −0.03 | 0.04 | 0.08 | 0.00 |
| 4 | 6 | 0.01 | 0.00 | 0.00 | 0.15 | −0.03 | 0.06 | −0.12 | 0.01 | −0.07 |
| 5 | 6 | −0.01 | −0.01 | 0.00 | −0.07 | −0.04 | 0.08 | 0.06 | 0.04 | −0.07 |
| 6 | 7 | −0.01 | −0.08 | −0.06 | −0.04 | 0.06 | −0.04 | −0.10 | 0.08 | −0.12 |
| 7 | 6 | 0.01 | 0.01 | 0.00 | −0.07 | 0.02 | 0.01 | −0.15 | 0.01 | 0.00 |
| 8 | 6 | 0.03 | 0.18 | 0.12 | −0.06 | −0.02 | 0.02 | −0.13 | −0.01 | 0.06 |
| 9 | 7 | −0.02 | −0.10 | −0.08 | 0.00 | 0.00 | 0.01 | 0.00 | −0.02 | 0.02 |
| 10 | 6 | 0.02 | 0.11 | 0.07 | 0.00 | 0.03 | −0.05 | 0.01 | 0.07 | −0.10 |
| 11 | 6 | 0.00 | 0.05 | 0.05 | −0.05 | 0.00 | 0.00 | −0.11 | 0.02 | 0.02 |
| 12 | 7 | −0.01 | −0.11 | −0.06 | 0.07 | 0.00 | 0.00 | 0.16 | −0.01 | −0.01 |
| 13 | 6 | 0.02 | 0.07 | 0.07 | 0.08 | −0.01 | −0.01 | 0.17 | −0.02 | −0.03 |
| 14 | 7 | 0.00 | −0.07 | −0.06 | 0.06 | −0.01 | 0.00 | 0.14 | −0.02 | 0.00 |
| 15 | 7 | −0.02 | −0.01 | −0.01 | −0.03 | −0.02 | 0.05 | −0.07 | −0.06 | 0.11 |
| 16 | 6 | 0.01 | 0.01 | −0.01 | 0.05 | 0.00 | −0.02 | −0.05 | 0.00 | 0.02 |
| 17 | 16 | 0.00 | 0.00 | 0.00 | −0.01 | 0.02 | 0.00 | 0.01 | −0.02 | 0.00 |
| 18 | 6 | 0.00 | 0.00 | 0.00 | −0.01 | 0.01 | 0.00 | 0.01 | −0.01 | 0.00 |
| 19 | 8 | −0.01 | 0.00 | 0.01 | −0.12 | 0.04 | 0.09 | 0.09 | −0.03 | −0.08 |
| 20 | 8 | 0.00 | 0.00 | 0.00 | 0.03 | 0.08 | −0.05 | −0.01 | −0.10 | 0.05 |
| 21 | 1 | 0.05 | 0.29 | 0.22 | 0.07 | −0.02 | −0.02 | 0.15 | −0.03 | −0.04 |
| 22 | 1 | 0.07 | 0.40 | 0.27 | 0.05 | 0.03 | −0.09 | 0.14 | 0.13 | −0.15 |
| 23 | 1 | −0.01 | −0.01 | 0.00 | 0.02 | −0.12 | −0.03 | −0.07 | 0.07 | 0.10 |
| 24 | 1 | 0.00 | 0.00 | −0.01 | 0.06 | 0.02 | −0.17 | −0.03 | −0.05 | 0.12 |
| 25 | 1 | −0.01 | −0.01 | 0.01 | −0.11 | −0.06 | 0.19 | 0.06 | 0.09 | −0.17 |
| 26 | 1 | 0.01 | 0.02 | −0.01 | 0.15 | 0.02 | 0.03 | −0.12 | −0.07 | −0.05 |
| 27 | 1 | 0.02 | −0.05 | −0.04 | −0.18 | 0.60 | 0.32 | −0.16 | 0.26 | 0.31 |
| 28 | 1 | 0.01 | 0.02 | −0.03 | 0.08 | 0.12 | −0.28 | −0.08 | −0.10 | 0.25 |
| 29 | 1 | −0.01 | 0.01 | −0.01 | −0.22 | −0.01 | 0.03 | 0.19 | 0.01 | −0.02 |
| 30 | 1 | 0.00 | 0.00 | 0.00 | −0.01 | 0.01 | 0.00 | 0.02 | −0.01 | 0.00 |
| 31 | 1 | 0.00 | 0.00 | 0.00 | −0.01 | 0.01 | 0.00 | 0.01 | −0.01 | 0.00 |
| 32 | 1 | 0.00 | 0.00 | 0.00 | −0.01 | 0.01 | 0.00 | 0.01 | −0.01 | 0.00 |
| 33 | 1 | 0.00 | 0.03 | 0.01 | −0.11 | −0.12 | 0.08 | 0.09 | −0.22 | −0.09 |
| 34 | 1 | −0.03 | −0.09 | −0.03 | 0.04 | −0.05 | 0.04 | 0.07 | −0.12 | 0.08 |
| 35 | 1 | 0.02 | 0.15 | 0.10 | −0.08 | −0.11 | 0.07 | −0.16 | −0.22 | 0.16 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.01 | −0.01 | −0.02 | 0.01 | −0.01 | −0.0.1 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.09 | −0.52 | −0.41 | 0.05 | −0.03 | 0.04 | 0.11 | −0.10 | 0.06 |
| 42 | 1 | 0.00 | 0.00 | 0.00 | 0.00 | 0.01 | 0.00 | 0.00 | 0.02 | 0.01 |
| 43 | 1 | 0.00 | 0.00 | 0.00 | 0.00 | 0.01 | 0.00 | 0.00 | 0.00 | 0.01 |
| 44 | 1 | 0.00 | 0.00 | 0.00 | 0.00 | 0.01 | 0.00 | 0.00 | 0.01 | 0.00 |

| | 46 | 47 | 48 |
|---|---|---|---|
| | ?A | ?A | ?A |
| Frequencies | 623.9326 | 656.5955 | 699.4724 |
| Red. masses | 1.4868 | 5.8027 | 9.3357 |
| Frc consts | 0.3410 | 1.4739 | 2.6912 |
| IR Inten | 81.1978 | 3.1945 | 2.2342 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.02 | −0.04 | 0.02 | 0.00 | 0.05 | 0.00 | 0.00 | 0.01 | −0.01 |
| 2 | 6 | 0.06 | 0.05 | −0.02 | −0.09 | −0.05 | 0.02 | −0.01 | 0.00 | 0.00 |
| 3 | 8 | −0.04 | 0.02 | 0.00 | 0.06 | 0.00 | −0.03 | 0.00 | −0.01 | −0.02 |
| 4 | 6 | −0.03 | 0.02 | −0.03 | 0.03 | −0.03 | 0.02 | 0.01 | −0.01 | 0.01 |
| 5 | 6 | 0.00 | −0.02 | −0.03 | 0.02 | 0.03 | 0.03 | 0.01 | 0.01 | 0.01 |
| 6 | 7 | 0.00 | −0.02 | 0.02 | −0.19 | 0.06 | −0.01 | 0.03 | 0.19 | 0.13 |
| 7 | 6 | 0.04 | 0.00 | −0.03 | −0.05 | 0.11 | −0.12 | −0.08 | −0.39 | −0.28 |
| 8 | 6 | 0.05 | 0.01 | −0.01 | 0.08 | −0.07 | 0.07 | −0.01 | −0.02 | −0.01 |
| 9 | 7 | −0.01 | 0.00 | 0.00 | −0.11 | −0.07 | 0.12 | −0.01 | −0.01 | 0.00 |
| 10 | 6 | −0.01 | −0.01 | 0.03 | −0.14 | 0.01 | 0.02 | −0.01 | −0.05 | −0.04 |
| 11 | 6 | 0.05 | −0.02 | 0.02 | 0.18 | −0.14 | 0.12 | 0.08 | 0.48 | 0.35 |
| 12 | 7 | −0.02 | −0.03 | 0.03 | 0.18 | −0.15 | 0.16 | −0.03 | −0.18 | −0.13 |
| 13 | 6 | −0.04 | 0.01 | 0.00 | −0.03 | 0.08 | −0.10 | 0.01 | 0.01 | 0.01 |
| 14 | 7 | −0.02 | 0.03 | −0.03 | 0.02 | 0.13 | −0.19 | 0.02 | 0.10 | 0.08 |
| 15 | 7 | 0.02 | 0.02 | −0.04 | 0.08 | 0.02 | −0.05 | 0.00 | −0.09 | −0.06 |
| 16 | 6 | 0.01 | 0.01 | −0.02 | −0.02 | −0.01 | 0.03 | 0.00 | −0.01 | 0.01 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.01 | 0.00 | −0.01 | 0.00 | −0.01 | 0.00 | 0.00 | 0.00 | 0.00 |
| 20 | 8 | 0.01 | −0.04 | 0.06 | 0.01 | 0.01 | −0.04 | 0.00 | 0.01 | −0.01 |
| 21 | 1 | −0.09 | 0.02 | 0.01 | −0.36 | 0.13 | −0.05 | 0.02 | 0.07 | 0.05 |
| 22 | 1 | −0.03 | −0.01 | 0.04 | −0.01 | 0.04 | −0.07 | −0.05 | −0.25 | −0.19 |
| 23 | 1 | 0.06 | 0.06 | −0.07 | −0.24 | −0.10 | 0.15 | 0.07 | 0.01 | 0.00 |
| 24 | 1 | −0.01 | −0.05 | 0.02 | 0.01 | 0.04 | −0.02 | 0.00 | 0.01 | −0.01 |
| 25 | 1 | 0.02 | −0.03 | −0.04 | −0.02 | 0.06 | 0.03 | 0.00 | 0.01 | 0.02 |
| 26 | 1 | −0.02 | 0.06 | −0.02 | 0.02 | −0.09 | 0.03 | 0.01 | −0.01 | 0.02 |
| 27 | 1 | −0.29 | 0.64 | 0.58 | 0.11 | −0.22 | −0.23 | 0.02 | −0.03 | −0.02 |
| 28 | 1 | 0.00 | 0.00 | 0.02 | −0.01 | 0.00 | 0.01 | 0.01 | 0.00 | −0.02 |
| 29 | 1 | 0.05 | 0.02 | −0.03 | −0.04 | −0.02 | 0.04 | −0.03 | −0.01 | 0.01 |
| 30 | 1 | 0.00 | 0.00 | 0.00 | 0.01 | 0.00 | 0.00 | 0.00 | 0.00 | 0.00 |
| 31 | 1 | 0.00 | 0.00 | 0.00 | 0.00 | 0.00 | 0.00 | 0.00 | 0.00 | 0.00 |
| 32 | 1 | 0.00 | 0.00 | 0.00 | 0.00 | 0.00 | 0.00 | 0.00 | 0.00 | 0.00 |
| 33 | 1 | 0.03 | −0.25 | −0.03 | −0.02 | 0.06 | 0.01 | −0.01 | 0.01 | 0.00 |
| 34 | 1 | −0.06 | 0.03 | −0.03 | −0.18 | 0.03 | −0.04 | −0.13 | −0.26 | −0.13 |
| 35 | 1 | 0.07 | 0.09 | −0.08 | 0.23 | 0.20 | −0.22 | 0.00 | −0.10 | −0.07 |
| 36 | 15 | 0.00 | 0.00 | 0.00 | 0.00 | 0.00 | 0.00 | 0.00 | 0.00 | 0.00 |
| 37 | 8 | 0.01 | −0.01 | −0.02 | −0.01 | 0.00 | 0.00 | 0.00 | 0.00 | 0.00 |
| 38 | 8 | −0.01 | −0.01 | 0.01 | 0.01 | 0.00 | −0.01 | 0.00 | 0.00 | 0.00 |
| 39 | 8 | 0.00 | 0.01 | −0.01 | 0.00 | −0.01 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.05 | 0.02 | −0.02 | −0.18 | −0.03 | 0.10 | 0.02 | 0.14 | 0.11 |
| 42 | 1 | 0.00 | 0.02 | 0.01 | 0.00 | −0.01 | 0.00 | 0.00 | 0.00 | 0.00 |
| 43 | 1 | 0.01 | 0.01 | 0.01 | 0.00 | 0.00 | 0.00 | 0.00 | 0.00 | 0.00 |
| 44 | 1 | −0.01 | 0.01 | 0.00 | 0.01 | −0.01 | 0.00 | 0.00 | 0.00 | 0.00 |

| | 49 | 50 | 51 |
|---|---|---|---|
| | ?A | ?A | ?A |
| Frequencies | 773.0299 | 798.3159 | 800.6097 |
| Red. masses | 5.7086 | 11.2257 | 10.3867 |
| Frc consts | 2.0099 | 4.2151 | 3.9226 |
| IR Inten | 10.9174 | 12.1597 | 46.8450 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.15 | −0.13 | −0.12 | −0.04 | 0.00 | −0.04 | 0.00 | 0.01 | 0.02 |
| 2 | 6 | 0.02 | −0.23 | 0.04 | 0.00 | −0.04 | 0.00 | 0.00 | 0.02 | 0.00 |
| 3 | 8 | 0.08 | 0.09 | 0.36 | 0.02 | −0.01 | 0.04 | −0.01 | 0.00 | −0.02 |
| 4 | 6 | −0.15 | 0.14 | −0.01 | −0.01 | 0.02 | 0.02 | 0.01 | −0.01 | −0.01 |
| 5 | 6 | −0.11 | −0.08 | −0.14 | 0.00 | 0.04 | 0.00 | 0.00 | 0.00 | 0.01 |
| 6 | 7 | 0.00 | 0.02 | 0.00 | 0.01 | 0.01 | −0.01 | −0.02 | −0.13 | −0.10 |
| 7 | 6 | −0.01 | 0.01 | 0.00 | −0.01 | −0.02 | −0.01 | 0.07 | 0.42 | 0.31 |
| 8 | 6 | −0.01 | −0.03 | 0.00 | 0.00 | 0.01 | 0.02 | −0.07 | −0.40 | −0.29 |
| 9 | 7 | −0.02 | −0.01 | 0.02 | −0.02 | −0.01 | 0.01 | 0.01 | 0.05 | 0.04 |
| 10 | 6 | −0.02 | 0.01 | −0.01 | −0.02 | 0.01 | −0.01 | 0.00 | 0.03 | 0.01 |
| 11 | 6 | 0.01 | 0.02 | 0.02 | 0.01 | −0.02 | −0.01 | 0.06 | 0.33 | 0.24 |
| 12 | 7 | 0.02 | −0.01 | 0.00 | 0.01 | 0.00 | 0.01 | −0.03 | −0.16 | −0.12 |
| 13 | 6 | 0.00 | 0.02 | −0.01 | 0.00 | 0.00 | 0.00 | 0.02 | 0.10 | 0.08 |
| 14 | 7 | 0.00 | 0.00 | −0.01 | 0.00 | 0.00 | 0.01 | −0.03 | −0.17 | −0.11 |
| 15 | 7 | 0.00 | −0.01 | 0.00 | 0.01 | 0.00 | 0.00 | 0.00 | −0.05 | −0.04 |
| 16 | 6 | −0.01 | 0.07 | −0.10 | −0.01 | 0.00 | 0.00 | 0.00 | 0.00 | 0.00 |
| 17 | 16 | 0.00 | −0.01 | 0.01 | 0.00 | 0.00 | 0.00 | 0.00 | 0.00 | 0.00 |
| 18 | 6 | 0.01 | −0.01 | 0.00 | 0.00 | 0.00 | 0.00 | 0.00 | 0.00 | 0.00 |
| 19 | 8 | −0.03 | 0.04 | 0.00 | 0.01 | −0.01 | −0.01 | 0.00 | 0.00 | 0.00 |
| 20 | 8 | 0.02 | 0.07 | −0.07 | 0.00 | 0.00 | −0.02 | 0.00 | −0.01 | 0.01 |
| 21 | 1 | −0.01 | 0.03 | −0.01 | 0.00 | −0.01 | −0.01 | 0.06 | 0.26 | 0.20 |
| 22 | 1 | −0.02 | 0.01 | −0.02 | −0.02 | 0.01 | −0.01 | 0.01 | 0.03 | 0.01 |
| 23 | 1 | −0.14 | −0.10 | −0.42 | 0.19 | 0.00 | −0.07 | −0.04 | 0.00 | 0.05 |
| 24 | 1 | 0.11 | 0.02 | −0.09 | −0.03 | 0.03 | −0.06 | 0.00 | −0.02 | 0.01 |
| 25 | 1 | 0.06 | −0.22 | −0.11 | 0.02 | 0.01 | 0.02 | −0.01 | 0.02 | 0.00 |
| 26 | 1 | −0.10 | 0.08 | −0.28 | −0.01 | −0.02 | 0.00 | 0.00 | 0.00 | 0.01 |
| 27 | 1 | 0.04 | 0.16 | −0.15 | 0.03 | −0.11 | −0.03 | −0.01 | 0.00 | 0.01 |
| 28 | 1 | −0.05 | −0.03 | 0.22 | −0.01 | −0.01 | 0.04 | 0.00 | 0.00 | −0.01 |
| 29 | 1 | 0.31 | 0.02 | −0.16 | 0.03 | −0.02 | −0.01 | −0.01 | 0.00 | 0.01 |
| 30 | 1 | 0.00 | 0.00 | 0.00 | 0.00 | 0.00 | 0.00 | 0.00 | 0.00 | 0.00 |
| 31 | 1 | 0.02 | 0.00 | 0.00 | 0.00 | 0.00 | 0.00 | 0.00 | 0.00 | 0.00 |
| 32 | 1 | 0.01 | 0.00 | 0.00 | 0.00 | 0.00 | 0.00 | 0.00 | 0.00 | 0.00 |
| 33 | 1 | 0.10 | 0.06 | −0.02 | 0.01 | 0.02 | −0.01 | −0.01 | 0.00 | 0.00 |
| 34 | 1 | 0.00 | −0.01 | 0.00 | 0.00 | 0.01 | 0.00 | −0.07 | −0.08 | −0.05 |
| 35 | 1 | 0.00 | −0.01 | 0.00 | 0.01 | 0.01 | −0.01 | 0.02 | 0.00 | −0.02 |
| 36 | 15 | 0.00 | 0.00 | 0.00 | 0.06 | 0.02 | 0.02 | 0.00 | 0.00 | 0.00 |
| 37 | 8 | −0.02 | 0.00 | 0.00 | 0.38 | 0.12 | 0.13 | 0.02 | 0.00 | 0.00 |
| 38 | 8 | 0.01 | 0.01 | −0.01 | −0.23 | −0.14 | 0.29 | −0.01 | −0.01 | 0.01 |
| 39 | 8 | 0.01 | −0.01 | 0.01 | −0.23 | 0.30 | −0.17 | −0.01 | 0.01 | −0.01 |
| 40 | 8 | 0.00 | 0.01 | 0.01 | 0.00 | −0.28 | −0.27 | 0.00 | −0.01 | −0.01 |
| 41 | 1 | −0.01 | −0.01 | 0.03 | −0.02 | −0.01 | 0.01 | 0.03 | 0.15 | 0.12 |
| 42 | 1 | 0.01 | 0.00 | −0.01 | −0.17 | −0.12 | 0.24 | −0.01 | 0.00 | 0.01 |
| 43 | 1 | 0.00 | 0.01 | 0.00 | 0.02 | −0.23 | −0.22 | 0.00 | −0.01 | −0.01 |
| 44 | 1 | 0.01 | −0.01 | 0.00 | −0.16 | 0.12 | −0.10 | −0.01 | 0.01 | 0.00 |

| | 52 | 53 | 54 |
|---|---|---|---|
| | ?A | ?A | ?A |
| Frequencies | 824.7269 | 830.6102 | 845.7988 |
| Red. masses | 4.3250 | 6.8581 | 3.9470 |
| Frc consts | 1.7333 | 2.7877 | 1.6636 |
| IR Inten | 5.9678 | 2.6007 | 9.4529 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.31 | −0.04 | 0.18 | −0.11 | 0.02 | −0.07 | 0.00 | −0.01 | −0.01 |
| 2 | 6 | −0.11 | 0.11 | 0.05 | 0.05 | −0.06 | −0.03 | 0.00 | 0.00 | −0.01 |
| 3 | 8 | −0.03 | 0.06 | −0.10 | 0.00 | −0.01 | 0.05 | 0.03 | 0.02 | 0.00 |
| 4 | 6 | 0.00 | −0.05 | −0.09 | 0.01 | 0.02 | 0.03 | −0.07 | −0.02 | 0.02 |
| 5 | 6 | −0.05 | −0.26 | −0.02 | 0.01 | 0.08 | −0.01 | 0.02 | 0.03 | 0.04 |
| 6 | 7 | −0.03 | −0.01 | 0.05 | −0.11 | −0.12 | 0.20 | 0.00 | −0.01 | 0.01 |
| 7 | 6 | 0.00 | 0.00 | −0.02 | 0.00 | 0.05 | −0.06 | 0.00 | 0.00 | 0.00 |
| 8 | 6 | 0.01 | 0.01 | −0.01 | 0.03 | 0.01 | −0.04 | 0.00 | 0.00 | 0.00 |
| 9 | 7 | 0.05 | −0.01 | 0.00 | 0.21 | −0.04 | 0.01 | 0.00 | −0.01 | 0.01 |
| 10 | 6 | 0.04 | −0.04 | 0.05 | 0.17 | −0.21 | 0.24 | 0.00 | −0.01 | 0.02 |
| 11 | 6 | −0.03 | 0.01 | −0.01 | −0.16 | 0.06 | −0.01 | −0.01 | 0.00 | 0.00 |
| 12 | 7 | 0.00 | 0.01 | −0.01 | 0.03 | 0.03 | −0.06 | 0.01 | 0.00 | 0.00 |
| 13 | 6 | 0.00 | 0.02 | −0.03 | 0.02 | 0.14 | −0.19 | 0.00 | 0.01 | −0.02 |
| 14 | 7 | 0.01 | 0.03 | −0.04 | 0.04 | 0.14 | −0.21 | 0.00 | 0.01 | −0.01 |
| 15 | 7 | −0.04 | −0.01 | 0.02 | −0.20 | −0.03 | 0.09 | −0.01 | 0.00 | 0.01 |
| 16 | 6 | 0.03 | 0.01 | −0.03 | −0.02 | 0.01 | 0.04 | 0.09 | −0.19 | −0.14 |
| 17 | 16 | 0.00 | 0.00 | 0.00 | −0.01 | −0.01 | −0.01 | 0.13 | 0.06 | 0.03 |
| 18 | 6 | −0.01 | 0.00 | 0.00 | 0.02 | −0.01 | 0.00 | −0.35 | 0.08 | 0.04 |
| 19 | 8 | −0.07 | 0.05 | 0.05 | 0.03 | −0.02 | −0.02 | 0.00 | 0.00 | −0.01 |
| 20 | 8 | −0.03 | 0.03 | −0.01 | 0.02 | 0.00 | 0.00 | 0.00 | 0.00 | 0.00 |
| 21 | 1 | −0.03 | 0.02 | −0.03 | −0.19 | 0.16 | −0.18 | −0.02 | 0.01 | −0.02 |
| 22 | 1 | 0.04 | −0.06 | 0.04 | 0.19 | −0.20 | 0.25 | 0.00 | −0.03 | 0.00 |
| 23 | 1 | −0.49 | 0.04 | 0.15 | −0.10 | −0.07 | −0.04 | 0.06 | 0.02 | −0.05 |
| 24 | 1 | 0.27 | −0.22 | 0.24 | −0.12 | 0.12 | −0.03 | 0.01 | −0.05 | −0.03 |
| 25 | 1 | −0.07 | −0.15 | −0.15 | 0.03 | 0.04 | 0.03 | 0.01 | 0.03 | 0.03 |
| 26 | 1 | 0.01 | 0.10 | −0.10 | 0.01 | −0.02 | 0.02 | −0.07 | −0.05 | 0.09 |
| 27 | 1 | 0.07 | 0.21 | −0.33 | −0.02 | −0.07 | 0.12 | 0.00 | −0.01 | 0.00 |
| 28 | 1 | 0.03 | 0.01 | −0.09 | −0.01 | 0.03 | 0.01 | 0.05 | −0.45 | −0.09 |
| 29 | 1 | −0.01 | 0.08 | −0.02 | −0.05 | 0.00 | 0.04 | 0.15 | −0.20 | −0.14 |
| 30 | 1 | −0.01 | 0.01 | 0.00 | 0.01 | 0.00 | 0.00 | −0.18 | −0.06 | −0.02 |
| 31 | 1 | 0.00 | 0.00 | 0.00 | 0.05 | 0.00 | 0.00 | −0.63 | 0.00 | 0.00 |
| 32 | 1 | −0.02 | 0.01 | 0.00 | 0.01 | 0.00 | 0.00 | −0.12 | −0.05 | −0.03 |
| 33 | 1 | −0.03 | 0.05 | 0.05 | 0.04 | −0.02 | −0.02 | −0.07 | 0.00 | 0.00 |
| 34 | 1 | 0.00 | −0.01 | 0.01 | −0.02 | −0.08 | 0.07 | 0.00 | −0.01 | 0.00 |
| 35 | 1 | −0.06 | −0.03 | 0.04 | −0.30 | −0.18 | 0.20 | −0.02 | −0.01 | 0.01 |
| 36 | 15 | 0.01 | 0.00 | 0.00 | 0.00 | 0.00 | 0.00 | 0.00 | 0.00 | 0.00 |
| 37 | 8 | 0.03 | 0.01 | 0.01 | 0.00 | 0.00 | 0.00 | 0.00 | 0.00 | 0.00 |
| 38 | 8 | −0.01 | −0.01 | 0.02 | 0.00 | 0.00 | 0.00 | 0.00 | 0.00 | 0.00 |
| 39 | 8 | −0.02 | 0.02 | −0.01 | 0.00 | 0.00 | 0.00 | 0.00 | 0.00 | 0.00 |
| 40 | 8 | 0.00 | −0.02 | −0.02 | 0.00 | 0.00 | 0.00 | 0.00 | 0.00 | 0.00 |
| 41 | 1 | 0.03 | 0.00 | −0.02 | 0.12 | 0.00 | −0.05 | 0.00 | −0.01 | 0.00 |
| 42 | 1 | −0.02 | −0.01 | 0.02 | 0.00 | 0.00 | 0.00 | 0.00 | 0.00 | 0.00 |
| 43 | 1 | 0.00 | −0.02 | −0.02 | 0.00 | 0.00 | 0.00 | 0.00 | 0.00 | 0.00 |
| 44 | 1 | −0.01 | 0.00 | −0.01 | 0.00 | 0.00 | 0.00 | 0.00 | 0.00 | 0.00 |

| | 55 | 56 | 57 |
|---|---|---|---|
| | ?A | ?A | ?A |
| Frequencies | 858.6405 | 897.5389 | 913.5223 |
| Red. masses | 2.0122 | 1.4572 | 3.2490 |
| Frc consts | 0.8741 | 0.6916 | 1.5975 |
| IR Inten | 4.2442 | 25.1221 | 5.3619 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.02 | 0.01 | 0.00 | 0.00 | 0.00 | 0.00 | 0.00 | 0.00 | 0.00 |
| 2 | 6 | 0.00 | 0.00 | 0.02 | 0.00 | 0.00 | 0.00 | 0.02 | −0.01 | 0.00 |
| 3 | 8 | −0.01 | 0.03 | 0.04 | 0.00 | 0.00 | 0.00 | −0.02 | −0.05 | −0.03 |
| 4 | 6 | 0.01 | 0.00 | 0.00 | 0.01 | 0.00 | 0.00 | 0.07 | 0.05 | −0.02 |
| 5 | 6 | −0.04 | −0.02 | −0.10 | 0.00 | 0.00 | 0.00 | 0.01 | −0.01 | 0.03 |
| 6 | 7 | 0.00 | 0.00 | −0.01 | 0.00 | 0.04 | 0.03 | −0.02 | 0.00 | 0.00 |
| 7 | 6 | 0.00 | 0.00 | 0.00 | 0.00 | 0.01 | 0.00 | −0.01 | 0.00 | 0.00 |
| 8 | 6 | 0.00 | 0.00 | 0.00 | 0.00 | 0.00 | 0.00 | 0.00 | 0.00 | −0.01 |
| 9 | 7 | 0.00 | 0.00 | 0.00 | 0.00 | 0.02 | 0.01 | 0.01 | 0.00 | −0.01 |
| 10 | 6 | 0.00 | 0.02 | 0.00 | −0.02 | −0.15 | −0.11 | 0.01 | 0.03 | 0.02 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | −0.01 | 0.00 | 0.00 | −0.02 | 0.00 | 0.00 |
| 13 | 6 | 0.00 | −0.01 | 0.01 | 0.00 | 0.00 | 0.01 | 0.00 | −0.02 | 0.02 |
| 14 | 7 | 0.00 | 0.00 | 0.01 | 0.00 | 0.00 | 0.00 | 0.01 | 0.00 | 0.00 |
| 15 | 7 | 0.01 | 0.00 | 0.00 | 0.00 | 0.00 | 0.00 | 0.01 | 0.00 | −0.01 |
| 16 | 6 | 0.06 | 0.03 | 0.20 | −0.01 | 0.02 | 0.01 | −0.17 | 0.25 | 0.05 |
| 17 | 16 | 0.04 | −0.03 | −0.03 | 0.01 | −0.01 | 0.00 | 0.11 | −0.10 | −0.04 |
| 18 | 6 | −0.12 | −0.01 | −0.01 | −0.02 | 0.00 | 0.00 | −0.16 | 0.04 | 0.00 |
| 19 | 8 | 0.03 | 0.00 | −0.02 | 0.00 | 0.00 | 0.00 | −0.02 | 0.00 | 0.02 |
| 20 | 8 | 0.00 | 0.00 | −0.01 | 0.00 | 0.00 | 0.00 | 0.00 | 0.00 | 0.01 |
| 21 | 1 | 0.01 | 0.00 | 0.01 | 0.00 | −0.03 | −0.01 | 0.01 | −0.01 | 0.03 |
| 22 | 1 | 0.00 | −0.05 | −0.06 | 0.14 | 0.78 | 0.55 | −0.01 | −0.17 | −0.13 |
| 23 | 1 | −0.07 | 0.01 | −0.03 | −0.03 | −0.01 | 0.00 | −0.08 | −0.07 | 0.15 |
| 24 | 1 | −0.04 | 0.12 | 0.04 | 0.00 | 0.01 | 0.01 | 0.00 | −0.02 | 0.00 |
| 25 | 1 | 0.01 | 0.00 | −0.17 | 0.00 | 0.00 | 0.00 | 0.00 | −0.06 | 0.13 |
| 26 | 1 | 0.04 | 0.14 | −0.16 | 0.01 | 0.00 | −0.01 | 0.06 | −0.05 | 0.01 |
| 27 | 1 | −0.01 | 0.01 | 0.01 | 0.00 | 0.00 | 0.00 | 0.00 | 0.00 | 0.01 |
| 28 | 1 | 0.10 | −0.07 | −0.47 | −0.01 | 0.05 | 0.02 | −0.15 | 0.45 | 0.51 |
| 29 | 1 | −0.57 | 0.22 | 0.31 | −0.01 | 0.01 | 0.01 | 0.31 | 0.12 | −0.06 |
| 30 | 1 | −0.14 | 0.05 | 0.03 | −0.01 | −0.01 | 0.00 | 0.06 | −0.09 | −0.01 |
| 31 | 1 | 0.10 | 0.03 | 0.02 | −0.03 | 0.00 | 0.00 | −0.33 | −0.03 | 0.01 |
| 32 | 1 | −0.21 | 0.06 | 0.03 | −0.02 | 0.00 | 0.00 | −0.08 | −0.07 | −0.01 |
| 33 | 1 | 0.15 | 0.00 | −0.04 | 0.01 | 0.00 | 0.00 | −0.02 | 0.01 | 0.02 |
| 34 | 1 | 0.00 | 0.00 | 0.00 | 0.00 | 0.01 | 0.00 | 0.01 | 0.01 | −0.01 |
| 35 | 1 | 0.01 | 0.01 | −0.01 | 0.01 | 0.00 | 0.00 | 0.01 | 0.01 | −0.01 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.00 | −0.01 | −0.01 | 0.01 | 0.12 | 0.08 | 0.00 | −0.02 | −0.03 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 58 | 59 | 60 |
|---|---|---|---|
| | ?A | ?A | ?A |
| Frequencies | 934.7264 | 956.1218 | 959.4850 |
| Red. masses | 4.1079 | 1.5379 | 1.1943 |
| Frc consts | 2.1146 | 0.8284 | 0.6478 |
| IR Inten | 43.0880 | 12.0735 | 0.4389 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.03 | 0.01 | −0.01 | 0.00 | 0.00 | 0.00 | 0.00 | 0.00 | 0.00 |
| 2 | 6 | 0.02 | −0.05 | −0.02 | 0.00 | 0.00 | 0.00 | 0.00 | 0.00 | 0.00 |
| 3 | 8 | 0.00 | 0.04 | 0.03 | 0.00 | 0.00 | 0.00 | 0.00 | 0.00 | 0.00 |
| 4 | 6 | 0.02 | −0.04 | 0.01 | 0.00 | 0.00 | 0.00 | 0.01 | 0.00 | 0.00 |
| 5 | 6 | −0.01 | 0.03 | −0.01 | 0.00 | 0.00 | 0.00 | −0.01 | −0.01 | −0.01 |
| 6 | 7 | −0.22 | 0.05 | 0.01 | 0.00 | 0.00 | 0.00 | 0.00 | 0.00 | 0.00 |
| 7 | 6 | −0.06 | 0.03 | −0.03 | 0.00 | 0.01 | 0.01 | 0.00 | 0.00 | 0.00 |
| 8 | 6 | 0.02 | 0.02 | −0.04 | 0.00 | 0.00 | 0.00 | 0.00 | 0.00 | 0.00 |
| 9 | 7 | 0.09 | 0.00 | −0.03 | 0.00 | 0.00 | 0.00 | 0.00 | 0.00 | −0.01 |
| 10 | 6 | 0.06 | −0.02 | 0.04 | 0.00 | −0.01 | 0.00 | 0.00 | 0.00 | 0.00 |
| 11 | 6 | 0.02 | 0.02 | −0.04 | 0.00 | 0.01 | 0.01 | 0.00 | 0.00 | 0.00 |
| 12 | 7 | −0.17 | 0.04 | −0.01 | 0.01 | 0.03 | 0.03 | 0.01 | 0.00 | 0.00 |
| 13 | 6 | 0.01 | −0.18 | 0.24 | −0.03 | −0.16 | −0.12 | 0.00 | 0.01 | −0.01 |
| 14 | 7 | 0.16 | 0.01 | −0.06 | 0.00 | 0.03 | 0.03 | −0.01 | 0.00 | 0.01 |
| 15 | 7 | 0.10 | 0.03 | −0.07 | 0.00 | 0.00 | 0.00 | 0.00 | 0.00 | 0.00 |
| 16 | 6 | 0.01 | −0.06 | −0.01 | 0.00 | 0.00 | 0.00 | −0.01 | 0.00 | 0.01 |
| 17 | 16 | −0.02 | 0.01 | 0.01 | 0.00 | 0.00 | 0.00 | 0.00 | 0.00 | −0.03 |
| 18 | 6 | 0.02 | 0.01 | 0.01 | 0.00 | 0.00 | 0.00 | 0.00 | −0.02 | 0.11 |
| 19 | 8 | 0.01 | 0.00 | −0.01 | 0.00 | 0.00 | 0.00 | 0.00 | 0.00 | 0.00 |
| 20 | 8 | 0.00 | −0.01 | 0.01 | 0.00 | 0.00 | 0.00 | 0.00 | 0.00 | 0.00 |
| 21 | 1 | −0.02 | −0.16 | 0.25 | 0.14 | 0.78 | 0.56 | 0.00 | 0.00 | −0.01 |
| 22 | 1 | 0.31 | −0.02 | −0.17 | 0.00 | 0.03 | 0.03 | −0.01 | −0.01 | 0.00 |
| 23 | 1 | −0.64 | −0.14 | 0.04 | −0.01 | 0.00 | 0.00 | −0.03 | 0.00 | 0.01 |
| 24 | 1 | −0.06 | 0.07 | 0.04 | 0.00 | 0.00 | 0.00 | −0.01 | 0.03 | 0.01 |
| 25 | 1 | −0.02 | 0.06 | −0.04 | 0.00 | 0.00 | 0.00 | 0.01 | −0.01 | −0.02 |
| 26 | 1 | 0.01 | −0.04 | 0.01 | 0.00 | 0.00 | 0.00 | 0.01 | 0.03 | 0.00 |
| 27 | 1 | −0.01 | −0.03 | 0.04 | 0.00 | 0.00 | 0.00 | 0.00 | 0.00 | 0.00 |
| 28 | 1 | 0.01 | −0.05 | −0.02 | 0.00 | 0.00 | 0.00 | 0.00 | 0.06 | 0.01 |
| 29 | 1 | −0.01 | −0.10 | −0.01 | 0.00 | 0.00 | 0.00 | −0.01 | −0.05 | 0.01 |
| 30 | 1 | 0.03 | −0.02 | −0.02 | 0.00 | 0.00 | 0.00 | −0.62 | 0.10 | −0.16 |
| 31 | 1 | −0.10 | −0.01 | −0.01 | −0.01 | 0.00 | 0.00 | −0.08 | 0.05 | −0.16 |
| 32 | 1 | 0.10 | −0.03 | −0.02 | 0.01 | 0.00 | 0.00 | 0.69 | −0.01 | −0.21 |
| 33 | 1 | 0.03 | −0.01 | −0.01 | 0.00 | 0.00 | 0.00 | 0.03 | 0.00 | 0.00 |
| 34 | 1 | 0.10 | 0.08 | −0.05 | −0.01 | −0.03 | −0.01 | 0.00 | 0.00 | 0.00 |
| 35 | 1 | 0.11 | 0.07 | −0.05 | 0.00 | −0.01 | 0.00 | 0.00 | 0.00 | 0.00 |
| 36 | 15 | 0.01 | 0.01 | 0.00 | 0.00 | 0.00 | 0.00 | 0.00 | 0.00 | −0.01 |
| 37 | 8 | 0.00 | 0.00 | 0.00 | 0.00 | 0.00 | 0.00 | 0.00 | 0.00 | 0.00 |
| 38 | 8 | −0.01 | 0.00 | 0.01 | 0.00 | 0.00 | 0.00 | 0.00 | 0.00 | 0.01 |
| 39 | 8 | 0.00 | 0.00 | 0.00 | 0.00 | 0.00 | 0.00 | 0.00 | 0.00 | 0.00 |
| 40 | 8 | 0.00 | −0.01 | −0.01 | 0.00 | 0.00 | 0.00 | 0.00 | 0.00 | 0.00 |
| 41 | 1 | 0.04 | 0.02 | −0.07 | 0.00 | 0.01 | 0.00 | 0.00 | 0.00 | −0.01 |
| 42 | 1 | −0.01 | −0.01 | 0.01 | 0.00 | 0.00 | 0.00 | 0.00 | 0.00 | 0.00 |
| 43 | 1 | 0.00 | −0.01 | −0.02 | 0.00 | 0.00 | 0.00 | 0.00 | 0.00 | 0.00 |
| 44 | 1 | 0.01 | −0.02 | 0.01 | 0.00 | 0.00 | 0.00 | 0.00 | 0.00 | 0.00 |

| | 61 | 62 | 63 |
|---|---|---|---|
| | ?A | ?A | ?A |
| Frequencies | 959.9197 | 961.4739 | 966.8371 |
| Red. masses | 12.4943 | 16.0216 | 1.5502 |
| Frc consts | 6.7832 | 8.7263 | 0.8538 |
| IR Inten | 98.2203 | 120.2601 | 32.1502 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | 0.00 | 0.02 | 0.01 | −0.01 | −0.02 |
| 2 | 6 | 0.00 | 0.00 | 0.00 | 0.00 | −0.01 | −0.02 | 0.00 | 0.00 | 0.01 |
| 3 | 8 | 0.00 | 0.00 | −0.01 | −0.01 | 0.02 | 0.01 | 0.02 | −0.05 | −0.03 |
| 4 | 6 | −0.01 | 0.01 | 0.00 | 0.03 | −0.04 | −0.01 | −0.06 | 0.06 | 0.00 |
| 5 | 6 | 0.00 | −0.01 | 0.00 | −0.01 | 0.02 | −0.01 | 0.02 | 0.00 | 0.04 |
| 6 | 7 | 0.01 | 0.00 | 0.00 | 0.01 | 0.00 | 0.00 | −0.01 | 0.00 | 0.00 |
| 7 | 6 | 0.00 | 0.00 | 0.01 | 0.00 | 0.00 | 0.01 | 0.01 | 0.01 | −0.02 |
| 8 | 6 | 0.00 | 0.00 | 0.00 | 0.00 | 0.00 | −0.01 | 0.00 | −0.01 | 0.01 |
| 9 | 7 | 0.00 | 0.01 | −0.01 | 0.00 | 0.01 | −0.02 | −0.01 | −0.03 | 0.04 |
| 10 | 6 | 0.00 | 0.00 | 0.00 | 0.00 | 0.00 | −0.01 | −0.01 | 0.00 | 0.00 |
| 11 | 6 | 0.00 | 0.00 | 0.00 | 0.00 | 0.00 | 0.01 | −0.01 | 0.01 | −0.01 |
| 12 | 7 | 0.02 | 0.00 | 0.00 | 0.03 | −0.01 | 0.01 | −0.04 | 0.02 | −0.02 |
| 13 | 6 | 0.00 | 0.00 | −0.02 | 0.00 | 0.01 | −0.03 | 0.00 | −0.03 | 0.03 |
| 14 | 7 | −0.01 | 0.00 | 0.01 | −0.03 | −0.01 | 0.02 | 0.04 | 0.02 | −0.03 |
| 15 | 7 | 0.00 | 0.00 | 0.00 | 0.00 | 0.00 | 0.01 | 0.00 | 0.00 | −0.01 |
| 16 | 6 | 0.00 | 0.00 | −0.01 | −0.01 | −0.02 | 0.01 | 0.00 | 0.04 | −0.03 |
| 17 | 16 | 0.00 | 0.00 | 0.00 | −0.01 | −0.01 | 0.00 | 0.02 | 0.02 | 0.00 |
| 18 | 6 | 0.00 | −0.01 | −0.02 | 0.01 | 0.03 | 0.02 | −0.05 | −0.11 | −0.02 |
| 19 | 8 | 0.00 | 0.00 | 0.00 | 0.00 | 0.00 | 0.00 | −0.01 | 0.00 | 0.01 |
| 20 | 8 | 0.00 | 0.00 | 0.00 | 0.00 | −0.01 | 0.01 | 0.00 | 0.00 | 0.00 |
| 21 | 1 | 0.01 | 0.06 | 0.02 | 0.02 | 0.06 | 0.01 | −0.02 | 0.00 | 0.04 |
| 22 | 1 | −0.02 | 0.00 | 0.01 | −0.04 | 0.01 | 0.03 | 0.06 | 0.03 | −0.02 |
| 23 | 1 | −0.01 | −0.02 | 0.05 | −0.10 | 0.02 | −0.15 | 0.19 | −0.03 | 0.16 |
| 24 | 1 | 0.01 | −0.02 | 0.00 | −0.01 | 0.02 | 0.04 | 0.04 | −0.11 | −0.06 |
| 25 | 1 | 0.01 | −0.02 | 0.01 | −0.06 | 0.09 | −0.06 | 0.06 | −0.08 | 0.12 |
| 26 | 1 | −0.01 | 0.01 | 0.01 | 0.03 | 0.00 | −0.02 | −0.06 | −0.03 | 0.02 |
| 27 | 1 | −0.01 | 0.00 | 0.01 | 0.00 | 0.03 | 0.00 | 0.00 | 0.00 | −0.01 |
| 28 | 1 | 0.00 | −0.04 | 0.00 | 0.01 | 0.07 | 0.00 | −0.03 | −0.17 | 0.04 |
| 29 | 1 | 0.01 | 0.02 | −0.01 | −0.03 | −0.06 | 0.01 | 0.09 | 0.18 | −0.04 |
| 30 | 1 | 0.03 | 0.01 | 0.03 | 0.08 | −0.05 | −0.03 | −0.45 | 0.20 | 0.05 |
| 31 | 1 | 0.08 | 0.00 | 0.02 | −0.21 | −0.02 | −0.01 | 0.65 | 0.09 | −0.02 |
| 32 | 1 | −0.11 | 0.02 | 0.03 | 0.14 | −0.06 | −0.03 | −0.22 | 0.18 | 0.02 |
| 33 | 1 | −0.01 | 0.00 | 0.00 | 0.03 | −0.02 | −0.01 | −0.09 | 0.01 | 0.02 |
| 34 | 1 | −0.01 | −0.01 | 0.00 | −0.02 | −0.01 | 0.00 | 0.02 | 0.01 | 0.00 |
| 35 | 1 | 0.00 | 0.00 | 0.00 | 0.00 | 0.00 | −0.01 | −0.01 | −0.01 | 0.02 |
| 36 | 15 | −0.02 | 0.36 | −0.19 | −0.18 | 0.19 | 0.38 | −0.01 | 0.00 | 0.02 |
| 37 | 8 | −0.04 | −0.03 | 0.00 | 0.00 | −0.01 | −0.02 | 0.00 | 0.00 | 0.00 |
| 38 | 8 | −0.23 | −0.15 | 0.27 | 0.28 | 0.15 | −0.33 | 0.01 | 0.01 | −0.01 |
| 39 | 8 | 0.29 | −0.35 | 0.21 | 0.07 | −0.09 | 0.04 | 0.00 | 0.00 | 0.00 |
| 40 | 8 | −0.01 | −0.15 | −0.12 | −0.01 | −0.42 | −0.42 | 0.00 | −0.01 | −0.01 |
| 41 | 1 | 0.00 | 0.01 | −0.01 | 0.00 | 0.02 | −0.02 | 0.01 | −0.03 | 0.05 |
| 42 | 1 | 0.00 | 0.03 | 0.11 | −0.03 | 0.09 | −0.10 | 0.00 | 0.00 | −0.01 |
| 43 | 1 | 0.05 | 0.02 | −0.08 | 0.10 | −0.11 | −0.04 | 0.00 | 0.00 | 0.00 |
| 44 | 1 | 0.29 | −0.45 | 0.23 | 0.04 | −0.07 | 0.10 | 0.00 | 0.00 | 0.00 |

| | 64 | 65 | 66 |
|---|---|---|---|
| | ?A | ?A | ?A |
| Frequencies | 980.0658 | 994.3866 | 1028.0170 |
| Red. masses | 3.2263 | 1.8879 | 1.8576 |
| Frc consts | 1.8259 | 1.0999 | 1.1566 |
| IR Inten | 29.8340 | 17.6134 | 22.0764 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.01 | 0.00 | 0.01 | −0.08 | 0.03 | −0.07 | 0.06 | −0.04 | −0.01 |
| 2 | 6 | −0.03 | 0.03 | 0.03 | 0.00 | 0.06 | 0.08 | −0.03 | −0.01 | 0.02 |
| 3 | 8 | 0.00 | 0.00 | 0.00 | 0.02 | −0.03 | −0.02 | 0.03 | −0.05 | −0.02 |
| 4 | 6 | 0.01 | 0.01 | 0.00 | −0.04 | 0.11 | 0.07 | −0.09 | 0.07 | −0.04 |
| 5 | 6 | −0.01 | −0.02 | −0.02 | −0.01 | −0.16 | −0.04 | 0.02 | 0.06 | 0.06 |
| 6 | 7 | 0.08 | −0.01 | −0.03 | −0.02 | 0.00 | 0.00 | −0.07 | 0.00 | 0.01 |
| 7 | 6 | 0.12 | 0.02 | −0.06 | −0.01 | 0.00 | 0.00 | −0.07 | 0.00 | 0.01 |
| 8 | 6 | 0.02 | −0.05 | 0.07 | 0.00 | 0.00 | −0.01 | −0.01 | 0.02 | −0.02 |
| 9 | 7 | −0.08 | −0.12 | 0.19 | 0.01 | 0.01 | −0.02 | 0.04 | 0.03 | −0.05 |
| 10 | 6 | −0.06 | 0.02 | −0.01 | 0.01 | −0.01 | 0.00 | 0.02 | −0.03 | 0.03 |
| 11 | 6 | −0.04 | 0.03 | −0.03 | 0.00 | 0.00 | 0.00 | 0.01 | 0.00 | 0.00 |
| 12 | 7 | −0.12 | 0.07 | −0.07 | 0.01 | −0.01 | 0.01 | 0.01 | −0.02 | 0.02 |
| 13 | 6 | −0.01 | −0.04 | 0.05 | 0.00 | 0.00 | 0.00 | 0.00 | −0.02 | 0.02 |
| 14 | 7 | 0.12 | 0.07 | −0.12 | 0.00 | 0.00 | 0.01 | 0.00 | 0.00 | 0.01 |
| 15 | 7 | −0.05 | 0.00 | 0.01 | 0.01 | 0.00 | 0.00 | 0.03 | 0.01 | −0.02 |
| 16 | 6 | 0.01 | 0.00 | 0.03 | 0.01 | −0.02 | −0.01 | 0.06 | 0.04 | 0.03 |
| 17 | 16 | −0.01 | −0.02 | −0.01 | −0.01 | 0.00 | 0.00 | −0.02 | −0.03 | −0.02 |
| 18 | 6 | 0.03 | 0.06 | 0.02 | 0.02 | 0.03 | 0.00 | 0.03 | 0.05 | 0.02 |
| 19 | 8 | 0.00 | 0.00 | 0.00 | 0.03 | 0.00 | −0.02 | −0.02 | 0.00 | 0.01 |
| 20 | 8 | 0.00 | 0.01 | −0.01 | 0.02 | 0.04 | −0.03 | 0.00 | −0.01 | 0.00 |
| 21 | 1 | −0.04 | −0.01 | 0.06 | 0.00 | 0.00 | 0.00 | −0.06 | −0.01 | 0.03 |
| 22 | 1 | 0.14 | 0.08 | −0.12 | 0.00 | 0.01 | 0.02 | −0.02 | −0.02 | 0.07 |
| 23 | 1 | 0.53 | 0.13 | −0.10 | 0.05 | −0.12 | 0.67 | 0.53 | 0.05 | 0.01 |
| 24 | 1 | 0.05 | −0.05 | −0.05 | −0.08 | 0.17 | −0.05 | 0.13 | −0.31 | −0.13 |
| 25 | 1 | 0.00 | −0.02 | −0.04 | 0.29 | −0.46 | 0.13 | −0.01 | 0.04 | 0.11 |
| 26 | 1 | 0.02 | 0.07 | −0.05 | −0.04 | 0.13 | 0.10 | −0.05 | 0.00 | −0.19 |
| 27 | 1 | 0.01 | 0.02 | −0.03 | −0.02 | 0.01 | 0.06 | 0.02 | 0.01 | −0.05 |
| 28 | 1 | 0.03 | 0.10 | −0.07 | 0.01 | −0.03 | −0.04 | 0.04 | −0.21 | −0.17 |
| 29 | 1 | −0.09 | −0.03 | 0.04 | −0.03 | −0.16 | −0.01 | −0.08 | 0.42 | 0.07 |
| 30 | 1 | 0.21 | −0.11 | −0.05 | 0.13 | −0.05 | −0.01 | 0.13 | −0.07 | −0.04 |
| 31 | 1 | −0.40 | −0.05 | 0.00 | −0.16 | −0.03 | 0.01 | −0.28 | −0.03 | −0.01 |
| 32 | 1 | 0.21 | −0.11 | −0.04 | 0.04 | −0.04 | 0.00 | 0.19 | −0.08 | −0.04 |
| 33 | 1 | 0.03 | 0.00 | −0.01 | 0.05 | 0.01 | −0.03 | −0.13 | 0.01 | 0.03 |
| 34 | 1 | 0.05 | 0.01 | 0.01 | 0.00 | 0.00 | 0.00 | 0.03 | 0.01 | −0.01 |
| 35 | 1 | −0.11 | −0.06 | 0.10 | 0.01 | 0.01 | −0.01 | 0.04 | 0.02 | −0.02 |
| 36 | 15 | −0.01 | 0.00 | 0.00 | 0.00 | 0.00 | 0.01 | −0.03 | −0.01 | −0.01 |
| 37 | 8 | 0.01 | 0.00 | 0.01 | 0.00 | 0.00 | 0.00 | 0.03 | 0.01 | 0.01 |
| 38 | 8 | 0.00 | 0.00 | 0.00 | 0.01 | 0.00 | −0.01 | 0.00 | 0.00 | −0.01 |
| 39 | 8 | 0.01 | −0.01 | 0.01 | 0.00 | 0.00 | 0.00 | 0.01 | −0.01 | 0.01 |
| 40 | 8 | 0.00 | 0.00 | 0.00 | 0.00 | −0.01 | −0.01 | 0.00 | 0.01 | 0.00 |
| 41 | 1 | 0.04 | −0.19 | 0.27 | 0.00 | 0.02 | −0.02 | −0.03 | 0.07 | −0.09 |
| 42 | 1 | 0.02 | 0.01 | −0.01 | −0.01 | 0.00 | 0.00 | 0.04 | 0.02 | −0.03 |
| 43 | 1 | 0.01 | 0.02 | 0.02 | 0.00 | −0.01 | 0.00 | 0.01 | 0.04 | 0.04 |
| 44 | 1 | 0.00 | 0.00 | 0.00 | 0.00 | 0.00 | 0.00 | −0.01 | 0.03 | −0.01 |

| | 67 | 68 | 69 |
|---|---|---|---|
| | ?A | ?A | ?A |
| Frequencies | 1038.2585 | 1052.0600 | 1084.5335 |
| Red. masses | 2.9503 | 1.8989 | 2.4419 |
| Frc consts | 1.8738 | 1.2383 | 1.6922 |
| IR Inten | 105.9096 | 38.3276 | 18.7276 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.02 | −0.04 | −0.03 | 0.01 | 0.02 | 0.04 | −0.11 | 0.05 | −0.04 |
| 2 | 6 | 0.05 | −0.01 | −0.02 | −0.07 | 0.00 | 0.02 | −0.02 | 0.06 | 0.08 |
| 3 | 8 | 0.00 | −0.03 | −0.02 | 0.01 | 0.01 | 0.00 | −0.03 | 0.07 | 0.09 |
| 4 | 6 | −0.07 | 0.05 | −0.03 | 0.03 | −0.02 | 0.02 | 0.08 | −0.17 | −0.14 |
| 5 | 6 | 0.02 | 0.07 | 0.05 | −0.02 | −0.04 | −0.04 | 0.04 | −0.11 | 0.09 |
| 6 | 7 | 0.05 | 0.01 | 0.00 | −0.05 | −0.01 | 0.00 | 0.00 | 0.00 | 0.00 |
| 7 | 6 | 0.05 | −0.01 | −0.01 | −0.06 | 0.01 | 0.01 | −0.01 | 0.00 | 0.00 |
| 8 | 6 | 0.01 | −0.01 | 0.01 | −0.01 | 0.01 | −0.01 | 0.00 | 0.00 | 0.00 |
| 9 | 7 | −0.02 | −0.02 | 0.03 | 0.02 | 0.02 | −0.03 | 0.00 | 0.00 | 0.00 |
| 10 | 6 | −0.01 | 0.02 | −0.03 | 0.02 | −0.03 | 0.03 | 0.00 | 0.00 | 0.00 |
| 11 | 6 | 0.00 | 0.00 | 0.00 | 0.00 | 0.00 | −0.01 | 0.00 | 0.00 | 0.00 |
| 12 | 7 | 0.00 | 0.01 | −0.01 | 0.00 | −0.02 | 0.02 | 0.00 | 0.00 | 0.00 |
| 13 | 6 | 0.00 | 0.01 | −0.02 | 0.00 | −0.02 | 0.02 | 0.00 | 0.00 | 0.00 |
| 14 | 7 | −0.01 | 0.00 | 0.00 | 0.01 | 0.00 | −0.01 | 0.00 | 0.00 | 0.00 |
| 15 | 7 | −0.02 | 0.00 | 0.01 | 0.03 | 0.01 | −0.02 | 0.00 | 0.00 | 0.00 |
| 16 | 6 | 0.04 | 0.02 | 0.02 | −0.02 | −0.01 | −0.01 | −0.01 | 0.00 | −0.02 |
| 17 | 16 | −0.02 | −0.02 | −0.01 | 0.01 | 0.01 | 0.01 | 0.00 | 0.00 | −0.01 |
| 18 | 6 | 0.02 | 0.03 | 0.01 | −0.01 | −0.02 | −0.01 | 0.00 | 0.00 | 0.00 |
| 19 | 8 | −0.01 | 0.00 | 0.01 | 0.01 | 0.00 | −0.01 | 0.02 | −0.01 | 0.01 |
| 20 | 8 | −0.01 | −0.02 | 0.02 | 0.01 | 0.01 | −0.02 | 0.03 | 0.05 | −0.05 |
| 21 | 1 | 0.05 | 0.01 | −0.02 | −0.09 | −0.01 | 0.03 | −0.02 | 0.00 | 0.00 |
| 22 | 1 | 0.03 | 0.04 | −0.05 | −0.05 | −0.05 | 0.07 | 0.01 | 0.00 | 0.00 |
| 23 | 1 | −0.49 | −0.15 | 0.22 | 0.73 | 0.18 | −0.26 | 0.04 | 0.17 | −0.21 |
| 24 | 1 | 0.03 | −0.18 | −0.03 | 0.02 | 0.06 | −0.01 | −0.03 | 0.10 | −0.15 |
| 25 | 1 | 0.01 | 0.02 | 0.13 | −0.03 | 0.03 | −0.13 | 0.21 | −0.29 | 0.16 |
| 26 | 1 | −0.04 | −0.04 | −0.12 | 0.03 | 0.07 | 0.05 | 0.03 | −0.25 | 0.02 |
| 27 | 1 | −0.01 | −0.03 | 0.03 | 0.02 | 0.04 | −0.06 | 0.00 | −0.03 | 0.05 |
| 28 | 1 | 0.01 | −0.25 | −0.11 | 0.01 | 0.24 | 0.05 | −0.05 | −0.26 | 0.06 |
| 29 | 1 | −0.03 | 0.35 | 0.05 | −0.01 | −0.29 | −0.02 | 0.13 | 0.66 | −0.01 |
| 30 | 1 | 0.08 | −0.04 | −0.02 | −0.04 | 0.02 | 0.01 | −0.01 | 0.00 | −0.01 |
| 31 | 1 | −0.17 | −0.01 | 0.00 | 0.08 | 0.01 | 0.00 | 0.00 | 0.01 | −0.01 |
| 32 | 1 | 0.12 | −0.05 | −0.02 | −0.06 | 0.02 | 0.01 | 0.02 | −0.01 | 0.00 |
| 33 | 1 | −0.11 | 0.00 | 0.02 | 0.07 | 0.00 | −0.02 | −0.14 | −0.03 | 0.03 |
| 34 | 1 | −0.03 | −0.01 | 0.01 | 0.05 | 0.01 | −0.02 | 0.02 | 0.00 | 0.00 |
| 35 | 1 | −0.02 | −0.01 | 0.01 | 0.02 | 0.01 | 0.00 | 0.00 | 0.00 | 0.01 |
| 36 | 15 | 0.13 | 0.03 | 0.05 | 0.08 | 0.02 | 0.03 | 0.01 | 0.00 | 0.00 |
| 37 | 8 | −0.20 | −0.06 | −0.07 | −0.13 | −0.04 | −0.04 | −0.01 | 0.00 | 0.00 |
| 38 | 8 | −0.01 | −0.02 | 0.04 | 0.00 | −0.01 | 0.02 | 0.00 | 0.00 | 0.00 |
| 39 | 8 | −0.04 | 0.07 | −0.04 | −0.02 | 0.04 | −0.02 | 0.00 | 0.00 | 0.00 |
| 40 | 8 | 0.01 | −0.03 | −0.02 | 0.01 | −0.02 | −0.01 | 0.00 | 0.00 | 0.00 |
| 41 | 1 | 0.02 | −0.04 | 0.05 | −0.02 | 0.05 | −0.06 | −0.01 | 0.01 | −0.01 |
| 42 | 1 | −0.24 | −0.10 | 0.17 | −0.15 | −0.06 | 0.10 | −0.02 | 0.00 | 0.01 |
| 43 | 1 | −0.07 | −0.19 | −0.22 | −0.05 | −0.12 | −0.14 | 0.00 | −0.01 | −0.02 |
| 44 | 1 | 0.05 | −0.17 | 0.07 | 0.03 | −0.11 | 0.04 | 0.00 | −0.01 | 0.00 |

| | 70 | 71 | 72 |
|---|---|---|---|
| | ?A | ?A | ?A |
| Frequencies | 1102.6122 | 1110.7797 | 1142.5842 |
| Red. masses | 1.5018 | 1.4486 | 1.2878 |
| Frc consts | 1.0758 | 1.0531 | 0.9906 |
| IR Inten | 2.3471 | 11.3462 | 30.0719 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.01 | 0.01 | 0.01 | 0.06 | 0.09 | 0.00 | 0.00 | 0.00 |
| 2 | 6 | −0.01 | −0.01 | 0.00 | −0.01 | −0.09 | −0.06 | −0.01 | 0.00 | 0.01 |
| 3 | 8 | 0.01 | 0.00 | 0.00 | 0.01 | −0.03 | −0.05 | 0.00 | 0.00 | 0.00 |
| 4 | 6 | 0.00 | 0.00 | 0.00 | −0.01 | 0.06 | 0.05 | 0.00 | 0.00 | 0.00 |
| 5 | 6 | 0.00 | −0.01 | 0.00 | 0.01 | −0.04 | −0.02 | 0.00 | 0.00 | 0.00 |
| 6 | 7 | −0.06 | 0.00 | 0.01 | 0.00 | 0.00 | 0.00 | −0.03 | −0.01 | 0.02 |
| 7 | 6 | 0.01 | 0.02 | −0.03 | 0.00 | 0.00 | 0.00 | 0.05 | −0.02 | 0.01 |
| 8 | 6 | 0.03 | 0.02 | −0.03 | 0.00 | 0.00 | 0.00 | 0.05 | 0.02 | −0.04 |
| 9 | 7 | 0.01 | 0.00 | 0.00 | 0.00 | 0.00 | 0.00 | −0.04 | 0.00 | 0.00 |
| 10 | 6 | −0.01 | −0.01 | 0.01 | 0.00 | 0.00 | 0.00 | −0.01 | −0.01 | 0.02 |
| 11 | 6 | 0.03 | −0.06 | 0.06 | 0.00 | 0.00 | 0.00 | −0.01 | 0.02 | −0.02 |
| 12 | 7 | −0.04 | 0.06 | −0.07 | 0.00 | 0.00 | 0.00 | 0.04 | −0.03 | 0.03 |
| 13 | 6 | −0.01 | 0.03 | −0.03 | 0.00 | 0.00 | 0.00 | 0.01 | 0.00 | 0.00 |
| 14 | 7 | 0.01 | −0.03 | 0.04 | 0.00 | 0.00 | 0.00 | −0.06 | −0.01 | 0.03 |
| 15 | 7 | −0.01 | −0.05 | 0.08 | 0.00 | 0.00 | −0.01 | −0.03 | 0.02 | −0.02 |
| 16 | 6 | 0.00 | 0.00 | 0.00 | 0.01 | 0.03 | −0.01 | 0.00 | 0.00 | 0.00 |
| 17 | 16 | 0.00 | 0.00 | 0.00 | −0.01 | −0.01 | −0.01 | 0.00 | 0.00 | 0.00 |
| 18 | 6 | 0.00 | 0.00 | 0.00 | 0.01 | 0.01 | 0.00 | 0.00 | 0.00 | 0.00 |
| 19 | 8 | 0.00 | 0.00 | 0.00 | 0.01 | 0.00 | 0.00 | 0.00 | 0.00 | 0.00 |
| 20 | 8 | 0.00 | 0.00 | 0.00 | −0.01 | −0.01 | 0.01 | 0.00 | 0.00 | 0.00 |
| 21 | 1 | 0.58 | −0.03 | −0.10 | −0.03 | 0.00 | 0.01 | 0.00 | 0.00 | 0.00 |
| 22 | 1 | 0.26 | 0.06 | −0.15 | 0.04 | 0.02 | −0.02 | 0.76 | 0.18 | −0.45 |
| 23 | 1 | 0.14 | 0.03 | −0.05 | 0.04 | −0.01 | −0.32 | 0.07 | 0.01 | 0.01 |
| 24 | 1 | 0.00 | 0.02 | 0.01 | −0.17 | 0.55 | 0.37 | 0.01 | −0.03 | −0.02 |
| 25 | 1 | 0.00 | −0.01 | −0.01 | 0.02 | −0.08 | 0.06 | −0.01 | 0.01 | −0.01 |
| 26 | 1 | 0.00 | 0.00 | 0.01 | −0.06 | 0.03 | 0.32 | 0.00 | 0.00 | −0.02 |
| 27 | 1 | 0.00 | 0.01 | −0.01 | 0.00 | 0.07 | −0.05 | 0.00 | 0.00 | −0.01 |
| 28 | 1 | 0.00 | 0.00 | 0.00 | −0.04 | −0.37 | 0.02 | 0.00 | 0.01 | 0.00 |
| 29 | 1 | 0.01 | 0.01 | 0.00 | 0.09 | 0.34 | −0.01 | 0.00 | −0.04 | 0.00 |
| 30 | 1 | 0.00 | 0.00 | 0.00 | 0.03 | −0.01 | 0.00 | 0.00 | 0.00 | 0.00 |
| 31 | 1 | 0.00 | 0.00 | 0.00 | −0.06 | −0.01 | 0.00 | 0.01 | 0.00 | 0.00 |
| 32 | 1 | 0.00 | 0.00 | 0.00 | 0.04 | −0.01 | −0.01 | 0.00 | 0.00 | 0.00 |
| 33 | 1 | 0.00 | 0.00 | 0.00 | 0.03 | 0.00 | −0.01 | 0.00 | 0.00 | 0.00 |
| 34 | 1 | −0.55 | −0.04 | 0.08 | 0.04 | 0.00 | −0.01 | 0.22 | 0.00 | −0.03 |
| 35 | 1 | 0.24 | 0.21 | −0.28 | −0.02 | −0.01 | 0.02 | −0.15 | −0.11 | 0.15 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.07 | 0.04 | −0.04 | 0.00 | −0.01 | 0.00 | −0.17 | 0.08 | −0.07 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 73 | 74 | 75 |
|---|---|---|---|
| | ?A | ?A | ?A |
| Frequencies | 1157.7258 | 1185.4757 | 1209.7755 |
| Red. masses | 1.2419 | 1.8875 | 1.2763 |
| Frc consts | 0.9807 | 1.5629 | 1.1006 |
| IR Inten | 20.3983 | 5.0954 | 10.4109 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | −0.03 | −0.03 | 0.06 | −0.06 | 0.00 | 0.00 | 0.00 | 0.00 |
| 2 | 6 | 0.00 | 0.01 | 0.01 | −0.02 | 0.06 | 0.04 | 0.00 | 0.00 | 0.00 |
| 3 | 8 | −0.01 | 0.03 | 0.02 | −0.01 | −0.02 | −0.02 | 0.00 | 0.01 | 0.00 |
| 4 | 6 | 0.08 | −0.01 | 0.05 | 0.14 | 0.05 | 0.00 | 0.00 | 0.00 | 0.00 |
| 5 | 6 | −0.01 | −0.01 | −0.08 | −0.07 | 0.11 | −0.09 | 0.00 | 0.00 | 0.00 |
| 6 | 7 | 0.00 | 0.00 | 0.00 | 0.00 | 0.00 | 0.00 | −0.05 | 0.03 | −0.03 |
| 7 | 6 | 0.00 | 0.00 | 0.00 | −0.01 | 0.00 | 0.00 | 0.03 | 0.02 | −0.04 |
| 8 | 6 | 0.00 | 0.00 | 0.00 | 0.00 | 0.00 | 0.00 | 0.06 | −0.02 | 0.01 |
| 9 | 7 | 0.00 | 0.00 | 0.00 | 0.00 | 0.00 | 0.00 | 0.02 | −0.01 | 0.01 |
| 10 | 6 | 0.00 | 0.00 | 0.00 | 0.00 | 0.00 | 0.00 | −0.01 | 0.00 | 0.01 |
| 11 | 6 | 0.00 | 0.00 | 0.00 | 0.00 | 0.00 | 0.00 | 0.01 | −0.01 | 0.02 |
| 12 | 7 | 0.00 | 0.00 | 0.00 | 0.00 | 0.00 | 0.00 | −0.01 | −0.03 | 0.04 |
| 13 | 6 | 0.00 | 0.00 | 0.00 | 0.00 | 0.00 | 0.00 | 0.00 | −0.01 | 0.02 |
| 14 | 7 | 0.00 | 0.00 | 0.00 | 0.00 | 0.00 | 0.00 | −0.05 | 0.02 | −0.01 |
| 15 | 7 | 0.00 | 0.00 | 0.00 | 0.00 | 0.00 | 0.00 | −0.04 | 0.03 | −0.04 |
| 16 | 6 | −0.02 | 0.00 | −0.01 | −0.10 | −0.09 | 0.03 | 0.00 | −0.01 | 0.00 |
| 17 | 16 | 0.00 | 0.00 | 0.00 | 0.00 | 0.01 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | −0.03 | 0.01 | 0.00 | −0.02 | 0.00 | 0.01 | 0.00 | 0.00 | 0.00 |
| 20 | 8 | 0.00 | 0.00 | 0.00 | 0.00 | −0.01 | 0.01 | 0.00 | 0.00 | 0.00 |
| 21 | 1 | 0.00 | 0.00 | 0.00 | −0.03 | 0.00 | 0.00 | 0.79 | −0.09 | −0.07 |
| 22 | 1 | −0.01 | 0.00 | 0.01 | 0.02 | 0.00 | −0.01 | −0.27 | −0.07 | 0.16 |
| 23 | 1 | 0.12 | −0.07 | 0.32 | 0.02 | 0.10 | −0.11 | 0.04 | −0.01 | 0.02 |
| 24 | 1 | 0.07 | −0.21 | −0.15 | 0.02 | −0.05 | 0.05 | 0.00 | 0.02 | 0.01 |
| 25 | 1 | −0.27 | 0.32 | −0.31 | 0.14 | −0.38 | 0.52 | 0.01 | −0.01 | 0.02 |
| 26 | 1 | −0.02 | 0.01 | 0.51 | 0.17 | 0.09 | −0.25 | 0.01 | 0.02 | −0.03 |
| 27 | 1 | −0.01 | −0.03 | 0.03 | 0.01 | 0.04 | −0.04 | 0.00 | 0.01 | −0.01 |
| 28 | 1 | −0.06 | −0.33 | 0.10 | −0.15 | −0.46 | 0.17 | −0.01 | −0.01 | 0.01 |
| 29 | 1 | 0.09 | 0.29 | −0.01 | 0.00 | 0.00 | 0.01 | 0.00 | 0.02 | 0.00 |
| 30 | 1 | 0.01 | 0.00 | 0.00 | −0.01 | 0.00 | 0.00 | 0.00 | 0.00 | 0.00 |
| 31 | 1 | −0.02 | 0.00 | 0.01 | 0.01 | 0.00 | 0.00 | 0.00 | 0.00 | 0.00 |
| 32 | 1 | 0.02 | 0.00 | 0.00 | −0.02 | 0.00 | 0.01 | 0.00 | 0.00 | 0.00 |
| 33 | 1 | 0.18 | 0.02 | −0.03 | 0.31 | 0.01 | −0.04 | 0.01 | 0.00 | 0.00 |
| 34 | 1 | 0.00 | 0.00 | 0.00 | −0.02 | 0.00 | 0.00 | 0.32 | 0.02 | −0.04 |
| 35 | 1 | 0.00 | 0.00 | 0.00 | 0.02 | 0.01 | −0.02 | −0.22 | −0.16 | 0.21 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.01 | 0.00 | 0.00 | −0.02 | 0.01 | −0.01 | −0.02 | 0.01 | −0.01 |
| 42 | 1 | −0.01 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 76 | 77 | 78 |
|---|---|---|---|
| | ?A | ?A | ?A |
| Frequencies | 1221.0274 | 1235.8179 | 1254.7817 |
| Red. masses | 1.2855 | 1.4931 | 1.0963 |
| Frc consts | 1.1292 | 1.3435 | 1.0170 |
| IR Inten | 0.5290 | 0.8420 | 26.6270 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.02 | 0.03 | 0.01 | 0.08 | −0.11 | −0.08 | 0.00 | 0.00 | 0.00 |
| 2 | 6 | 0.01 | −0.03 | −0.04 | −0.03 | 0.01 | −0.02 | 0.00 | 0.00 | 0.00 |
| 3 | 8 | 0.00 | 0.02 | 0.00 | −0.03 | 0.06 | 0.01 | 0.00 | 0.00 | 0.00 |
| 4 | 6 | 0.04 | 0.01 | 0.01 | 0.01 | −0.05 | 0.00 | 0.00 | 0.00 | 0.00 |
| 5 | 6 | 0.02 | −0.06 | 0.08 | −0.02 | 0.04 | 0.03 | 0.00 | 0.00 | 0.00 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.01 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.01 | 0.00 | 0.00 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | 0.00 | 0.00 | 0.00 | 0.00 | 0.00 | 0.00 | 0.00 | 0.00 | 0.00 |
| 16 | 6 | −0.07 | −0.03 | −0.01 | 0.02 | −0.01 | −0.01 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.04 | 0.00 | −0.01 | 0.02 | 0.01 | −0.01 | 0.00 | 0.00 | 0.00 |
| 20 | 8 | −0.01 | 0.00 | 0.00 | −0.04 | −0.03 | 0.04 | 0.00 | 0.00 | 0.00 |
| 21 | 1 | −0.03 | 0.00 | 0.00 | −0.03 | 0.00 | 0.00 | 0.00 | 0.00 | 0.00 |
| 22 | 1 | 0.00 | 0.00 | 0.00 | 0.01 | 0.00 | 0.00 | 0.00 | 0.00 | 0.00 |
| 23 | 1 | 0.07 | −0.08 | 0.15 | 0.23 | −0.10 | 0.44 | 0.00 | 0.00 | 0.00 |
| 24 | 1 | 0.00 | 0.08 | −0.04 | −0.09 | 0.70 | 0.08 | 0.00 | 0.00 | 0.00 |
| 25 | 1 | −0.15 | 0.24 | −0.21 | −0.13 | 0.08 | 0.10 | 0.00 | 0.00 | 0.00 |
| 26 | 1 | 0.19 | 0.58 | −0.56 | 0.01 | −0.28 | −0.14 | 0.00 | 0.00 | 0.00 |
| 27 | 1 | −0.01 | 0.00 | 0.00 | 0.00 | 0.13 | −0.12 | 0.00 | 0.00 | 0.00 |
| 28 | 1 | −0.11 | −0.29 | 0.10 | 0.03 | 0.12 | −0.01 | 0.00 | 0.00 | 0.00 |
| 29 | 1 | 0.04 | 0.03 | −0.03 | 0.01 | 0.09 | 0.00 | 0.00 | 0.00 | 0.00 |
| 30 | 1 | 0.00 | 0.01 | 0.01 | 0.00 | −0.01 | −0.01 | 0.00 | 0.00 | 0.00 |
| 31 | 1 | 0.00 | −0.01 | 0.00 | 0.02 | 0.01 | −0.01 | 0.00 | 0.00 | 0.00 |
| 32 | 1 | −0.01 | 0.01 | 0.00 | 0.00 | 0.00 | 0.00 | 0.00 | 0.00 | 0.00 |
| 33 | 1 | −0.15 | 0.00 | 0.02 | −0.02 | 0.01 | 0.00 | 0.00 | 0.00 | 0.00 |
| 34 | 1 | −0.01 | 0.00 | 0.00 | −0.03 | 0.00 | 0.00 | 0.00 | 0.00 | 0.00 |
| 35 | 1 | 0.01 | 0.01 | −0.01 | 0.01 | 0.01 | −0.01 | 0.00 | 0.00 | 0.00 |
| 36 | 15 | 0.00 | 0.00 | 0.00 | 0.00 | 0.00 | 0.00 | −0.01 | 0.00 | 0.02 |
| 37 | 8 | 0.00 | 0.00 | 0.00 | 0.00 | 0.00 | 0.00 | 0.00 | 0.00 | −0.01 |
| 38 | 8 | 0.00 | 0.00 | 0.00 | 0.00 | 0.00 | 0.00 | −0.05 | −0.01 | −0.01 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.04 | 0.01 | 0.02 |
| 41 | 1 | 0.01 | 0.00 | 0.00 | −0.03 | 0.01 | −0.01 | 0.00 | 0.00 | 0.00 |
| 42 | 1 | 0.01 | 0.00 | 0.00 | −0.01 | 0.00 | 0.01 | 0.58 | 0.25 | −0.37 |
| 43 | 1 | 0.00 | 0.00 | 0.00 | 0.00 | 0.00 | 0.00 | −0.19 | −0.45 | −0.47 |
| 44 | 1 | 0.00 | 0.00 | 0.00 | 0.00 | 0.00 | 0.00 | 0.00 | −0.01 | 0.03 |

| | 79 | 80 | 81 |
|---|---|---|---|
| | ?A | ?A | ?A |
| Frequencies | 1264.3515 | 1275.0694 | 1278.5881 |
| Red. masses | 1.2722 | 1.1152 | 1.7094 |
| Frc consts | 1.1982 | 1.0682 | 1.6465 |
| IR Inten | 3.7257 | 45.3989 | 15.5824 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.02 | −0.04 | −0.03 | 0.00 | 0.00 | 0.00 | 0.00 | 0.04 | 0.05 |
| 2 | 6 | 0.01 | −0.01 | 0.05 | 0.00 | −0.01 | −0.01 | −0.01 | −0.07 | −0.09 |
| 3 | 8 | 0.03 | 0.00 | −0.02 | 0.00 | 0.01 | 0.00 | −0.02 | 0.07 | 0.01 |
| 4 | 6 | −0.04 | −0.02 | −0.05 | 0.00 | 0.00 | 0.00 | 0.00 | −0.04 | 0.01 |
| 5 | 6 | 0.01 | 0.04 | 0.00 | 0.00 | 0.00 | 0.00 | 0.00 | 0.03 | −0.03 |
| 6 | 7 | 0.00 | 0.00 | 0.00 | 0.00 | 0.00 | 0.00 | −0.03 | −0.03 | 0.05 |
| 7 | 6 | 0.00 | 0.00 | 0.00 | 0.00 | 0.00 | 0.00 | 0.05 | 0.02 | −0.05 |
| 8 | 6 | 0.00 | 0.00 | 0.00 | 0.00 | 0.00 | 0.00 | −0.07 | 0.02 | −0.02 |
| 9 | 7 | 0.00 | 0.00 | 0.00 | 0.00 | 0.00 | 0.00 | 0.04 | 0.01 | −0.02 |
| 10 | 6 | 0.00 | 0.00 | 0.00 | 0.00 | 0.00 | 0.00 | −0.06 | 0.02 | −0.02 |
| 11 | 6 | 0.00 | 0.00 | 0.00 | 0.00 | 0.00 | 0.00 | 0.01 | −0.02 | 0.03 |
| 12 | 7 | 0.00 | 0.00 | 0.00 | 0.00 | 0.00 | 0.00 | 0.04 | 0.01 | −0.02 |
| 13 | 6 | 0.00 | 0.00 | 0.00 | 0.00 | 0.00 | 0.00 | −0.07 | 0.01 | 0.01 |
| 14 | 7 | 0.00 | 0.00 | 0.00 | 0.00 | 0.00 | 0.00 | 0.01 | −0.02 | 0.03 |
| 15 | 7 | 0.00 | 0.00 | 0.00 | 0.00 | 0.00 | 0.00 | 0.01 | 0.01 | −0.01 |
| 16 | 6 | −0.02 | −0.10 | 0.00 | 0.00 | 0.00 | 0.00 | 0.00 | −0.03 | −0.01 |
| 17 | 16 | 0.01 | 0.00 | 0.00 | 0.00 | 0.00 | 0.00 | 0.00 | 0.00 | 0.00 |
| 18 | 6 | 0.00 | −0.01 | −0.01 | 0.00 | 0.00 | 0.00 | 0.00 | 0.00 | 0.00 |
| 19 | 8 | −0.01 | 0.00 | 0.01 | 0.00 | 0.00 | 0.00 | −0.02 | −0.01 | 0.00 |
| 20 | 8 | 0.00 | 0.00 | 0.00 | 0.00 | 0.00 | 0.00 | 0.02 | 0.01 | −0.01 |
| 21 | 1 | −0.01 | 0.00 | 0.00 | 0.00 | 0.00 | 0.00 | −0.02 | 0.00 | 0.00 |
| 22 | 1 | 0.02 | 0.00 | −0.02 | 0.00 | 0.00 | 0.00 | 0.04 | 0.04 | −0.07 |
| 23 | 1 | −0.03 | 0.00 | −0.03 | 0.02 | −0.02 | 0.03 | 0.24 | −0.16 | 0.32 |
| 24 | 1 | −0.01 | 0.11 | 0.00 | −0.01 | −0.03 | 0.03 | −0.09 | −0.31 | 0.36 |
| 25 | 1 | 0.03 | −0.04 | 0.10 | 0.01 | −0.01 | 0.01 | 0.17 | −0.16 | 0.07 |
| 26 | 1 | −0.07 | 0.69 | 0.35 | 0.00 | 0.01 | −0.01 | 0.01 | −0.07 | −0.12 |
| 27 | 1 | 0.00 | 0.02 | −0.03 | 0.00 | 0.00 | 0.01 | 0.00 | −0.08 | 0.09 |
| 28 | 1 | 0.03 | 0.44 | 0.13 | 0.00 | 0.01 | 0.00 | 0.02 | 0.15 | 0.05 |
| 29 | 1 | −0.08 | 0.35 | 0.04 | 0.00 | 0.01 | 0.00 | −0.01 | 0.17 | 0.01 |
| 30 | 1 | −0.03 | 0.04 | 0.03 | 0.00 | 0.00 | 0.00 | −0.01 | 0.00 | 0.00 |
| 31 | 1 | −0.04 | −0.03 | 0.03 | 0.00 | 0.00 | 0.00 | 0.01 | 0.00 | 0.00 |
| 32 | 1 | −0.03 | 0.03 | 0.00 | 0.00 | 0.00 | 0.00 | −0.01 | 0.00 | 0.00 |
| 33 | 1 | −0.04 | 0.00 | 0.01 | 0.01 | 0.00 | 0.00 | 0.06 | 0.00 | −0.01 |
| 34 | 1 | 0.00 | 0.00 | 0.00 | 0.00 | 0.00 | 0.00 | 0.10 | 0.01 | −0.01 |
| 35 | 1 | 0.00 | 0.00 | 0.00 | 0.00 | 0.00 | 0.00 | −0.02 | −0.03 | 0.03 |
| 36 | 15 | 0.00 | 0.00 | 0.00 | −0.01 | 0.02 | −0.01 | 0.00 | 0.00 | 0.00 |
| 37 | 8 | 0.00 | 0.00 | 0.00 | 0.01 | 0.00 | 0.01 | 0.00 | 0.00 | 0.00 |
| 38 | 8 | 0.00 | 0.00 | 0.00 | 0.03 | 0.01 | 0.01 | 0.00 | 0.00 | 0.00 |
| 39 | 8 | 0.00 | 0.00 | 0.00 | −0.01 | −0.05 | 0.01 | 0.00 | 0.01 | 0.00 |
| 40 | 8 | 0.00 | 0.00 | 0.00 | 0.03 | 0.01 | 0.02 | 0.00 | 0.00 | 0.00 |
| 41 | 1 | 0.02 | −0.01 | 0.01 | 0.02 | −0.01 | 0.01 | 0.48 | −0.25 | 0.22 |
| 42 | 1 | 0.00 | 0.00 | 0.00 | −0.37 | −0.15 | 0.24 | 0.04 | 0.01 | −0.02 |
| 43 | 1 | 0.00 | 0.00 | 0.00 | −0.15 | −0.34 | −0.37 | 0.01 | 0.03 | 0.03 |
| 44 | 1 | 0.00 | 0.00 | 0.00 | −0.23 | 0.61 | −0.26 | 0.02 | −0.06 | 0.03 |

| | 82 | 83 | 84 |
|---|---|---|---|
| | ?A | ?A | ?A |
| Frequencies | 1280.9346 | 1291.8679 | 1299.4713 |
| Red. masses | 1.8940 | 1.3962 | 1.1890 |
| Frc consts | 1.8310 | 1.3729 | 1.1829 |
| IR Inten | 22.2679 | 35.8943 | 1.4753 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | −0.07 | −0.07 | 0.01 | −0.09 | −0.09 | 0.01 | −0.04 | 0.01 |
| 2 | 6 | 0.00 | 0.07 | 0.10 | −0.03 | 0.04 | 0.06 | 0.00 | 0.00 | 0.00 |
| 3 | 8 | 0.02 | −0.06 | −0.02 | 0.01 | −0.02 | −0.01 | 0.00 | 0.00 | −0.01 |
| 4 | 6 | 0.01 | 0.03 | −0.01 | 0.01 | 0.01 | −0.01 | 0.02 | −0.03 | 0.01 |
| 5 | 6 | 0.00 | −0.02 | 0.03 | 0.03 | 0.00 | 0.02 | −0.01 | 0.08 | 0.00 |
| 6 | 7 | −0.03 | −0.04 | 0.06 | 0.01 | 0.00 | −0.01 | 0.00 | 0.00 | 0.00 |
| 7 | 6 | 0.04 | 0.03 | −0.06 | −0.02 | 0.00 | 0.01 | 0.00 | 0.00 | 0.00 |
| 8 | 6 | −0.09 | 0.03 | −0.02 | 0.00 | 0.00 | 0.00 | 0.00 | 0.00 | 0.00 |
| 9 | 7 | 0.04 | 0.01 | −0.03 | −0.01 | 0.00 | 0.00 | 0.00 | 0.00 | 0.00 |
| 10 | 6 | −0.07 | 0.02 | −0.01 | 0.01 | 0.00 | 0.00 | 0.00 | 0.00 | 0.00 |
| 11 | 6 | 0.01 | −0.03 | 0.04 | 0.00 | 0.00 | 0.00 | 0.00 | 0.00 | 0.00 |
| 12 | 7 | 0.05 | 0.01 | −0.02 | −0.01 | 0.00 | 0.01 | 0.00 | 0.00 | 0.00 |
| 13 | 6 | −0.08 | 0.01 | 0.01 | 0.01 | 0.00 | 0.00 | 0.00 | 0.00 | 0.00 |
| 14 | 7 | 0.02 | −0.02 | 0.02 | 0.00 | 0.01 | −0.01 | 0.00 | 0.00 | 0.00 |
| 15 | 7 | 0.01 | 0.01 | −0.02 | 0.00 | 0.00 | 0.00 | 0.00 | 0.00 | 0.00 |
| 16 | 6 | 0.00 | 0.04 | 0.02 | −0.01 | 0.01 | 0.01 | 0.00 | 0.03 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.02 | 0.01 | 0.00 | −0.02 | 0.00 | 0.02 | −0.05 | −0.03 | 0.02 |
| 20 | 8 | −0.01 | 0.00 | 0.00 | 0.04 | 0.06 | −0.04 | 0.00 | 0.00 | 0.01 |
| 21 | 1 | 0.07 | −0.01 | 0.00 | 0.03 | 0.00 | 0.00 | 0.00 | 0.00 | 0.00 |
| 22 | 1 | 0.06 | 0.05 | −0.08 | 0.01 | 0.00 | 0.00 | 0.00 | 0.00 | 0.00 |
| 23 | 1 | −0.12 | 0.17 | −0.29 | 0.00 | 0.07 | −0.07 | 0.01 | −0.01 | 0.04 |
| 24 | 1 | 0.04 | 0.30 | −0.22 | −0.34 | −0.11 | 0.79 | 0.04 | 0.13 | −0.11 |
| 25 | 1 | −0.21 | 0.19 | −0.06 | −0.02 | 0.14 | −0.18 | 0.69 | −0.16 | −0.58 |
| 26 | 1 | 0.00 | 0.03 | 0.09 | 0.02 | 0.02 | −0.02 | 0.02 | 0.10 | 0.06 |
| 27 | 1 | 0.00 | 0.06 | −0.06 | −0.05 | −0.19 | 0.29 | 0.03 | 0.09 | −0.11 |
| 28 | 1 | −0.02 | −0.19 | −0.06 | −0.01 | −0.07 | −0.01 | −0.02 | −0.15 | −0.03 |
| 29 | 1 | 0.02 | −0.21 | −0.01 | 0.00 | −0.07 | 0.00 | 0.04 | −0.08 | −0.01 |
| 30 | 1 | 0.01 | 0.00 | 0.00 | 0.00 | 0.00 | 0.00 | 0.01 | 0.00 | 0.00 |
| 31 | 1 | 0.00 | 0.00 | 0.00 | 0.00 | 0.00 | 0.00 | 0.00 | 0.00 | 0.01 |
| 32 | 1 | 0.01 | 0.00 | 0.00 | 0.00 | 0.00 | 0.00 | 0.01 | −0.01 | 0.00 |
| 33 | 1 | −0.06 | 0.01 | 0.01 | −0.14 | −0.01 | 0.03 | −0.23 | −0.05 | 0.04 |
| 34 | 1 | 0.15 | 0.02 | −0.01 | −0.01 | 0.00 | 0.00 | 0.00 | 0.00 | 0.00 |
| 35 | 1 | −0.03 | −0.04 | 0.05 | 0.01 | 0.01 | −0.01 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.54 | −0.27 | 0.24 | −0.07 | 0.04 | −0.03 | 0.01 | −0.01 | 0.01 |
| 42 | 1 | −0.01 | 0.00 | 0.01 | 0.00 | 0.00 | 0.00 | 0.00 | 0.00 | 0.00 |
| 43 | 1 | 0.00 | −0.01 | −0.01 | 0.00 | 0.00 | 0.00 | 0.00 | 0.00 | 0.00 |
| 44 | 1 | −0.01 | 0.02 | −0.01 | 0.00 | 0.00 | 0.00 | 0.00 | 0.00 | 0.00 |

| | 85 | 86 | 87 |
|---|---|---|---|
| | ?A | ?A | ?A |
| Frequencies | 1306.7722 | 1335.0131 | 1343.5500 |
| Red. masses | 1.5805 | 1.2846 | 3.0882 |
| Frc consts | 1.5902 | 1.3489 | 3.2845 |
| IR Inten | 68.7837 | 101.9485 | 36.4228 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.01 | −0.02 | 0.01 | 0.00 | 0.00 | 0.00 | 0.00 | 0.02 | 0.00 |
| 2 | 6 | −0.02 | 0.07 | 0.00 | 0.00 | 0.00 | 0.00 | 0.01 | 0.00 | −0.02 |
| 3 | 8 | 0.01 | −0.06 | 0.00 | 0.00 | 0.00 | 0.00 | −0.01 | 0.01 | 0.00 |
| 4 | 6 | 0.02 | 0.08 | 0.13 | 0.00 | 0.00 | −0.01 | 0.00 | −0.01 | 0.00 |
| 5 | 6 | −0.03 | −0.02 | 0.01 | 0.00 | 0.00 | 0.00 | 0.00 | 0.00 | 0.00 |
| 6 | 7 | 0.00 | 0.00 | 0.00 | 0.00 | 0.00 | 0.00 | 0.13 | −0.08 | 0.09 |
| 7 | 6 | −0.01 | 0.00 | 0.00 | 0.00 | 0.00 | 0.00 | −0.12 | −0.03 | 0.07 |
| 8 | 6 | −0.01 | 0.00 | 0.00 | 0.00 | 0.00 | 0.00 | −0.12 | −0.01 | 0.04 |
| 9 | 7 | 0.00 | 0.00 | 0.00 | 0.00 | 0.00 | 0.00 | −0.09 | 0.06 | −0.06 |
| 10 | 6 | 0.00 | 0.00 | 0.00 | 0.00 | 0.00 | 0.00 | 0.04 | 0.00 | −0.01 |
| 11 | 6 | 0.00 | 0.00 | 0.01 | 0.00 | 0.00 | 0.00 | −0.05 | 0.04 | −0.04 |
| 12 | 7 | 0.00 | 0.00 | 0.00 | 0.00 | 0.00 | 0.00 | −0.07 | −0.09 | 0.14 |
| 13 | 6 | −0.01 | 0.00 | 0.00 | 0.00 | 0.00 | 0.00 | 0.04 | 0.01 | −0.02 |
| 14 | 7 | 0.00 | 0.00 | 0.00 | 0.00 | 0.00 | 0.00 | 0.01 | 0.10 | −0.15 |
| 15 | 7 | 0.00 | 0.00 | 0.00 | 0.00 | 0.00 | 0.00 | 0.09 | 0.00 | −0.02 |
| 16 | 6 | −0.01 | −0.11 | −0.04 | 0.00 | 0.00 | 0.00 | 0.00 | 0.00 | 0.00 |
| 17 | 16 | 0.01 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.01 | 0.00 | −0.02 | 0.00 | 0.00 | 0.00 | 0.00 | 0.00 | 0.00 |
| 20 | 8 | 0.01 | 0.01 | −0.01 | 0.00 | 0.00 | 0.00 | −0.01 | −0.01 | 0.01 |
| 21 | 1 | 0.02 | 0.00 | 0.00 | 0.01 | 0.00 | 0.00 | 0.65 | −0.05 | −0.09 |
| 22 | 1 | 0.03 | 0.00 | −0.01 | 0.00 | 0.00 | 0.00 | 0.32 | 0.07 | −0.18 |
| 23 | 1 | −0.13 | 0.10 | −0.17 | 0.01 | 0.00 | 0.01 | −0.02 | −0.02 | 0.05 |
| 24 | 1 | 0.02 | 0.02 | −0.06 | 0.00 | 0.00 | 0.00 | 0.02 | −0.01 | −0.03 |
| 25 | 1 | 0.09 | 0.09 | −0.35 | 0.00 | 0.00 | 0.00 | 0.02 | −0.02 | 0.01 |
| 26 | 1 | 0.08 | −0.36 | −0.31 | 0.00 | 0.01 | 0.01 | 0.00 | 0.00 | 0.00 |
| 27 | 1 | 0.00 | 0.02 | 0.00 | 0.00 | −0.01 | −0.01 | 0.00 | 0.01 | −0.01 |
| 28 | 1 | 0.02 | 0.38 | 0.24 | 0.00 | −0.01 | 0.00 | 0.00 | 0.02 | 0.00 |
| 29 | 1 | −0.06 | 0.55 | 0.03 | 0.00 | −0.01 | 0.00 | 0.00 | 0.01 | 0.00 |
| 30 | 1 | −0.01 | −0.02 | −0.02 | 0.00 | 0.00 | 0.00 | 0.00 | 0.00 | 0.00 |
| 31 | 1 | 0.02 | 0.01 | −0.03 | −0.01 | 0.00 | 0.00 | 0.00 | 0.00 | 0.00 |
| 32 | 1 | −0.03 | 0.00 | 0.01 | 0.00 | 0.00 | 0.00 | 0.00 | 0.00 | 0.00 |
| 33 | 1 | 0.02 | −0.01 | −0.02 | −0.01 | 0.00 | 0.00 | 0.01 | 0.00 | 0.00 |
| 34 | 1 | 0.02 | 0.00 | 0.00 | −0.01 | 0.00 | 0.00 | −0.07 | 0.00 | −0.02 |
| 35 | 1 | 0.00 | 0.00 | 0.01 | 0.01 | 0.00 | −0.01 | 0.21 | 0.12 | −0.18 |
| 36 | 15 | 0.00 | 0.00 | 0.00 | 0.06 | 0.01 | 0.02 | 0.00 | 0.00 | 0.00 |
| 37 | 8 | 0.00 | 0.00 | 0.00 | −0.04 | −0.01 | −0.01 | 0.00 | 0.00 | 0.00 |
| 38 | 8 | 0.00 | 0.00 | 0.00 | −0.04 | −0.01 | 0.00 | 0.00 | 0.00 | 0.00 |
| 39 | 8 | 0.00 | 0.00 | 0.00 | −0.02 | −0.05 | 0.00 | 0.00 | 0.00 | 0.00 |
| 40 | 8 | 0.00 | 0.00 | 0.00 | −0.03 | −0.02 | −0.03 | 0.00 | 0.00 | 0.00 |
| 41 | 1 | 0.00 | 0.00 | 0.00 | 0.01 | −0.01 | 0.01 | 0.31 | −0.17 | 0.16 |
| 42 | 1 | 0.01 | 0.00 | 0.00 | 0.37 | 0.15 | −0.24 | −0.02 | −0.01 | 0.01 |
| 43 | 1 | 0.00 | 0.00 | 0.00 | 0.13 | 0.31 | 0.35 | −0.01 | −0.01 | −0.02 |
| 44 | 1 | −0.01 | 0.03 | −0.01 | −0.25 | 0.63 | −0.27 | 0.01 | −0.03 | 0.01 |

| | 88 | 89 | 90 |
|---|---|---|---|
| | ?A | ?A | ?A |
| Frequencies | 1351.0672 | 1358.2855 | 1367.4047 |
| Red. masses | 2.7853 | 1.1431 | 2.3726 |
| Frc consts | 2.9956 | 1.2426 | 2.6138 |
| IR Inten | 36.8101 | 8.3893 | 9.2471 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | −0.01 | 0.00 | 0.00 | 0.00 | 0.00 | −0.02 | −0.06 | 0.02 |
| 2 | 6 | −0.01 | 0.00 | 0.02 | 0.00 | 0.00 | 0.01 | 0.00 | 0.03 | 0.03 |
| 3 | 8 | 0.00 | −0.01 | −0.01 | 0.00 | 0.00 | 0.00 | 0.01 | −0.01 | −0.04 |
| 4 | 6 | 0.00 | 0.01 | 0.01 | −0.01 | −0.01 | 0.01 | −0.06 | −0.18 | 0.19 |
| 5 | 6 | 0.00 | 0.00 | 0.00 | 0.00 | 0.00 | 0.00 | −0.13 | 0.05 | 0.02 |
| 6 | 7 | −0.10 | 0.05 | −0.05 | 0.00 | 0.00 | 0.00 | 0.00 | 0.00 | 0.00 |
| 7 | 6 | 0.10 | −0.07 | 0.07 | 0.00 | 0.00 | 0.00 | 0.00 | 0.00 | 0.00 |
| 8 | 6 | 0.14 | 0.01 | −0.05 | 0.00 | 0.00 | 0.00 | 0.00 | 0.00 | 0.00 |
| 9 | 7 | 0.01 | −0.01 | 0.01 | 0.00 | 0.00 | 0.00 | 0.00 | 0.00 | 0.00 |
| 10 | 6 | −0.07 | 0.00 | 0.01 | 0.00 | 0.00 | 0.00 | 0.00 | 0.00 | 0.00 |
| 11 | 6 | −0.06 | 0.09 | −0.12 | 0.00 | 0.00 | 0.00 | 0.00 | 0.00 | 0.00 |
| 12 | 7 | 0.00 | −0.04 | 0.05 | 0.00 | 0.00 | 0.00 | 0.00 | 0.00 | 0.00 |
| 13 | 6 | 0.17 | −0.04 | 0.02 | 0.00 | 0.00 | 0.00 | 0.00 | 0.00 | 0.00 |
| 14 | 7 | −0.15 | 0.04 | −0.01 | 0.00 | 0.00 | 0.00 | 0.00 | 0.00 | 0.00 |
| 15 | 7 | 0.00 | −0.03 | 0.05 | 0.00 | 0.00 | 0.00 | 0.00 | 0.00 | 0.00 |
| 16 | 6 | 0.00 | 0.00 | 0.00 | 0.00 | 0.00 | −0.01 | 0.01 | 0.10 | −0.03 |
| 17 | 16 | 0.00 | 0.00 | 0.00 | −0.01 | 0.00 | 0.00 | 0.00 | 0.00 | 0.00 |
| 18 | 6 | 0.00 | 0.00 | 0.00 | −0.10 | 0.05 | 0.00 | 0.01 | 0.00 | −0.02 |
| 19 | 8 | 0.00 | 0.00 | 0.00 | 0.00 | 0.00 | 0.00 | 0.07 | 0.01 | −0.06 |
| 20 | 8 | 0.01 | 0.01 | −0.01 | 0.00 | 0.00 | 0.00 | 0.02 | 0.03 | −0.03 |
| 21 | 1 | −0.14 | −0.01 | 0.05 | 0.00 | 0.00 | 0.00 | 0.00 | 0.00 | 0.00 |
| 22 | 1 | −0.15 | −0.01 | 0.05 | 0.00 | 0.00 | 0.00 | 0.00 | 0.00 | 0.00 |
| 23 | 1 | 0.02 | 0.03 | −0.06 | 0.00 | 0.00 | −0.01 | −0.07 | 0.05 | −0.06 |
| 24 | 1 | 0.00 | −0.01 | −0.01 | 0.00 | 0.00 | 0.00 | −0.01 | −0.09 | 0.01 |
| 25 | 1 | −0.01 | 0.02 | −0.02 | 0.00 | −0.01 | 0.02 | −0.02 | −0.06 | 0.10 |
| 26 | 1 | 0.01 | −0.03 | −0.02 | 0.00 | 0.07 | 0.01 | 0.04 | 0.30 | −0.14 |
| 27 | 1 | 0.00 | 0.00 | 0.00 | 0.00 | 0.00 | 0.00 | 0.00 | 0.00 | 0.02 |
| 28 | 1 | 0.00 | −0.01 | 0.02 | 0.00 | 0.02 | 0.01 | 0.05 | 0.03 | −0.45 |
| 29 | 1 | −0.02 | 0.01 | 0.00 | 0.00 | 0.02 | 0.00 | 0.57 | 0.19 | −0.14 |
| 30 | 1 | 0.01 | −0.02 | −0.01 | 0.35 | −0.26 | −0.08 | −0.08 | 0.12 | 0.07 |
| 31 | 1 | 0.02 | 0.01 | 0.00 | 0.60 | 0.10 | 0.21 | −0.08 | −0.07 | 0.14 |
| 32 | 1 | 0.01 | −0.01 | 0.00 | 0.41 | −0.43 | −0.13 | 0.00 | −0.03 | 0.00 |
| 33 | 1 | 0.00 | 0.00 | 0.00 | 0.01 | 0.00 | 0.00 | 0.29 | 0.01 | −0.08 |
| 34 | 1 | −0.34 | −0.04 | 0.04 | 0.00 | 0.00 | 0.00 | 0.00 | 0.00 | 0.00 |
| 35 | 1 | 0.15 | 0.13 | −0.19 | 0.00 | 0.00 | 0.00 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.60 | −0.36 | 0.33 | −0.01 | 0.01 | −0.01 | −0.01 | 0.00 | 0.00 |
| 42 | 1 | −0.01 | 0.00 | 0.01 | 0.00 | 0.00 | 0.00 | 0.01 | 0.00 | −0.01 |
| 43 | 1 | 0.00 | −0.01 | −0.01 | 0.00 | 0.00 | 0.00 | 0.00 | 0.01 | 0.01 |
| 44 | 1 | 0.00 | −0.01 | 0.00 | 0.00 | 0.00 | 0.00 | 0.00 | 0.01 | 0.00 |

| | 91 | 92 | 93 |
|---|---|---|---|
| | ?A | ?A | ?A |
| Frequencies | 1370.2928 | 1378.2023 | 1381.2838 |
| Red. masses | 1.0904 | 1.1122 | 1.4245 |
| Frc consts | 1.2063 | 1.2447 | 1.6013 |
| IR Inten | 10.8590 | 10.0357 | 8.1974 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | −0.01 | 0.00 | −0.01 | −0.02 | 0.01 |
| 2 | 6 | 0.00 | 0.00 | −0.01 | 0.00 | 0.00 | 0.00 | 0.00 | 0.00 | 0.02 |
| 3 | 8 | 0.00 | 0.00 | 0.00 | 0.00 | 0.00 | 0.00 | 0.00 | 0.00 | −0.02 |
| 4 | 6 | 0.01 | 0.02 | −0.02 | −0.01 | −0.02 | 0.01 | −0.03 | −0.08 | 0.05 |
| 5 | 6 | 0.01 | −0.01 | 0.00 | −0.01 | 0.00 | 0.00 | −0.05 | 0.02 | 0.01 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.00 | 0.00 | 0.00 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | 0.00 | 0.00 | 0.00 | 0.00 | 0.00 | 0.00 | 0.00 | 0.00 | 0.00 |
| 16 | 6 | 0.00 | −0.01 | 0.00 | 0.03 | 0.03 | −0.03 | 0.08 | 0.09 | −0.08 |
| 17 | 16 | 0.00 | 0.00 | 0.00 | 0.00 | 0.00 | 0.00 | 0.00 | −0.01 | 0.00 |
| 18 | 6 | 0.03 | 0.03 | −0.07 | −0.04 | −0.05 | −0.04 | 0.02 | 0.02 | 0.02 |
| 19 | 8 | 0.00 | 0.00 | 0.00 | 0.01 | 0.00 | −0.01 | 0.03 | 0.00 | −0.02 |
| 20 | 8 | 0.00 | 0.00 | 0.00 | 0.00 | 0.00 | 0.00 | 0.01 | 0.01 | −0.01 |
| 21 | 1 | 0.00 | 0.00 | 0.00 | 0.00 | 0.00 | 0.00 | 0.00 | 0.00 | 0.00 |
| 22 | 1 | 0.00 | 0.00 | 0.00 | 0.00 | 0.00 | 0.00 | 0.00 | 0.00 | −0.01 |
| 23 | 1 | 0.01 | 0.00 | 0.01 | −0.01 | 0.00 | 0.00 | −0.02 | 0.01 | −0.01 |
| 24 | 1 | 0.00 | 0.01 | 0.00 | 0.00 | 0.00 | 0.00 | 0.00 | −0.03 | 0.00 |
| 25 | 1 | 0.00 | 0.01 | −0.02 | 0.00 | −0.01 | 0.01 | −0.01 | −0.03 | 0.05 |
| 26 | 1 | 0.00 | −0.07 | −0.01 | 0.00 | −0.02 | −0.03 | 0.00 | 0.11 | −0.01 |
| 27 | 1 | 0.00 | 0.00 | 0.00 | 0.00 | 0.00 | 0.00 | 0.00 | 0.00 | 0.00 |
| 28 | 1 | −0.01 | −0.01 | 0.06 | −0.02 | −0.06 | 0.24 | −0.05 | −0.16 | 0.60 |
| 29 | 1 | −0.08 | −0.02 | 0.02 | −0.24 | −0.02 | 0.04 | −0.59 | −0.05 | 0.09 |
| 30 | 1 | −0.35 | 0.31 | 0.08 | 0.28 | 0.34 | 0.54 | −0.13 | −0.17 | −0.26 |
| 31 | 1 | −0.09 | −0.25 | 0.66 | −0.06 | −0.10 | 0.17 | 0.00 | 0.05 | −0.12 |
| 32 | 1 | −0.03 | −0.48 | 0.07 | 0.36 | 0.39 | −0.24 | −0.18 | −0.15 | 0.12 |
| 33 | 1 | −0.03 | 0.00 | 0.01 | 0.01 | 0.00 | 0.00 | 0.05 | 0.00 | −0.02 |
| 34 | 1 | 0.00 | 0.00 | 0.00 | 0.00 | 0.00 | 0.00 | 0.00 | 0.00 | 0.00 |
| 35 | 1 | 0.00 | 0.00 | 0.00 | 0.00 | 0.00 | 0.00 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.00 | 0.00 | 0.00 | 0.00 | 0.00 | 0.00 | −0.01 | 0.00 | 0.00 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 94 | 95 | 96 |
|---|---|---|---|
| | ?A | ?A | ?A |
| Frequencies | 1422.5977 | 1425.5132 | 1436.6256 |
| Red. masses | 5.9275 | 4.6666 | 6.4700 |
| Frc consts | 7.0678 | 5.5871 | 7.8676 |
| IR Inten | 33.3011 | 32.4640 | 24.0951 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.02 | 0.03 | −0.03 | −0.10 | −0.10 | 0.08 | 0.23 | 0.36 | −0.35 |
| 2 | 6 | 0.01 | 0.00 | −0.01 | 0.00 | 0.04 | 0.02 | −0.02 | 0.02 | 0.08 |
| 3 | 8 | 0.00 | 0.00 | 0.00 | 0.00 | 0.00 | 0.00 | 0.01 | −0.01 | 0.00 |
| 4 | 6 | 0.00 | −0.01 | 0.00 | −0.08 | −0.09 | 0.11 | −0.02 | −0.11 | 0.15 |
| 5 | 6 | −0.03 | 0.00 | 0.01 | 0.38 | 0.00 | −0.18 | 0.09 | −0.04 | −0.08 |
| 6 | 7 | 0.02 | 0.03 | −0.05 | 0.00 | 0.00 | 0.00 | −0.01 | 0.00 | 0.00 |
| 7 | 6 | 0.17 | 0.06 | −0.12 | 0.01 | 0.00 | −0.01 | −0.02 | 0.00 | 0.00 |
| 8 | 6 | −0.24 | −0.03 | 0.10 | −0.02 | 0.00 | 0.01 | 0.03 | 0.01 | −0.02 |
| 9 | 7 | 0.00 | −0.01 | 0.02 | 0.00 | 0.00 | 0.00 | 0.00 | 0.00 | 0.00 |
| 10 | 6 | 0.08 | −0.08 | 0.10 | 0.01 | 0.00 | 0.00 | −0.01 | 0.00 | 0.00 |
| 11 | 6 | −0.12 | 0.08 | −0.09 | −0.01 | 0.00 | −0.01 | 0.01 | −0.01 | 0.01 |
| 12 | 7 | 0.29 | 0.02 | −0.10 | 0.02 | 0.00 | −0.01 | −0.01 | 0.00 | 0.00 |
| 13 | 6 | −0.10 | −0.14 | 0.23 | −0.01 | −0.01 | 0.01 | 0.01 | 0.01 | −0.01 |
| 14 | 7 | −0.20 | 0.10 | −0.08 | −0.01 | 0.01 | −0.01 | 0.01 | −0.01 | 0.01 |
| 15 | 7 | 0.09 | −0.04 | 0.04 | 0.01 | 0.00 | 0.00 | −0.01 | 0.00 | 0.00 |
| 16 | 6 | 0.00 | 0.00 | 0.00 | 0.03 | 0.04 | −0.03 | 0.03 | 0.05 | −0.04 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.02 | 0.00 | −0.01 | −0.23 | −0.01 | 0.08 | −0.08 | 0.00 | 0.02 |
| 20 | 8 | −0.01 | −0.02 | 0.01 | 0.03 | 0.04 | −0.04 | −0.16 | −0.19 | 0.15 |
| 21 | 1 | 0.21 | −0.17 | 0.18 | 0.01 | −0.01 | 0.01 | −0.02 | 0.01 | 0.00 |
| 22 | 1 | 0.07 | −0.09 | 0.11 | 0.00 | −0.01 | 0.01 | −0.01 | 0.00 | 0.00 |
| 23 | 1 | −0.04 | 0.00 | −0.01 | −0.07 | 0.04 | 0.01 | −0.01 | 0.14 | −0.28 |
| 24 | 1 | 0.00 | −0.05 | 0.03 | 0.03 | 0.36 | −0.23 | 0.00 | −0.10 | 0.17 |
| 25 | 1 | 0.03 | −0.03 | −0.01 | −0.11 | 0.12 | 0.23 | 0.20 | −0.14 | −0.02 |
| 26 | 1 | 0.00 | 0.02 | 0.04 | 0.07 | 0.14 | −0.50 | 0.07 | 0.11 | −0.25 |
| 27 | 1 | −0.01 | −0.03 | 0.03 | 0.02 | 0.01 | −0.05 | −0.16 | −0.30 | 0.34 |
| 28 | 1 | 0.00 | −0.01 | 0.01 | 0.01 | 0.03 | 0.02 | 0.00 | −0.02 | 0.03 |
| 29 | 1 | −0.01 | 0.00 | 0.00 | 0.00 | 0.07 | −0.01 | 0.05 | 0.11 | −0.03 |
| 30 | 1 | 0.00 | 0.00 | 0.00 | −0.02 | 0.00 | −0.01 | −0.01 | 0.00 | −0.01 |
| 31 | 1 | 0.00 | 0.00 | 0.00 | −0.01 | 0.00 | 0.00 | −0.01 | 0.00 | 0.00 |
| 32 | 1 | 0.00 | 0.00 | 0.00 | −0.02 | −0.01 | 0.01 | −0.01 | 0.00 | 0.00 |
| 33 | 1 | −0.02 | 0.00 | 0.00 | 0.36 | 0.01 | −0.04 | 0.08 | 0.03 | −0.01 |
| 34 | 1 | −0.26 | −0.02 | 0.04 | −0.02 | 0.00 | 0.00 | 0.02 | 0.00 | 0.00 |
| 35 | 1 | 0.27 | 0.16 | −0.22 | 0.02 | 0.01 | −0.01 | −0.02 | −0.01 | 0.02 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.39 | 0.20 | −0.18 | −0.04 | 0.02 | −0.02 | 0.06 | −0.03 | 0.03 |
| 42 | 1 | 0.00 | 0.00 | 0.00 | 0.01 | 0.00 | −0.01 | −0.01 | −0.01 | 0.01 |
| 43 | 1 | 0.00 | 0.00 | 0.00 | 0.00 | 0.01 | 0.01 | 0.00 | 0.00 | −0.01 |
| 44 | 1 | 0.00 | 0.00 | 0.00 | 0.00 | 0.01 | 0.00 | 0.00 | −0.01 | 0.00 |

| | 97 | 98 | 99 |
|---|---|---|---|
| | ?A | ?A | ?A |
| Frequencies | 1468.7313 | 1507.9372 | 1544.9266 |
| Red. masses | 9.8376 | 1.4553 | 5.6170 |
| Frc consts | 12.5033 | 1.9497 | 7.8990 |
| IR Inten | 20.0376 | 17.7890 | 95.6990 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.01 | 0.01 | −0.01 | 0.01 | 0.00 | −0.01 | −0.01 | 0.00 | −0.01 |
| 2 | 6 | −0.01 | −0.01 | 0.02 | 0.00 | −0.01 | −0.01 | 0.00 | 0.00 | −0.01 |
| 3 | 8 | 0.00 | 0.00 | −0.01 | 0.01 | −0.01 | 0.01 | 0.00 | 0.00 | 0.00 |
| 4 | 6 | 0.00 | 0.01 | 0.01 | −0.02 | 0.03 | −0.06 | 0.00 | 0.00 | 0.00 |
| 5 | 6 | 0.00 | 0.00 | 0.00 | 0.01 | −0.02 | 0.16 | 0.00 | 0.00 | 0.00 |
| 6 | 7 | −0.16 | 0.11 | −0.12 | 0.00 | 0.00 | 0.00 | 0.10 | −0.04 | 0.04 |
| 7 | 6 | 0.44 | −0.14 | 0.08 | 0.00 | 0.00 | 0.00 | −0.07 | −0.02 | 0.04 |
| 8 | 6 | −0.26 | −0.24 | 0.39 | 0.00 | 0.00 | 0.00 | −0.03 | −0.01 | 0.01 |
| 9 | 7 | 0.05 | 0.16 | −0.23 | 0.00 | 0.00 | 0.00 | 0.39 | −0.09 | 0.03 |
| 10 | 6 | −0.07 | −0.05 | 0.08 | 0.00 | 0.00 | 0.00 | −0.42 | 0.09 | −0.02 |
| 11 | 6 | 0.07 | −0.02 | 0.01 | 0.00 | 0.00 | 0.00 | −0.08 | 0.03 | −0.03 |
| 12 | 7 | −0.21 | −0.02 | 0.08 | 0.00 | 0.00 | 0.00 | −0.01 | −0.02 | 0.03 |
| 13 | 6 | 0.08 | 0.12 | −0.18 | 0.00 | 0.00 | 0.00 | 0.04 | −0.01 | 0.00 |
| 14 | 7 | 0.09 | −0.01 | −0.01 | 0.00 | 0.00 | 0.00 | −0.02 | 0.04 | −0.05 |
| 15 | 7 | 0.01 | 0.01 | −0.01 | 0.00 | 0.00 | 0.00 | 0.04 | 0.00 | −0.01 |
| 16 | 6 | 0.00 | 0.00 | 0.00 | 0.00 | 0.00 | 0.01 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.00 | 0.00 | 0.00 | −0.07 | 0.00 | −0.05 | 0.00 | 0.00 | 0.00 |
| 20 | 8 | 0.00 | 0.00 | 0.00 | −0.01 | 0.00 | 0.00 | 0.01 | −0.01 | 0.01 |
| 21 | 1 | −0.12 | 0.14 | −0.16 | 0.00 | 0.00 | 0.00 | 0.07 | −0.01 | 0.00 |
| 22 | 1 | 0.13 | 0.02 | −0.06 | 0.00 | 0.00 | 0.00 | −0.03 | 0.20 | −0.27 |
| 23 | 1 | 0.02 | 0.02 | −0.05 | 0.00 | −0.01 | 0.00 | −0.01 | 0.00 | 0.01 |
| 24 | 1 | −0.01 | 0.00 | 0.02 | −0.01 | 0.05 | 0.03 | −0.03 | −0.04 | 0.03 |
| 25 | 1 | 0.00 | 0.00 | −0.01 | 0.17 | 0.04 | −0.18 | 0.00 | 0.00 | 0.00 |
| 26 | 1 | 0.00 | −0.02 | −0.02 | −0.02 | 0.01 | 0.03 | 0.00 | 0.01 | 0.00 |
| 27 | 1 | −0.01 | −0.02 | 0.03 | −0.02 | −0.03 | 0.05 | 0.04 | 0.10 | −0.12 |
| 28 | 1 | 0.00 | 0.00 | 0.00 | 0.00 | 0.00 | 0.01 | 0.00 | −0.01 | 0.00 |
| 29 | 1 | 0.00 | 0.01 | 0.00 | −0.04 | −0.05 | 0.01 | 0.00 | 0.00 | 0.00 |
| 30 | 1 | 0.00 | 0.00 | 0.00 | 0.00 | 0.00 | 0.00 | 0.00 | 0.00 | 0.00 |
| 31 | 1 | 0.00 | 0.00 | 0.00 | 0.00 | 0.00 | 0.00 | 0.00 | 0.00 | 0.00 |
| 32 | 1 | 0.00 | 0.00 | 0.00 | 0.00 | 0.00 | 0.00 | 0.00 | 0.00 | 0.00 |
| 33 | 1 | −0.01 | 0.00 | 0.00 | 0.92 | 0.03 | −0.17 | 0.02 | 0.00 | 0.00 |
| 34 | 1 | 0.03 | 0.00 | −0.02 | 0.00 | 0.00 | 0.00 | −0.02 | 0.01 | −0.01 |
| 35 | 1 | −0.03 | −0.04 | 0.06 | 0.00 | 0.00 | 0.00 | 0.09 | 0.06 | −0.07 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.08 | 0.20 | −0.30 | 0.00 | 0.00 | 0.00 | −0.40 | 0.35 | −0.40 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 100 | 101 | 102 |
|---|---|---|---|
| | ?A | ?A | ?A |
| Frequencies | 1546.0112 | 1564.4680 | 1600.7451 |
| Red. masses | 1.2370 | 6.8428 | 6.3785 |
| Frc consts | 1.7421 | 9.8677 | 9.6297 |
| IR Inten | 55.4487 | 150.4572 | 252.5464 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.07 | −0.02 | 0.07 | 0.02 | 0.00 | 0.01 | 0.00 | 0.01 | 0.02 |
| 2 | 6 | 0.00 | 0.00 | −0.02 | 0.01 | −0.05 | 0.07 | −0.08 | 0.18 | −0.24 |
| 3 | 8 | 0.00 | 0.00 | 0.00 | −0.03 | 0.04 | −0.04 | 0.10 | −0.12 | 0.15 |
| 4 | 6 | 0.00 | 0.00 | −0.01 | 0.00 | 0.02 | 0.01 | −0.01 | −0.06 | −0.03 |
| 5 | 6 | −0.02 | 0.01 | 0.00 | −0.01 | 0.00 | 0.00 | 0.00 | 0.00 | −0.01 |
| 6 | 7 | 0.01 | 0.01 | −0.01 | −0.01 | −0.14 | 0.20 | 0.14 | 0.10 | −0.18 |
| 7 | 6 | 0.00 | 0.00 | 0.00 | −0.10 | 0.02 | 0.00 | −0.15 | 0.00 | 0.04 |
| 8 | 6 | −0.01 | 0.01 | −0.01 | 0.14 | −0.19 | 0.23 | 0.04 | −0.02 | 0.02 |
| 9 | 7 | 0.01 | −0.01 | 0.01 | −0.01 | 0.11 | −0.15 | −0.03 | 0.08 | −0.10 |
| 10 | 6 | −0.02 | −0.01 | 0.01 | −0.01 | 0.13 | −0.18 | −0.17 | −0.17 | 0.27 |
| 11 | 6 | −0.01 | 0.00 | 0.00 | 0.24 | −0.03 | −0.01 | 0.13 | −0.09 | 0.10 |
| 12 | 7 | 0.00 | 0.00 | 0.01 | 0.04 | 0.11 | −0.16 | 0.02 | 0.07 | −0.11 |
| 13 | 6 | 0.00 | 0.00 | −0.01 | 0.12 | −0.12 | 0.14 | 0.05 | −0.05 | 0.06 |
| 14 | 7 | 0.01 | 0.00 | 0.00 | −0.15 | 0.11 | −0.11 | −0.04 | 0.05 | −0.05 |
| 15 | 7 | 0.01 | 0.00 | 0.00 | −0.15 | −0.01 | 0.05 | −0.05 | 0.01 | 0.00 |
| 16 | 6 | 0.00 | 0.00 | 0.00 | 0.00 | −0.01 | 0.00 | 0.00 | 0.03 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.01 | 0.00 | 0.00 | 0.00 | 0.00 | 0.00 | 0.00 | 0.00 | 0.00 |
| 20 | 8 | −0.04 | 0.03 | −0.06 | −0.01 | 0.01 | −0.02 | 0.00 | −0.01 | 0.02 |
| 21 | 1 | 0.01 | 0.00 | −0.01 | −0.13 | −0.09 | 0.16 | −0.06 | −0.03 | 0.06 |
| 22 | 1 | 0.01 | 0.00 | 0.00 | −0.01 | 0.15 | −0.21 | 0.32 | −0.06 | 0.01 |
| 23 | 1 | 0.01 | −0.01 | 0.02 | 0.08 | −0.01 | −0.04 | −0.13 | 0.05 | 0.14 |
| 24 | 1 | 0.18 | 0.24 | −0.26 | 0.07 | 0.03 | −0.10 | −0.06 | 0.03 | 0.10 |
| 25 | 1 | 0.03 | −0.03 | 0.01 | 0.01 | 0.00 | −0.02 | −0.01 | −0.03 | 0.05 |
| 26 | 1 | −0.01 | 0.00 | 0.02 | 0.00 | −0.08 | −0.05 | −0.01 | 0.22 | 0.15 |
| 27 | 1 | −0.23 | −0.57 | 0.66 | −0.06 | −0.14 | 0.16 | 0.02 | 0.05 | −0.04 |
| 28 | 1 | 0.00 | 0.00 | 0.00 | 0.00 | 0.02 | 0.00 | 0.00 | −0.03 | 0.00 |
| 29 | 1 | 0.00 | −0.01 | 0.00 | 0.00 | 0.01 | 0.00 | −0.01 | −0.03 | 0.00 |
| 30 | 1 | 0.00 | 0.00 | 0.00 | 0.00 | 0.01 | 0.01 | 0.00 | −0.01 | −0.01 |
| 31 | 1 | 0.00 | 0.00 | 0.00 | 0.00 | 0.00 | 0.00 | 0.00 | 0.00 | 0.00 |
| 32 | 1 | 0.00 | 0.00 | 0.00 | 0.00 | 0.00 | 0.00 | 0.00 | −0.01 | 0.00 |
| 33 | 1 | −0.06 | 0.00 | 0.01 | 0.00 | 0.00 | 0.00 | −0.04 | 0.00 | 0.01 |
| 34 | 1 | 0.01 | 0.00 | 0.00 | −0.21 | −0.02 | 0.05 | 0.01 | 0.02 | 0.01 |
| 35 | 1 | 0.01 | 0.01 | −0.01 | −0.32 | −0.19 | 0.31 | −0.19 | −0.11 | 0.20 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.01 | 0.00 | 0.00 | 0.07 | 0.07 | −0.12 | 0.39 | −0.18 | 0.14 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 103 | 104 | 105 |
|---|---|---|---|
| | ?A | ?A | ?A |
| Frequencies | 1638.3335 | 1698.9548 | 1737.8654 |
| Red. masses | 8.3020 | 1.3788 | 3.8851 |
| Frc consts | 13.1292 | 2.3449 | 6.9132 |
| IR Inten | 198.6892 | 2.7060 | 146.2970 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.03 | −0.01 | −0.02 | 0.00 | 0.00 | 0.00 | 0.00 | 0.00 | 0.00 |
| 2 | 6 | 0.19 | −0.29 | 0.36 | 0.00 | 0.00 | 0.00 | 0.00 | 0.00 | 0.00 |
| 3 | 8 | −0.16 | 0.21 | −0.23 | 0.00 | 0.00 | 0.00 | 0.00 | 0.00 | 0.00 |
| 4 | 6 | 0.01 | 0.08 | 0.04 | 0.00 | 0.00 | 0.00 | 0.00 | 0.00 | 0.00 |
| 5 | 6 | 0.01 | 0.00 | 0.02 | 0.00 | 0.00 | 0.00 | 0.00 | 0.00 | 0.00 |
| 6 | 7 | 0.15 | 0.11 | −0.19 | −0.01 | −0.01 | 0.01 | −0.01 | −0.02 | 0.03 |
| 7 | 6 | −0.10 | −0.02 | 0.05 | 0.01 | 0.00 | −0.01 | 0.04 | 0.02 | −0.04 |
| 8 | 6 | −0.01 | 0.04 | −0.05 | 0.01 | −0.01 | 0.01 | −0.04 | 0.03 | −0.03 |
| 9 | 7 | −0.02 | 0.03 | −0.04 | −0.01 | 0.00 | 0.00 | −0.01 | −0.02 | 0.03 |
| 10 | 6 | −0.12 | −0.15 | 0.24 | 0.01 | 0.01 | −0.01 | 0.04 | 0.02 | −0.03 |
| 11 | 6 | 0.05 | −0.08 | 0.10 | −0.06 | −0.05 | 0.10 | −0.18 | −0.14 | 0.23 |
| 12 | 7 | 0.01 | 0.04 | −0.06 | −0.02 | 0.03 | −0.04 | −0.06 | 0.04 | −0.05 |
| 13 | 6 | 0.02 | −0.02 | 0.02 | 0.08 | −0.01 | 0.00 | 0.22 | −0.02 | −0.03 |
| 14 | 7 | −0.01 | 0.03 | −0.04 | −0.05 | 0.02 | −0.01 | −0.11 | 0.03 | −0.01 |
| 15 | 7 | −0.01 | 0.01 | −0.02 | −0.03 | 0.00 | 0.01 | 0.19 | 0.05 | −0.13 |
| 16 | 6 | 0.00 | −0.04 | −0.01 | 0.00 | 0.00 | 0.00 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | −0.01 | 0.00 | 0.00 | 0.00 | 0.00 | 0.00 | 0.00 | 0.00 | 0.00 |
| 20 | 8 | 0.01 | 0.01 | −0.02 | 0.00 | 0.00 | 0.00 | 0.00 | 0.00 | 0.00 |
| 21 | 1 | −0.01 | −0.01 | 0.02 | −0.06 | 0.01 | 0.01 | −0.12 | 0.02 | 0.00 |
| 22 | 1 | 0.26 | −0.07 | 0.04 | −0.01 | 0.00 | 0.00 | −0.04 | 0.00 | 0.01 |
| 23 | 1 | 0.01 | −0.14 | −0.16 | 0.00 | 0.00 | 0.00 | 0.01 | 0.00 | 0.00 |
| 24 | 1 | 0.05 | −0.08 | −0.10 | 0.00 | 0.00 | 0.00 | 0.00 | 0.00 | 0.00 |
| 25 | 1 | 0.00 | 0.05 | −0.06 | 0.00 | 0.00 | 0.00 | 0.00 | 0.00 | 0.00 |
| 26 | 1 | 0.01 | −0.29 | −0.22 | 0.00 | 0.00 | 0.00 | 0.00 | 0.00 | 0.00 |
| 27 | 1 | 0.01 | 0.01 | −0.04 | 0.00 | 0.00 | 0.00 | 0.00 | 0.00 | 0.00 |
| 28 | 1 | 0.00 | 0.03 | 0.02 | 0.00 | 0.00 | 0.00 | 0.00 | 0.00 | 0.00 |
| 29 | 1 | 0.00 | 0.04 | 0.00 | 0.00 | 0.00 | 0.00 | 0.00 | 0.00 | 0.00 |
| 30 | 1 | 0.00 | 0.01 | 0.01 | 0.00 | 0.00 | 0.00 | 0.00 | 0.00 | 0.00 |
| 31 | 1 | 0.00 | 0.00 | 0.00 | 0.00 | 0.00 | 0.00 | 0.00 | 0.00 | 0.00 |
| 32 | 1 | 0.00 | 0.01 | 0.00 | 0.00 | 0.00 | 0.00 | 0.00 | 0.00 | 0.00 |
| 33 | 1 | 0.07 | −0.01 | −0.02 | 0.00 | 0.00 | 0.00 | 0.00 | 0.00 | 0.00 |
| 34 | 1 | 0.10 | 0.01 | −0.01 | 0.72 | −0.10 | −0.05 | −0.46 | 0.21 | 0.00 |
| 35 | 1 | −0.06 | −0.04 | 0.06 | 0.30 | 0.23 | −0.54 | −0.25 | −0.22 | 0.60 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.24 | −0.13 | 0.11 | −0.02 | 0.01 | −0.01 | −0.06 | 0.01 | 0.00 |
| 42 | 1 | 0.01 | 0.00 | −0.01 | 0.00 | 0.00 | 0.00 | 0.00 | 0.00 | 0.00 |
| 43 | 1 | 0.00 | 0.00 | 0.01 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 106 | 107 | 108 |
|---|---|---|---|
| | ?A | ?A | ?A |
| Frequencies | 1775.1760 | 1783.2618 | 3023.2336 |
| Red. masses | 11.2051 | 10.7337 | 1.0834 |
| Frc consts | 20.8041 | 20.1109 | 5.8344 |
| IR Inten | 147.7554 | 576.9616 | 6.5286 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | 0.00 | 0.00 | 0.00 | 0.00 | 0.00 |
| 2 | 6 | −0.02 | 0.01 | −0.01 | −0.02 | 0.01 | −0.01 | 0.00 | 0.00 | 0.00 |
| 3 | 8 | 0.01 | −0.01 | 0.01 | 0.01 | −0.01 | 0.01 | 0.00 | 0.00 | 0.00 |
| 4 | 6 | 0.00 | 0.00 | 0.00 | 0.00 | 0.00 | 0.00 | −0.04 | 0.00 | −0.01 |
| 5 | 6 | 0.00 | 0.00 | 0.00 | 0.00 | 0.00 | 0.00 | 0.00 | 0.01 | 0.00 |
| 6 | 7 | −0.13 | 0.05 | −0.03 | −0.03 | 0.00 | 0.01 | 0.00 | 0.00 | 0.00 |
| 7 | 6 | 0.09 | −0.42 | 0.56 | 0.11 | −0.12 | 0.14 | 0.00 | 0.00 | 0.00 |
| 8 | 6 | 0.11 | 0.04 | −0.09 | −0.46 | 0.29 | −0.29 | 0.00 | 0.00 | 0.00 |
| 9 | 7 | 0.00 | 0.00 | −0.01 | 0.07 | −0.12 | 0.16 | 0.00 | 0.00 | 0.00 |
| 10 | 6 | 0.03 | 0.08 | −0.12 | 0.09 | 0.02 | −0.05 | 0.00 | 0.00 | 0.00 |
| 11 | 6 | −0.12 | −0.10 | 0.17 | 0.50 | −0.08 | 0.00 | 0.00 | 0.00 | 0.00 |
| 12 | 7 | 0.19 | 0.09 | −0.17 | −0.09 | −0.01 | 0.04 | 0.00 | 0.00 | 0.00 |
| 13 | 6 | −0.34 | 0.04 | 0.04 | 0.21 | −0.05 | 0.01 | 0.00 | 0.00 | 0.00 |
| 14 | 7 | 0.10 | 0.15 | −0.23 | −0.14 | 0.10 | −0.11 | 0.00 | 0.00 | 0.00 |
| 15 | 7 | 0.05 | 0.01 | −0.04 | −0.16 | −0.01 | 0.06 | 0.00 | 0.00 | 0.00 |
| 16 | 6 | 0.00 | 0.00 | 0.00 | 0.00 | 0.00 | 0.00 | 0.05 | −0.01 | 0.05 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.00 | 0.00 | 0.00 | 0.00 | 0.00 | 0.00 | 0.00 | 0.00 | 0.00 |
| 20 | 8 | 0.00 | 0.00 | 0.00 | 0.00 | 0.00 | 0.00 | 0.00 | 0.00 | 0.00 |
| 21 | 1 | 0.21 | 0.01 | −0.07 | −0.07 | −0.01 | 0.04 | 0.00 | 0.00 | 0.00 |
| 22 | 1 | −0.15 | 0.07 | −0.06 | −0.08 | −0.04 | 0.08 | 0.00 | 0.00 | 0.00 |
| 23 | 1 | 0.03 | 0.02 | 0.00 | 0.01 | 0.01 | 0.00 | 0.00 | −0.01 | 0.00 |
| 24 | 1 | 0.00 | −0.01 | 0.00 | 0.00 | 0.00 | 0.01 | 0.03 | 0.00 | 0.01 |
| 25 | 1 | 0.00 | 0.00 | 0.00 | 0.00 | 0.00 | 0.00 | −0.05 | −0.06 | −0.03 |
| 26 | 1 | 0.00 | 0.01 | 0.01 | 0.00 | 0.01 | 0.01 | 0.58 | −0.04 | 0.12 |
| 27 | 1 | 0.00 | 0.00 | 0.00 | 0.00 | 0.00 | 0.01 | 0.00 | 0.00 | 0.00 |
| 28 | 1 | 0.00 | 0.00 | 0.00 | 0.00 | 0.00 | 0.00 | −0.52 | 0.07 | −0.04 |
| 29 | 1 | 0.00 | 0.00 | 0.00 | 0.00 | 0.00 | 0.00 | −0.11 | 0.03 | −0.59 |
| 30 | 1 | 0.00 | 0.00 | 0.00 | 0.00 | 0.00 | 0.00 | 0.00 | 0.00 | 0.00 |
| 31 | 1 | 0.00 | 0.00 | 0.00 | 0.00 | 0.00 | 0.00 | 0.00 | 0.00 | 0.00 |
| 32 | 1 | 0.00 | 0.00 | 0.00 | 0.00 | 0.00 | 0.00 | 0.00 | 0.00 | 0.00 |
| 33 | 1 | 0.00 | 0.00 | 0.00 | −0.01 | 0.00 | 0.00 | 0.00 | 0.00 | 0.00 |
| 34 | 1 | 0.01 | 0.07 | 0.01 | 0.11 | −0.09 | 0.00 | 0.00 | 0.00 | 0.00 |
| 35 | 1 | −0.05 | −0.03 | 0.12 | −0.15 | −0.11 | 0.04 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | −0.03 | 0.04 | −0.04 | −0.24 | 0.04 | 0.00 | 0.00 | 0.00 | 0.00 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 109 | 110 | 111 |
|---|---|---|---|
| | ?A | ?A | ?A |
| Frequencies | 3037.7377 | 3052.5143 | 3060.3930 |
| Red. masses | 1.0784 | 1.0705 | 1.0710 |
| Frc consts | 5.8630 | 5.8771 | 5.9101 |
| IR Inten | 75.0503 | 16.3849 | 81.8239 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | −0.01 | 0.00 | 0.00 | −0.05 | 0.00 | −0.02 | −0.04 | −0.01 | −0.02 |
| 2 | 6 | 0.00 | 0.00 | 0.00 | 0.00 | 0.00 | 0.00 | 0.00 | 0.00 | 0.00 |
| 3 | 8 | 0.00 | 0.00 | 0.00 | 0.00 | 0.00 | 0.00 | 0.00 | 0.00 | 0.00 |
| 4 | 6 | −0.06 | 0.00 | −0.01 | 0.02 | 0.00 | 0.01 | −0.01 | 0.00 | 0.00 |
| 5 | 6 | 0.01 | 0.01 | 0.01 | 0.02 | 0.03 | 0.02 | −0.03 | −0.04 | −0.03 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.00 | 0.00 | 0.00 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | 0.00 | 0.00 | 0.00 | 0.00 | 0.00 | 0.00 | 0.00 | 0.00 | 0.00 |
| 16 | 6 | −0.03 | 0.00 | −0.04 | 0.00 | 0.00 | 0.01 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.00 | 0.00 | 0.00 | 0.00 | 0.00 | 0.00 | 0.00 | 0.00 | 0.00 |
| 20 | 8 | 0.00 | 0.00 | 0.00 | 0.00 | 0.00 | 0.00 | 0.00 | 0.00 | 0.00 |
| 21 | 1 | 0.00 | 0.00 | 0.00 | 0.00 | 0.00 | 0.00 | 0.00 | 0.00 | 0.01 |
| 22 | 1 | 0.00 | 0.00 | 0.00 | 0.00 | 0.00 | 0.00 | 0.00 | 0.00 | 0.00 |
| 23 | 1 | 0.00 | −0.03 | −0.01 | 0.00 | −0.03 | −0.01 | 0.00 | 0.00 | 0.00 |
| 24 | 1 | 0.08 | 0.01 | 0.04 | 0.72 | 0.08 | 0.31 | 0.55 | 0.06 | 0.23 |
| 25 | 1 | −0.14 | −0.18 | −0.09 | −0.32 | −0.40 | −0.22 | 0.44 | 0.56 | 0.31 |
| 26 | 1 | 0.74 | −0.05 | 0.14 | −0.21 | 0.02 | −0.04 | 0.14 | −0.01 | 0.03 |
| 27 | 1 | −0.01 | 0.00 | 0.00 | −0.02 | 0.00 | −0.01 | −0.02 | 0.00 | −0.01 |
| 28 | 1 | 0.29 | −0.03 | 0.01 | −0.03 | 0.00 | 0.00 | 0.00 | 0.00 | 0.00 |
| 29 | 1 | 0.10 | −0.03 | 0.50 | −0.01 | 0.00 | −0.07 | 0.01 | 0.00 | 0.05 |
| 30 | 1 | 0.00 | 0.00 | 0.00 | 0.00 | 0.00 | 0.00 | −0.02 | −0.03 | 0.03 |
| 31 | 1 | 0.00 | 0.00 | 0.00 | 0.00 | 0.00 | 0.00 | 0.01 | −0.02 | −0.01 |
| 32 | 1 | 0.00 | 0.00 | 0.00 | 0.00 | 0.00 | 0.00 | 0.01 | 0.00 | 0.01 |
| 33 | 1 | 0.00 | 0.00 | 0.00 | 0.01 | 0.00 | 0.00 | −0.01 | 0.00 | −0.01 |
| 34 | 1 | 0.00 | 0.00 | 0.00 | 0.00 | 0.00 | 0.00 | 0.00 | 0.00 | 0.00 |
| 35 | 1 | 0.00 | 0.00 | 0.00 | 0.00 | 0.00 | 0.00 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.00 | 0.00 | 0.00 | 0.00 | 0.00 | 0.00 | 0.00 | 0.00 | 0.00 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 112 | 113 | 114 |
|---|---|---|---|
| | ?A | ?A | ?A |
| Frequencies | 3064.1183 | 3069.4823 | 3074.3290 |
| Red. masses | 1.0945 | 1.0825 | 1.0948 |
| Frc consts | 6.0547 | 6.0091 | 6.0964 |
| IR Inten | 19.3551 | 172.5200 | 26.1397 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | 0.00 | 0.00 | 0.00 | 0.00 | 0.00 |
| 2 | 6 | 0.00 | 0.00 | 0.00 | 0.00 | 0.00 | 0.00 | 0.00 | 0.00 | 0.00 |
| 3 | 8 | 0.00 | 0.00 | 0.00 | 0.00 | 0.00 | 0.00 | 0.00 | 0.00 | 0.00 |
| 4 | 6 | 0.00 | 0.00 | 0.00 | 0.00 | 0.00 | 0.00 | 0.00 | 0.00 | 0.00 |
| 5 | 6 | 0.00 | 0.00 | 0.00 | 0.00 | 0.00 | 0.00 | 0.00 | 0.00 | 0.00 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.00 | 0.05 | −0.07 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | 0.00 | 0.00 | 0.00 | 0.00 | 0.00 | 0.00 | 0.00 | 0.00 | 0.00 |
| 16 | 6 | 0.00 | 0.00 | 0.00 | 0.00 | 0.00 | 0.00 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | −0.01 | −0.07 | 0.05 | 0.00 | 0.00 | 0.00 | −0.01 | −0.05 | −0.07 |
| 19 | 8 | 0.00 | 0.00 | 0.00 | 0.00 | 0.00 | 0.00 | 0.00 | 0.00 | 0.00 |
| 20 | 8 | 0.00 | 0.00 | 0.00 | 0.00 | 0.00 | 0.00 | 0.00 | 0.00 | 0.00 |
| 21 | 1 | 0.00 | 0.00 | 0.00 | 0.04 | −0.59 | 0.81 | 0.00 | 0.00 | 0.01 |
| 22 | 1 | 0.00 | 0.00 | 0.00 | 0.00 | −0.01 | 0.01 | 0.00 | 0.00 | 0.00 |
| 23 | 1 | 0.00 | 0.00 | 0.00 | 0.00 | 0.01 | 0.00 | 0.00 | 0.00 | 0.00 |
| 24 | 1 | 0.03 | 0.00 | 0.01 | −0.01 | 0.00 | 0.00 | 0.00 | 0.00 | 0.00 |
| 25 | 1 | 0.02 | 0.03 | 0.01 | 0.00 | 0.00 | 0.00 | 0.00 | 0.00 | 0.00 |
| 26 | 1 | 0.00 | 0.00 | 0.00 | 0.00 | 0.00 | 0.00 | 0.00 | 0.00 | 0.00 |
| 27 | 1 | 0.00 | 0.00 | 0.00 | 0.00 | 0.00 | 0.00 | 0.00 | 0.00 | 0.00 |
| 28 | 1 | 0.00 | 0.00 | 0.00 | 0.00 | 0.00 | 0.00 | −0.02 | 0.00 | 0.00 |
| 29 | 1 | 0.00 | 0.00 | 0.00 | 0.00 | 0.00 | −0.01 | 0.00 | 0.00 | 0.01 |
| 30 | 1 | 0.32 | 0.57 | −0.54 | 0.00 | 0.00 | 0.00 | −0.05 | −0.11 | 0.05 |
| 31 | 1 | −0.11 | 0.40 | 0.18 | 0.00 | 0.00 | 0.00 | −0.15 | 0.61 | 0.18 |
| 32 | 1 | −0.11 | −0.07 | −0.23 | 0.00 | 0.00 | −0.01 | 0.30 | 0.09 | 0.67 |
| 33 | 1 | 0.00 | 0.00 | 0.00 | 0.00 | 0.00 | 0.00 | 0.00 | 0.00 | 0.00 |
| 34 | 1 | 0.00 | 0.00 | 0.00 | 0.00 | 0.00 | 0.00 | 0.00 | 0.00 | 0.00 |
| 35 | 1 | 0.00 | 0.00 | 0.00 | 0.00 | 0.00 | 0.00 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.00 | 0.00 | 0.00 | 0.00 | 0.00 | 0.00 | 0.00 | 0.00 | 0.00 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 115 | 116 | 117 |
|---|---|---|---|
| | ?A | ?A | ?A |
| Frequencies | 3094.4433 | 3095.6942 | 3136.2264 |
| Red. masses | 1.0488 | 1.0815 | 1.0950 |
| Frc consts | 5.9174 | 6.1064 | 6.3457 |
| IR Inten | 16.9717 | 147.7529 | 175.1450 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | 0.00 | 0.00 | 0.00 | 0.00 | 0.00 |
| 2 | 6 | 0.00 | 0.00 | 0.00 | 0.01 | −0.08 | −0.02 | 0.00 | 0.00 | 0.00 |
| 3 | 8 | 0.00 | 0.00 | 0.00 | 0.00 | 0.00 | 0.00 | 0.00 | 0.00 | 0.00 |
| 4 | 6 | −0.01 | 0.00 | 0.00 | 0.00 | 0.00 | 0.00 | 0.00 | 0.00 | 0.00 |
| 5 | 6 | 0.00 | 0.00 | 0.00 | 0.00 | 0.00 | 0.00 | 0.00 | 0.00 | 0.00 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | −0.05 | 0.05 | −0.06 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.00 | 0.00 | 0.00 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | 0.00 | 0.00 | 0.00 | 0.00 | 0.00 | 0.00 | 0.00 | 0.00 | 0.00 |
| 16 | 6 | −0.05 | 0.00 | 0.04 | 0.00 | 0.00 | 0.00 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.00 | 0.00 | 0.00 | 0.00 | 0.00 | 0.00 | 0.00 | 0.00 | 0.00 |
| 20 | 8 | 0.00 | 0.00 | 0.00 | 0.00 | 0.00 | 0.00 | 0.00 | 0.00 | 0.00 |
| 21 | 1 | 0.00 | 0.00 | 0.00 | 0.00 | 0.01 | −0.01 | 0.00 | 0.01 | −0.01 |
| 22 | 1 | −0.01 | 0.01 | −0.01 | 0.00 | 0.00 | −0.01 | 0.52 | −0.55 | 0.65 |
| 23 | 1 | 0.00 | 0.00 | 0.00 | −0.11 | 0.95 | 0.29 | 0.00 | 0.01 | 0.00 |
| 24 | 1 | 0.01 | 0.00 | 0.00 | 0.03 | 0.00 | 0.01 | 0.01 | 0.00 | 0.00 |
| 25 | 1 | 0.01 | 0.01 | 0.00 | −0.01 | −0.02 | −0.01 | 0.00 | 0.00 | 0.00 |
| 26 | 1 | 0.10 | 0.00 | 0.02 | 0.02 | 0.00 | 0.01 | 0.00 | 0.00 | 0.00 |
| 27 | 1 | 0.00 | 0.00 | 0.00 | 0.00 | 0.00 | 0.00 | 0.00 | 0.00 | 0.00 |
| 28 | 1 | 0.77 | −0.10 | 0.10 | 0.00 | 0.00 | 0.00 | 0.01 | 0.00 | 0.00 |
| 29 | 1 | −0.15 | 0.04 | −0.58 | 0.00 | 0.00 | 0.00 | 0.00 | 0.00 | 0.00 |
| 30 | 1 | 0.00 | 0.00 | 0.00 | 0.00 | 0.00 | 0.00 | 0.00 | 0.00 | 0.00 |
| 31 | 1 | 0.00 | 0.01 | 0.00 | 0.00 | 0.00 | 0.00 | 0.00 | 0.00 | 0.00 |
| 32 | 1 | 0.01 | 0.00 | 0.02 | 0.00 | 0.00 | 0.00 | 0.00 | 0.00 | 0.00 |
| 33 | 1 | 0.00 | 0.00 | 0.00 | 0.00 | 0.00 | 0.00 | 0.00 | 0.00 | 0.00 |
| 34 | 1 | 0.00 | 0.00 | 0.00 | 0.00 | 0.00 | 0.00 | 0.00 | 0.00 | 0.00 |
| 35 | 1 | 0.00 | 0.00 | 0.00 | 0.00 | 0.00 | 0.00 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.00 | 0.00 | 0.00 | 0.00 | 0.00 | 0.00 | 0.01 | 0.01 | −0.01 |
| 42 | 1 | 0.00 | 0.00 | 0.00 | 0.00 | 0.00 | 0.00 | 0.00 | 0.00 | 0.00 |
| 43 | 1 | 0.00 | 0.00 | 0.00 | 0.00 | 0.00 | 0.00 | 0.00 | 0.00 | 0.00 |
| 44 | 1 | 0.00 | 0.00 | 0.00 | 0.00 | 0.00 | 0.00 | 0.00 | 0.00 | 0.00 |

| | 118 | 119 | 120 |
|---|---|---|---|
| | ?A | ?A | ?A |
| Frequencies | 3155.1409 | 3396.9638 | 3450.1847 |
| Red. masses | 1.0253 | 1.0643 | 1.0644 |
| Frc consts | 6.0137 | 7.2363 | 7.4655 |
| IR Inten | 14.9810 | 182.2300 | 115.5013 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | 0.00 | 0.00 | 0.00 | 0.00 | 0.00 |
| 2 | 6 | 0.00 | 0.00 | 0.00 | 0.00 | 0.00 | 0.00 | 0.00 | 0.00 | 0.00 |
| 3 | 8 | 0.00 | 0.00 | 0.00 | 0.00 | 0.00 | 0.00 | 0.00 | 0.00 | 0.00 |
| 4 | 6 | 0.00 | 0.00 | 0.00 | 0.00 | 0.00 | 0.00 | 0.00 | 0.00 | 0.00 |
| 5 | 6 | 0.00 | 0.00 | 0.00 | 0.00 | 0.00 | 0.00 | 0.00 | 0.00 | 0.00 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.00 | 0.00 | 0.00 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | 0.00 | 0.00 | 0.00 | 0.00 | 0.00 | 0.00 | 0.00 | 0.00 | 0.00 |
| 16 | 6 | 0.00 | 0.00 | 0.00 | 0.00 | 0.00 | 0.00 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.04 | −0.01 | 0.00 | 0.00 | 0.00 | 0.00 | 0.00 | 0.00 | 0.00 |
| 19 | 8 | 0.00 | 0.00 | 0.00 | 0.00 | 0.00 | 0.00 | −0.01 | 0.00 | −0.06 |
| 20 | 8 | 0.00 | 0.00 | 0.00 | −0.06 | −0.01 | −0.02 | 0.00 | 0.00 | 0.00 |
| 21 | 1 | 0.00 | 0.00 | 0.00 | 0.00 | 0.00 | 0.00 | 0.00 | 0.00 | 0.00 |
| 22 | 1 | 0.00 | 0.00 | 0.00 | 0.00 | 0.00 | 0.00 | 0.00 | 0.00 | 0.00 |
| 23 | 1 | 0.00 | 0.00 | 0.00 | 0.00 | 0.00 | 0.00 | 0.00 | 0.00 | 0.00 |
| 24 | 1 | 0.00 | 0.00 | 0.00 | 0.03 | 0.01 | 0.00 | 0.00 | 0.00 | 0.00 |
| 25 | 1 | 0.00 | 0.00 | 0.00 | 0.00 | 0.00 | 0.00 | 0.01 | 0.01 | 0.00 |
| 26 | 1 | 0.00 | 0.00 | 0.00 | 0.00 | 0.00 | 0.00 | 0.00 | 0.00 | 0.00 |
| 27 | 1 | 0.00 | 0.00 | 0.00 | 0.92 | 0.13 | 0.35 | −0.01 | 0.00 | 0.00 |
| 28 | 1 | 0.00 | 0.00 | 0.00 | 0.00 | 0.00 | 0.00 | 0.00 | 0.00 | 0.00 |
| 29 | 1 | 0.00 | 0.00 | 0.00 | 0.00 | 0.00 | 0.00 | 0.00 | 0.00 | 0.00 |
| 30 | 1 | −0.17 | −0.35 | 0.33 | 0.00 | 0.00 | 0.00 | 0.00 | 0.00 | 0.00 |
| 31 | 1 | −0.11 | 0.55 | 0.20 | 0.00 | 0.00 | 0.00 | 0.00 | 0.00 | 0.00 |
| 32 | 1 | −0.22 | −0.09 | −0.56 | 0.00 | 0.00 | 0.00 | 0.00 | 0.00 | 0.00 |
| 33 | 1 | 0.00 | 0.00 | 0.00 | 0.00 | 0.00 | 0.01 | 0.13 | −0.07 | 0.99 |
| 34 | 1 | 0.00 | 0.00 | 0.00 | 0.00 | 0.00 | 0.00 | 0.00 | 0.00 | 0.00 |
| 35 | 1 | 0.00 | 0.00 | 0.00 | 0.00 | 0.00 | 0.00 | −0.01 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.00 | 0.00 | 0.00 |
| 39 | 8 | 0.00 | 0.00 | 0.00 | 0.00 | 0.00 | 0.00 | 0.00 | 0.00 | 0.00 |
| 40 | 8 | 0.00 | 0.00 | 0.00 | 0.00 | 0.00 | 0.00 | 0.00 | 0.00 | 0.00 |
| 41 | 1 | 0.00 | 0.00 | 0.00 | 0.00 | 0.00 | 0.00 | 0.00 | 0.00 | 0.00 |
| 42 | 1 | 0.00 | 0.00 | 0.00 | 0.00 | 0.00 | 0.00 | 0.00 | 0.00 | −0.01 |
| 43 | 1 | 0.00 | 0.00 | 0.00 | −0.01 | 0.00 | 0.00 | 0.00 | 0.00 | 0.00 |
| 44 | 1 | 0.00 | 0.00 | 0.00 | 0.01 | 0.01 | 0.00 | 0.01 | 0.00 | 0.00 |

| | 121 | 122 | 123 |
|---|---|---|---|
| | ?A | ?A | ?A |
| Frequencies | 3470.0243 | 3471.7031 | 3491.3635 |
| Red. masses | 1.0645 | 1.0771 | 1.0644 |
| Frc consts | 7.5522 | 7.6491 | 7.6446 |
| IR Inten | 291.2747 | 260.0642 | 181.0602 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | 0.00 | 0.00 | 0.00 | 0.00 | 0.00 |
| 2 | 6 | 0.00 | 0.00 | 0.00 | 0.00 | 0.00 | 0.00 | 0.00 | 0.00 | 0.00 |
| 3 | 8 | 0.00 | 0.00 | 0.00 | 0.00 | 0.00 | 0.00 | 0.00 | 0.00 | 0.00 |
| 4 | 6 | 0.00 | 0.00 | 0.00 | 0.00 | 0.00 | 0.00 | 0.00 | 0.00 | 0.00 |
| 5 | 6 | 0.00 | 0.00 | 0.00 | 0.00 | 0.00 | 0.00 | 0.00 | 0.00 | 0.00 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.00 | 0.00 | 0.00 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | −0.01 | 0.00 | 0.00 | −0.06 | 0.03 | −0.03 | 0.00 | 0.00 | 0.00 |
| 16 | 6 | 0.00 | 0.00 | 0.00 | 0.00 | 0.00 | 0.00 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.00 | 0.00 | 0.00 | 0.00 | 0.00 | 0.00 | 0.00 | 0.00 | 0.00 |
| 20 | 8 | 0.00 | 0.00 | 0.00 | 0.00 | 0.00 | 0.00 | 0.00 | 0.00 | 0.00 |
| 21 | 1 | 0.00 | 0.00 | 0.00 | 0.00 | 0.00 | 0.00 | 0.00 | 0.00 | 0.00 |
| 22 | 1 | 0.00 | 0.00 | 0.00 | 0.00 | 0.00 | 0.00 | 0.00 | 0.00 | 0.00 |
| 23 | 1 | 0.00 | 0.00 | 0.00 | 0.00 | 0.00 | 0.00 | 0.00 | 0.00 | 0.00 |
| 24 | 1 | 0.00 | 0.00 | 0.00 | 0.00 | 0.00 | 0.00 | 0.00 | 0.00 | 0.00 |
| 25 | 1 | 0.00 | 0.00 | 0.00 | 0.00 | 0.00 | 0.00 | 0.00 | 0.00 | 0.00 |
| 26 | 1 | 0.00 | 0.00 | 0.00 | 0.00 | 0.00 | 0.00 | 0.00 | 0.00 | 0.00 |
| 27 | 1 | −0.01 | 0.00 | −0.01 | 0.00 | 0.00 | 0.00 | 0.00 | 0.00 | 0.00 |
| 28 | 1 | 0.00 | 0.00 | 0.00 | 0.00 | 0.00 | 0.00 | 0.00 | 0.00 | 0.00 |
| 29 | 1 | 0.00 | 0.00 | 0.00 | 0.00 | 0.00 | 0.00 | 0.00 | 0.00 | 0.00 |
| 30 | 1 | 0.00 | 0.00 | 0.00 | 0.00 | 0.00 | 0.00 | 0.00 | 0.00 | 0.00 |
| 31 | 1 | 0.00 | 0.00 | 0.00 | 0.00 | 0.00 | 0.00 | 0.00 | 0.00 | 0.00 |
| 32 | 1 | 0.00 | 0.00 | 0.00 | 0.00 | 0.00 | 0.00 | 0.00 | 0.00 | 0.00 |
| 33 | 1 | 0.00 | 0.00 | −0.01 | 0.00 | 0.00 | 0.01 | 0.00 | 0.00 | −0.01 |
| 34 | 1 | 0.00 | 0.00 | 0.01 | −0.02 | −0.04 | 0.12 | 0.00 | 0.00 | 0.00 |
| 35 | 1 | 0.08 | −0.04 | 0.03 | 0.84 | −0.41 | 0.31 | 0.00 | 0.00 | 0.00 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | 0.00 | 0.00 | 0.00 | 0.03 | −0.01 | 0.05 |
| 39 | 8 | −0.05 | −0.03 | −0.01 | 0.00 | 0.00 | 0.00 | 0.00 | 0.00 | 0.00 |
| 40 | 8 | 0.00 | 0.00 | 0.00 | 0.00 | 0.00 | 0.00 | −0.02 | 0.01 | 0.00 |
| 41 | 1 | 0.01 | 0.00 | −0.01 | −0.01 | −0.01 | 0.01 | 0.01 | 0.00 | −0.01 |
| 42 | 1 | 0.05 | −0.01 | 0.08 | −0.01 | 0.00 | −0.01 | −0.49 | 0.12 | −0.75 |
| 43 | 1 | 0.07 | −0.03 | −0.01 | −0.01 | 0.00 | 0.00 | 0.39 | −0.14 | −0.05 |
| 44 | 1 | 0.88 | 0.39 | 0.22 | −0.08 | −0.04 | −0.02 | 0.05 | 0.02 | 0.01 |

| | 124 | 125 | 126 |
|---|---|---|---|
| | ?A | ?A | ?A |
| Frequencies | 3494.1852 | 3499.6454 | 3530.5000 |
| Red. masses | 1.0758 | 1.0649 | 1.0599 |
| Frc consts | 7.7387 | 7.6847 | 7.7839 |
| IR Inten | 276.4614 | 121.1129 | 237.7755 |
| Raman Activ | 0.0000 | 0.0000 | 0.0000 |
| Depolar | 0.0000 | 0.0000 | 0.0000 |

| Atom | AN | X | Y | Z | X | Y | Z | X | Y | Z |
|---|---|---|---|---|---|---|---|---|---|---|
| 1 | 6 | 0.00 | 0.00 | 0.00 | 0.00 | 0.00 | 0.00 | 0.00 | 0.00 | 0.00 |
| 2 | 6 | 0.00 | 0.00 | 0.00 | 0.00 | 0.00 | 0.00 | 0.00 | 0.00 | 0.00 |
| 3 | 8 | 0.00 | 0.00 | 0.00 | 0.00 | 0.00 | 0.00 | 0.00 | 0.00 | 0.00 |
| 4 | 6 | 0.00 | 0.00 | 0.00 | 0.00 | 0.00 | 0.00 | 0.00 | 0.00 | 0.00 |
| 5 | 6 | 0.00 | 0.00 | 0.00 | 0.00 | 0.00 | 0.00 | 0.00 | 0.00 | 0.00 |
| 6 | 7 | 0.00 | 0.00 | 0.00 | 0.00 | 0.00 | 0.00 | 0.00 | 0.00 | 0.00 |
| 7 | 6 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 7 | −0.04 | −0.03 | 0.05 | 0.00 | 0.00 | 0.00 | 0.00 | 0.00 | −0.01 |
| 10 | 6 | 0.00 | 0.00 | 0.00 | 0.00 | 0.00 | 0.00 | 0.00 | 0.00 | 0.00 |
| 11 | 6 | 0.00 | 0.00 | 0.00 | 0.00 | 0.00 | 0.00 | 0.00 | 0.00 | 0.00 |
| 12 | 7 | 0.00 | 0.00 | 0.00 | 0.00 | 0.00 | 0.00 | 0.00 | 0.00 | 0.00 |
| 13 | 6 | 0.00 | 0.00 | 0.00 | 0.00 | 0.00 | 0.00 | 0.00 | 0.00 | 0.00 |
| 14 | 7 | 0.00 | 0.00 | 0.00 | 0.00 | 0.00 | 0.00 | 0.00 | 0.00 | 0.00 |
| 15 | 7 | 0.00 | 0.00 | −0.01 | 0.00 | 0.00 | 0.00 | 0.01 | 0.02 | −0.06 |
| 16 | 6 | 0.00 | 0.00 | 0.00 | 0.00 | 0.00 | 0.00 | 0.00 | 0.00 | 0.00 |
| 17 | 16 | 0.00 | 0.00 | 0.00 | 0.00 | 0.00 | 0.00 | 0.00 | 0.00 | 0.00 |
| 18 | 6 | 0.00 | 0.00 | 0.00 | 0.00 | 0.00 | 0.00 | 0.00 | 0.00 | 0.00 |
| 19 | 8 | 0.00 | 0.00 | 0.00 | 0.00 | 0.00 | 0.00 | 0.00 | 0.00 | 0.00 |
| 20 | 8 | 0.00 | 0.00 | 0.00 | 0.00 | 0.00 | 0.00 | 0.00 | 0.00 | 0.00 |
| 21 | 1 | 0.00 | 0.00 | 0.00 | 0.00 | 0.00 | 0.00 | 0.00 | 0.00 | 0.00 |
| 22 | 1 | −0.01 | 0.01 | −0.01 | 0.00 | 0.00 | 0.00 | 0.00 | 0.00 | 0.00 |
| 23 | 1 | 0.00 | 0.00 | 0.00 | 0.00 | 0.00 | 0.00 | 0.00 | 0.00 | 0.00 |
| 24 | 1 | 0.00 | 0.00 | 0.00 | 0.00 | 0.00 | 0.00 | 0.00 | 0.00 | 0.00 |
| 25 | 1 | 0.00 | 0.00 | 0.00 | 0.00 | 0.00 | 0.00 | 0.00 | 0.00 | 0.00 |
| 26 | 1 | 0.00 | 0.00 | 0.00 | 0.00 | 0.00 | 0.00 | 0.00 | 0.00 | 0.00 |
| 27 | 1 | 0.00 | 0.00 | 0.00 | 0.01 | 0.00 | 0.01 | 0.00 | 0.00 | 0.00 |
| 28 | 1 | 0.00 | 0.00 | 0.00 | 0.00 | 0.00 | 0.00 | 0.00 | 0.00 | 0.00 |
| 29 | 1 | 0.00 | 0.00 | 0.00 | 0.00 | 0.00 | 0.00 | 0.00 | 0.00 | 0.00 |
| 30 | 1 | 0.00 | 0.00 | 0.00 | 0.00 | 0.00 | 0.00 | 0.00 | 0.00 | 0.00 |
| 31 | 1 | 0.00 | 0.00 | 0.00 | 0.00 | 0.00 | 0.00 | 0.00 | 0.00 | 0.00 |
| 32 | 1 | 0.00 | 0.00 | 0.00 | 0.00 | 0.00 | 0.00 | 0.00 | 0.00 | 0.00 |
| 33 | 1 | 0.00 | 0.00 | 0.01 | 0.00 | 0.00 | 0.00 | 0.00 | 0.00 | 0.00 |
| 34 | 1 | 0.00 | −0.04 | 0.10 | 0.00 | 0.00 | 0.00 | 0.02 | −0.40 | 0.90 |
| 35 | 1 | 0.00 | 0.00 | 0.00 | 0.00 | 0.00 | 0.00 | −0.12 | 0.06 | −0.07 |
| 36 | 15 | 0.00 | 0.00 | 0.00 | 0.00 | 0.00 | 0.00 | 0.00 | 0.00 | 0.00 |
| 37 | 8 | 0.00 | 0.00 | 0.00 | 0.00 | 0.00 | 0.00 | 0.00 | 0.00 | 0.00 |
| 38 | 8 | 0.00 | 0.00 | 0.00 | −0.01 | 0.00 | −0.02 | 0.00 | 0.00 | 0.00 |
| 39 | 8 | 0.00 | 0.00 | 0.00 | 0.01 | 0.00 | 0.00 | 0.00 | 0.00 | 0.00 |
| 40 | 8 | 0.00 | 0.00 | 0.00 | −0.05 | 0.02 | 0.01 | 0.00 | 0.00 | 0.00 |
| 41 | 1 | 0.60 | 0.41 | −0.67 | 0.01 | 0.00 | −0.01 | −0.07 | −0.05 | 0.07 |
| 42 | 1 | 0.00 | 0.00 | 0.00 | 0.22 | −0.06 | 0.34 | 0.00 | 0.00 | 0.00 |
| 43 | 1 | −0.01 | 0.01 | 0.00 | 0.84 | −0.32 | −0.10 | 0.00 | 0.00 | 0.00 |
| 44 | 1 | −0.01 | 0.00 | 0.00 | −0.09 | −0.04 | −0.02 | 0.00 | 0.00 | 0.00 |

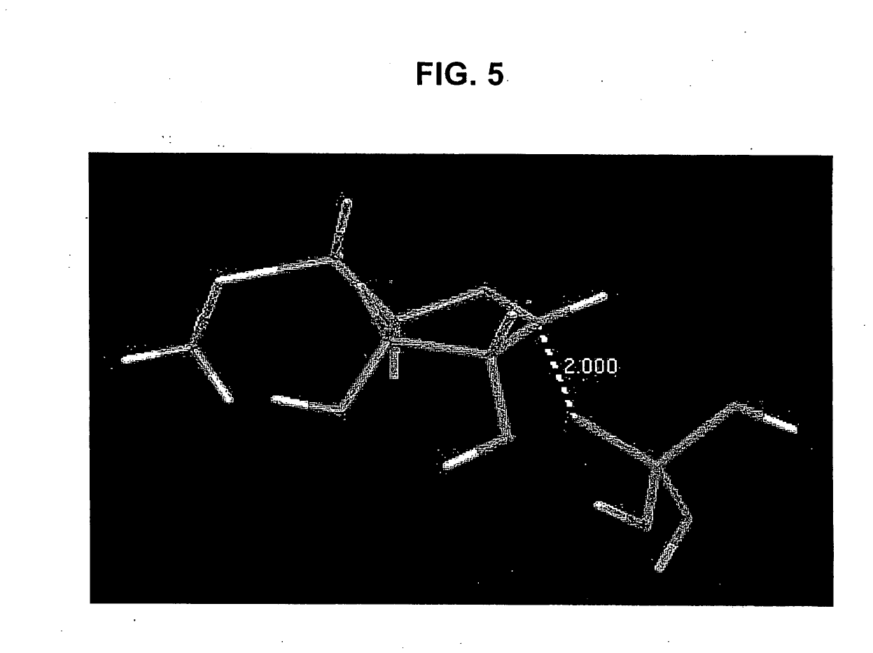

Frequencies for transition state of human MTAP as a phosphate nucleophile (B1LYP/6-31G**), as shown in FIG. 5.

The 5′-methylthio group and the phosphate was constrained during the calculation using following torsion angles and bond lengths:

### 1. O4′-C4′-C5′-S

### 2. C4′-C5′-S—C^{s}

### 3. O4′-C1′-O—P

### 4. C1′-O^{phosphate}

****** 2 imaginary frequencies (negative Signs) ******
Harmonic frequencies (cm** − 1), IR intensities (KM/Mole),
Raman scattering activities (A**4/AMU), Raman depolarization ratios,
reduced masses (AMU), force constants (mDyne/A) and normal coordinates:

| | | |
|---|---|---|
| 1 | 2 | 3 |
| ?A | ?A | ?A |

| | | | |
|---|---|---|---|
| Frequencies -- | −91.4416 | −14.8028 | 34.3924 |
| Red. masses -- | 9.9409 | 6.1664 | 5.7920 |
| Frc consts -- | 0.0490 | 0.0008 | 0.0040 |
| IR Inten -- | 155.1689 | 0.8871 | 0.1322 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.09 | −0.09 | 0.15 | 0.00 | 0.02 | −0.04 | −0.01 | 0.09 | −0.14 |
| 2 | 6 | 0.24 | −0.17 | 0.30 | 0.00 | 0.02 | −0.01 | 0.00 | 0.08 | −0.11 |
| 3 | 6 | 0.15 | −0.09 | 0.11 | −0.02 | 0.02 | −0.03 | 0.00 | 0.07 | −0.08 |
| 4 | 6 | 0.12 | −0.05 | 0.01 | 0.03 | −0.02 | 0.13 | 0.00 | 0.06 | −0.10 |
| 5 | 6 | 0.11 | −0.05 | 0.07 | 0.00 | −0.02 | −0.02 | 0.02 | 0.06 | −0.10 |
| 6 | 8 | 0.04 | 0.01 | 0.15 | 0.06 | 0.15 | −0.16 | −0.01 | 0.03 | −0.05 |
| 7 | 8 | 0.03 | 0.03 | −0.04 | 0.20 | −0.18 | 0.24 | −0.03 | 0.08 | −0.12 |
| 8 | 6 | 0.10 | −0.02 | 0.04 | −0.01 | 0.06 | −0.08 | 0.01 | 0.03 | −0.03 |
| 9 | 16 | 0.07 | 0.00 | −0.03 | 0.00 | 0.00 | 0.00 | 0.05 | −0.06 | 0.13 |
| 10 | 6 | 0.01 | 0.03 | −0.02 | 0.02 | −0.11 | −0.08 | 0.28 | −0.11 | 0.13 |
| 11 | 1 | 0.04 | −0.05 | 0.08 | 0.01 | 0.03 | 0.00 | 0.01 | 0.09 | −0.10 |
| 12 | 1 | 0.23 | −0.16 | 0.07 | −0.16 | −0.04 | −0.09 | 0.02 | 0.10 | −0.05 |
| 13 | 1 | 0.18 | −0.08 | −0.07 | −0.07 | 0.10 | 0.29 | 0.00 | 0.04 | −0.12 |
| 14 | 1 | 0.00 | 0.02 | 0.13 | −0.03 | −0.11 | −0.05 | 0.07 | 0.06 | −0.11 |
| 15 | 1 | −0.03 | 0.09 | 0.06 | 0.13 | 0.03 | −0.06 | −0.02 | 0.05 | −0.06 |
| 16 | 1 | 0.01 | −0.01 | −0.09 | 0.23 | 0.00 | 0.29 | −0.04 | −0.03 | −0.09 |
| 17 | 1 | 0.15 | −0.04 | 0.03 | −0.01 | 0.17 | −0.06 | −0.08 | 0.08 | −0.01 |
| 18 | 1 | 0.08 | −0.02 | 0.06 | −0.03 | 0.05 | −0.19 | 0.06 | 0.02 | −0.08 |
| 19 | 1 | −0.01 | 0.02 | 0.01 | 0.02 | −0.15 | −0.08 | 0.35 | −0.07 | 0.03 |
| 20 | 1 | 0.00 | 0.04 | −0.04 | 0.02 | −0.15 | −0.04 | 0.32 | −0.17 | 0.23 |
| 21 | 1 | 0.01 | 0.02 | −0.01 | 0.02 | −0.09 | −0.15 | 0.31 | −0.11 | 0.12 |
| 22 | 15 | −0.10 | 0.08 | −0.10 | −0.04 | 0.01 | 0.01 | −0.06 | −0.03 | 0.04 |
| 23 | 8 | −0.16 | 0.17 | −0.26 | −0.06 | 0.08 | 0.02 | −0.05 | 0.01 | −0.09 |
| 24 | 8 | −0.35 | −0.06 | −0.05 | −0.12 | 0.07 | 0.14 | −0.17 | −0.08 | 0.10 |
| 25 | 8 | −0.13 | 0.03 | −0.05 | 0.02 | 0.08 | −0.16 | −0.07 | −0.05 | 0.08 |
| 26 | 1 | −0.18 | 0.02 | 0.02 | 0.04 | 0.09 | −0.22 | −0.04 | −0.03 | 0.03 |
| 27 | 8 | −0.04 | 0.03 | −0.05 | −0.02 | −0.19 | 0.03 | 0.07 | −0.04 | 0.10 |
| 28 | 1 | −0.03 | 0.02 | −0.04 | −0.01 | −0.24 | 0.02 | 0.08 | −0.07 | 0.19 |
| 29 | 1 | −0.33 | −0.03 | −0.05 | −0.13 | 0.19 | 0.12 | −0.25 | −0.08 | 0.09 |

| | | |
|---|---|---|
| 4 | 5 | 6 |
| ?A | ?A | ?A |

| | | | |
|---|---|---|---|
| Frequencies -- | 44.7646 | 56.7315 | 74.5695 |
| Red. masses -- | 3.6451 | 6.4539 | 4.9583 |
| Frc consts -- | 0.0043 | 0.0122 | 0.0162 |
| IR Inten -- | 6.5992 | 0.9767 | 8.0312 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | 0.02 | 0.03 | 0.01 | 0.08 | −0.01 | −0.02 | 0.02 | −0.08 |
| 2 | 6 | 0.01 | 0.01 | 0.01 | −0.01 | 0.10 | 0.04 | −0.05 | 0.05 | −0.12 |
| 3 | 6 | 0.02 | 0.02 | 0.00 | −0.02 | 0.06 | 0.14 | −0.06 | 0.02 | −0.02 |
| 4 | 6 | 0.01 | 0.05 | −0.05 | −0.05 | 0.03 | 0.02 | −0.03 | −0.05 | 0.04 |
| 5 | 6 | 0.01 | 0.05 | 0.02 | −0.01 | 0.03 | 0.00 | 0.03 | −0.05 | 0.02 |
| 6 | 8 | 0.01 | −0.02 | 0.03 | −0.08 | −0.07 | 0.26 | −0.03 | −0.03 | −0.01 |
| 7 | 8 | −0.04 | 0.13 | −0.10 | −0.18 | 0.03 | 0.00 | 0.00 | −0.09 | 0.07 |
| 8 | 6 | 0.01 | 0.03 | 0.04 | 0.02 | −0.02 | −0.03 | 0.01 | −0.10 | 0.12 |
| 9 | 16 | 0.03 | 0.03 | 0.10 | −0.04 | −0.06 | −0.08 | 0.01 | 0.04 | 0.01 |
| 10 | 6 | −0.21 | −0.26 | −0.12 | 0.12 | 0.10 | 0.04 | −0.20 | 0.12 | 0.04 |
| 11 | 1 | 0.00 | 0.00 | 0.00 | −0.01 | 0.16 | 0.02 | −0.09 | 0.11 | −0.17 |
| 12 | 1 | 0.07 | 0.03 | 0.01 | 0.05 | 0.16 | 0.22 | −0.12 | 0.07 | 0.02 |
| 13 | 1 | 0.04 | 0.00 | −0.11 | 0.00 | 0.01 | −0.04 | −0.08 | −0.06 | 0.08 |
| 14 | 1 | 0.01 | 0.08 | 0.03 | 0.00 | 0.02 | −0.01 | 0.13 | −0.07 | −0.01 |
| 15 | 1 | 0.00 | −0.01 | 0.02 | −0.11 | −0.10 | 0.24 | −0.02 | −0.02 | 0.00 |
| 16 | 1 | −0.05 | −0.13 | −0.02 | −0.19 | 0.01 | −0.09 | 0.00 | −0.08 | 0.08 |
| 17 | 1 | −0.01 | 0.03 | 0.05 | 0.03 | −0.02 | −0.03 | 0.01 | −0.23 | 0.09 |
| 18 | 1 | 0.01 | 0.03 | 0.05 | 0.05 | −0.02 | −0.03 | −0.01 | −0.08 | 0.25 |
| 19 | 1 | −0.23 | −0.54 | −0.07 | 0.12 | 0.30 | 0.03 | −0.25 | 0.07 | 0.12 |
| 20 | 1 | −0.17 | −0.22 | −0.07 | 0.07 | 0.04 | 0.00 | −0.21 | 0.22 | −0.05 |
| 21 | 1 | −0.38 | −0.16 | −0.34 | 0.27 | 0.03 | 0.17 | −0.27 | 0.12 | 0.07 |
| 22 | 15 | 0.02 | −0.01 | −0.01 | 0.04 | −0.02 | −0.05 | 0.05 | 0.01 | −0.03 |
| 23 | 8 | 0.02 | 0.01 | 0.00 | 0.03 | 0.02 | −0.01 | 0.08 | −0.01 | −0.22 |
| 24 | 8 | 0.00 | 0.02 | 0.04 | −0.01 | 0.06 | 0.10 | −0.08 | 0.00 | 0.08 |
| 25 | 8 | 0.04 | 0.02 | −0.08 | 0.09 | 0.06 | −0.25 | −0.05 | 0.00 | 0.01 |
| 26 | 1 | 0.02 | 0.01 | −0.05 | 0.01 | 0.00 | −0.09 | −0.06 | −0.02 | −0.03 |
| 27 | 8 | 0.03 | −0.09 | 0.00 | 0.07 | −0.26 | −0.02 | 0.27 | 0.03 | 0.08 |
| 28 | 1 | 0.04 | −0.11 | 0.00 | 0.08 | −0.33 | −0.03 | 0.27 | 0.04 | 0.23 |
| 29 | 1 | 0.00 | 0.08 | 0.04 | 0.01 | 0.22 | 0.08 | −0.24 | −0.01 | 0.07 |

| | | |
|---|---|---|
| 7 | 8 | 9 |
| ?A | ?A | ?A |

| | | | |
|---|---|---|---|
| Frequencies -- | 103.1703 | 110.9876 | 160.5863 |
| Red. masses -- | 4.5153 | 4.8074 | 1.6664 |
| Frc consts -- | 0.0283 | 0.0349 | 0.0253 |
| IR Inten -- | 0.6367 | 6.8965 | 6.9323 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.06 | −0.10 | 0.06 | 0.05 | 0.00 | −0.16 | 0.00 | 0.02 | −0.04 |
| 2 | 6 | −0.02 | −0.13 | 0.05 | 0.04 | 0.01 | −0.05 | 0.02 | −0.01 | −0.01 |
| 3 | 6 | 0.01 | −0.11 | 0.02 | 0.01 | −0.02 | 0.03 | 0.04 | 0.00 | 0.00 |
| 4 | 6 | −0.01 | −0.03 | −0.01 | 0.01 | −0.08 | −0.04 | 0.04 | 0.02 | 0.00 |
| 5 | 6 | −0.11 | −0.01 | −0.04 | 0.06 | −0.09 | −0.10 | 0.03 | 0.02 | −0.01 |
| 6 | 8 | −0.02 | −0.12 | 0.05 | −0.06 | −0.06 | 0.10 | 0.02 | −0.02 | 0.03 |
| 7 | 8 | 0.01 | −0.04 | 0.00 | −0.06 | −0.15 | −0.02 | 0.05 | 0.01 | 0.00 |
| 8 | 6 | −0.13 | 0.15 | −0.15 | 0.02 | −0.06 | −0.01 | 0.02 | 0.02 | 0.02 |
| 9 | 16 | −0.05 | 0.10 | 0.05 | 0.16 | 0.12 | 0.07 | 0.00 | 0.00 | 0.02 |
| 10 | 6 | 0.14 | −0.05 | −0.03 | −0.01 | 0.07 | 0.01 | −0.03 | −0.01 | 0.01 |
| 11 | 1 | −0.07 | −0.15 | 0.01 | 0.11 | 0.05 | 0.00 | 0.02 | −0.01 | −0.01 |
| 12 | 1 | 0.07 | −0.12 | 0.02 | 0.03 | 0.04 | 0.07 | 0.06 | 0.01 | 0.01 |
| 13 | 1 | 0.03 | 0.03 | −0.01 | 0.02 | −0.08 | −0.05 | 0.04 | 0.03 | 0.00 |
| 14 | 1 | −0.25 | −0.05 | −0.01 | 0.13 | −0.12 | −0.14 | 0.05 | 0.01 | −0.02 |
| 15 | 1 | −0.03 | −0.12 | 0.04 | −0.08 | −0.20 | 0.12 | 0.01 | −0.02 | 0.02 |
| 16 | 1 | 0.01 | −0.03 | 0.01 | −0.08 | −0.40 | 0.01 | 0.05 | 0.06 | 0.00 |
| 17 | 1 | −0.13 | 0.36 | −0.11 | −0.04 | −0.10 | −0.02 | 0.01 | 0.02 | 0.02 |
| 18 | 1 | −0.21 | 0.13 | −0.34 | −0.06 | −0.04 | 0.06 | 0.03 | 0.02 | 0.02 |
| 19 | 1 | 0.24 | −0.15 | −0.16 | 0.08 | −0.25 | −0.07 | −0.26 | 0.38 | 0.28 |
| 20 | 1 | 0.24 | −0.08 | 0.14 | 0.18 | 0.30 | 0.12 | −0.38 | −0.35 | −0.28 |
| 21 | 1 | 0.06 | −0.01 | −0.11 | −0.35 | 0.15 | −0.04 | 0.47 | −0.11 | −0.02 |
| 22 | 15 | 0.04 | 0.03 | −0.02 | −0.06 | 0.01 | 0.02 | −0.03 | −0.01 | 0.00 |
| 23 | 8 | 0.07 | −0.08 | −0.09 | −0.05 | −0.04 | 0.08 | 0.00 | −0.10 | −0.02 |
| 24 | 8 | 0.04 | 0.10 | 0.06 | 0.03 | 0.09 | 0.02 | 0.04 | 0.07 | 0.03 |
| 25 | 8 | −0.08 | 0.05 | −0.03 | −0.07 | 0.04 | −0.04 | −0.11 | 0.00 | −0.02 |
| 26 | 1 | −0.12 | −0.01 | −0.04 | −0.10 | 0.01 | −0.01 | −0.14 | −0.04 | −0.04 |
| 27 | 8 | 0.14 | 0.05 | 0.03 | −0.14 | −0.01 | −0.02 | −0.03 | 0.00 | −0.01 |
| 28 | 1 | 0.12 | 0.15 | 0.09 | −0.14 | 0.01 | −0.08 | −0.03 | 0.04 | −0.01 |
| 29 | 1 | −0.02 | 0.12 | 0.05 | 0.15 | 0.20 | 0.01 | 0.11 | 0.17 | 0.02 |

| | | |
|---|---|---|
| 10 | 11 | 12 |
| ?A | ?A | ?A |

| | | | |
|---|---|---|---|
| Frequencies -- | 172.6202 | 193.1862 | 224.4305 |
| Red. masses -- | 2.5095 | 4.3488 | 1.3369 |
| Frc consts -- | 0.0441 | 0.0956 | 0.0397 |
| IR Inten -- | 5.9129 | 20.3232 | 70.8234 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | 0.04 | 0.03 | 0.05 | −0.03 | 0.11 | 0.01 | 0.01 | −0.02 |
| 2 | 6 | 0.01 | 0.02 | 0.02 | 0.00 | 0.03 | 0.09 | 0.02 | 0.00 | 0.01 |
| 3 | 6 | 0.01 | 0.03 | 0.01 | −0.11 | 0.03 | 0.00 | 0.02 | 0.00 | 0.01 |
| 4 | 6 | 0.01 | 0.04 | 0.03 | −0.08 | −0.05 | 0.03 | 0.01 | 0.01 | −0.01 |
| 5 | 6 | 0.02 | 0.04 | 0.05 | 0.00 | −0.05 | 0.05 | 0.00 | 0.01 | −0.02 |
| 6 | 8 | 0.00 | 0.07 | 0.00 | −0.13 | 0.22 | −0.08 | 0.00 | 0.01 | 0.01 |
| 7 | 8 | 0.04 | 0.07 | 0.03 | −0.15 | −0.02 | 0.00 | 0.01 | −0.01 | 0.00 |
| 8 | 6 | 0.05 | −0.02 | 0.03 | 0.03 | −0.01 | −0.02 | 0.00 | 0.00 | −0.01 |
| 9 | 16 | 0.00 | −0.04 | −0.05 | 0.11 | 0.09 | 0.01 | −0.01 | −0.01 | 0.01 |
| 10 | 6 | 0.02 | 0.00 | −0.01 | 0.08 | −0.02 | −0.08 | −0.02 | 0.01 | 0.02 |
| 11 | 1 | 0.00 | 0.02 | 0.01 | 0.00 | 0.06 | 0.08 | 0.04 | −0.01 | 0.04 |
| 12 | 1 | 0.01 | 0.01 | −0.01 | −0.18 | −0.05 | −0.07 | 0.04 | −0.01 | 0.00 |
| 13 | 1 | 0.00 | 0.04 | 0.04 | −0.10 | −0.12 | 0.00 | 0.02 | 0.03 | −0.01 |
| 14 | 1 | 0.03 | 0.05 | 0.05 | −0.05 | −0.04 | 0.07 | 0.01 | 0.01 | −0.02 |
| 15 | 1 | −0.01 | 0.12 | −0.02 | −0.13 | 0.23 | −0.08 | 0.00 | −0.02 | 0.02 |
| 16 | 1 | 0.05 | 0.15 | 0.02 | −0.17 | −0.13 | −0.03 | 0.01 | 0.00 | 0.00 |
| 17 | 1 | 0.08 | −0.07 | 0.02 | 0.07 | 0.01 | −0.02 | −0.02 | 0.01 | 0.00 |
| 18 | 1 | 0.09 | −0.02 | 0.07 | −0.06 | 0.00 | −0.02 | 0.01 | 0.00 | −0.01 |
| 19 | 1 | 0.23 | −0.34 | −0.27 | −0.05 | 0.16 | 0.09 | −0.02 | 0.00 | 0.02 |
| 20 | 1 | 0.34 | 0.33 | 0.24 | −0.13 | −0.29 | −0.21 | −0.01 | 0.04 | 0.01 |
| 21 | 1 | −0.45 | 0.08 | 0.04 | 0.40 | −0.06 | −0.19 | −0.04 | 0.01 | 0.04 |
| 22 | 15 | −0.02 | −0.04 | −0.01 | 0.03 | −0.05 | −0.01 | 0.00 | 0.00 | 0.01 |
| 23 | 8 | 0.01 | −0.14 | −0.04 | 0.02 | −0.03 | 0.01 | 0.00 | −0.05 | 0.06 |
| 24 | 8 | 0.07 | 0.06 | 0.01 | 0.07 | −0.05 | −0.06 | 0.00 | −0.02 | −0.01 |
| 25 | 8 | −0.18 | −0.03 | −0.02 | −0.09 | −0.03 | −0.01 | −0.01 | 0.04 | −0.09 |
| 26 | 1 | −0.23 | −0.09 | −0.04 | −0.14 | −0.09 | 0.01 | −0.03 | 0.01 | −0.09 |
| 27 | 8 | 0.00 | −0.02 | −0.01 | 0.06 | −0.05 | 0.01 | 0.02 | 0.01 | 0.02 |
| 28 | 1 | 0.00 | −0.01 | 0.01 | 0.08 | −0.16 | 0.05 | −0.07 | 0.53 | −0.02 |
| 29 | 1 | 0.10 | 0.12 | 0.01 | −0.16 | −0.32 | −0.04 | −0.57 | −0.58 | 0.01 |

| | | |
|---|---|---|
| 13 | 14 | 15 |
| ?A | ?A | ?A |

| | | | |
|---|---|---|---|
| Frequencies -- | 231.1177 | 269.0983 | 304.6734 |
| Red. masses -- | 4.0410 | 1.8470 | 2.9710 |
| Frc consts -- | 0.1272 | 0.0788 | 0.1625 |
| IR Inten -- | 56.3490 | 85.4662 | 15.4206 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.06 | 0.02 | −0.07 | −0.02 | 0.00 | −0.01 | 0.01 | −0.03 | 0.09 |
| 2 | 6 | 0.14 | −0.03 | 0.12 | −0.02 | −0.01 | −0.04 | −0.06 | 0.04 | −0.04 |
| 3 | 6 | 0.08 | 0.00 | 0.02 | 0.00 | −0.01 | −0.02 | −0.06 | 0.02 | −0.02 |
| 4 | 6 | 0.03 | 0.01 | −0.08 | 0.01 | 0.01 | 0.00 | −0.03 | 0.01 | 0.04 |
| 5 | 6 | −0.02 | 0.02 | −0.14 | 0.00 | 0.01 | 0.01 | −0.03 | −0.02 | 0.02 |
| 6 | 8 | −0.02 | 0.18 | 0.00 | 0.00 | −0.09 | 0.03 | −0.01 | −0.08 | −0.01 |
| 7 | 8 | −0.03 | −0.11 | −0.04 | 0.06 | 0.01 | 0.01 | 0.03 | 0.10 | 0.01 |
| 8 | 6 | −0.07 | 0.06 | −0.10 | 0.00 | −0.01 | 0.03 | 0.04 | 0.06 | −0.19 |
| 9 | 16 | −0.08 | −0.06 | 0.07 | 0.00 | 0.01 | −0.01 | 0.13 | −0.08 | 0.01 |
| 10 | 6 | −0.15 | 0.05 | 0.14 | 0.02 | −0.01 | −0.02 | −0.09 | 0.06 | 0.08 |
| 11 | 1 | 0.15 | 0.00 | 0.12 | −0.02 | −0.02 | −0.03 | −0.06 | 0.03 | −0.04 |
| 12 | 1 | 0.12 | −0.09 | −0.04 | 0.02 | 0.03 | 0.01 | −0.10 | 0.06 | 0.01 |
| 13 | 1 | 0.09 | 0.08 | −0.08 | −0.01 | 0.01 | 0.02 | −0.07 | −0.02 | 0.06 |
| 14 | 1 | −0.07 | 0.02 | −0.13 | 0.03 | 0.02 | 0.01 | −0.15 | −0.08 | 0.03 |
| 15 | 1 | −0.06 | 0.03 | 0.03 | −0.02 | −0.05 | 0.00 | 0.01 | 0.01 | −0.02 |
| 16 | 1 | −0.04 | −0.16 | −0.06 | 0.07 | 0.11 | 0.01 | 0.03 | −0.08 | 0.14 |
| 17 | 1 | −0.19 | 0.19 | −0.06 | 0.01 | −0.07 | 0.02 | 0.03 | 0.43 | −0.11 |
| 18 | 1 | −0.03 | 0.04 | −0.23 | 0.01 | −0.01 | 0.08 | 0.05 | 0.02 | −0.56 |
| 19 | 1 | −0.15 | 0.03 | 0.15 | 0.03 | −0.01 | −0.03 | −0.19 | 0.06 | 0.22 |
| 20 | 1 | −0.11 | 0.20 | 0.08 | 0.02 | −0.03 | 0.00 | −0.14 | 0.25 | −0.13 |
| 21 | 1 | −0.26 | 0.05 | 0.25 | 0.03 | −0.01 | −0.04 | −0.18 | 0.05 | 0.20 |
| 22 | 15 | 0.04 | 0.00 | 0.00 | −0.02 | 0.04 | 0.01 | −0.02 | 0.00 | 0.00 |
| 23 | 8 | 0.01 | 0.04 | 0.00 | −0.03 | 0.08 | 0.03 | −0.01 | 0.00 | −0.01 |
| 24 | 8 | 0.08 | −0.01 | −0.05 | 0.07 | 0.03 | −0.09 | −0.01 | 0.01 | 0.00 |
| 25 | 8 | −0.05 | −0.02 | 0.04 | −0.08 | 0.04 | 0.02 | −0.02 | 0.00 | 0.00 |
| 26 | 1 | −0.09 | −0.05 | 0.07 | −0.11 | −0.01 | 0.01 | −0.01 | 0.00 | −0.02 |
| 27 | 8 | 0.04 | −0.05 | 0.01 | 0.02 | −0.05 | 0.04 | −0.01 | 0.00 | 0.00 |
| 28 | 1 | 0.11 | −0.48 | 0.04 | 0.13 | −0.73 | 0.14 | 0.00 | −0.06 | 0.02 |
| 29 | 1 | 0.13 | −0.03 | −0.04 | −0.31 | −0.47 | −0.06 | −0.04 | −0.02 | 0.00 |

| | | |
|---|---|---|
| 16 | 17 | 18 |
| ?A | ?A | ?A |

| | | | |
|---|---|---|---|
| Frequencies -- | 330.7181 | 341.8624 | 357.6717 |
| Red. masses -- | 2.0629 | 1.4620 | 3.6462 |
| Frc consts -- | 0.1329 | 0.1007 | 0.2748 |
| IR Inten -- | 33.4767 | 152.6057 | 30.7929 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.03 | 0.01 | −0.09 | 0.01 | 0.00 | 0.00 | −0.03 | 0.02 | −0.07 |
| 2 | 6 | 0.03 | 0.02 | −0.04 | 0.01 | 0.00 | 0.01 | −0.01 | −0.02 | 0.02 |
| 3 | 6 | 0.00 | −0.03 | 0.03 | −0.01 | 0.00 | −0.03 | 0.01 | −0.04 | 0.05 |
| 4 | 6 | 0.04 | −0.11 | 0.09 | −0.02 | 0.02 | −0.03 | 0.01 | −0.03 | 0.05 |
| 5 | 6 | 0.07 | −0.11 | 0.07 | −0.03 | 0.02 | −0.04 | 0.00 | −0.04 | 0.02 |
| 6 | 8 | 0.06 | −0.01 | −0.02 | −0.10 | −0.02 | 0.04 | 0.04 | 0.03 | −0.01 |
| 7 | 8 | −0.09 | 0.04 | 0.00 | 0.06 | −0.04 | 0.02 | −0.01 | 0.07 | −0.01 |
| 8 | 6 | 0.01 | 0.07 | 0.00 | −0.01 | −0.02 | −0.01 | −0.01 | −0.01 | 0.00 |
| 9 | 16 | −0.04 | 0.01 | 0.01 | 0.03 | 0.00 | 0.02 | 0.00 | 0.00 | −0.01 |
| 10 | 6 | −0.01 | −0.01 | 0.00 | −0.02 | −0.01 | 0.00 | 0.01 | 0.00 | 0.00 |
| 11 | 1 | 0.03 | 0.07 | −0.05 | 0.03 | −0.01 | 0.03 | 0.04 | −0.03 | 0.07 |
| 12 | 1 | −0.11 | −0.02 | 0.03 | 0.03 | −0.01 | −0.03 | −0.02 | −0.06 | 0.02 |
| 13 | 1 | 0.05 | −0.24 | 0.01 | −0.04 | 0.07 | 0.02 | 0.04 | −0.07 | 0.01 |
| 14 | 1 | 0.13 | −0.25 | −0.01 | −0.04 | 0.06 | −0.02 | 0.04 | −0.12 | −0.03 |
| 15 | 1 | 0.04 | 0.45 | −0.21 | −0.22 | 0.25 | −0.15 | 0.07 | 0.03 | 0.02 |
| 16 | 1 | −0.10 | 0.47 | −0.31 | 0.11 | 0.82 | −0.26 | −0.02 | −0.14 | 0.06 |
| 17 | 1 | 0.07 | 0.29 | 0.04 | −0.05 | −0.08 | −0.02 | 0.01 | 0.02 | 0.00 |
| 18 | 1 | −0.09 | 0.05 | −0.22 | 0.01 | −0.02 | 0.04 | −0.03 | −0.02 | −0.04 |
| 19 | 1 | 0.01 | −0.03 | −0.03 | −0.03 | −0.03 | 0.02 | 0.00 | 0.01 | 0.00 |
| 20 | 1 | 0.02 | 0.01 | 0.04 | −0.02 | 0.00 | −0.02 | 0.00 | 0.00 | −0.01 |
| 21 | 1 | −0.05 | 0.00 | 0.00 | −0.04 | 0.00 | −0.02 | 0.01 | 0.00 | 0.00 |
| 22 | 15 | 0.00 | 0.01 | 0.01 | 0.01 | −0.01 | 0.00 | −0.02 | 0.00 | 0.02 |
| 23 | 8 | 0.00 | 0.01 | −0.01 | 0.01 | 0.01 | 0.03 | 0.00 | −0.10 | 0.14 |
| 24 | 8 | 0.02 | 0.03 | 0.00 | −0.02 | −0.04 | −0.01 | −0.10 | −0.14 | −0.05 |
| 25 | 8 | 0.01 | 0.00 | 0.01 | 0.01 | 0.01 | −0.03 | 0.00 | 0.08 | −0.18 |
| 26 | 1 | 0.00 | 0.00 | 0.04 | −0.07 | −0.06 | 0.10 | −0.06 | 0.01 | −0.12 |
| 27 | 8 | −0.02 | −0.02 | 0.00 | 0.02 | 0.01 | 0.00 | 0.09 | 0.20 | 0.05 |
| 28 | 1 | −0.03 | 0.02 | −0.03 | 0.01 | 0.07 | 0.00 | 0.23 | −0.64 | 0.30 |
| 29 | 1 | −0.10 | −0.08 | 0.01 | 0.16 | 0.10 | −0.01 | 0.39 | 0.16 | −0.05 |

| | | |
|---|---|---|
| 19 | 20 | 21 |
| ?A | ?A | ?A |

| | | | |
|---|---|---|---|
| Frequencies -- | 368.5625 | 380.8503 | 402.2122 |
| Red. masses -- | 5.6670 | 2.6919 | 3.2646 |
| Frc consts -- | 0.4536 | 0.2301 | 0.3112 |
| IR Inten -- | 10.6786 | 7.8016 | 42.2210 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.20 | −0.08 | 0.18 | 0.02 | 0.00 | −0.06 | −0.04 | 0.02 | 0.02 |
| 2 | 6 | 0.02 | 0.11 | −0.17 | 0.01 | 0.01 | −0.01 | −0.02 | −0.02 | 0.01 |
| 3 | 6 | −0.01 | 0.12 | −0.15 | −0.03 | −0.02 | 0.01 | 0.03 | 0.00 | −0.01 |
| 4 | 6 | 0.05 | −0.02 | −0.08 | 0.01 | −0.07 | 0.03 | 0.00 | 0.06 | 0.00 |
| 5 | 6 | 0.17 | 0.03 | 0.10 | 0.02 | −0.09 | 0.02 | −0.02 | 0.07 | −0.01 |
| 6 | 8 | −0.09 | −0.08 | 0.01 | −0.10 | 0.01 | 0.04 | 0.16 | −0.08 | −0.06 |
| 7 | 8 | −0.01 | −0.17 | −0.03 | 0.15 | 0.12 | −0.05 | −0.15 | −0.08 | 0.05 |
| 8 | 6 | 0.11 | 0.19 | 0.07 | −0.03 | −0.02 | 0.00 | 0.02 | 0.01 | 0.00 |
| 9 | 16 | −0.11 | 0.01 | 0.01 | −0.04 | 0.01 | −0.01 | 0.04 | −0.01 | 0.00 |
| 10 | 6 | −0.01 | −0.01 | 0.01 | 0.01 | 0.00 | 0.00 | 0.00 | 0.00 | 0.00 |
| 11 | 1 | 0.03 | 0.07 | −0.15 | 0.02 | 0.05 | −0.01 | −0.03 | −0.05 | 0.01 |
| 12 | 1 | 0.01 | 0.25 | −0.04 | −0.06 | 0.00 | 0.02 | 0.03 | 0.00 | 0.00 |
| 13 | 1 | −0.05 | −0.10 | −0.04 | −0.04 | −0.13 | 0.03 | 0.05 | 0.10 | −0.02 |
| 14 | 1 | 0.15 | 0.13 | 0.14 | 0.06 | −0.18 | −0.03 | −0.04 | 0.12 | 0.01 |
| 15 | 1 | −0.12 | −0.32 | 0.07 | −0.16 | 0.04 | −0.02 | 0.25 | 0.08 | −0.05 |
| 16 | 1 | −0.02 | −0.14 | −0.08 | 0.15 | −0.36 | 0.30 | −0.17 | 0.03 | −0.15 |
| 17 | 1 | 0.17 | 0.39 | 0.10 | 0.00 | 0.03 | 0.01 | 0.00 | −0.04 | −0.01 |
| 18 | 1 | 0.05 | 0.17 | −0.15 | −0.09 | −0.02 | −0.05 | 0.07 | 0.01 | 0.05 |
| 19 | 1 | 0.05 | −0.02 | −0.07 | 0.03 | 0.02 | −0.03 | −0.02 | −0.01 | 0.02 |
| 20 | 1 | 0.04 | −0.01 | 0.10 | 0.02 | −0.03 | 0.03 | −0.01 | 0.02 | −0.03 |
| 21 | 1 | −0.03 | −0.01 | 0.02 | 0.04 | 0.00 | 0.00 | −0.02 | 0.00 | 0.00 |
| 22 | 15 | −0.01 | −0.02 | 0.02 | 0.02 | −0.01 | 0.01 | −0.03 | 0.00 | 0.01 |
| 23 | 8 | 0.00 | 0.00 | 0.04 | −0.02 | 0.12 | 0.03 | −0.07 | 0.12 | 0.04 |
| 24 | 8 | −0.04 | −0.09 | −0.01 | 0.03 | −0.06 | −0.05 | 0.05 | −0.04 | −0.10 |
| 25 | 8 | −0.07 | 0.03 | −0.08 | −0.06 | 0.01 | −0.01 | −0.10 | 0.02 | −0.04 |
| 26 | 1 | −0.14 | −0.06 | −0.06 | −0.21 | −0.13 | 0.11 | −0.18 | −0.09 | −0.06 |
| 27 | 8 | −0.01 | 0.06 | 0.01 | 0.05 | −0.08 | 0.03 | 0.10 | −0.07 | 0.08 |
| 28 | 1 | 0.01 | −0.06 | 0.04 | −0.06 | 0.55 | −0.02 | 0.00 | 0.56 | 0.17 |
| 29 | 1 | 0.19 | 0.06 | −0.01 | 0.37 | 0.13 | −0.05 | 0.51 | 0.16 | −0.09 |

| | | |
|---|---|---|
| 22 | 23 | 24 |
| ?A | ?A | ?A |

| | | | |
|---|---|---|---|
| Frequencies -- | 429.9528 | 444.9801 | 455.6163 |
| Red. masses -- | 1.8546 | 3.6170 | 3.5320 |
| Frc consts -- | 0.2020 | 0.4220 | 0.4320 |
| IR Inten -- | 4.3637 | 17.0904 | 36.3103 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.07 | 0.02 | 0.03 | −0.07 | 0.01 | −0.04 | 0.11 | −0.02 | 0.04 |
| 2 | 6 | 0.04 | 0.06 | 0.04 | −0.04 | −0.04 | −0.05 | 0.01 | 0.09 | −0.01 |
| 3 | 6 | −0.03 | 0.02 | −0.02 | 0.02 | −0.03 | 0.01 | −0.01 | 0.05 | 0.03 |
| 4 | 6 | −0.03 | 0.02 | 0.00 | 0.00 | 0.01 | 0.03 | −0.01 | −0.03 | 0.02 |
| 5 | 6 | −0.03 | 0.01 | −0.06 | −0.01 | 0.01 | 0.02 | −0.04 | −0.08 | −0.04 |
| 6 | 8 | 0.06 | −0.11 | −0.02 | 0.03 | 0.03 | −0.03 | 0.10 | 0.00 | −0.01 |
| 7 | 8 | 0.00 | 0.00 | 0.01 | −0.05 | 0.02 | 0.02 | 0.01 | 0.05 | −0.02 |
| 8 | 6 | −0.04 | −0.06 | −0.03 | 0.01 | 0.02 | 0.01 | −0.07 | −0.10 | −0.06 |
| 9 | 16 | −0.02 | 0.02 | 0.01 | 0.03 | −0.01 | −0.01 | −0.02 | 0.02 | 0.02 |
| 10 | 6 | 0.00 | 0.00 | 0.01 | 0.00 | 0.00 | −0.01 | 0.00 | −0.01 | 0.01 |
| 11 | 1 | 0.07 | 0.09 | 0.07 | −0.04 | −0.07 | −0.04 | 0.06 | 0.12 | 0.03 |
| 12 | 1 | −0.07 | 0.04 | −0.02 | 0.02 | −0.02 | 0.02 | −0.05 | 0.12 | 0.08 |
| 13 | 1 | −0.04 | 0.05 | 0.02 | 0.03 | 0.00 | 0.00 | 0.00 | −0.05 | 0.00 |
| 14 | 1 | −0.07 | 0.03 | −0.04 | 0.03 | −0.02 | 0.00 | −0.11 | −0.13 | −0.04 |
| 15 | 1 | 0.03 | 0.71 | −0.34 | 0.11 | −0.55 | 0.26 | 0.22 | −0.50 | 0.28 |
| 16 | 1 | −0.01 | −0.34 | 0.15 | −0.05 | 0.24 | −0.11 | 0.04 | 0.38 | −0.11 |
| 17 | 1 | −0.09 | −0.17 | −0.04 | 0.03 | 0.05 | 0.01 | −0.12 | −0.18 | −0.07 |
| 18 | 1 | −0.03 | −0.04 | 0.11 | 0.02 | 0.01 | −0.04 | −0.11 | −0.08 | 0.05 |
| 19 | 1 | 0.01 | 0.01 | 0.00 | −0.01 | −0.01 | 0.01 | 0.00 | −0.01 | 0.00 |
| 20 | 1 | 0.00 | −0.03 | 0.02 | 0.00 | 0.02 | −0.02 | 0.00 | −0.04 | 0.03 |
| 21 | 1 | 0.03 | 0.00 | 0.00 | −0.02 | 0.00 | 0.00 | 0.02 | −0.01 | −0.02 |
| 22 | 15 | 0.04 | 0.00 | 0.01 | 0.14 | 0.04 | 0.03 | 0.00 | −0.07 | −0.08 |
| 23 | 8 | 0.05 | 0.00 | 0.00 | 0.14 | 0.06 | 0.03 | −0.07 | 0.11 | 0.14 |
| 24 | 8 | −0.03 | −0.03 | 0.04 | −0.08 | −0.14 | 0.07 | 0.00 | 0.07 | 0.07 |
| 25 | 8 | −0.05 | 0.02 | −0.01 | −0.16 | 0.07 | 0.03 | −0.07 | −0.12 | 0.00 |
| 26 | 1 | −0.16 | −0.11 | 0.05 | −0.32 | −0.16 | −0.12 | −0.02 | −0.09 | −0.14 |
| 27 | 8 | −0.05 | 0.02 | −0.04 | −0.10 | −0.03 | −0.10 | 0.08 | 0.09 | −0.07 |
| 28 | 1 | −0.03 | −0.12 | −0.15 | −0.10 | −0.06 | −0.45 | 0.09 | 0.00 | 0.06 |
| 29 | 1 | −0.09 | −0.02 | 0.04 | −0.05 | −0.05 | 0.06 | −0.29 | 0.17 | 0.04 |

| | | |
|---|---|---|
| 25 | 26 | 27 |
| ?A | ?A | ?A |

| | | | |
|---|---|---|---|
| Frequencies -- | 461.5377 | 481.1776 | 523.0637 |
| Red. masses -- | 3.6003 | 10.9368 | 3.8966 |
| Frc consts -- | 0.4519 | 1.4919 | 0.6281 |
| IR Inten -- | 17.4552 | 112.9280 | 107.3430 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.11 | 0.02 | −0.07 | 0.10 | −0.01 | 0.04 | 0.09 | 0.05 | −0.10 |
| 2 | 6 | −0.02 | −0.09 | 0.02 | −0.01 | 0.08 | −0.03 | 0.17 | −0.03 | 0.19 |
| 3 | 6 | 0.01 | −0.05 | −0.02 | −0.01 | 0.05 | 0.00 | −0.13 | −0.07 | −0.06 |
| 4 | 6 | 0.02 | 0.03 | −0.02 | −0.01 | −0.01 | 0.00 | −0.12 | 0.05 | 0.05 |
| 5 | 6 | 0.05 | 0.08 | 0.05 | 0.00 | −0.03 | −0.02 | 0.06 | 0.05 | −0.04 |
| 6 | 8 | −0.08 | −0.03 | 0.00 | 0.05 | 0.00 | −0.01 | −0.10 | −0.16 | −0.14 |
| 7 | 8 | −0.01 | −0.05 | 0.01 | 0.01 | 0.01 | −0.01 | −0.05 | 0.09 | 0.09 |
| 8 | 6 | 0.09 | 0.12 | 0.07 | −0.02 | −0.04 | −0.03 | 0.07 | 0.05 | 0.03 |
| 9 | 16 | 0.02 | −0.02 | −0.02 | −0.02 | 0.01 | 0.01 | −0.02 | 0.01 | 0.00 |
| 10 | 6 | 0.00 | 0.01 | −0.01 | 0.00 | 0.00 | 0.01 | 0.00 | 0.00 | 0.00 |
| 11 | 1 | 0.00 | −0.15 | 0.06 | 0.10 | 0.03 | 0.09 | 0.29 | 0.06 | 0.28 |
| 12 | 1 | 0.06 | −0.12 | −0.08 | −0.04 | 0.09 | 0.03 | −0.14 | −0.16 | −0.14 |
| 13 | 1 | 0.01 | 0.03 | −0.01 | −0.02 | −0.03 | 0.00 | −0.20 | 0.06 | 0.13 |
| 14 | 1 | 0.16 | 0.11 | 0.03 | −0.02 | −0.05 | −0.02 | 0.15 | 0.04 | −0.07 |
| 15 | 1 | −0.18 | 0.42 | −0.25 | 0.07 | −0.07 | 0.04 | 0.05 | −0.54 | 0.12 |
| 16 | 1 | −0.04 | −0.35 | 0.08 | 0.02 | 0.09 | −0.03 | −0.04 | 0.13 | 0.13 |
| 17 | 1 | 0.14 | 0.22 | 0.09 | −0.04 | −0.07 | −0.03 | 0.05 | 0.05 | 0.03 |
| 18 | 1 | 0.12 | 0.10 | −0.07 | −0.04 | −0.03 | 0.02 | 0.07 | 0.05 | 0.02 |
| 19 | 1 | 0.00 | 0.00 | 0.00 | 0.01 | 0.00 | 0.00 | 0.01 | 0.00 | −0.02 |
| 20 | 1 | 0.01 | 0.04 | −0.02 | 0.00 | −0.02 | 0.02 | 0.01 | −0.01 | 0.03 |
| 21 | 1 | −0.02 | 0.00 | 0.02 | 0.01 | 0.00 | −0.01 | 0.00 | 0.00 | 0.00 |
| 22 | 15 | 0.03 | −0.03 | −0.10 | −0.03 | 0.21 | −0.14 | 0.01 | 0.00 | 0.00 |
| 23 | 8 | −0.03 | 0.07 | 0.19 | 0.10 | −0.33 | 0.26 | −0.01 | −0.02 | −0.02 |
| 24 | 8 | −0.04 | 0.06 | 0.07 | −0.28 | 0.03 | −0.20 | 0.01 | 0.01 | −0.01 |
| 25 | 8 | −0.02 | −0.10 | 0.04 | −0.01 | 0.09 | 0.27 | 0.01 | −0.01 | 0.02 |
| 26 | 1 | −0.09 | −0.14 | 0.21 | 0.11 | 0.23 | 0.27 | 0.16 | 0.11 | −0.24 |
| 27 | 8 | 0.09 | 0.05 | −0.09 | 0.16 | −0.29 | −0.07 | 0.01 | 0.00 | 0.01 |
| 28 | 1 | 0.10 | 0.00 | −0.02 | 0.08 | 0.26 | 0.01 | 0.01 | 0.00 | 0.01 |
| 29 | 1 | −0.33 | 0.18 | 0.03 | 0.02 | 0.28 | −0.22 | 0.06 | 0.00 | 0.00 |

| | | |
|---|---|---|
| 28 | 29 | 30 |
| ?A | ?A | ?A |

| | | | |
|---|---|---|---|
| Frequencies -- | 589.6155 | 687.8443 | 697.1515 |
| Red. masses -- | 6.2799 | 4.9963 | 4.5755 |
| Frc consts -- | 1.2863 | 1.3928 | 1.3102 |
| IR Inten -- | 31.5408 | 25.8451 | 23.0645 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.15 | 0.29 | 0.06 | −0.07 | 0.18 | 0.08 | 0.00 | 0.19 | 0.08 |
| 2 | 6 | −0.02 | 0.15 | 0.26 | 0.07 | −0.01 | −0.04 | 0.14 | 0.00 | −0.07 |
| 3 | 6 | −0.06 | 0.05 | 0.01 | 0.11 | −0.02 | −0.11 | 0.15 | −0.01 | −0.16 |
| 4 | 6 | −0.08 | −0.16 | −0.12 | 0.09 | −0.04 | 0.07 | 0.11 | −0.05 | 0.08 |
| 5 | 6 | −0.02 | −0.07 | 0.13 | 0.00 | −0.01 | 0.06 | −0.06 | −0.02 | 0.06 |
| 6 | 8 | 0.07 | −0.07 | −0.05 | −0.07 | −0.03 | −0.04 | −0.08 | −0.04 | −0.04 |
| 7 | 8 | 0.13 | −0.18 | −0.23 | −0.04 | 0.01 | 0.04 | −0.06 | 0.03 | 0.06 |
| 8 | 6 | −0.01 | 0.02 | 0.05 | 0.06 | −0.10 | −0.16 | −0.14 | −0.06 | −0.01 |
| 9 | 16 | 0.02 | −0.01 | −0.01 | −0.01 | 0.11 | −0.10 | 0.00 | −0.07 | 0.09 |
| 10 | 6 | 0.01 | 0.02 | −0.03 | −0.05 | −0.18 | 0.31 | 0.04 | 0.14 | −0.24 |
| 11 | 1 | −0.04 | 0.11 | 0.26 | −0.03 | −0.22 | −0.10 | 0.05 | −0.24 | −0.11 |
| 12 | 1 | −0.08 | 0.03 | 0.00 | 0.08 | −0.02 | −0.11 | 0.10 | 0.02 | −0.14 |
| 13 | 1 | −0.11 | −0.31 | −0.18 | 0.10 | −0.08 | 0.04 | 0.12 | −0.07 | 0.06 |
| 14 | 1 | 0.02 | −0.05 | 0.13 | −0.13 | −0.12 | 0.05 | −0.14 | −0.16 | 0.03 |
| 15 | 1 | 0.17 | −0.16 | 0.07 | −0.17 | −0.19 | −0.07 | −0.22 | −0.25 | −0.08 |
| 16 | 1 | 0.19 | 0.11 | −0.08 | −0.08 | −0.09 | −0.10 | −0.12 | −0.17 | −0.07 |
| 17 | 1 | 0.11 | 0.29 | 0.09 | 0.17 | −0.25 | −0.20 | −0.20 | −0.04 | 0.00 |
| 18 | 1 | 0.10 | −0.01 | −0.23 | 0.05 | −0.08 | 0.02 | −0.13 | −0.06 | −0.01 |
| 19 | 1 | 0.00 | 0.00 | −0.02 | 0.02 | −0.15 | 0.20 | −0.03 | 0.12 | −0.13 |
| 20 | 1 | 0.01 | 0.02 | −0.03 | −0.05 | −0.23 | 0.34 | 0.03 | 0.17 | −0.27 |
| 21 | 1 | 0.00 | 0.01 | −0.02 | 0.02 | −0.16 | 0.17 | −0.01 | 0.13 | −0.13 |
| 22 | 15 | 0.02 | 0.00 | 0.01 | 0.00 | 0.00 | 0.00 | −0.01 | 0.00 | 0.00 |
| 23 | 8 | 0.00 | −0.02 | −0.01 | −0.01 | 0.00 | 0.01 | −0.01 | −0.01 | 0.01 |
| 24 | 8 | 0.01 | −0.01 | 0.00 | 0.00 | 0.00 | 0.00 | 0.00 | 0.01 | 0.00 |
| 25 | 8 | −0.01 | 0.00 | 0.01 | 0.02 | 0.00 | −0.01 | 0.03 | 0.00 | −0.01 |
| 26 | 1 | 0.09 | 0.07 | −0.23 | −0.07 | −0.06 | 0.18 | −0.14 | −0.12 | 0.34 |
| 27 | 8 | 0.00 | 0.00 | 0.00 | 0.00 | 0.00 | 0.00 | 0.00 | 0.00 | 0.01 |
| 28 | 1 | 0.00 | 0.00 | −0.03 | 0.00 | 0.00 | 0.01 | 0.00 | −0.01 | 0.01 |
| 29 | 1 | 0.05 | −0.01 | 0.00 | −0.02 | 0.00 | 0.00 | −0.04 | 0.01 | 0.00 |

| | | 31 | | | 32 | | | 33 | | |
|---|---|---|---|---|---|---|---|---|---|---|
| | | ?A | | | ?A | | | ?A | | |
| Frequencies -- | | 725.3578 | | | 756.9012 | | | 776.2191 | | |
| Red. masses -- | | 1.1857 | | | 5.3889 | | | 4.1088 | | |
| Frc consts -- | | 0.3676 | | | 1.8190 | | | 14586 | | |
| IR Inten -- | | 106.7801 | | | 11.3548 | | | 52.46223 | | |
| Raman Activ -- | | 0.0000 | | | 0.0000 | | | 0.0000 | | |
| Depolar -- | | 0.0000 | | | 0.0000 | | | 0.0000 | | |
| Atom | AN | X | Y | Z | X | Y | Z | X | Y | Z |
| 1 | 8 | 0.01 | −0.01 | −0.01 | −0.09 | 0.01 | 0.01 | −0.10 | −0.08 | −0.04 |
| 2 | 6 | −0.01 | 0.01 | 0.02 | −0.06 | −0.04 | 0.02 | −0.03 | −0.07 | 0.14 |
| 3 | 6 | −0.03 | −0.01 | 0.00 | −0.01 | 0.00 | 0.02 | 0.04 | 0.33 | −0.21 |
| 4 | 6 | −0.05 | 0.01 | −0.01 | −0.05 | 0.04 | −0.02 | 0.00 | 0.08 | −0.02 |
| 5 | 6 | 0.00 | 0.00 | 0.00 | 0.10 | −0.01 | −0.01 | 0.10 | −0.17 | −0.04 |
| 6 | 8 | 0.05 | 0.01 | −0.02 | 0.02 | 0.01 | 0.01 | 0.04 | −0.01 | 0.06 |
| 7 | 8 | 0.00 | 0.00 | −0.01 | 0.01 | −0.01 | 0.00 | −0.03 | 0.03 | 0.07 |
| 8 | 6 | 0.01 | 0.01 | 0.01 | 0.34 | −0.22 | −0.24 | −0.07 | −0.05 | 0.04 |
| 9 | 16 | 0.00 | 0.00 | −0.01 | −0.13 | 0.05 | 0.15 | 0.01 | 0.00 | −0.02 |
| 10 | 6 | 0.00 | −0.01 | 0.01 | 0.01 | 0.10 | −0.13 | −0.01 | −0.01 | 0.02 |
| 11 | 1 | 0.02 | 0.04 | 0.04 | −0.12 | −0.08 | −0.03 | 0.03 | −0.32 | 0.26 |
| 12 | 1 | −0.01 | 0.00 | 0.01 | 0.07 | −0.03 | 0.00 | 0.30 | 0.36 | −0.16 |
| 13 | 1 | −0.07 | 0.00 | 0.00 | −0.09 | 0.02 | −0.01 | −0.23 | −0.07 | 0.11 |
| 14 | 1 | 0.03 | 0.02 | 0.00 | 0.10 | 0.08 | 0.03 | 0.17 | −0.20 | −0.07 |
| 15 | 1 | 0.05 | −0.18 | 0.05 | 0.03 | 0.05 | 0.01 | −0.02 | 0.03 | 0.00 |
| 16 | 1 | 0.02 | 0.05 | 0.05 | 0.02 | 0.06 | 0.05 | −0.04 | 0.01 | 0.01 |
| 17 | 1 | 0.03 | 0.05 | 0.02 | 0.42 | −0.08 | −0.22 | −0.05 | 0.19 | 0.08 |
| 18 | 1 | 0.00 | 0.00 | −0.04 | 0.44 | −0.24 | −0.29 | −0.22 | −0.08 | −0.23 |
| 19 | 1 | 0.00 | −0.01 | 0.00 | 0.01 | 0.10 | −0.14 | 0.02 | −0.01 | −0.02 |
| 20 | 1 | 0.00 | −0.01 | 0.02 | 0.02 | 0.01 | −0.05 | 0.00 | −0.06 | 0.06 |
| 21 | 1 | 0.00 | 0.00 | 0.00 | 0.03 | 0.09 | −0.11 | 0.02 | −0.01 | −0.02 |
| 22 | 15 | 0.00 | 0.01 | −0.01 | 0.01 | 0.00 | 0.00 | 0.02 | 0.01 | 0.00 |
| 23 | 8 | −0.01 | −0.02 | −0.02 | −0.01 | 0.00 | 0.00 | −0.02 | −0.02 | −0.01 |
| 24 | 8 | 0.00 | 0.02 | 0.00 | 0.00 | 0.00 | 0.00 | 0.01 | −0.01 | 0.01 |
| 25 | 8 | 0.05 | 0.01 | −0.03 | 0.00 | 0.00 | 0.00 | 0.00 | 0.00 | 0.00 |
| 26 | 1 | −0.42 | −0.37 | 0.78 | 0.01 | 0.01 | −0.02 | 0.02 | 0.02 | −0.03 |
| 27 | 8 | −0.01 | 0.00 | 0.02 | 0.00 | 0.00 | 0.00 | 0.01 | 0.00 | 0.00 |
| 28 | 1 | −0.01 | −0.03 | 0.00 | 0.00 | 0.00 | −0.01 | 0.01 | −0.01 | −0.05 |
| 29 | 1 | −0.07 | 0.02 | 0.00 | 0.01 | 0.00 | 0.01 | 0.02 | −0.01 | 0.01 |

| | | |
|---|---|---|
| 34 | 35 | 36 |
| ?A | ?A | ?A |

| | | | |
|---|---|---|---|
| Frequencies -- | 806.6552 | 855.9299 | 876.4632 |
| Red. masses -- | 2.5783 | 11.7829 | 2.5180 |
| Frc consts -- | 0.9885 | 5.0860 | 1.1397 |
| IR Inten -- | 30.3922 | 23.3399 | 5.1806 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.07 | −0.02 | 0.05 | −0.02 | 0.00 | 0.00 | 0.03 | −0.05 | −0.04 |
| 2 | 6 | −0.10 | 0.04 | 0.09 | −0.01 | −0.01 | −0.01 | −0.11 | 0.10 | 0.09 |
| 3 | 6 | −0.07 | 0.05 | 0.02 | −0.01 | 0.02 | −0.01 | 0.00 | −0.03 | 0.04 |
| 4 | 6 | 0.18 | −0.06 | 0.07 | 0.00 | 0.00 | 0.00 | 0.21 | 0.10 | −0.01 |
| 5 | 6 | 0.10 | −0.01 | −0.15 | 0.01 | −0.01 | −0.02 | −0.03 | 0.00 | 0.05 |
| 6 | 8 | −0.04 | −0.03 | −0.04 | −0.01 | 0.00 | 0.01 | −0.07 | −0.04 | −0.07 |
| 7 | 8 | 0.01 | 0.02 | −0.02 | 0.00 | 0.00 | 0.00 | 0.00 | −0.01 | 0.02 |
| 8 | 6 | 0.04 | 0.13 | −0.05 | 0.01 | 0.01 | −0.01 | −0.03 | −0.13 | 0.03 |
| 9 | 16 | −0.01 | −0.02 | 0.02 | 0.00 | 0.00 | 0.00 | 0.00 | 0.01 | −0.01 |
| 10 | 6 | 0.01 | 0.00 | −0.02 | 0.00 | 0.00 | 0.00 | −0.02 | 0.02 | 0.01 |
| 11 | 1 | −0.09 | 0.04 | 0.12 | 0.03 | −0.12 | 0.06 | −0.01 | 0.38 | 0.14 |
| 12 | 1 | −0.16 | 0.04 | 0.00 | −0.01 | 0.04 | 0.00 | 0.05 | −0.14 | −0.06 |
| 13 | 1 | 0.19 | −0.12 | 0.03 | −0.03 | −0.02 | 0.01 | 0.35 | 0.21 | −0.07 |
| 14 | 1 | −0.11 | −0.20 | −0.16 | 0.02 | −0.03 | −0.03 | 0.07 | 0.08 | 0.05 |
| 15 | 1 | 0.03 | −0.08 | 0.03 | 0.00 | 0.05 | 0.00 | −0.01 | −0.07 | −0.02 |
| 16 | 1 | −0.04 | −0.05 | −0.23 | 0.00 | 0.01 | 0.00 | −0.07 | −0.12 | −0.29 |
| 17 | 1 | −0.09 | −0.45 | −0.15 | 0.00 | −0.03 | −0.01 | −0.03 | 0.30 | 0.12 |
| 18 | 1 | 0.11 | 0.20 | 0.52 | 0.00 | 0.02 | 0.04 | 0.01 | −0.19 | −0.40 |
| 19 | 1 | −0.04 | 0.02 | 0.05 | 0.00 | 0.00 | 0.00 | 0.05 | −0.02 | −0.09 |
| 20 | 1 | 0.00 | 0.11 | −0.11 | 0.00 | 0.01 | −0.01 | 0.00 | −0.14 | 0.14 |
| 21 | 1 | −0.05 | −0.02 | 0.08 | 0.00 | 0.00 | 0.01 | 0.07 | 0.04 | −0.13 |
| 22 | 15 | 0.01 | 0.00 | 0.00 | 0.10 | 0.02 | −0.01 | 0.00 | −0.01 | 0.00 |
| 23 | 8 | −0.01 | −0.01 | −0.01 | 0.31 | 0.07 | 0.04 | 0.02 | 0.00 | −0.01 |
| 24 | 8 | 0.01 | 0.00 | 0.01 | −0.25 | 0.28 | −0.24 | 0.00 | 0.00 | −0.01 |
| 25 | 8 | 0.01 | 0.01 | 0.00 | −0.05 | −0.39 | −0.16 | 0.00 | 0.01 | 0.00 |
| 26 | 1 | −0.07 | −0.06 | 0.13 | 0.10 | −0.23 | −0.24 | −0.02 | −0.02 | 0.06 |
| 27 | 8 | 0.00 | 0.00 | 0.00 | −0.18 | 0.02 | 0.42 | −0.01 | 0.00 | 0.01 |
| 28 | 1 | 0.00 | −0.01 | −0.03 | −0.21 | −0.05 | −0.21 | −0.01 | 0.00 | −0.01 |
| 29 | 1 | 0.00 | 0.00 | 0.01 | 0.04 | 0.04 | −0.22 | −0.01 | 0.02 | −0.01 |

| | | |
|---|---|---|
| 37 | 38 | 39 |
| ?A | ?A | ?A |

| | | | |
|---|---|---|---|
| Frequencies -- | 902.9391 | 945.7644 | 954.4221 |
| Red. masses -- | 3.2479 | 2.1867 | 1.7752 |
| Frc consts -- | 1.5602 | 1.1524 | 0.9528 |
| IR Inten -- | 57.4390 | 254.1102 | 5.9365 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.05 | −0.09 | −0.15 | 0.00 | 0.00 | 0.00 | 0.01 | 0.00 | 0.00 |
| 2 | 6 | 0.01 | −0.01 | 0.05 | 0.00 | 0.00 | 0.00 | −0.08 | 0.09 | 0.05 |
| 3 | 6 | −0.01 | 0.11 | −0.07 | 0.01 | 0.00 | 0.00 | 0.12 | −0.08 | −0.02 |
| 4 | 6 | 0.06 | −0.02 | 0.01 | −0.01 | 0.00 | 0.00 | −0.07 | 0.06 | −0.05 |
| 5 | 6 | −0.21 | 0.08 | 0.26 | 0.01 | −0.01 | 0.00 | 0.05 | −0.09 | 0.03 |
| 6 | 8 | 0.01 | −0.01 | 0.02 | 0.00 | 0.00 | 0.00 | 0.00 | 0.00 | −0.02 |
| 7 | 8 | 0.01 | −0.03 | −0.02 | 0.00 | 0.00 | 0.00 | −0.02 | 0.01 | 0.03 |
| 8 | 6 | 0.03 | 0.04 | −0.10 | 0.00 | 0.01 | 0.00 | −0.02 | 0.04 | −0.03 |
| 9 | 16 | −0.01 | 0.00 | 0.02 | 0.00 | 0.00 | 0.00 | −0.01 | 0.00 | 0.00 |
| 10 | 6 | 0.04 | −0.02 | −0.01 | 0.00 | 0.00 | 0.00 | 0.04 | −0.05 | −0.02 |
| 11 | 1 | 0.25 | 0.06 | 0.26 | −0.06 | 0.04 | −0.07 | −0.16 | 0.35 | −0.09 |
| 12 | 1 | −0.03 | 0.18 | −0.02 | 0.04 | −0.02 | −0.01 | 0.44 | −0.27 | −0.15 |
| 13 | 1 | 0.01 | −0.13 | −0.01 | −0.02 | 0.00 | 0.00 | −0.11 | 0.05 | −0.02 |
| 14 | 1 | −0.24 | −0.06 | 0.21 | 0.01 | −0.01 | 0.00 | 0.09 | −0.08 | 0.02 |
| 15 | 1 | 0.02 | −0.01 | 0.03 | −0.01 | 0.01 | −0.01 | −0.12 | −0.04 | −0.10 |
| 16 | 1 | −0.01 | −0.06 | −0.10 | 0.00 | 0.01 | 0.02 | 0.01 | 0.03 | 0.15 |
| 17 | 1 | 0.32 | −0.32 | −0.21 | 0.01 | −0.02 | −0.01 | 0.16 | −0.15 | −0.09 |
| 18 | 1 | 0.09 | 0.08 | 0.30 | −0.03 | 0.01 | 0.02 | −0.27 | 0.08 | 0.17 |
| 19 | 1 | −0.09 | 0.01 | 0.16 | −0.01 | 0.00 | 0.01 | −0.08 | 0.06 | 0.14 |
| 20 | 1 | 0.00 | 0.20 | −0.23 | 0.00 | 0.02 | −0.02 | 0.02 | 0.24 | −0.25 |
| 21 | 1 | −0.10 | −0.04 | 0.15 | −0.01 | −0.01 | 0.02 | −0.11 | −0.09 | 0.25 |
| 22 | 15 | 0.00 | 0.00 | 0.00 | 0.01 | 0.01 | −0.09 | 0.00 | −0.01 | 0.01 |
| 23 | 8 | 0.01 | 0.00 | −0.01 | −0.01 | −0.01 | 0.00 | 0.01 | 0.00 | 0.00 |
| 24 | 8 | 0.00 | 0.00 | 0.00 | 0.10 | −0.12 | 0.01 | −0.01 | 0.01 | 0.00 |
| 25 | 8 | 0.00 | 0.00 | 0.00 | 0.00 | 0.05 | 0.02 | −0.01 | 0.00 | 0.00 |
| 26 | 1 | −0.01 | −0.01 | 0.01 | 0.05 | 0.10 | −0.08 | 0.03 | 0.03 | −0.01 |
| 27 | 8 | 0.00 | 0.00 | 0.00 | −0.07 | 0.00 | 0.16 | 0.00 | 0.00 | −0.01 |
| 28 | 1 | 0.00 | 0.00 | −0.01 | −0.08 | 0.00 | −0.03 | 0.00 | 0.01 | 0.04 |
| 29 | 1 | −0.02 | 0.02 | 0.00 | −0.61 | 0.70 | −0.13 | 0.05 | −0.06 | 0.01 |

| | | |
|---|---|---|
| 40 | 41 | 42 |
| ?A | ?A | ?A |

| | | | |
|---|---|---|---|
| Frequencies -- | 992.1694 | 997.2091 | 1004.7576 |
| Red. masses -- | 1.1993 | 1.5169 | 1.5615 |
| Frc consts -- | 0.6956 | 0.8887 | 0.9288 |
| IR Inten -- | 14.8374 | 357.4788 | 21.9321 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | 0.00 | 0.00 | −0.01 | 0.00 | 0.00 | 0.01 | −0.03 | −0.02 |
| 2 | 6 | −0.01 | 0.01 | 0.01 | 0.05 | −0.03 | −0.01 | −0.04 | 0.05 | 0.03 |
| 3 | 6 | 0.02 | −0.01 | −0.01 | −0.05 | 0.02 | 0.03 | 0.09 | −0.01 | −0.06 |
| 4 | 6 | −0.01 | −0.01 | 0.00 | 0.02 | 0.03 | −0.02 | −0.04 | −0.06 | 0.02 |
| 5 | 6 | 0.00 | 0.00 | 0.00 | 0.03 | 0.00 | 0.02 | −0.06 | 0.01 | −0.01 |
| 6 | 8 | 0.00 | 0.00 | 0.00 | −0.01 | −0.01 | −0.01 | 0.01 | 0.00 | 0.01 |
| 7 | 8 | 0.00 | 0.00 | 0.00 | 0.01 | −0.01 | 0.00 | −0.01 | 0.01 | 0.01 |
| 8 | 6 | 0.01 | 0.01 | 0.00 | −0.03 | −0.02 | −0.01 | 0.05 | 0.05 | 0.00 |
| 9 | 16 | −0.02 | −0.01 | −0.01 | −0.01 | 0.00 | 0.00 | 0.02 | −0.02 | 0.00 |
| 10 | 6 | 0.08 | 0.07 | 0.06 | 0.04 | −0.01 | 0.00 | −0.07 | 0.06 | 0.01 |
| 11 | 1 | 0.00 | 0.02 | 0.02 | −0.17 | 0.13 | −0.28 | −0.08 | 0.21 | −0.04 |
| 12 | 1 | 0.06 | −0.03 | −0.02 | −0.11 | 0.05 | 0.05 | 0.19 | −0.06 | −0.09 |
| 13 | 1 | −0.04 | −0.03 | 0.01 | 0.04 | 0.06 | −0.02 | −0.10 | −0.11 | 0.04 |
| 14 | 1 | −0.01 | −0.03 | −0.01 | 0.04 | 0.10 | 0.06 | −0.19 | −0.12 | −0.03 |
| 15 | 1 | −0.03 | −0.01 | −0.02 | 0.06 | 0.03 | 0.04 | −0.11 | −0.05 | −0.08 |
| 16 | 1 | 0.00 | 0.01 | 0.02 | 0.00 | −0.01 | −0.05 | 0.01 | 0.01 | 0.10 |
| 17 | 1 | −0.01 | −0.01 | 0.00 | 0.05 | −0.01 | −0.02 | −0.11 | −0.05 | 0.01 |
| 18 | 1 | 0.05 | 0.01 | 0.02 | −0.14 | −0.02 | −0.03 | 0.26 | 0.05 | 0.10 |
| 19 | 1 | −0.14 | −0.52 | 0.43 | −0.08 | −0.08 | 0.17 | 0.15 | 0.04 | −0.30 |
| 20 | 1 | −0.15 | −0.13 | −0.15 | −0.02 | 0.11 | −0.18 | 0.01 | −0.29 | 0.39 |
| 21 | 1 | −0.11 | 0.27 | −0.59 | −0.09 | 0.01 | 0.02 | 0.19 | 0.06 | −0.21 |
| 22 | 15 | 0.00 | 0.00 | 0.00 | 0.03 | 0.00 | −0.02 | 0.01 | 0.00 | −0.01 |
| 23 | 8 | 0.01 | 0.00 | 0.00 | −0.11 | −0.03 | 0.02 | −0.04 | −0.01 | 0.00 |
| 24 | 8 | 0.00 | 0.00 | 0.00 | −0.01 | 0.02 | 0.01 | 0.00 | 0.00 | 0.01 |
| 25 | 8 | 0.00 | 0.00 | 0.00 | 0.01 | 0.00 | 0.01 | 0.00 | 0.00 | 0.00 |
| 26 | 1 | 0.00 | 0.01 | 0.00 | 0.01 | 0.01 | 0.01 | 0.05 | 0.07 | 0.00 |
| 27 | 8 | 0.00 | 0.00 | −0.01 | 0.04 | 0.02 | 0.07 | 0.02 | 0.01 | 0.03 |
| 28 | 1 | 0.00 | 0.01 | 0.06 | 0.02 | −0.08 | −0.77 | 0.01 | −0.04 | −0.44 |
| 29 | 1 | −0.01 | 0.01 | 0.00 | 0.20 | −0.18 | 0.05 | 0.09 | −0.08 | 0.02 |

| | | |
|---|---|---|
| 43 | 44 | 45 |
| ?A | ?A | ?A |

| | | | |
|---|---|---|---|
| Frequencies -- | 1019.4166 | 1040.7871 | 1064.2484 |
| Red. masses -- | 7.9432 | 1.4543 | 2.0272 |
| Frc consts -- | 4.8635 | 0.9282 | 1.3528 |
| IR Inten -- | 192.6997 | 235.2832 | 37.6707 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | −0.01 | −0.01 | 0.02 | 0.01 | 0.02 | 0.00 | 0.02 | 0.00 |
| 2 | 6 | 0.02 | 0.00 | 0.02 | −0.07 | 0.04 | −0.02 | 0.00 | 0.02 | −0.03 |
| 3 | 6 | −0.03 | −0.01 | −0.02 | 0.03 | −0.04 | −0.01 | −0.05 | −0.03 | 0.04 |
| 4 | 6 | 0.02 | 0.01 | −0.01 | 0.00 | −0.04 | 0.02 | 0.00 | 0.11 | −0.03 |
| 5 | 6 | 0.00 | 0.01 | 0.02 | −0.03 | 0.01 | −0.05 | 0.09 | −0.04 | 0.10 |
| 6 | 8 | 0.00 | 0.00 | 0.00 | 0.01 | 0.01 | 0.01 | 0.00 | 0.00 | 0.00 |
| 7 | 8 | 0.00 | 0.00 | 0.00 | −0.01 | 0.01 | 0.00 | 0.01 | −0.02 | −0.01 |
| 8 | 6 | −0.01 | 0.00 | −0.01 | 0.03 | −0.02 | 0.05 | −0.06 | 0.04 | −0.10 |
| 9 | 16 | 0.00 | 0.00 | 0.00 | −0.01 | 0.02 | 0.00 | 0.02 | −0.03 | 0.01 |
| 10 | 6 | 0.00 | 0.00 | 0.00 | 0.04 | −0.04 | −0.01 | −0.05 | 0.06 | 0.02 |
| 11 | 1 | −0.18 | 0.22 | −0.24 | 0.35 | −0.30 | 0.48 | 0.16 | −0.14 | 0.15 |
| 12 | 1 | −0.06 | 0.00 | −0.01 | −0.01 | −0.02 | 0.00 | 0.01 | −0.06 | 0.02 |
| 13 | 1 | 0.03 | 0.02 | 0.00 | 0.04 | 0.01 | 0.01 | −0.02 | 0.06 | −0.03 |
| 14 | 1 | −0.02 | 0.04 | 0.04 | 0.05 | −0.01 | −0.09 | 0.17 | −0.04 | 0.08 |
| 15 | 1 | −0.03 | −0.04 | −0.01 | −0.08 | −0.03 | −0.05 | 0.07 | 0.03 | 0.04 |
| 16 | 1 | −0.01 | −0.02 | −0.05 | 0.00 | 0.01 | 0.03 | 0.00 | −0.01 | −0.04 |
| 17 | 1 | 0.00 | −0.01 | −0.01 | 0.00 | 0.12 | 0.08 | 0.10 | −0.24 | −0.18 |
| 18 | 1 | −0.01 | 0.00 | 0.00 | 0.08 | −0.04 | −0.10 | −0.36 | 0.09 | 0.20 |
| 19 | 1 | 0.01 | 0.01 | −0.02 | −0.07 | 0.01 | 0.13 | 0.10 | −0.03 | −0.19 |
| 20 | 1 | 0.00 | −0.01 | 0.02 | 0.01 | 0.16 | −0.19 | −0.02 | −0.24 | 0.29 |
| 21 | 1 | 0.01 | 0.00 | 0.00 | −0.09 | −0.05 | 0.14 | 0.14 | 0.08 | −0.22 |
| 22 | 15 | −0.05 | 0.31 | −0.04 | −0.05 | 0.01 | 0.02 | −0.03 | 0.01 | 0.07 |
| 23 | 8 | −0.01 | −0.03 | 0.02 | 0.02 | 0.02 | −0.02 | 0.02 | 0.01 | −0.01 |
| 24 | 8 | 0.15 | −0.19 | 0.20 | 0.02 | −0.02 | 0.00 | 0.01 | −0.01 | −0.04 |
| 25 | 8 | −0.05 | −0.36 | −0.14 | −0.01 | −0.03 | −0.02 | 0.00 | −0.04 | −0.02 |
| 26 | 1 | 0.23 | 0.14 | 0.17 | 0.09 | 0.12 | 0.08 | 0.01 | −0.01 | 0.02 |
| 27 | 8 | −0.01 | −0.02 | −0.03 | 0.05 | 0.00 | 0.00 | 0.05 | 0.00 | −0.04 |
| 28 | 1 | −0.01 | 0.08 | 0.30 | 0.04 | −0.05 | −0.55 | 0.04 | −0.03 | −0.31 |
| 29 | 1 | 0.33 | −0.35 | 0.24 | −0.08 | 0.11 | −0.02 | −0.26 | 0.30 | −0.09 |

| | | |
|---|---|---|
| 46 | 47 | 48 |
| ?A | ?A | ?A |

| | | | |
|---|---|---|---|
| Frequencies -- | 1076.4147 | 1132.5984 | 1147.2343 |
| Red. masses -- | 2.0322 | 2.7274 | 2.7664 |
| Frc consts -- | 1.3873 | 2.0614 | 2.1452 |
| IR Inten -- | 76.9204 | 65.1542 | 86.0512 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.01 | −0.02 | −0.01 | −0.02 | −0.02 | 0.00 | −0.01 | −0.05 | −0.03 |
| 2 | 6 | 0.03 | 0.00 | 0.03 | −0.06 | 0.05 | 0.01 | 0.00 | −0.03 | 0.05 |
| 3 | 6 | 0.00 | 0.01 | −0.04 | −0.05 | −0.11 | −0.13 | 0.10 | 0.12 | 0.09 |
| 4 | 6 | 0.01 | −0.05 | 0.00 | 0.02 | −0.14 | −0.03 | −0.04 | −0.07 | 0.12 |
| 5 | 6 | −0.04 | 0.02 | −0.03 | 0.12 | 0.14 | 0.02 | 0.12 | 0.15 | 0.02 |
| 6 | 8 | 0.00 | 0.00 | 0.01 | 0.06 | 0.05 | 0.10 | −0.03 | −0.04 | −0.08 |
| 7 | 8 | 0.00 | 0.01 | 0.01 | 0.00 | 0.04 | 0.06 | −0.01 | −0.02 | −0.07 |
| 8 | 6 | 0.02 | −0.01 | 0.04 | −0.12 | −0.04 | −0.07 | −0.11 | −0.05 | −0.06 |
| 9 | 16 | −0.01 | 0.01 | 0.00 | 0.01 | 0.00 | 0.00 | 0.00 | 0.01 | 0.00 |
| 10 | 6 | 0.02 | −0.02 | −0.01 | −0.01 | 0.00 | 0.00 | 0.00 | −0.01 | 0.00 |
| 11 | 1 | −0.26 | 0.31 | −0.33 | 0.18 | 0.13 | 0.26 | −0.21 | 0.12 | −0.21 |
| 12 | 1 | 0.00 | 0.02 | −0.03 | −0.07 | −0.08 | −0.10 | 0.06 | 0.05 | 0.03 |
| 13 | 1 | 0.00 | −0.04 | 0.02 | −0.18 | −0.27 | 0.08 | −0.09 | −0.25 | 0.06 |
| 14 | 1 | −0.13 | 0.05 | 0.01 | 0.33 | 0.58 | 0.13 | 0.22 | 0.33 | 0.07 |
| 15 | 1 | −0.02 | −0.02 | 0.00 | −0.09 | −0.05 | 0.00 | −0.03 | −0.05 | −0.07 |
| 16 | 1 | −0.01 | −0.01 | −0.03 | −0.06 | −0.07 | −0.17 | 0.07 | 0.12 | 0.28 |
| 17 | 1 | −0.06 | 0.09 | 0.07 | −0.03 | −0.08 | −0.10 | −0.21 | −0.05 | −0.05 |
| 18 | 1 | 0.17 | −0.04 | −0.08 | −0.06 | −0.04 | −0.06 | 0.12 | −0.07 | −0.09 |
| 19 | 1 | −0.04 | 0.01 | 0.07 | 0.01 | 0.01 | −0.03 | −0.01 | 0.00 | 0.01 |
| 20 | 1 | 0.01 | 0.09 | −0.10 | −0.01 | −0.01 | 0.01 | 0.00 | 0.02 | −0.03 |
| 21 | 1 | −0.05 | −0.03 | 0.08 | 0.01 | 0.00 | −0.01 | −0.01 | −0.01 | 0.04 |
| 22 | 15 | 0.03 | 0.03 | 0.12 | 0.04 | 0.01 | −0.01 | −0.05 | −0.02 | 0.01 |
| 23 | 8 | −0.05 | −0.03 | 0.00 | −0.06 | −0.02 | −0.01 | 0.09 | 0.03 | 0.01 |
| 24 | 8 | −0.01 | 0.01 | −0.08 | −0.01 | 0.01 | 0.00 | 0.01 | −0.01 | 0.00 |
| 25 | 8 | 0.01 | −0.06 | −0.03 | 0.01 | 0.00 | 0.01 | −0.04 | 0.01 | −0.02 |
| 26 | 1 | −0.07 | −0.17 | −0.14 | −0.07 | −0.13 | −0.18 | 0.20 | 0.37 | 0.36 |
| 27 | 8 | 0.04 | −0.01 | −0.08 | −0.02 | 0.00 | 0.01 | 0.02 | 0.00 | −0.02 |
| 28 | 1 | 0.05 | 0.00 | −0.04 | −0.01 | 0.01 | 0.12 | 0.01 | −0.01 | −0.11 |
| 29 | 1 | −0.45 | 0.52 | −0.17 | 0.03 | −0.04 | 0.01 | −0.05 | 0.05 | −0.02 |

| | | |
|---|---|---|
| 49 | 50 | 51 |
| ?A | ?A | ?A |

| | | | |
|---|---|---|---|
| Frequencies -- | 1154.2363 | 1175.0206 | 1196.3212 |
| Red. masses -- | 2.8839 | 1.9602 | 1.3297 |
| Frc consts -- | 2.2637 | 1.5945 | 1.1213 |
| IR Inten -- | 16.9743 | 71.7136 | 89.1500 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.02 | 0.00 | 0.01 | 0.00 | −0.03 | 0.00 | 0.01 | 0.02 | 0.00 |
| 2 | 6 | −0.02 | −0.02 | −0.01 | −0.04 | 0.04 | −0.02 | −0.01 | −0.03 | −0.02 |
| 3 | 6 | −0.04 | −0.07 | −0.09 | 0.12 | 0.04 | 0.08 | −0.03 | 0.02 | 0.06 |
| 4 | 6 | −0.02 | 0.16 | 0.24 | −0.04 | −0.01 | 0.07 | 0.01 | 0.01 | −0.08 |
| 5 | 6 | 0.04 | 0.05 | 0.02 | 0.02 | 0.02 | 0.00 | 0.04 | 0.02 | 0.05 |
| 6 | 8 | 0.02 | 0.02 | 0.06 | −0.05 | −0.02 | −0.07 | −0.03 | 0.00 | −0.03 |
| 7 | 8 | 0.01 | −0.08 | −0.18 | −0.02 | −0.02 | −0.05 | 0.01 | 0.01 | 0.05 |
| 8 | 6 | −0.02 | −0.03 | 0.00 | −0.02 | 0.00 | −0.01 | −0.03 | 0.00 | −0.01 |
| 9 | 16 | 0.00 | 0.01 | 0.00 | 0.00 | 0.00 | 0.00 | 0.00 | 0.01 | −0.01 |
| 10 | 6 | 0.01 | −0.01 | −0.01 | 0.00 | 0.01 | 0.00 | 0.01 | −0.02 | 0.00 |
| 11 | 1 | 0.03 | 0.11 | 0.01 | 0.18 | −0.21 | 0.26 | 0.03 | −0.04 | 0.02 |
| 12 | 1 | 0.09 | −0.20 | −0.18 | −0.03 | 0.15 | 0.14 | 0.36 | −0.08 | 0.02 |
| 13 | 1 | −0.32 | 0.13 | 0.49 | 0.22 | 0.08 | −0.11 | −0.16 | −0.32 | −0.12 |
| 14 | 1 | 0.06 | −0.39 | −0.16 | 0.12 | 0.08 | −0.01 | −0.06 | −0.24 | −0.02 |
| 15 | 1 | −0.01 | 0.01 | 0.03 | −0.07 | 0.01 | −0.08 | 0.20 | 0.11 | 0.12 |
| 16 | 1 | 0.01 | −0.07 | −0.22 | 0.08 | 0.15 | 0.39 | −0.01 | −0.01 | −0.02 |
| 17 | 1 | −0.28 | 0.02 | 0.04 | 0.08 | −0.02 | −0.03 | −0.42 | −0.02 | 0.03 |
| 18 | 1 | 0.15 | −0.05 | −0.04 | −0.09 | 0.00 | 0.00 | 0.43 | −0.04 | −0.03 |
| 19 | 1 | −0.02 | 0.02 | 0.03 | 0.00 | −0.02 | −0.01 | −0.02 | 0.02 | 0.03 |
| 20 | 1 | 0.01 | 0.05 | −0.06 | −0.01 | −0.02 | 0.02 | 0.01 | 0.05 | −0.05 |
| 21 | 1 | −0.03 | −0.02 | 0.05 | 0.01 | 0.00 | 0.00 | −0.03 | −0.02 | 0.03 |
| 22 | 15 | 0.01 | 0.01 | −0.01 | 0.05 | 0.04 | 0.00 | −0.03 | 0.01 | −0.01 |
| 23 | 8 | −0.02 | −0.01 | 0.00 | −0.11 | −0.04 | −0.02 | 0.02 | 0.00 | 0.00 |
| 24 | 8 | 0.00 | 0.00 | 0.00 | −0.01 | 0.01 | 0.00 | 0.01 | 0.00 | 0.01 |
| 25 | 8 | 0.01 | 0.00 | 0.01 | 0.04 | −0.03 | 0.01 | 0.03 | 0.00 | 0.01 |
| 26 | 1 | −0.07 | −0.13 | −0.13 | −0.24 | −0.47 | −0.40 | −0.17 | −0.31 | −0.22 |
| 27 | 8 | 0.00 | 0.00 | 0.01 | −0.01 | 0.00 | 0.01 | 0.01 | 0.00 | 0.00 |
| 28 | 1 | 0.00 | 0.00 | 0.00 | −0.01 | 0.01 | 0.12 | 0.01 | −0.01 | −0.08 |
| 29 | 1 | 0.02 | −0.02 | 0.01 | 0.01 | 0.00 | 0.00 | 0.02 | −0.03 | 0.01 |

| | | |
|---|---|---|
| 52 | 53 | 54 |
| ?A | ?A | ?A |

| | | | |
|---|---|---|---|
| Frequencies -- | 1225.3585 | 1252.1639 | 1273.4879 |
| Red. masses -- | 1.3952 | 1.6203 | 1.1989 |
| Frc consts -- | 1.2343 | 1.4968 | 1.1456 |
| IR Inten -- | 188.9300 | 321.1660 | 6.2939 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.01 | −0.01 | 0.00 | 0.00 | −0.01 | 0.00 | 0.02 | 0.01 | 0.03 |
| 2 | 6 | 0.00 | 0.01 | −0.01 | −0.01 | 0.03 | −0.05 | −0.01 | −0.01 | 0.01 |
| 3 | 6 | −0.02 | −0.04 | −0.02 | 0.04 | 0.01 | 0.09 | −0.01 | 0.00 | 0.01 |
| 4 | 6 | 0.02 | 0.06 | −0.04 | −0.03 | 0.00 | −0.01 | 0.00 | 0.00 | −0.02 |
| 5 | 6 | 0.05 | −0.01 | 0.06 | 0.00 | −0.01 | 0.01 | −0.04 | 0.03 | −0.02 |
| 6 | 8 | 0.03 | 0.01 | 0.02 | −0.03 | −0.01 | −0.05 | 0.00 | 0.00 | 0.00 |
| 7 | 8 | −0.02 | −0.02 | 0.01 | 0.03 | 0.01 | 0.01 | 0.00 | 0.00 | 0.01 |
| 8 | 6 | −0.02 | 0.00 | −0.02 | 0.01 | 0.00 | 0.00 | −0.10 | 0.01 | 0.02 |
| 9 | 16 | 0.00 | 0.01 | 0.00 | 0.00 | 0.00 | 0.00 | 0.00 | 0.00 | 0.01 |
| 10 | 6 | 0.01 | −0.01 | 0.00 | 0.00 | 0.00 | 0.00 | −0.01 | 0.00 | 0.01 |
| 11 | 1 | 0.11 | 0.03 | 0.10 | 0.17 | −0.15 | 0.17 | −0.08 | −0.03 | −0.06 |
| 12 | 1 | −0.29 | 0.03 | 0.01 | 0.29 | 0.05 | 0.14 | 0.04 | 0.01 | 0.02 |
| 13 | 1 | 0.20 | −0.05 | −0.27 | −0.36 | −0.04 | 0.26 | −0.01 | −0.04 | −0.03 |
| 14 | 1 | −0.05 | −0.28 | −0.02 | −0.10 | 0.05 | 0.06 | 0.42 | −0.31 | −0.31 |
| 15 | 1 | −0.18 | −0.08 | −0.12 | 0.12 | 0.05 | 0.05 | 0.02 | 0.01 | 0.01 |
| 16 | 1 | 0.07 | 0.14 | 0.38 | −0.09 | −0.18 | −0.49 | 0.00 | −0.01 | −0.02 |
| 17 | 1 | −0.36 | −0.04 | 0.02 | −0.07 | 0.01 | 0.01 | 0.61 | −0.12 | −0.11 |
| 18 | 1 | 0.30 | −0.02 | −0.01 | 0.01 | 0.00 | 0.01 | 0.40 | −0.04 | −0.13 |
| 19 | 1 | −0.02 | 0.00 | 0.03 | 0.00 | 0.01 | 0.00 | 0.03 | 0.03 | −0.05 |
| 20 | 1 | 0.01 | 0.02 | −0.02 | 0.00 | 0.02 | −0.02 | −0.02 | 0.02 | −0.02 |
| 21 | 1 | −0.02 | −0.01 | 0.03 | −0.01 | 0.00 | 0.00 | 0.02 | 0.02 | −0.06 |
| 22 | 15 | 0.06 | −0.01 | 0.01 | 0.08 | 0.01 | 0.02 | 0.01 | 0.00 | 0.00 |
| 23 | 8 | −0.06 | −0.01 | −0.01 | −0.10 | −0.02 | −0.02 | −0.02 | 0.00 | 0.00 |
| 24 | 8 | −0.01 | 0.01 | −0.01 | −0.02 | 0.01 | −0.02 | 0.00 | 0.00 | 0.00 |
| 25 | 8 | −0.03 | 0.00 | −0.02 | −0.04 | −0.02 | −0.02 | −0.01 | 0.00 | 0.00 |
| 26 | 1 | 0.17 | 0.33 | 0.23 | 0.18 | 0.33 | 0.28 | 0.03 | 0.06 | 0.05 |
| 27 | 8 | −0.01 | 0.00 | 0.00 | −0.02 | 0.00 | 0.00 | 0.00 | 0.00 | 0.00 |
| 28 | 1 | −0.01 | 0.01 | 0.13 | −0.01 | 0.02 | 0.16 | 0.00 | 0.00 | 0.02 |
| 29 | 1 | −0.02 | 0.03 | −0.02 | −0.03 | 0.04 | −0.02 | 0.00 | 0.01 | 0.00 |

| | | |
|---|---|---|
| 55 | 56 | 57 |
| ?A | ?A | ?A |

| | | | |
|---|---|---|---|
| Frequencies -- | 1306.9840 | 1313.2043 | 1334.9905 |
| Red. masses -- | 1.3739 | 1.3425 | 1.3648 |
| Frc consts -- | 1.3827 | 1.3641 | 1.4331 |
| IR Inten -- | 36.8923 | 75.6051 | 25.9452 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.02 | −0.01 | −0.02 | −0.03 | 0.00 | 0.00 | −0.09 | −0.03 | 0.03 |
| 2 | 6 | 0.02 | 0.04 | 0.02 | 0.05 | 0.02 | −0.05 | 0.07 | −0.01 | −0.04 |
| 3 | 6 | 0.07 | −0.01 | 0.01 | −0.08 | 0.01 | −0.02 | 0.05 | −0.02 | 0.02 |
| 4 | 6 | −0.05 | 0.00 | −0.03 | 0.02 | −0.01 | 0.06 | 0.02 | 0.04 | −0.03 |
| 5 | 6 | 0.04 | −0.10 | 0.03 | 0.02 | −0.07 | −0.03 | 0.03 | 0.03 | −0.03 |
| 6 | 8 | 0.01 | 0.00 | 0.00 | 0.00 | 0.00 | 0.01 | 0.00 | 0.00 | −0.02 |
| 7 | 8 | 0.01 | 0.01 | 0.00 | −0.02 | −0.01 | −0.03 | 0.00 | 0.00 | 0.00 |
| 8 | 6 | −0.06 | 0.04 | −0.01 | −0.05 | 0.02 | 0.02 | −0.01 | −0.02 | 0.02 |
| 9 | 16 | 0.00 | 0.00 | 0.00 | 0.00 | 0.00 | 0.00 | 0.00 | 0.00 | 0.00 |
| 10 | 6 | 0.00 | −0.01 | 0.01 | 0.00 | 0.00 | 0.00 | 0.00 | 0.00 | 0.00 |
| 11 | 1 | −0.03 | −0.06 | −0.01 | 0.22 | 0.10 | 0.09 | 0.40 | 0.58 | 0.14 |
| 12 | 1 | −0.39 | 0.11 | 0.06 | 0.49 | −0.16 | −0.10 | −0.19 | 0.22 | 0.18 |
| 13 | 1 | −0.04 | 0.43 | 0.22 | 0.20 | 0.07 | −0.05 | −0.29 | −0.25 | 0.08 |
| 14 | 1 | −0.05 | 0.21 | 0.20 | −0.33 | 0.34 | 0.25 | −0.25 | −0.18 | −0.03 |
| 15 | 1 | −0.15 | −0.07 | −0.10 | 0.13 | 0.07 | 0.09 | −0.09 | −0.03 | −0.07 |
| 16 | 1 | −0.05 | −0.11 | −0.26 | 0.05 | 0.10 | 0.25 | 0.00 | 0.00 | 0.02 |
| 17 | 1 | −0.05 | −0.13 | −0.05 | 0.27 | −0.08 | −0.04 | 0.24 | −0.01 | −0.01 |
| 18 | 1 | 0.55 | −0.01 | −0.05 | 0.30 | −0.03 | −0.09 | −0.09 | −0.02 | −0.08 |
| 19 | 1 | 0.01 | 0.04 | −0.02 | 0.01 | 0.00 | −0.01 | 0.00 | 0.00 | −0.01 |
| 20 | 1 | 0.00 | 0.05 | −0.04 | 0.00 | 0.01 | 0.00 | 0.00 | 0.00 | 0.00 |
| 21 | 1 | 0.00 | 0.01 | −0.04 | 0.01 | 0.00 | −0.01 | 0.00 | 0.00 | 0.00 |
| 22 | 15 | −0.02 | 0.00 | 0.00 | 0.01 | 0.00 | 0.00 | −0.02 | 0.00 | −0.01 |
| 23 | 8 | 0.03 | 0.00 | 0.01 | 0.00 | 0.00 | 0.00 | 0.03 | 0.00 | 0.01 |
| 24 | 8 | 0.00 | 0.00 | 0.00 | 0.00 | 0.00 | 0.00 | 0.00 | 0.00 | 0.00 |
| 25 | 8 | 0.01 | 0.00 | 0.01 | −0.01 | 0.00 | 0.00 | 0.01 | 0.00 | 0.01 |
| 26 | 1 | −0.06 | −0.11 | −0.10 | 0.03 | 0.07 | 0.06 | −0.03 | −0.06 | −0.06 |
| 27 | 8 | 0.01 | 0.00 | 0.00 | 0.00 | 0.00 | 0.00 | 0.00 | 0.00 | 0.00 |
| 28 | 1 | 0.00 | −0.01 | −0.04 | 0.00 | 0.00 | 0.01 | 0.00 | 0.00 | −0.02 |
| 29 | 1 | 0.00 | −0.01 | 0.00 | 0.00 | 0.00 | 0.00 | 0.00 | 0.00 | 0.01 |

| | | |
|---|---|---|
| 58 | 59 | 60 |
| ?A | ?A | ?A |

| | | | |
|---|---|---|---|
| Frequencies -- | 1357.3953 | 1390.6003 | 1395.8872 |
| Red. masses -- | 1.2283 | 1.1551 | 1.3477 |
| Frc consts -- | 1.3334 | 1.3161 | 1.5471 |
| IR Inten -- | 14.3262 | 2.5617 | 13.2756 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.04 | −0.01 | 0.03 | 0.00 | 0.00 | 0.00 | 0.04 | 0.02 | −0.03 |
| 2 | 6 | 0.03 | −0.02 | −0.01 | 0.00 | −0.01 | 0.00 | −0.03 | 0.01 | 0.06 |
| 3 | 6 | −0.02 | −0.02 | −0.05 | 0.00 | 0.00 | 0.00 | −0.02 | −0.11 | −0.05 |
| 4 | 6 | −0.07 | −0.05 | 0.03 | 0.01 | 0.01 | 0.01 | 0.03 | 0.05 | 0.02 |
| 5 | 6 | −0.01 | 0.00 | 0.03 | −0.01 | −0.01 | 0.00 | 0.02 | −0.01 | −0.01 |
| 6 | 8 | −0.01 | 0.01 | −0.01 | 0.00 | 0.00 | 0.00 | 0.00 | 0.02 | −0.02 |
| 7 | 8 | 0.02 | 0.01 | 0.01 | 0.00 | 0.00 | 0.00 | −0.01 | −0.01 | −0.01 |
| 8 | 6 | 0.02 | 0.01 | −0.03 | 0.02 | 0.00 | 0.00 | −0.01 | 0.00 | 0.01 |
| 9 | 16 | 0.00 | 0.00 | 0.00 | 0.00 | 0.00 | 0.01 | 0.00 | 0.00 | 0.00 |
| 10 | 6 | 0.00 | 0.00 | 0.00 | −0.02 | −0.06 | 0.09 | 0.00 | 0.00 | 0.00 |
| 11 | 1 | 0.18 | 0.34 | 0.05 | 0.02 | 0.03 | 0.01 | −0.24 | −0.23 | −0.07 |
| 12 | 1 | 0.32 | 0.33 | 0.25 | 0.00 | 0.00 | 0.00 | 0.21 | 0.63 | 0.55 |
| 13 | 1 | 0.37 | 0.36 | −0.13 | 0.00 | −0.06 | −0.03 | −0.09 | −0.17 | 0.00 |
| 14 | 1 | 0.38 | −0.09 | −0.13 | 0.03 | 0.05 | 0.01 | −0.18 | 0.09 | 0.09 |
| 15 | 1 | 0.09 | 0.05 | 0.05 | 0.00 | 0.00 | 0.00 | −0.03 | 0.02 | −0.05 |
| 16 | 1 | −0.03 | −0.06 | −0.16 | 0.01 | 0.01 | 0.04 | 0.02 | 0.03 | 0.10 |
| 17 | 1 | −0.24 | 0.03 | 0.01 | −0.09 | −0.01 | 0.01 | 0.07 | −0.04 | −0.01 |
| 18 | 1 | 0.01 | 0.03 | 0.09 | −0.07 | 0.00 | 0.00 | 0.02 | −0.01 | −0.05 |
| 19 | 1 | −0.01 | 0.00 | 0.01 | 0.26 | 0.28 | −0.37 | 0.00 | 0.00 | 0.00 |
| 20 | 1 | 0.00 | −0.01 | 0.00 | −0.08 | 0.45 | −0.40 | 0.00 | 0.00 | 0.00 |
| 21 | 1 | −0.01 | 0.00 | 0.01 | 0.11 | 0.10 | −0.54 | 0.01 | 0.00 | 0.00 |
| 22 | 15 | −0.01 | 0.00 | 0.00 | 0.00 | 0.00 | 0.00 | −0.01 | 0.00 | 0.00 |
| 23 | 8 | 0.01 | 0.00 | 0.00 | 0.00 | 0.00 | 0.00 | 0.01 | 0.00 | 0.00 |
| 24 | 8 | 0.00 | 0.00 | 0.00 | 0.00 | 0.00 | 0.00 | 0.00 | 0.00 | 0.00 |
| 25 | 8 | 0.00 | 0.00 | 0.00 | 0.00 | 0.00 | 0.00 | 0.00 | 0.00 | 0.00 |
| 26 | 1 | 0.01 | 0.04 | 0.02 | 0.00 | 0.00 | 0.00 | 0.01 | 0.03 | 0.00 |
| 27 | 8 | 0.00 | 0.00 | 0.00 | 0.00 | 0.00 | 0.00 | 0.00 | 0.00 | 0.00 |
| 28 | 1 | 0.00 | 0.00 | −0.01 | 0.00 | 0.00 | 0.00 | 0.00 | 0.00 | −0.01 |
| 29 | 1 | 0.00 | 0.00 | 0.00 | 0.00 | 0.00 | 0.00 | 0.00 | 0.00 | 0.00 |

| | | |
|---|---|---|
| 61 | 62 | 63 |
| ?A | ?A | ?A |

| | | | |
|---|---|---|---|
| Frequencies -- | 1406.4838 | 1440.8556 | 1468.2121 |
| Red. masses -- | 1.5367 | 1.4508 | 1.2497 |
| Frc consts -- | 1.7910 | 1.7746 | 1.5872 |
| IR Inten -- | 9.0736 | 2.8133 | 218.5989 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | 0.03 | 0.03 | 0.01 | 0.01 | −0.01 | 0.00 | −0.01 | 0.00 |
| 2 | 6 | 0.00 | −0.04 | −0.01 | −0.01 | −0.01 | 0.00 | −0.02 | 0.02 | 0.01 |
| 3 | 6 | −0.01 | 0.00 | 0.02 | −0.05 | 0.01 | 0.03 | 0.08 | −0.04 | −0.07 |
| 4 | 6 | 0.12 | 0.01 | −0.03 | 0.06 | −0.10 | −0.09 | 0.01 | −0.02 | −0.03 |
| 5 | 6 | −0.11 | −0.10 | −0.01 | 0.05 | 0.07 | 0.03 | 0.02 | 0.01 | 0.01 |
| 6 | 8 | 0.00 | 0.00 | −0.01 | 0.02 | 0.00 | −0.01 | −0.06 | 0.00 | 0.01 |
| 7 | 8 | −0.04 | 0.00 | −0.02 | −0.04 | 0.01 | −0.01 | −0.02 | 0.00 | −0.01 |
| 8 | 6 | 0.03 | 0.03 | −0.03 | −0.01 | −0.03 | 0.00 | 0.00 | 0.00 | 0.00 |
| 9 | 16 | 0.00 | 0.00 | 0.00 | 0.00 | 0.00 | 0.00 | 0.00 | 0.00 | 0.00 |
| 10 | 6 | 0.00 | 0.01 | −0.01 | 0.00 | −0.01 | 0.01 | 0.00 | 0.00 | 0.00 |
| 11 | 1 | 0.09 | 0.17 | 0.02 | −0.02 | −0.04 | 0.00 | −0.02 | 0.02 | 0.02 |
| 12 | 1 | 0.01 | 0.05 | 0.06 | 0.20 | 0.02 | 0.06 | −0.27 | 0.04 | −0.05 |
| 13 | 1 | −0.35 | −0.07 | 0.35 | −0.11 | 0.58 | 0.50 | −0.04 | 0.15 | 0.12 |
| 14 | 1 | 0.55 | 0.36 | −0.02 | −0.22 | −0.32 | −0.04 | −0.07 | −0.06 | 0.00 |
| 15 | 1 | −0.01 | 0.00 | −0.01 | −0.15 | −0.05 | −0.12 | 0.66 | 0.31 | 0.51 |
| 16 | 1 | 0.05 | 0.13 | 0.34 | 0.03 | 0.11 | 0.27 | 0.04 | 0.09 | 0.19 |
| 17 | 1 | −0.21 | 0.03 | 0.01 | 0.06 | 0.10 | 0.02 | 0.01 | −0.02 | 0.00 |
| 18 | 1 | 0.13 | 0.04 | 0.12 | −0.10 | −0.02 | 0.06 | −0.01 | −0.01 | −0.03 |
| 19 | 1 | −0.02 | −0.03 | 0.03 | 0.02 | 0.03 | −0.03 | 0.01 | 0.00 | −0.01 |
| 20 | 1 | 0.00 | −0.05 | 0.03 | 0.00 | 0.04 | −0.03 | 0.00 | 0.01 | −0.01 |
| 21 | 1 | −0.02 | −0.01 | 0.06 | 0.00 | 0.01 | −0.05 | 0.01 | 0.00 | −0.01 |
| 22 | 15 | 0.00 | 0.00 | 0.00 | 0.00 | 0.00 | 0.00 | 0.00 | 0.00 | 0.00 |
| 23 | 8 | −0.01 | 0.00 | 0.00 | 0.00 | 0.00 | 0.00 | 0.00 | 0.00 | 0.00 |
| 24 | 8 | 0.00 | 0.00 | 0.00 | 0.00 | 0.00 | 0.00 | 0.00 | 0.00 | 0.00 |
| 25 | 8 | 0.00 | 0.00 | 0.00 | 0.00 | 0.00 | 0.00 | 0.00 | 0.00 | 0.00 |
| 26 | 1 | 0.00 | 0.01 | 0.01 | 0.00 | −0.01 | −0.02 | 0.02 | 0.06 | 0.10 |
| 27 | 8 | 0.00 | 0.00 | 0.00 | 0.00 | 0.00 | 0.00 | 0.00 | 0.00 | 0.00 |
| 28 | 1 | 0.00 | 0.00 | 0.00 | 0.00 | 0.00 | 0.00 | 0.00 | 0.00 | 0.01 |
| 29 | 1 | 0.00 | 0.00 | 0.00 | 0.00 | 0.00 | 0.00 | 0.00 | 0.00 | 0.00 |

| | | |
|---|---|---|
| 64 | 65 | 66 |
| ?A | ?A | ?A |

| | | | |
|---|---|---|---|
| Frequencies -- | 1489.9288 | 1494.7560 | 1501.2203 |
| Red. masses -- | 1.1032 | 1.0644 | 1.0671 |
| Frc consts -- | 1.4428 | 1.4011 | 1.4169 |
| IR Inten -- | 6.8061 | 23.0963 | 21.0080 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.01 | −0.01 | 0.00 | 0.00 | 0.00 | 0.00 | 0.01 | 0.01 | 0.00 |
| 2 | 6 | 0.02 | 0.03 | 0.00 | 0.00 | 0.01 | 0.00 | −0.02 | −0.02 | 0.00 |
| 3 | 6 | 0.00 | −0.01 | 0.00 | 0.00 | 0.00 | 0.00 | 0.00 | 0.00 | 0.00 |
| 4 | 6 | 0.00 | 0.02 | 0.01 | 0.00 | 0.01 | 0.00 | 0.00 | 0.00 | 0.00 |
| 5 | 6 | 0.01 | −0.01 | −0.01 | 0.00 | 0.00 | −0.01 | 0.00 | 0.00 | 0.00 |
| 6 | 8 | 0.00 | 0.00 | 0.00 | 0.00 | 0.00 | 0.00 | 0.00 | 0.00 | 0.00 |
| 7 | 8 | 0.00 | 0.00 | 0.00 | 0.00 | 0.00 | 0.00 | 0.00 | 0.00 | 0.00 |
| 8 | 6 | −0.03 | −0.04 | −0.04 | −0.02 | −0.03 | −0.03 | 0.01 | 0.01 | 0.01 |
| 9 | 16 | 0.00 | 0.00 | 0.00 | 0.00 | 0.00 | 0.00 | 0.00 | 0.00 | 0.00 |
| 10 | 6 | 0.03 | 0.01 | 0.02 | −0.03 | −0.03 | −0.03 | 0.04 | −0.04 | −0.01 |
| 11 | 1 | −0.02 | −0.06 | −0.01 | 0.00 | −0.01 | 0.00 | 0.01 | 0.05 | 0.01 |
| 12 | 1 | −0.02 | 0.00 | 0.00 | −0.02 | 0.00 | 0.00 | −0.01 | 0.01 | 0.00 |
| 13 | 1 | −0.01 | −0.06 | −0.04 | 0.00 | −0.04 | −0.02 | 0.01 | 0.01 | 0.00 |
| 14 | 1 | −0.02 | 0.01 | 0.01 | 0.00 | 0.01 | 0.00 | −0.01 | 0.02 | 0.01 |
| 15 | 1 | 0.02 | 0.01 | 0.02 | 0.01 | 0.01 | 0.01 | 0.00 | 0.00 | 0.00 |
| 16 | 1 | 0.00 | 0.00 | −0.01 | 0.00 | 0.00 | 0.00 | 0.00 | 0.00 | 0.01 |
| 17 | 1 | 0.14 | 0.56 | 0.07 | 0.09 | 0.33 | 0.04 | −0.01 | −0.14 | −0.02 |
| 18 | 1 | 0.15 | 0.03 | 0.56 | 0.08 | 0.02 | 0.33 | −0.05 | 0.00 | −0.13 |
| 19 | 1 | 0.14 | 0.10 | −0.17 | −0.27 | 0.12 | 0.32 | −0.14 | 0.63 | 0.16 |
| 20 | 1 | −0.21 | −0.26 | −0.15 | 0.39 | 0.37 | 0.34 | 0.10 | −0.21 | 0.23 |
| 21 | 1 | −0.32 | 0.06 | 0.07 | 0.30 | −0.02 | −0.27 | −0.56 | 0.16 | −0.26 |
| 22 | 15 | 0.00 | 0.00 | 0.00 | 0.00 | 0.00 | 0.00 | 0.00 | 0.00 | 0.00 |
| 23 | 8 | 0.00 | 0.00 | 0.00 | 0.00 | 0.00 | 0.00 | 0.00 | 0.00 | 0.00 |
| 24 | 8 | 0.00 | 0.00 | 0.00 | 0.00 | 0.00 | 0.00 | 0.00 | 0.00 | 0.00 |
| 25 | 8 | 0.00 | 0.00 | 0.00 | 0.00 | 0.00 | 0.00 | 0.00 | 0.00 | 0.00 |
| 26 | 1 | 0.00 | 0.00 | 0.00 | 0.00 | 0.00 | 0.00 | 0.00 | 0.00 | 0.00 |
| 27 | 8 | 0.00 | 0.00 | 0.00 | 0.00 | 0.00 | 0.00 | 0.00 | 0.00 | 0.00 |
| 28 | 1 | 0.00 | 0.00 | 0.00 | 0.00 | 0.00 | 0.00 | 0.00 | 0.00 | 0.00 |
| 29 | 1 | 0.00 | 0.00 | 0.00 | 0.00 | 0.00 | 0.00 | 0.00 | 0.00 | 0.00 |

| | | |
|---|---|---|
| 67 | 68 | 69 |
| ?A | ?A | ?A |

| | | | |
|---|---|---|---|
| Frequencies -- | 1514.2329 | 3046.8507 | 3079.2307 |
| Red. masses -- | 3.7224 | 1.0848 | 1.0300 |
| Frc consts -- | 5.0288 | 5.9333 | 5.7540 |
| IR Inten -- | 138.1324 | 5.5970 | 10.5124 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | −0.16 | −0.16 | 0.00 | 0.00 | 0.00 | 0.00 | 0.00 | 0.00 | 0.00 |
| 2 | 6 | 0.24 | 0.32 | 0.01 | 0.00 | 0.00 | 0.00 | 0.00 | 0.00 | 0.00 |
| 3 | 6 | −0.05 | −0.05 | 0.01 | 0.01 | 0.05 | −0.06 | 0.00 | 0.00 | 0.00 |
| 4 | 6 | 0.05 | 0.02 | −0.01 | 0.01 | −0.01 | 0.01 | 0.00 | 0.00 | 0.00 |
| 5 | 6 | −0.06 | 0.04 | 0.02 | 0.00 | 0.00 | 0.00 | 0.00 | 0.00 | 0.00 |
| 6 | 8 | 0.00 | 0.00 | 0.00 | 0.00 | 0.00 | 0.00 | 0.00 | 0.00 | 0.00 |
| 7 | 8 | −0.02 | −0.01 | −0.01 | 0.00 | 0.00 | 0.00 | 0.00 | 0.00 | 0.00 |
| 8 | 6 | 0.04 | 0.00 | 0.00 | 0.00 | 0.00 | 0.00 | 0.00 | 0.00 | 0.00 |
| 9 | 16 | 0.00 | 0.00 | 0.00 | 0.00 | 0.00 | 0.00 | 0.00 | 0.00 | 0.00 |
| 10 | 6 | 0.01 | −0.01 | 0.00 | 0.00 | 0.00 | 0.00 | 0.00 | 0.03 | −0.04 |
| 11 | 1 | −0.15 | −0.59 | −0.18 | 0.02 | 0.00 | −0.01 | 0.00 | 0.00 | 0.00 |
| 12 | 1 | 0.18 | −0.11 | −0.02 | −0.07 | −0.60 | 0.78 | 0.00 | 0.00 | 0.00 |
| 13 | 1 | −0.12 | −0.06 | 0.10 | −0.08 | 0.06 | −0.10 | −0.01 | 0.00 | −0.01 |
| 14 | 1 | 0.31 | −0.12 | −0.15 | 0.00 | 0.00 | 0.01 | 0.00 | 0.00 | −0.01 |
| 15 | 1 | 0.01 | 0.00 | 0.01 | 0.01 | 0.00 | −0.01 | 0.00 | 0.00 | 0.00 |
| 16 | 1 | 0.02 | 0.05 | 0.12 | 0.00 | 0.00 | 0.00 | 0.00 | 0.00 | 0.00 |
| 17 | 1 | −0.14 | −0.18 | −0.02 | 0.00 | 0.00 | 0.00 | −0.01 | 0.01 | −0.05 |
| 18 | 1 | −0.13 | −0.01 | −0.17 | 0.00 | 0.00 | 0.00 | 0.00 | −0.04 | 0.00 |
| 19 | 1 | −0.06 | 0.13 | 0.07 | 0.00 | 0.00 | 0.00 | 0.51 | 0.05 | 0.35 |
| 20 | 1 | 0.06 | 0.01 | 0.07 | 0.00 | 0.00 | 0.00 | −0.40 | 0.20 | 0.25 |
| 21 | 1 | −0.07 | 0.03 | −0.08 | 0.00 | 0.00 | 0.00 | −0.12 | −0.55 | −0.16 |
| 22 | 15 | −0.02 | 0.00 | 0.00 | 0.00 | 0.00 | 0.00 | 0.00 | 0.00 | 0.00 |
| 23 | 8 | 0.03 | 0.00 | 0.01 | 0.00 | 0.00 | 0.00 | 0.00 | 0.00 | 0.00 |
| 24 | 8 | 0.00 | 0.00 | 0.00 | 0.00 | 0.00 | 0.00 | 0.00 | 0.00 | 0.00 |
| 25 | 8 | 0.00 | 0.00 | 0.00 | 0.00 | 0.00 | 0.00 | 0.00 | 0.00 | 0.00 |
| 26 | 1 | −0.02 | −0.03 | −0.02 | −0.01 | 0.01 | 0.00 | 0.00 | 0.00 | 0.00 |
| 27 | 8 | 0.00 | 0.00 | 0.00 | 0.00 | 0.00 | 0.00 | 0.00 | 0.00 | 0.00 |
| 28 | 1 | 0.00 | 0.00 | −0.01 | 0.00 | 0.00 | 0.00 | 0.00 | 0.00 | 0.00 |
| 29 | 1 | 0.00 | 0.00 | 0.00 | 0.00 | 0.00 | 0.00 | 0.00 | 0.00 | 0.00 |

| | | |
|---|---|---|
| 70 | 71 | 72 |
| ?A | ?A | ?A |

| | | | |
|---|---|---|---|
| Frequencies -- | 3088.7897 | 3093.7566 | 3133.6971 |
| Red. masses -- | 1.0862 | 1.0612 | 1.0885 |
| Frc consts -- | 6.1058 | 5.9845 | 6.2979 |
| IR Inten -- | 9.2775 | 3.3840 | 1.7273 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | 0.00 | 0.00 | 0.00 | 0.00 | 0.00 | 0.00 | 0.00 | 0.00 |
| 2 | 6 | 0.00 | 0.00 | 0.00 | 0.00 | 0.00 | 0.00 | 0.00 | 0.00 | 0.00 |
| 3 | 6 | 0.00 | 0.01 | −0.01 | 0.00 | 0.00 | 0.00 | 0.00 | 0.00 | 0.00 |
| 4 | 6 | −0.05 | 0.03 | −0.05 | −0.01 | 0.01 | −0.01 | −0.01 | 0.00 | −0.01 |
| 5 | 6 | 0.00 | 0.00 | 0.01 | 0.00 | 0.00 | 0.01 | −0.02 | 0.03 | −0.07 |
| 6 | 8 | 0.00 | 0.00 | 0.00 | 0.00 | 0.00 | 0.00 | 0.00 | 0.00 | 0.00 |
| 7 | 8 | 0.00 | 0.00 | 0.00 | 0.00 | 0.00 | 0.00 | 0.00 | 0.00 | 0.00 |
| 8 | 6 | 0.00 | 0.01 | 0.01 | −0.01 | −0.03 | −0.06 | 0.00 | −0.03 | 0.01 |
| 9 | 16 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 1 | 0.01 | 0.00 | −0.01 | 0.00 | 0.00 | 0.00 | −0.01 | 0.00 | 0.01 |
| 12 | 1 | −0.01 | −0.08 | 0.11 | 0.00 | −0.01 | 0.02 | 0.00 | 0.00 | 0.01 |
| 13 | 1 | 0.60 | −0.39 | 0.65 | 0.09 | −0.06 | 0.10 | 0.06 | −0.04 | 0.07 |
| 14 | 1 | −0.03 | 0.03 | −0.08 | −0.02 | 0.03 | −0.08 | 0.25 | −0.35 | 0.84 |
| 15 | 1 | 0.00 | 0.00 | 0.00 | 0.00 | 0.00 | 0.00 | 0.00 | 0.00 | 0.00 |
| 16 | 1 | 0.00 | −0.01 | 0.00 | 0.00 | 0.00 | 0.00 | 0.00 | 0.00 | 0.00 |
| 17 | 1 | −0.01 | 0.03 | −0.12 | 0.10 | −0.18 | 0.78 | −0.01 | 0.02 | −0.10 |
| 18 | 1 | 0.00 | −0.09 | 0.01 | 0.04 | 0.55 | −0.08 | 0.02 | 0.28 | −0.04 |
| 19 | 1 | 0.00 | 0.00 | 0.00 | 0.04 | 0.00 | 0.03 | 0.00 | 0.00 | 0.00 |
| 20 | 1 | 0.00 | 0.00 | 0.00 | −0.02 | 0.01 | 0.01 | −0.02 | 0.01 | 0.01 |
| 21 | 1 | 0.00 | 0.00 | 0.00 | 0.00 | −0.02 | −0.01 | 0.00 | −0.02 | −0.01 |
| 22 | 15 | 0.00 | 0.00 | 0.00 | 0.00 | 0.00 | 0.00 | 0.00 | 0.00 | 0.00 |
| 23 | 8 | 0.00 | 0.00 | 0.00 | 0.00 | 0.00 | 0.00 | 0.00 | 0.00 | 0.00 |
| 24 | 8 | 0.00 | 0.00 | 0.00 | 0.00 | 0.00 | 0.00 | 0.00 | 0.00 | 0.00 |
| 25 | 8 | 0.00 | 0.00 | 0.00 | 0.00 | 0.00 | 0.00 | 0.00 | 0.00 | 0.00 |
| 26 | 1 | 0.00 | 0.00 | 0.00 | 0.00 | 0.00 | 0.00 | 0.00 | 0.00 | 0.00 |
| 27 | 8 | 0.00 | 0.00 | 0.00 | 0.00 | 0.00 | 0.00 | 0.00 | 0.00 | 0.00 |
| 28 | 1 | 0.00 | 0.00 | 0.00 | 0.00 | 0.00 | 0.00 | 0.00 | 0.00 | 0.00 |
| 29 | 1 | 0.00 | 0.00 | 0.00 | 0.00 | 0.00 | 0.00 | 0.00 | 0.00 | 0.00 |

| | | 73 | | | 74 | | | 75 | | |
|---|---|---|---|---|---|---|---|---|---|---|
| | | ?A | | | ?A | | | ?A | | |
| Frequencies -- | | 3157.0533 | | | 3172.8685 | | | 3179.5260 | | |
| Red. masses -- | | 1.1056 | | | 1.1085 | | | 1.1054 | | |
| Frc consts -- | | 6.4928 | | | 6.5750 | | | 6.5838 | | |
| IR Inten -- | | 1.3500 | | | 4.7203 | | | 1.17353 | | |
| Raman Activ -- | | 0.0000 | | | 0.0000 | | | 0.0000 | | |
| Depolar -- | | 0.0000 | | | 0.0000 | | | 0.0000 | | |
| Atom | AN | X | Y | Z | X | Y | Z | X | Y | Z |
| 1 | 8 | 0.00 | 0.00 | 0.00 | 0.00 | 0.00 | 0.00 | 0.00 | 0.00 | 0.00 |
| 2 | 6 | 0.00 | 0.00 | 0.00 | 0.00 | 0.00 | 0.00 | 0.00 | 0.00 | 0.00 |
| 3 | 6 | 0.00 | 0.00 | 0.00 | 0.00 | 0.00 | 0.00 | 0.00 | 0.00 | 0.00 |
| 4 | 6 | 0.00 | 0.00 | 0.00 | 0.00 | 0.00 | 0.00 | 0.00 | 0.00 | 0.00 |
| 5 | 6 | 0.01 | −0.01 | 0.02 | 0.00 | 0.00 | 0.00 | 0.00 | 0.00 | 0.00 |
| 6 | 8 | 0.00 | 0.00 | 0.00 | 0.00 | 0.00 | 0.00 | 0.00 | 0.00 | 0.00 |
| 7 | 8 | 0.00 | 0.00 | 0.00 | 0.00 | 0.00 | 0.00 | 0.00 | 0.00 | 0.00 |
| 8 | 6 | 0.00 | −0.07 | 0.05 | 0.00 | 0.01 | 0.00 | 0.00 | 0.00 | 0.00 |
| 9 | 16 | 0.00 | 0.00 | 0.00 | 0.00 | 0.00 | 0.00 | 0.00 | 0.00 | 0.00 |
| 10 | 6 | −0.01 | 0.00 | 0.00 | −0.08 | −0.04 | −0.04 | −0.05 | 0.07 | 0.04 |
| 11 | 1 | 0.00 | 0.00 | 0.00 | 0.00 | 0.00 | 0.00 | 0.00 | 0.00 | 0.00 |
| 12 | 1 | 0.00 | 0.00 | 0.00 | 0.00 | 0.00 | 0.00 | 0.00 | 0.00 | 0.00 |
| 13 | 1 | −0.02 | 0.02 | −0.02 | 0.00 | 0.00 | 0.01 | 0.00 | 0.00 | 0.00 |
| 14 | 1 | −0.08 | 0.11 | −0.26 | 0.01 | −0.02 | 0.04 | 0.00 | 0.00 | 0.00 |
| 15 | 1 | 0.00 | 0.00 | 0.00 | 0.00 | 0.00 | 0.00 | 0.00 | 0.00 | 0.00 |
| 16 | 1 | 0.00 | 0.00 | 0.00 | 0.00 | 0.00 | 0.00 | 0.00 | 0.00 | 0.00 |
| 17 | 1 | −0.07 | 0.11 | −0.53 | 0.00 | −0.01 | 0.03 | 0.00 | 0.00 | 0.00 |
| 18 | 1 | 0.06 | 0.76 | −0.09 | −0.01 | −0.07 | 0.01 | 0.00 | 0.00 | 0.00 |
| 19 | 1 | 0.06 | 0.00 | 0.04 | 0.62 | 0.05 | 0.43 | 0.10 | 0.02 | 0.08 |
| 20 | 1 | 0.02 | −0.01 | −0.01 | 0.21 | −0.12 | −0.15 | 0.64 | −0.30 | −0.40 |
| 21 | 1 | 0.01 | 0.05 | 0.01 | 0.10 | 0.54 | 0.15 | −0.12 | −0.53 | −0.14 |
| 22 | 15 | 0.00 | 0.00 | 0.00 | 0.00 | 0.00 | 0.00 | 0.00 | 0.00 | 0.00 |
| 23 | 8 | 0.00 | 0.00 | 0.00 | 0.00 | 0.00 | 0.00 | 0.00 | 0.00 | 0.00 |
| 24 | 8 | 0.00 | 0.00 | 0.00 | 0.00 | 0.00 | 0.00 | 0.00 | 0.00 | 0.00 |
| 25 | 8 | 0.00 | 0.00 | 0.00 | 0.00 | 0.00 | 0.00 | 0.00 | 0.00 | 0.00 |
| 26 | 1 | 0.00 | 0.00 | 0.00 | 0.00 | 0.00 | 0.00 | 0.00 | 0.00 | 0.00 |
| 27 | 8 | 0.00 | 0.00 | 0.00 | 0.00 | 0.00 | 0.00 | 0.00 | 0.00 | 0.00 |
| 28 | 1 | 0.00 | 0.00 | 0.00 | 0.00 | 0.00 | 0.00 | 0.00 | 0.00 | 0.00 |
| 29 | 1 | 0.00 | 0.00 | 0.00 | 0.00 | 0.00 | 0.00 | 0.00 | 0.00 | 0.00 |

| | | |
|---|---|---|
| 76 | 77 | 78 |
| ?A | ?A | ?A |

| | | | |
|---|---|---|---|
| Frequencies -- | 3239.2854 | 3464.9953 | 3647.7001 |
| Red. masses -- | 1.0965 | 1.0669 | 1.0652 |
| Frc consts -- | 6.7786 | 7.5471 | 8.3506 |
| IR Inten -- | 2.1181 | 764.4653 | 174.3794 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | 0.00 | 0.00 | 0.00 | 0.00 | 0.00 | 0.00 | 0.00 | 0.00 |
| 2 | 6 | 0.06 | −0.02 | −0.06 | 0.00 | 0.00 | 0.00 | 0.00 | 0.00 | 0.00 |
| 3 | 6 | 0.00 | 0.00 | 0.00 | 0.00 | 0.00 | 0.00 | 0.00 | 0.00 | 0.00 |
| 4 | 6 | 0.00 | 0.00 | 0.00 | 0.00 | 0.00 | 0.00 | 0.00 | 0.00 | 0.00 |
| 5 | 6 | 0.00 | 0.00 | 0.00 | 0.00 | 0.00 | 0.00 | 0.00 | 0.00 | 0.00 |
| 6 | 8 | 0.00 | 0.00 | 0.00 | 0.00 | 0.00 | 0.01 | 0.04 | −0.02 | −0.05 |
| 7 | 8 | 0.00 | 0.00 | 0.00 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 16 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 1 | −0.69 | 0.17 | 0.70 | 0.00 | 0.00 | 0.00 | 0.00 | 0.00 | 0.00 |
| 12 | 1 | 0.00 | −0.02 | 0.01 | 0.00 | 0.00 | 0.01 | 0.00 | 0.00 | 0.01 |
| 13 | 1 | 0.00 | 0.00 | 0.00 | 0.00 | 0.00 | 0.00 | 0.00 | 0.00 | −0.01 |
| 14 | 1 | 0.00 | 0.00 | −0.01 | 0.00 | 0.00 | 0.00 | 0.00 | 0.00 | 0.00 |
| 15 | 1 | 0.00 | 0.00 | 0.00 | 0.05 | −0.02 | −0.05 | −0.61 | 0.27 | 0.74 |
| 16 | 1 | 0.00 | 0.00 | 0.00 | 0.01 | 0.00 | 0.00 | −0.05 | 0.00 | 0.00 |
| 17 | 1 | 0.00 | 0.00 | 0.00 | 0.00 | 0.00 | 0.00 | 0.00 | 0.00 | 0.00 |
| 18 | 1 | 0.00 | 0.00 | 0.00 | 0.00 | 0.00 | 0.00 | 0.00 | 0.00 | 0.00 |
| 19 | 1 | 0.00 | 0.00 | 0.00 | 0.00 | 0.00 | 0.00 | 0.00 | 0.00 | 0.00 |
| 20 | 1 | 0.00 | 0.00 | 0.00 | 0.00 | 0.00 | 0.00 | 0.00 | 0.00 | 0.00 |
| 21 | 1 | 0.00 | 0.00 | 0.00 | 0.00 | 0.00 | 0.00 | 0.00 | 0.00 | 0.00 |
| 22 | 15 | 0.00 | 0.00 | 0.00 | 0.00 | 0.00 | 0.00 | 0.00 | 0.00 | 0.00 |
| 23 | 8 | 0.00 | 0.00 | 0.00 | 0.00 | 0.00 | 0.00 | 0.00 | 0.00 | 0.00 |
| 24 | 8 | 0.00 | 0.00 | 0.00 | 0.00 | 0.00 | 0.00 | 0.00 | 0.00 | 0.00 |
| 25 | 8 | 0.00 | 0.00 | 0.00 | −0.05 | 0.04 | −0.01 | 0.00 | 0.00 | 0.00 |
| 26 | 1 | −0.01 | 0.00 | 0.00 | 0.77 | −0.61 | 0.15 | 0.06 | −0.04 | 0.01 |
| 27 | 8 | 0.00 | 0.00 | 0.00 | 0.00 | 0.00 | 0.00 | 0.00 | 0.00 | 0.00 |
| 28 | 1 | 0.00 | 0.00 | 0.00 | 0.01 | 0.00 | 0.00 | 0.00 | 0.00 | 0.00 |
| 29 | 1 | 0.00 | 0.00 | 0.00 | 0.00 | 0.01 | 0.01 | 0.00 | 0.00 | 0.00 |

| | | |
|---|---|---|
| 79 | 80 | 81 |
| ?A | ?A | ?A |

| | | | |
|---|---|---|---|
| Frequencies -- | 3785.2521 | 3822.7767 | 3833.5359 |
| Red. masses -- | 1.0666 | 1.0664 | 1.0669 |
| Frc consts -- | 9.0040 | 9.1815 | 9.2377 |
| IR Inten -- | 140.1081 | 205.4310 | 204.3383 |
| Raman Activ -- | 0.0000 | 0.0000 | 0.0000 |
| Depolar -- | 0.0000 | 0.0000 | 0.0000 |

| Atom | AN | X | Y | Z | X | Y | Z | X | Y | Z |
|---|---|---|---|---|---|---|---|---|---|---|
| 1 | 8 | 0.00 | 0.00 | 0.00 | 0.00 | 0.00 | 0.00 | 0.00 | 0.00 | 0.00 |
| 2 | 6 | 0.00 | 0.00 | 0.00 | 0.00 | 0.00 | 0.00 | 0.00 | 0.00 | 0.00 |
| 3 | 6 | 0.00 | 0.00 | 0.00 | 0.00 | 0.00 | 0.00 | 0.00 | 0.00 | 0.00 |
| 4 | 6 | 0.00 | 0.00 | 0.00 | 0.00 | 0.00 | 0.00 | 0.00 | 0.00 | 0.00 |
| 5 | 6 | 0.00 | 0.00 | 0.00 | 0.00 | 0.00 | 0.00 | 0.00 | 0.00 | 0.00 |
| 6 | 8 | 0.00 | 0.00 | 0.00 | 0.00 | 0.00 | 0.00 | 0.00 | 0.00 | 0.00 |
| 7 | 8 | −0.06 | 0.01 | 0.01 | 0.00 | 0.00 | 0.00 | 0.00 | 0.00 | 0.00 |
| 8 | 6 | 0.00 | 0.00 | 0.00 | 0.00 | 0.00 | 0.00 | 0.00 | 0.00 | 0.00 |
| 9 | 16 | 0.00 | 0.00 | 0.00 | 0.00 | 0.00 | 0.00 | 0.00 | 0.00 | 0.00 |
| 10 | 6 | 0.00 | 0.00 | 0.00 | 0.00 | 0.00 | 0.00 | 0.00 | 0.00 | 0.00 |
| 11 | 1 | 0.00 | 0.00 | 0.00 | 0.00 | 0.00 | 0.00 | 0.00 | 0.00 | 0.00 |
| 12 | 1 | 0.00 | 0.00 | 0.00 | 0.00 | 0.00 | 0.00 | 0.00 | 0.00 | 0.00 |
| 13 | 1 | 0.00 | 0.00 | −0.01 | 0.00 | 0.00 | 0.00 | 0.00 | 0.00 | 0.00 |
| 14 | 1 | 0.00 | 0.00 | 0.00 | 0.00 | 0.00 | 0.00 | 0.00 | 0.00 | 0.00 |
| 15 | 1 | −0.03 | 0.01 | 0.04 | 0.00 | 0.00 | 0.00 | 0.00 | 0.00 | 0.00 |
| 16 | 1 | 0.97 | −0.12 | −0.19 | 0.01 | 0.00 | 0.00 | −0.01 | 0.00 | 0.00 |
| 17 | 1 | 0.00 | 0.00 | 0.00 | 0.00 | 0.00 | 0.00 | 0.00 | 0.00 | 0.00 |
| 18 | 1 | 0.00 | 0.00 | 0.00 | 0.00 | 0.00 | 0.00 | 0.00 | 0.00 | 0.00 |
| 19 | 1 | 0.00 | 0.00 | 0.00 | 0.00 | 0.00 | 0.00 | 0.00 | 0.00 | 0.00 |
| 20 | 1 | 0.00 | 0.00 | 0.00 | 0.00 | 0.00 | 0.00 | 0.00 | 0.00 | 0.00 |
| 21 | 1 | 0.00 | 0.00 | 0.00 | 0.00 | 0.00 | 0.00 | 0.00 | 0.00 | 0.00 |
| 22 | 15 | 0.00 | 0.00 | 0.00 | 0.00 | 0.00 | 0.00 | 0.00 | 0.00 | 0.00 |
| 23 | 8 | 0.00 | 0.00 | 0.00 | 0.00 | 0.00 | 0.00 | 0.00 | 0.00 | 0.00 |
| 24 | 8 | 0.00 | 0.00 | 0.00 | 0.00 | 0.00 | −0.01 | 0.00 | −0.01 | −0.06 |
| 25 | 8 | 0.00 | 0.00 | 0.00 | 0.00 | 0.00 | 0.00 | 0.00 | 0.00 | 0.00 |
| 26 | 1 | 0.00 | 0.00 | 0.00 | 0.01 | −0.01 | 0.00 | −0.01 | 0.01 | 0.00 |
| 27 | 8 | 0.00 | 0.00 | 0.00 | 0.06 | 0.01 | 0.00 | −0.01 | 0.00 | 0.00 |
| 28 | 1 | 0.01 | 0.00 | 0.00 | −0.97 | −0.16 | 0.04 | 0.16 | 0.03 | −0.01 |
| 29 | 1 | 0.00 | 0.00 | 0.01 | −0.01 | 0.02 | 0.16 | −0.07 | 0.11 | 0.98 |

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method of designing a putative inhibitor of a human 5′-methylthioadenosine phosphorylase (MTAP), the method comprising designing a chemically stable compound that resembles (a) the molecular electrostatic potential at the van der Walls surface computed from the wave function of the transition state of the MTAP and (b) the geometric atomic volume of the MTAP transition state, wherein the compound is the putative inhibitor.

2. The method of claim 1, wherein the compound comprises a purine moiety.

3. The method of claim 1, wherein the compound comprises a deazapurine moiety.

4. The method of claim 1, wherein the compound comprises a moiety resembling the molecular electrostatic potential surface of the ribosyl group at the transition state.

5. The method of claim 4, wherein the compound comprises a moiety resembling methylthioribose at the transition state.

6. The method of claim 4, wherein the compound comprises a moiety resembling S-homocysteinyl ribose at the transition state.

7. The method of claim 4, wherein the moiety resembling the molecular electrostatic potential surface of the ribosyl group at the transition state is a substituted iminoribitol, a substituted hydroxypyrrolidine, a substituted pyridine or a substituted imidazole.

8. The method of claim 7, wherein the substituent is an aryl- or alkyl-substituted thiol group.

9. The method of claim 8, wherein the substituent is a methylthiol group.

10. The method of claim 1, wherein the compound comprises an atomic moiety to provide a compound that mimics the C1′-N9 ribosyl bond distance of a 5′-methylthioadenosine at the transition state.

11. The method of claim 10, wherein the atomic moiety is a methylene, a substituted methylene, an ethyl, or a substituted ethyl bridge.

12. The method of claim 1, wherein the compound exhibits a similarity value Sₑ to the transition state greater than to either substrate.

13. The method of claim 1, further comprising synthesizing the compound and testing the compound for inhibitory activity to 5′-methylthioadenosine phosphorylase.

14. A method of inhibiting a human MTAP, the method comprising identifying a compound that has inhibitory activity to the MTAP by the method of claim 13, then contacting the MTAP with the compound.

15. The method of claim 14, wherein the MTAP is in a human cell.

16. The method of claim 15, wherein the human cell is a cancer cell in a human.

17. The method of claim 16, wherein the human is also treated with an inhibitor of de novo adenosine monophosphate synthesis.

18. The method of claim 17, wherein the inhibitor of de novo adenosine monophosphate synthesis is L-alanosine.

19. The method of claim 17, wherein the inhibitor of de novo adenosine monophosphate synthesis is an anti-folate.
